(19) United States
(12) Patent Application Publication
(10) Pub. No.: US 2026/0272948 A1
(43) Pub. Date: Sep. 17, 2026

(54) METHODS OF TREATING CANCER WITH PI3K/AKT/mTOR PATHWAY INHIBITORS

(71) Applicant: Faeth Therapeutics, Inc., Austin, TX (US)

(72) Inventors: Oliver D.K. MADDOCKS, Glasgow (GB); Petros A. TYRAKIS, Cambridge (GB); Sarah D. CHIRNOMAS, Woodbridge, CT (US)

(21) Appl. No.: 19/565,330

(22) Filed: Mar. 12, 2026

Related U.S. Application Data
(60) Provisional application No. 63/968,628, filed on Jan. 26, 2026.

(30) Foreign Application Priority Data
Mar. 12, 2025 (EP) 25300023.6

(51) Int. Cl. A61K 31/565; A61K 9/00; A61K 31/337; A61K 31/519; A61K 31/5377; A61P 35/00

(52) U.S. Cl. CPC A61K 31/565; A61K 9/0019; A61K 9/0053; A61K 31/337; A61K 31/519; A61K 31/5377; A61P 35/00

(57) ABSTRACT

Disclosed herein are combinations of compounds and/or diet for inhibiting the PI3K/AKT/mTOR molecular pathway for the treatment of cancer. While the PI3K/AKT/mTOR signaling plays a role in cancer, targeting this pathway with single node inhibitors (e.g., molecules targeting single enzymes within the signaling pathway) has shown limited clinical efficacy. Approaches that simultaneously target multiple nodes of the pathway show improved ability to inhibit pathway signaling and inhibit cancer cell proliferation and tumor growth.

### CROSS-REFERENCE TO RELATED APPLICATIONS

This application claims priority to European Application No. 25300023.6, filed Mar. 12, 2025, and U.S. Provisional Application Ser. No. 63/968,628, filed Jan. 26, 2026, each of which is herein incorporated by reference in their entirety for all purposes.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### FIELD OF DISCLOSURE

The present disclosure relates to the use of combinations of compounds and/or diet for inhibiting the PI3K/AKT/mTOR molecular pathway for the treatment of cancer. While the PI3K/AKT/mTOR signaling plays a role in cancer, targeting this pathway with single node inhibitors (e.g., molecules targeting single enzymes within the signaling pathway) has shown limited clinical efficacy. Approaches that simultaneously target multiple nodes of the pathway show improved ability to inhibit pathway signaling and inhibit cancer cell proliferation and tumor growth.

### BACKGROUND OF THE INVENTION

The PI3K/AKT/mTOR signaling pathway represents the major control hub for metabolic signaling in mammalian biology. This multi-node pathway senses nutrient and growth factor levels, and under favorable conditions, signals to activate protein translation to support growth and proliferation, while simultaneously promoting cell cycle progression and inhibiting apoptosis. Consistent with its central role in controlling cellular growth and proliferation, mutations that activate PI3K/AKT/mTOR signaling are often associated with cancer. While approximately 80% of endometrial cancers carry a PI3K/AKT/mTOR pathway mutation, there are no approved PI3K/AKT/mTOR pathway-targeted agents for this disease.

Currently, single node inhibitors (SNIs) of the PI3K/AKT/mTOR pathway, such as alpelisib (targeting PI3Kα), capivasertib (targeting AKT) and everolimus (mTORC1 inhibitor), have received regulatory approval for the treatment of a limited set of cancers. However, these SNIs have modest therapeutic benefit. Moreover, a well-known issue of PI3K/AKT SNIs is the emergence of treatment-induced hyperglycemia. Approaches that utilize multi-node inhibition of the PI3K/AKT/mTOR pathway may have clinical benefits for the treatment of cancer.

### BRIEF SUMMARY OF THE INVENTION

The present disclosure provides methods of treating cancer in a subject in need thereof, comprising administering to the subject in need thereof:
(a) a therapeutically effective amount of one or more compounds that inhibit two or more enzymes of the PI3K/AKT/mTOR pathway, and
(b) a therapeutically effective amount of one or more anti-cancer agents that do not directly inhibit enzymes in the PI3K/AKT/mTOR pathway.

The present disclosure also provides methods of treating cancer in a subject in need thereof, comprising administering to the subject in need thereof:
(a) a therapeutically effective amount of one or more compounds that inhibit two or more enzymes of the PI3K/AKT/mTOR pathway,
(b) a therapeutically effective amount of one or more anti-cancer agents that do not directly inhibit enzymes in the PI3K/AKT/mTOR pathway, and
(c) an insulin-suppressing diet.

The present disclosure also provides methods of treating cancer in a subject in need thereof, comprising administering to the subject in need thereof:
(a) a therapeutically effective amount of a dual mTORC1 and mTORC2 inhibitor,
(b) a therapeutically effective amount of a PI3K-alpha inhibitor, and
(c) a therapeutically effective amount of one or more anti-cancer agents that do not directly inhibit enzymes in the PI3K/AKT/mTOR pathway.

The present disclosure also provides methods of treating cancer in a subject in need thereof, comprising administering to the subject in need thereof:
(a) a therapeutically effective amount of a dual mTORC1 and mTORC2 inhibitor,
(b) a therapeutically effective amount of a PI3K-alpha inhibitor,
(c) a therapeutically effective amount of one or more anti-cancer agents that do not directly inhibit enzymes in the PI3K/AKT/mTOR pathway, and
(d) an insulin-suppressing diet.

Also provided are uses, compounds for use in treatment, and compounds for use in the preparation of medicaments for the treatment of the indications disclosed herein, consistent with the description provided for the methods of treatment described herein.

For example, the present disclosure also provides use of compounds (e.g., one or more compounds that inhibit two or more enzymes of the PI3K/AKT/mTOR pathway (such as a dual mTORC1 and mTORC2 inhibitor, such as sapanisertib) and one or more anti-cancer agents that do not directly inhibit enzymes in the PI3K/AKT/mTOR pathway (e.g., paclitaxel)) in the preparation of medicament(s) for the treatment of cancer in a subject in need thereof.

Also for example, the present disclosure provides use of compounds (e.g., one or more compounds that inhibit two or more enzymes of the PI3K/AKT/mTOR pathway (such as a dual mTORC1 and mTORC2 inhibitor, such as sapanisertib) and one or more anti-cancer agents that do not directly inhibit enzymes in the PI3K/AKT/mTOR (e.g., paclitaxel)) for the treatment of cancer in a subject in need thereof.

Also for example, the present disclosure provides compounds (e.g., one or more compounds that inhibit two or more enzymes of the PI3K/AKT/mTOR pathway (such as a dual mTORC1 and mTORC2 inhibitor, such as sapanisertib) and one or more anti-cancer agents that do not directly inhibit enzymes in the PI3K/AKT/mTOR pathway (e.g., paclitaxel)) for use in the treatment of cancer in a subject in need thereof.

The disclosure also provides a method of treating endometrial cancer in a human subject in need thereof, the method comprising administering to the subject a therapeutically effective amount of a combination comprising: an amount of orally administered sapanisertib, an amount of orally administered serabelisib, and an amount of intravenously administered paclitaxel.

The disclosure also provides a method of treating endometrial cancer in a human subject in need thereof without inducing grade 4 stomatitis or without inducing grade 4 hyperglycemia, the method comprising intravenously administering to the subject 40-100 mg/m² paclitaxel on day 1, day 8, and day 15 of a four-week treatment interval; and orally administering to the subject 100-300 mg serabelisib and 1-4 mg sapanisertib on day 2, day 3, day 4, day 9, day 10, day 11, day 16, day 17, day 18, day 23, day 24 and day 25 of the four-week treatment interval.

The present disclosure also provides a method of treating breast cancer in a human subject in need thereof, comprising administering to the human subject in need thereof an effective amount of: orally administered sapanisertib and orally administered serabelisib in combination with one or more of a hormone receptor targeting therapeutic agent, a cyclin-dependent kinase (CDK) inhibitor, and a lysine acetyltransferase 6 (KAT6) inhibitor.

The present disclosure also provides a method of increasing efficacy of a hormone receptor targeting therapeutic agent in a human subject in need thereof having breast cancer, the method comprising administering to the human subject an effective amount of: orally administered sapanisertib and orally administered serabelisib in combination with the hormone receptor targeting therapeutic agent.

The present disclosure also provides a method of increasing efficacy of a CDK inhibitor in a human subject in need thereof having breast cancer, the method comprising administering to the human subject an effective amount of: orally administered sapanisertib and orally administered serabelisib in combination with the CDK inhibitor.

Any of the features of the methods described herein are also applicable to uses of compounds for treating cancer, uses in the manufacture of a medicament for treating cancer, compounds for use in treating cancer, and kits comprising compounds for treating cancer.

### BRIEF DESCRIPTION OF THE DRAWINGS

FIG. 1A shows an overview of PI3K/AKT/mTOR pathway.

FIG. 1B shows the frequency of PI3K/AKT/mTOR pathway mutations in common human cancers. Data sourced from MSK-IMPACT clinical sequencing cohort, n=10,945 samples via cBioPortal (MSK, Nature Medicine 2017). Amp=amplification, Mut=mutation, Del=homozygous deletion. The 20 tumor types with the highest cumulative mutation frequency are shown. Note that multiple mutations can occur within the same tumor sample, hence cumulative total can exceed 100%.

FIG. 1C shows that a multi-node targeting strategy utilizing sapanisertib and serabelisib achieves improved PI3K/AKT/mTOR pathway inhibition versus single node inhibitors. Shown are Western blots of PI3K/AKT/mTOR pathway output (phosS6-S235/236 and phos4EBP1-T37/46) in endometrial and breast cancer cell lines treated with the indicated inhibitors for 3-4 hours followed by stimulation with 10 ng/ml insulin for 10 minutes. Drug concentrations were selected to represent clinical C-maximum and C-average for each drug and the mid-point of these.

FIG. 1D shows dose-response curves for indicated drugs in the indicated cancer cell lines at 72 hours post-treatment.

FIG. 1E shows IC₅₀ values of sapanisertib compared with serabelisib+sapanisertib in breast and endometrial cancer cell lines; unpaired t-test. Error bars denote standard deviation (SD).

FIG. 2A shows that a multi-node targeting strategy utilizing sapanisertib and serabelisib achieves improved PI3K/AKT/mTOR pathway inhibition versus single node inhibitors. Shown are Western blot of PI3K/AKT/mTOR pathway output (phosS6-S235/236 and phos4EBP1-T37/46) in HEC1B endometrial cancer cell line treated with the indicated inhibitors for 3-4 hours followed by stimulation with 10 ng/ml insulin for 10 minutes.

FIG. 2B shows dose-response curves for indicated drugs in the indicated cancer cell lines at 72 hours post-treatment. Error bars denote SD.

FIG. 2C shows dose response curves for indicated drugs in the indicated cancer cell lines at 72 hours post-treatment. Error bars denote SD.

FIG. 3A shows that the combination of sapanisertib and serabelisib more completely inhibits PI3K/AKT/mTOR signaling than mutant-specific PI3Kα inhibitors. Shown are Western blots of PI3K/AKT/mTOR pathway activity (phosAKT-S473, phosS6-S235/236 and phos4E-BP1-T37/46) in endometrial cancer cell lines, stimulated with 10 ng/ml insulin followed by treatment with the indicated inhibitors for 3-4 hours.

FIG. 3B shows that the combination of sapanisertib and serabelisib more completely inhibits PI3K/AKT/mTOR signaling than mutant-specific PI3Kα inhibitors. Shown are Western blots of PI3K/AKT/mTOR pathway activity (phosAKT-S473, phosS6-S235/236 and phos4E-BP1-T37/46) in breast cancer cell lines, stimulated with 10 ng/ml insulin followed by treatment with the indicated inhibitors for 3-4 hours.

FIG. 3C shows dose-response curves for serabelisib+sapanisertib against indicated mutant-specific PI3Kα inhibitors in the indicated breast and endometrial cancer cell lines at 72 hours post-treatment. Error bars denote SD.

FIG. 3D shows correlation plots of PI3K/AKT/mTOR pathway inhibitors comparing the in vitro potency (IC₅₀) of indicated drugs with the in vitro levels of phos-4E-BP1-T37/46 and phos-S6-S235/236 after 3-4 hours treatment with indicated drugs at the average free-drug plasma concentration (C-average) achieved in humans/in vivo models; data are means for breast and endometrial cancer cell lines used in this study. A diagram depicting the function of 4E-BP1 and phopho-4E-BP1 on eIF4E is also shown.

FIG. 4A shows dose-response curves for standard-of-care agents in breast cancer cell lines for the indicated standard of care drugs at 72 hours post-treatment. Error bars denote SD.

FIG. 5A shows dose-response curves for carboplatin with different concentrations of serabelisib plus sapanisertib at 72 hours post-treatment in endometrial cancer cell lines. Error bars denote SD.

FIG. 5B shows dose-response curves for paclitaxel with different concentrations of serabelisib plus sapanisertib at 72 hours post-treatment in endometrial cancer cell lines. Error bars denote SD.

FIG. 5C shows dose-response curves for selinexor with different concentrations of serabelisib plus sapanisertib at 72 hours post-treatment in endometrial cancer cell lines. Error bars denote SD.

FIG. 5D shows dose-response curves for palbociclib with different concentrations of serabelisib plus sapanisertib at 72 hours post-treatment in endometrial cancer cell lines. Error bars denote SD.

FIG. 6A shows dose-response curves for paclitaxel with different concentrations of serabelisib+sapanisertib in the indicated cancer cell lines at 72 hours post-treatment. Error bars denote SD.

FIG. 6B shows dose-response curves for palbociclib with different concentrations of serabelisib+sapanisertib in the indicated cancer cell lines at 72 hours post-treatment. Error bars denote SD.

FIG. 6C shows dose-response curves for selinexor with different concentrations of serabelisib+sapanisertib in the indicated cancer cell lines at 72 hours post-treatment. Error bars denote SD.

FIG. 6D shows dose-response curves for fulvestrant or elacestrant in ER-positive HER2-negative breast cancer cell lines in the presence of 0.1 nM E2, with different concentrations of serabelisib+sapanisertib in the indicated cancer cell lines at 72 hours post-treatment. Error bars denote SD.

FIG. 7A shows how paclitaxel-resistant cell lines were generated by exposure to paclitaxel for five months. Shown are dose-response curves of paclitaxel parental and paclitaxel-resistant cancer cell lines at 72 hours post-treatment. Error bars denote SD.

FIG. 7B shows dose-response curves of sapanisertib plus serabelisib in parental and paclitaxel-resistant cancer cell lines at 72 hours post-treatment. Error bars denote SD.

FIG. 7C shows western blots of PI3K/AKT/mTOR pathway activity (phosAKT-S473, phosS6-S235/236 and phos4EBP1-T37/46) in AN3CA parental or paclitaxel-resistant cell lines, stimulated with 10 ng/ml insulin followed by treatment with the indicated inhibitors for 3-4 hours.

FIG. 7D shows quantification of the indicated phospho-AKT/S6/4EBP1 levels in vehicle-treated AN3CA parental or paclitaxel-resistant samples.

FIG. 8A shows taxane resistance activates the PI3K/AKT/mTOR pathway. Western blots of PI3K/AKT/mTOR pathway activity (phosAKT-S473, phosS6-S235/236 and phos4EBP1-T37/46) in MFE296 parental or paclitaxel-resistant cell lines, stimulated with 10 ng/ml insulin followed by treatment with the indicated inhibitors for 3-4 hours.

FIG. 8B shows quantification of the phospho-AKT/S6/4EBP1 levels in vehicle-treated MFE296 and MFE296 paclitaxel-resistant samples.

FIG. 8C shows that serabelisib, sapanisertib and paclitaxel combination is highly effective when given in a clinically relevant dosing schedule. Cells were treated with 10 nM paclitaxel or vehicle on day 1. After 24 hours (day 2), media was replaced, followed by the addition of 5.9 μM serabelisib, 30 nM sapanisertib or 5.9 μM serabelisib+30 nM sapanisertib until day 4. Error bars denote SD.

FIG. 9A shows that sapanisertib and serabelisib in combination with paclitaxel strongly suppress in vivo tumor growth. Shown are efficacy of sapanisertib (0.5 mg/kg), serabelisib (75 mg/kg), paclitaxel (15 mg/kg) and their combinations in MDA-MB-361 xenografts. Drugs were initiated when tumor volume reached 300 mm³; n=7-8 mice per group. Error bars denote SEM.

FIG. 9B shows that sapanisertib and serabelisib in combination with paclitaxel strongly suppress in vivo tumor growth. Shown are efficacy of sapanisertib (0.5 mg/kg), serabelisib (75 mg/kg), paclitaxel (15 mg/kg) and their combinations in AN3CA xenografts. Drugs were initiated when tumor volume reached 300 mm³; n=8 mice per group. Error bars denote SEM.

FIG. 9C shows that sapanisertib and serabelisib in combination with paclitaxel strongly suppress in vivo tumor growth, which is further enhanced by an insulin-suppressing diet (ISD). Shown are efficacy of sapanisertib (0.5 mg/kg), serabelisib (75 mg/kg), paclitaxel (3.5 mg/kg) and their combinations, with and without an insulin-suppressing diet (ISD) in MDA-MB-361 xenografts. Drugs were initiated when tumor volume reached 250 mm³, n=5-8 mice per group. Error bars denote SEM.

FIG. 9D shows that sapanisertib and serabelisib in combination with paclitaxel strongly suppress in vivo tumor growth, which is further enhanced by an insulin-suppressing diet (ISD). Efficacy of sapanisertib (0.5 mg/kg), serabelisib (75 mg/kg), paclitaxel (10 mg/kg) and their combinations, with and without ISD in AN3CA xenografts. Drugs were initiated when tumor volume reached 250 mm³, n=6-8 mice per group. Error bars denote SEM.

FIG. 9E shows AN3CA and MDA-MB-361 tumors randomly harvested from chow and ISD groups treated with sapanisertib, sapanisertib and paclitaxel at study endpoint; n=6 per group. Unpaired t-test with Welch's correction. Error bars denote SEM.

FIG. 9F shows representative Western blots of PI3K/AKT/mTOR pathway activity (phosAKT-S473, phosS6-S235/236 and phos4EBP1-T37/46) in AN3CA and MDA-MB-361 tumors from indicated groups, 1 hour post drug dosing; n=3 tumors per group were randomly harvested and processed for Western blot.

FIG. 10A shows body weight of MDA-MB-361 tumor-bearing mice treated with indicated drugs and doses, n=5-8 mice per group. Error bars denote SEM.

FIG. 10B shows body weight of AN3CA tumor-bearing mice treated with indicated drugs and doses, n=5-8 mice per group. Error bars denote SEM.

FIG. 10C shows body weight of MDA-MB-361 tumor-bearing mice on chow or ISD diets, treated with indicated drugs and doses, n=5-8 mice per group. Error bars denote SEM.

FIG. 10D shows body weight of AN3CA tumor-bearing mice on chow or ISD diets, treated with indicated drugs and doses, n=5-8 mice per group. Error bars denote SEM.

FIG. 10E shows a diagram outlining rational combination of multi-node inhibition in combination with an insulin-suppressing diet (ISD).

FIG. 11 shows a dose escalation, trial schema for Cohort A. EoT=end of treatment. Treatment is as follows: Sapanisertib and serabelisib taken orally QD on Days 1-3, 8-10, 15-17, and 22-24 of a 28-day cycle with food; fulvestrant 500 mg IM injection on Days 1 and 15 of Cycle 1, and then once monthly.

FIG. 12 shows a dose escalation, trial design schema for Cohort A. EoT=end of treatment. Treatment is as follows: Sapanisertib and serabelisib taken orally QD on Days 1-3, 8-10, 15-17, and 22-24 of a 28-day cycle with food; fulvestrant 500 mg IM injection on Days 1 and 15 of Cycle 1, and then once monthly; palbociclib 125 mg QD for 21 days on and seven days off in a 28-day cycle.

### DETAILED DESCRIPTION OF THE INVENTION

The following description sets forth numerous exemplary compounds, methods, uses, and the like. It should be recognized, however, that such description is not intended as a limitation on the scope of the present disclosure, but is instead provided as a description of exemplary embodiments.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein, the terms “including,” “containing,” and “comprising” are used in their open, non-limiting sense.

The articles “a” and “an,” as used herein, refer to one or more than one (i.e., to at least one) of the grammatical object of the article. By way of example, “an element” refers to one element or more than one element.

Unless otherwise indicated, the word “or” in the specification and claims is considered to be the inclusive “or” rather than the exclusive or, and indicates at least one of and any combination of items it conjoins.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

The efficacy of single node inhibitors is limited by several factors which promote persistent or reactivated PI3K/AKT/mTOR signaling, including (i) pathway reactivation by feedback activation or bypass signaling, (ii) the presence of co-occurring pathway mutations beyond the targeted node, and (iii) systemic perturbations in glucose homeostasis that lead to hyperinsulinemia and pathway reactivation. Disclosed herein are multi-node inhibitor (MNI) approaches to blocking PI3K/AKT/mTOR signaling may overcome these issues.

The disclosure provides methods of treating cancer in a subject in need thereof, comprising administering to the subject:
(a) a therapeutically effective amount of one or more compounds that inhibit two or more enzymes of the PI3K/AKT/mTOR pathway, and
(b) a therapeutically effective amount of one or more anti-cancer agents that do not directly inhibit enzymes in the PI3K/AKT/mTOR pathway.

Also provided are uses, compounds for use in treatment, and compounds for use in the preparation of medicaments for the treatment of the indications disclosed herein, consistent with the description provided for the methods of treatment described herein.

### Inhibitors of the PI3K/AKT/mTOR Pathway

The phrase “enzymes of the PI3K/AKT/mTOR pathway,” as used herein, refers to the kinases and regulatory proteins involved in the phosphoinositide 3-kinase (PI3K)/protein kinase B (AKT)/mechanistic target of rapamycin (mTOR) signaling cascade, which regulates cell growth, proliferation, survival, metabolism, and apoptosis. This includes, but is not limited to, phosphoinositide 3-kinases (PI3Ks), PI3K isoforms (e.g., PI3Kα, PI3Kβ, PI3Kγ, and PI3Kδ), AKT isoforms (AKT1, AKT2, and AKT3), and mTOR complexes (mTORC1 and mTORC2), as well as associated upstream and downstream effectors such as S6K, 4E-BP1, MTOR, PIK3R1, PTEN, TSC1, TSC2, AKT1, AKT2, AKT3, PPP2R1A, PIK3C2B, INPP4B, PDK1, RHEB, STK11, RPTOR, DEPTOR, RAPTOR, RICTOR, MLST8, AKT1S1, DEPDC5, NPRL2, NPRL3, AMPK and GSK3. The term encompasses wild-type and mutant forms of these enzymes, as well as their isoforms and post-translationally modified variants. In some embodiments, the enzyme of the PI3K/AKT/mTOR pathway is a wild-type enzyme. In some embodiments, the enzyme of the PI3K/AKT/mTOR pathway is a mutant enzyme.

The term “inhibit,” as used herein, refers to reducing, suppressing, delaying, or otherwise limiting the activity, function, expression, or progression of a biological process, disease, disorder, or condition. A compound, molecule, or agent may directly or indirectly inhibit the activity, function, or signaling of an enzyme. Inhibition may be partial or complete and may occur at the molecular, cellular, tissue, or organismal level. The term encompasses direct and indirect effects, including interference with biochemical pathways, modulation of gene expression, receptor blocking, enzyme suppression, or disruption of cellular signaling. Unless otherwise specified, “inhibit” does not require absolute prevention but includes any measurable decrease in the targeted activity or condition.

In some embodiments, the ability to inhibit is expressed by IC₅₀. As used herein, the term “IC₅₀” refers to the half-maximal inhibitory concentration of a compound, molecule, or agent, which is the concentration required to inhibit a specific biological or biochemical activity by 50% relative to an untreated control under defined experimental conditions. IC₅₀ values are commonly used to quantify the potency of inhibitors, including but not limited to enzymatic inhibitors, receptor antagonists, and signaling pathway modulators. The IC₅₀ may be determined using in vitro, ex vivo, or in vivo assays, including biochemical enzyme activity assays, cell-based functional assays, or other quantitative methods that measure the reduction of a target activity. Cell-based assays often yield higher IC₅₀ values compared to biochemical assays due to factors like cell permeability, metabolism, and efflux. Unless otherwise specified, IC₅₀ values may be expressed in molar concentrations (e.g., nanomolar or micromolar) and may be determined using dose-response curves, statistical regression models, or other analytical techniques to evaluate the efficacy of an inhibitory agent in a biological system.

The IC₅₀ may be used to express the potency of a compound, molecule, or agent against a protein or enzyme. However, there is no absolute universal cutoff to define an inhibitor, as it depends on the biological context and the assay conditions. That said, general guidelines include: Strong Inhibitors: IC₅₀≤100 nM; Moderate Inhibitors: IC₅₀ between 100 nM and 1 μM; Weak Inhibitors: IC₅₀ between 1 μM and 10 μM; Very Weak or Non-Inhibitors: IC₅₀>10 μM. Some regulatory agencies (e.g., FDA for drug-drug interactions) define strong enzyme inhibitors as having IC₅₀ values <1 μM. In some embodiments, compounds herein have an IC₅₀ (e.g., as measured by an in vitro or cell-based assay) for the target protein that is <1 μM (e.g., between about 1 nM and 1 μM.) For a molecule to be considered a practical drug candidate, an IC₅₀ in the low nanomolar (nM) range is usually preferred. In some embodiments, compounds herein have an IC₅₀ (e.g., as measured by an in vitro or cell-based assay) for the target protein that is <500 nM (e.g., between about 1 nM to about 500 nM.) In some embodiments, compounds herein have an IC₅₀ (e.g., as measured by an in vitro or cell-based assay) for the target protein that is <100 nM (e.g., between about 1 nM to about 100 nM.)

The term “selective inhibitor,” as used herein, refers to a compound, molecule, or agent that preferentially inhibits the activity, function, or expression of a specific target, such as an enzyme, receptor, signaling pathway, or biomolecule, while exerting minimal or no effect on non-targeted molecules or pathways. Selectivity may be based on binding affinity, structural specificity, differential expression in tissues or cell types, or preferential biochemical interactions. A selective inhibitor may exhibit partial or complete inhibition of its target while maintaining reduced off-target effects, thereby enhancing therapeutic efficacy and minimizing undesired toxicity or side effects. For example, a selective mTORC1 inhibitor refers to a compound, molecule, or agent that preferentially inhibits the activity, function, or expression of mTORC1 while exerting minimal or no effect on molecules or pathways other than mTORC1.

The phrase “one or more compounds that inhibit two or more enzymes of the PI3K/AKT/mTOR pathway,” as used herein, refers to a single compound or a combination of compounds capable of selectively or non-selectively inhibiting the activity, function, or expression of at least two distinct enzymes within the PI3K/AKT/mTOR signaling cascade. Such compounds may act by directly binding to and inhibiting enzymatic activity, modulating upstream or downstream regulatory mechanisms, or disrupting protein-protein interactions within the pathway. The inhibition may be competitive, non-competitive, allosteric, covalent, or reversible, and may target enzymes including, but is not limited to, phosphoinositide 3-kinases (PI3Ks), PI3K isoforms (e.g., PI3Kα, PI3Kβ, PI3Kγ, and PI3Kδ), AKT isoforms (AKT1, AKT2, and AKT3), and mTOR complexes (mTORC1 and mTORC2), as well as associated upstream and downstream effectors such as S6K, 4E-BP1, MTOR, PIK3R1, PTEN, TSC1, TSC2, AKT1, AKT2, AKT3, PPP2R1A, PIK3C2B, INPP4B, PDK1, RHEB, STK11, RPTOR, DEPTOR, RAPTOR, RICTOR, MLST8, AKT1S1, DEPDC5, NPRL2, NPRL3, AMPK and GSK3. The term encompasses small molecules, biologics, peptides, nucleic acid-based agents, or any other therapeutic modality that achieves inhibition of two or more enzymes within the pathway.

In some embodiments, one or more compounds that inhibit two or more enzymes of the PI3K/AKT/mTOR pathway comprise an mTOR inhibitor. The term “mTOR inhibitor,” as used herein, refers to a compound, molecule, or agent that directly or indirectly inhibits the activity, function, or expression of the mechanistic target of rapamycin (mTOR), a serine/threonine kinase involved in regulating cell growth, proliferation, metabolism, and survival. mTOR inhibitors may target either or both of the mTOR-containing protein complexes, mTORC1 and mTORC2, through competitive, allosteric, covalent, or indirect mechanisms. The term encompasses rapamycin and its analogs (rapalogs), ATP-competitive mTOR kinase inhibitors, dual PI3K/mTOR inhibitors, and any other pharmacological agents, biologics, or nucleic acid-based therapies that modulate mTOR signaling for therapeutic purposes.

In some embodiments, the mTOR inhibitor is temsirolimus. Some embodiments comprise administering about 10 mg/week to about 50 mg/week of the temsirolimus. Some embodiments comprise administering about 25 mg/week of the temsirolimus.

Some embodiments comprise administering about 10 mg/week to about 50 mg/week, about 10 mg/week to about 40 mg/week, about 10 mg/week to about 30 mg/week, about 10 mg/week to about 20 mg/week, about 20 mg/week to about 50 mg/week, about 20 mg/week to about 40 mg/week, about 20 mg/week to about 30 mg/week, about 30 mg/week to about 50 mg/week, or about 30 mg/week to about 40 mg/week of the temsirolimus. Some embodiments comprise administering about 20 mg/week to about 30 mg/week of the temsirolimus. Some embodiments comprise administering about 10 mg/week, about 15 mg/week, about 20 mg/week, about 25 mg/week, about 30 mg/week, about 35 mg/week, about 40 mg/week, about 45 mg/week, or about 50 mg/week of the temsirolimus.

Temsirolimus refers to a compound of the following structure. Some embodiments are directed to a salt or enantiomer of temsirolimus.

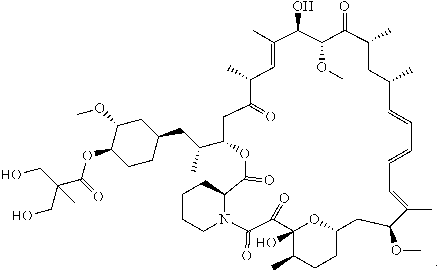 [SMILES: CO[C@H]1C[C@@H]2CC[C@@H](C)[C@@](O)(O2)C(=O)C(=O)N2CCCC[C@H]2C(=O)O[C@H]([C@H](C)C[C@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the mTOR inhibitor is an mTORC1 inhibitor. The term “mTORC1 inhibitor,” as used herein, refers to a compound, molecule, or agent that inhibits the activity, function, or signaling of the mechanistic target of rapamycin complex 1 (mTORC1), a protein kinase complex that regulates cell growth, metabolism, and protein synthesis in response to nutrients, growth factors, and energy status. mTORC1 inhibitors may act through direct binding to mTOR kinase, allosteric modulation, or disruption of regulatory interactions within the complex. The term includes, but is not limited to, rapamycin and its analogs (rapalogs), ATP-competitive mTOR kinase inhibitors with mTORC1 selectivity, and other pharmacological agents or biologics that inhibit mTORC1-mediated phosphorylation of downstream effectors such as S6K1 and 4E-BP 1, with or without affecting mTORC2 activity. In some embodiments, the mTORC1 inhibitor is not a selective mTORC1 inhibitor (e.g., in some embodiments the compound acts on mTORC1 and also has activity for another enzyme, receptor, signaling pathway, or biomolecule other than mTORC1.) In some embodiments, the mTORC1 inhibitor is a selective mTORC1 inhibitor.

In some embodiments, the mTORC1 inhibitor is everolimus. Some embodiments comprise administering about 2 mg/day to about 10 mg/day of the everolimus. Some embodiments comprise administering about 5 mg/day of the everolimus.

Some embodiments comprise administering about 2 mg/day to about 10 mg/day, about 2 mg/day to about 8 mg/day, about 2 mg/day to about 6 mg/day, about 2 mg/day to about 4 mg/day, about 4 mg/day to about 10 mg/day, about 4 mg/day to about 8 mg/day, about 4 mg/day to about 6 mg/day, about 6 mg/day to about 10 mg/day, or about 6 mg/day to about 8 mg/day of the everolimus. Some embodiments comprise administering about 4 mg/day to about 6 mg/day of the everolimus. Some embodiments comprise administering about 2 mg/day, about 3 mg/day, about 4 mg/day, about 5 mg/day, about 6 mg/day, about 7 mg/day, about 8 mg/day, about 9 mg/day, or about 10 mg/day of the everolimus.

Everolimus refers to a compound of the following structure. Some embodiments are directed to a salt or enantiomer of everolimus.

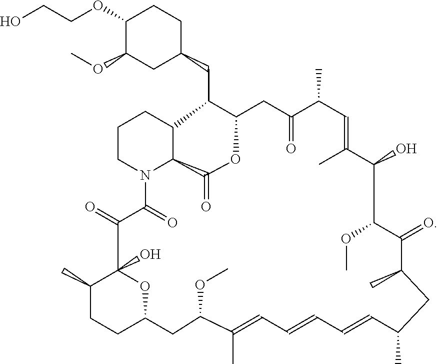 [SMILES: CO[C@H]1C[C@@H]2CC[C@@H](C)[C@@](O)(O2)C(=O)C(=O)N2CCCC3[C@H]2C(=O)O[C@@H](CC(=O)[C@H](C)/C=C(\C)[C@@H](O)[C@@H](OC)C(=O)[C@H](C)C[C@H](C)/C=C/C=C/C=C/1C)[C@H]3C[C@@H]1CC[C@@H](OCCO)[C@H](OC)C1]

In some embodiments, the mTOR inhibitor is an mTORC2 inhibitor. The term “mTORC2 inhibitor,” as used herein, refers to a compound, molecule, or agent that inhibits the activity, function, or signaling of the mechanistic target of rapamycin complex 2 (mTORC2), a protein kinase complex involved in the regulation of cell survival, cytoskeletal organization, and AKT activation. mTORC2 inhibitors may act through direct binding to mTOR kinase, allosteric modulation, or disruption of complex assembly and regulatory interactions. The term includes, but is not limited to, ATP-competitive mTOR kinase inhibitors with mTORC2 selectivity, dual mTORC1/mTORC2 inhibitors, and other pharmacological agents or biologics that inhibit mTORC2-mediated phosphorylation of downstream effectors such as AKT (Ser473), SGK1, and PKC, with or without affecting mTORC1 activity. In some embodiments, the mTORC2 inhibitor is not a selective mTORC2 inhibitor (i.e., in some embodiments the compound acts on mTORC1 and also has activity for another enzyme, receptor, signaling pathway, or biomolecule other than mTORC1.) In some embodiments, the mTORC2 inhibitor is a selective mTORC2 inhibitor.

In some embodiments, the mTOR inhibitor is a dual mTORC1 and mTORC2 inhibitor. The term “dual mTORC1 and mTORC2 inhibitor,” as used herein, refers to a compound, molecule, or agent that inhibits the activity, function, or signaling of both the mechanistic target of rapamycin complex 1 (mTORC1) and mechanistic target of rapamycin complex 2 (mTORC2). Such inhibitors may act through direct binding to the mTOR kinase domain, allosteric modulation, or disruption of protein-protein interactions within the complexes. The term includes, but is not limited to, ATP-competitive mTOR kinase inhibitors, dual PI3K/mTOR inhibitors, and other pharmacological agents, biologics, or nucleic acid-based therapies that concurrently suppress mTORC1-mediated phosphorylation of downstream effectors such as S6K1 and 4E-BP1, as well as mTORC2-mediated phosphorylation of AKT (Ser473), SGK1, and PKC. Unless otherwise specified, dual mTORC1 and mTORC2 inhibitors may exhibit varying degrees of selectivity or potency for each complex. In some embodiments, the dual mTORC1 and mTORC2 inhibitor is not a selective dual mTORC1 and mTORC2 inhibitor (e.g., in some embodiments the compound acts on mTORC1 and mTORC2, and also has activity for another enzyme, receptor, signaling pathway, or biomolecule other than mTORC1.) In some embodiments, the dual mTORC1 and mTORC2 inhibitor is a selective dual mTORC1 and mTORC2 inhibitor.

In some embodiments, the dual mTORC1 and mTORC2 inhibitor is sapanisertib, vistusertib, onatasertib, XP-105 (also known as BI 860585), AVTX-006, or a combination thereof.

Sapanisertib refers to a compound of the following structure. Some embodiments are directed to a salt or enantiomer of sapanisertib.

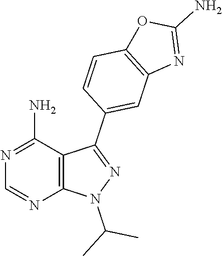 [SMILES: CC(C)n1nc(-c2ccc3oc(N)nc3c2)c2c(N)ncnc21]

Vistusertib refers to a compound of the following structure. Some embodiments are directed to a salt or enantiomer of vistusertib.

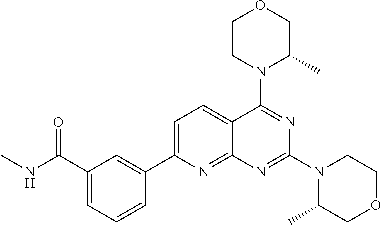 [SMILES: CNC(=O)c1cccc(-c2ccc3c(N4CCOC[C@@H]4C)nc(N4CCOC[C@@H]4C)nc3n2)c1]

Onatasertib refers to a compound of the following structure. Some embodiments are directed to a salt or enantiomer of onatasertib.

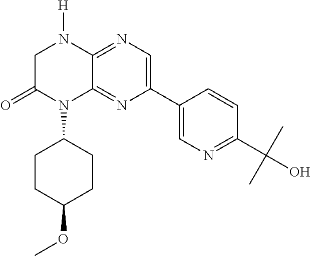 [SMILES: CO[C@H]1CC[C@H](N2C(=O)CNc3ncc(-c4ccc(C(C)(C)O)nc4)nc32)CC1]

AVTX-006, also known as CERC-006, AEVI006, and OSI-027, refers to a compound of the following structure. Some embodiments are directed to a salt or enantiomer of onatasertib.

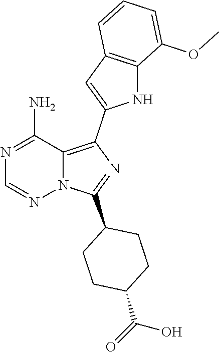 [SMILES: COc1cccc2cc(-c3nc([C@H]4CC[C@H](C(=O)O)CC4)n4ncnc(N)c34)[nH]c12]

In some embodiments, the dual mTORC1 and mTORC2 inhibitor is sapanisertib. In some embodiments, the sapanisertib is administered at a dose of about 1 mg/day to about 6 mg/day. In some embodiments, the sapanisertib is administered at a dose of about 3 mg/day.

Some embodiments comprise administering about 1 mg/day to about 6 mg/day, about 1 mg/day to about 5 mg/day, about 1 mg/day to about 4 mg/day, about 1 mg/day to about 3 mg/day, about 2 mg/day to about 6 mg/day, about 2 mg/day to about 5 mg/day, about 2 mg/day to about 4 mg/day, about 3 mg/day to about 6 mg/day, or about 3 mg/day to about 5 mg/day of the sapanisertib. Some embodiments comprise administering about 1 mg/day, about 2 mg/day, about 3 mg/day, about 4 mg/day, about 5 mg/day, or about 6 mg/day of the sapanisertib. Some embodiments comprise administering about 1 mg/day of the sapanisertib. Some embodiments comprise administering about 2 mg/day of the sapanisertib. Some embodiments comprise administering about 3 mg/day of the sapanisertib. Some embodiments comprise administering about 4 mg/day of the sapanisertib. Some embodiments comprise administering about 5 mg/day of the sapanisertib. Some embodiments comprise administering about 6 mg/day of the sapanisertib.

In some embodiments, the sapanisertib is administered for two days per week. In some embodiments, the sapanisertib is administered for three days per week. In some embodiments, the sapanisertib is administered for four days per week. In some embodiments, the sapanisertib is administered for two consecutive days per week. In some embodiments, the sapanisertib is administered for three consecutive days per week. In some embodiments, the sapanisertib is administered for four consecutive days per week.

In some embodiments, the sapanisertib is administered at a dose of about 1 mg/day to about 3 mg/day for three consecutive days, followed the next day by four consecutive days without administering the sapanisertib.

In some embodiments, the one or more compounds that inhibit two or more enzymes of the PI3K/AKT/mTOR pathway comprise an AKT inhibitor. The term “AKT inhibitor,” as used herein, refers to a compound, molecule, or agent that directly or indirectly inhibits the activity, function, or signaling of protein kinase B (AKT), a serine/threonine kinase involved in cell survival, proliferation, metabolism, and apoptosis. AKT inhibitors may target one or more AKT isoforms (AKT1, AKT2, and AKT3) through competitive ATP binding, allosteric modulation, covalent interaction, or interference with upstream or downstream regulatory mechanisms. The term includes, but is not limited to, ATP-competitive AKT kinase inhibitors, allosteric AKT inhibitors, lipid-based inhibitors that disrupt AKT membrane localization, and other pharmacological agents, biologics, or nucleic acid-based therapies that reduce AKT activation, phosphorylation, or signaling for therapeutic applications. In some embodiments, the AKT inhibitor is not a selective AKT inhibitor (e.g., in some embodiments a compound acts on AKT and also has activity for another enzyme, receptor, signaling pathway, or biomolecule other than AKT.) In some embodiments, the AKT inhibitor is a selective AKT inhibitor.

In some embodiments, the AKT inhibitor is capivasertib, ipatasertib, MK-2206, triciribine, ABTL0812, or combinations thereof.

Capivasertib refers to a compound of the following structure. Some embodiments are directed to a salt or enantiomer of capivasertib.

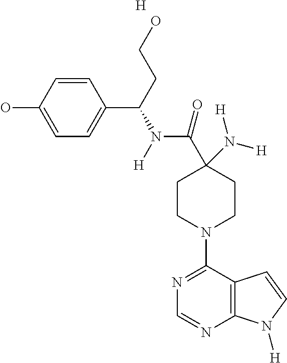

Ipatasertib refers to a compound of the following structure. Some embodiments are directed to a salt or enantiomer of ipatasertib.

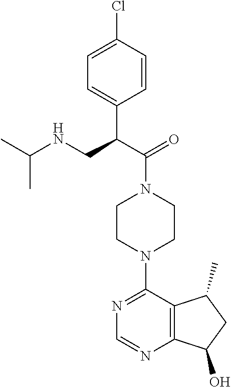 [SMILES: CC(C)NC[C@H](C(=O)N1CCN(c2ncnc3c2[C@H](C)C[C@H]3O)CC1)c1ccc(Cl)cc1]

MK-2206 refers to a compound of the following structure. Some embodiments are directed to a salt or enantiomer of MK-2206.

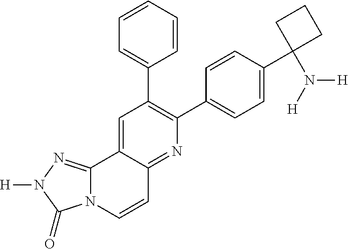 [SMILES: NC1(c2ccc(-c3nc4ccn5c(=O)[nH]nc5c4cc3-c3ccccc3)cc2)CCC1]

Triciribine refers to a compound of the following structure. Some embodiments are directed to a salt or enantiomer of triciribine.

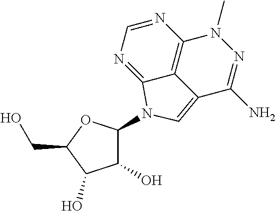 [SMILES: CN1N=C(N)c2cn([C@@H]3O[C@H](CO)[C@@H](O)[C@H]3O)c3ncnc1c23]

Ibrilatazar, also known as ABTL0812, refers to a compound of the following structure. Some embodiments are directed to a salt or enantiomer of ibrilatazar.

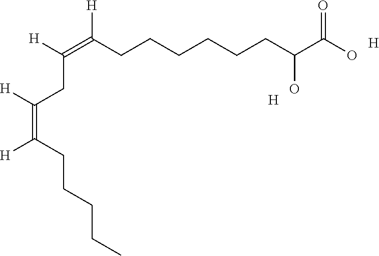

In some embodiments, the one or more compounds that inhibit two or more enzymes of the PI3K/AKT/mTOR pathway comprise a PI3K inhibitor. The term “PI3K inhibitor,” as used herein, refers to a compound, molecule, or agent that directly or indirectly inhibits the activity, function, or signaling of phosphoinositide 3-kinase (PI3K), a lipid kinase involved in cell growth, proliferation, metabolism, and survival. PI3K inhibitors may selectively target one or more PI3K isoforms (PI3Kα, PI3Kβ, PI3Kγ, and PI3Kδ) or act as pan-PI3K inhibitors affecting multiple isoforms. Such inhibitors may function through competitive ATP binding, allosteric modulation, covalent interaction, or disruption of upstream or downstream regulatory mechanisms. The term includes, but is not limited to, isoform-specific inhibitors, pan-PI3K inhibitors, dual PI3K/mTOR inhibitors, and other pharmacological agents, biologics, or nucleic acid-based therapies that suppress PI3K-mediated phosphorylation of downstream effectors such as AKT and mTOR for therapeutic applications. In some embodiments, the PI3K inhibitor is not a selective PI3K inhibitor (e.g., in some embodiments a compound acts on PI3K and also has activity for another enzyme, receptor, signaling pathway, or biomolecule other than PI3K.) In some embodiments, the PI3K inhibitor is a selective PI3K inhibitor.

In some embodiments, the one or more compounds that inhibit two or more enzymes of the PI3K/AKT/mTOR pathway comprise a Pan-PI3K-Pan-mTOR inhibitor. The term “Pan-PI3K-Pan-mTOR inhibitor,” as used herein, refers to a compound, molecule, or agent that inhibits the activity, function, or signaling of all Class I phosphoinositide 3-kinase (PI3K) isoforms (PI3Kα, PI3Kβ, PI3Kγ, and PI3Kδ) as well as both mechanistic target of rapamycin complexes (mTORC1 and mTORC2). Such inhibitors may act through competitive ATP binding, allosteric modulation, covalent interaction, or disruption of regulatory interactions within the PI3K/AKT/mTOR pathway. The term encompasses pharmacological agents, biologics, or nucleic acid-based therapies that broadly suppress PI3K- and mTOR-mediated phosphorylation of downstream effectors, including AKT, S6K1, 4E-BP1, and SGK1. Unless otherwise specified, pan-PI3K-pan-mTOR inhibitors may exhibit varying degrees of potency across isoforms and complexes and may be used for therapeutic applications targeting hyperactivated PI3K/mTOR signaling in cancer and other diseases.

In some embodiments, Pan-PI3K-Pan-mTOR inhibitor is apitolisib, bimiralisib, dactolisib, gedatolisib, omipalisib, PQR309, SF1126, voxtalisib, paxalisib, or combinations thereof.

PQR309 refers to a compound of the following structure. Some embodiments are directed to a salt or enantiomer of PQR309.

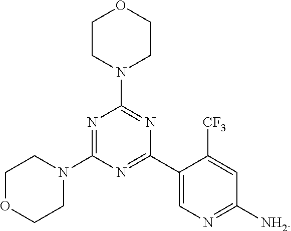 [SMILES: Nc1cc(C(F)(F)F)c(-c2nc(N3CCOCC3)nc(N3CCOCC3)n2)cn1]

SF1126 refers to a compound of the following structure. Some embodiments are directed to a salt or enantiomer of SF1126.

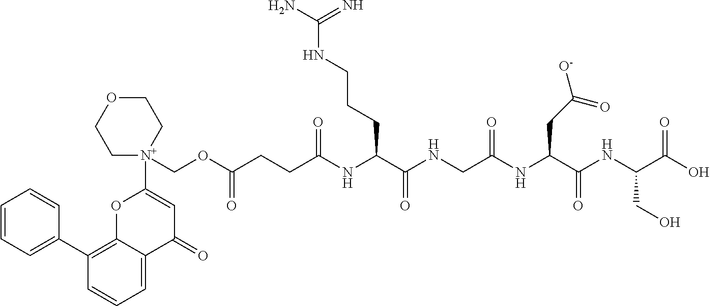 [SMILES: N=C(N)NCCC[C@H](NC(=O)CCC(=O)OC[N+]1(c2cc(=O)c3cccc(-c4ccccc4)c3o2)CCOCC1)C(=O)NCC(=O)N[C@@H](CC(=O)[O-])C(=O)N[C@@H](CO)C(=O)O]

In some embodiments, the PI3K inhibitor is a PI3K-alpha inhibitor. The term “PI3K-alpha inhibitor,” as used herein, refers to a compound, molecule, or agent that inhibits the activity, function, or signaling of phosphoinositide 3-kinase alpha (PI3Kα), a Class I PI3K isoform encoded by the PIK3CA gene and involved in cell growth, proliferation, metabolism, and survival. PI3Kα inhibitors may act through competitive ATP binding, allosteric modulation, covalent interaction, or disruption of regulatory interactions within the PI3K/AKT/mTOR signaling pathway. The term includes, but is not limited to, isoform-specific PI3Kα inhibitors, dual PI3Kα/mTOR inhibitors, and other pharmacological agents, biologics, or nucleic acid-based therapies that selectively reduce PI3Kα activation or downstream signaling for therapeutic applications. Unless otherwise specified, PI3Kα inhibitors may be irreversible or reversible and may exhibit varying degrees of selectivity over other PI3K isoforms. In some embodiments, the PI3K-alpha inhibitor is not a selective PI3K-alpha inhibitor (e.g., in some embodiments a compound acts on PI3K-alpha and also has activity for another enzyme, receptor, signaling pathway, biomolecule, or PI3K isoform other than PI3K-alpha.) In some embodiments, the PI3K-alpha inhibitor is a selective PI3K-alpha inhibitor.

In some embodiments, the PI3K-alpha inhibitor is alpelisib, buparlisib, copanlisib, duvelisib, idelalisib, inavolisib, linperlisib, leniolisib, pictilisib, samotolisib, serabelisib, taselisib, ZSTK474, or a combination thereof. In some embodiments, the PI3K-alpha inhibitor is alpelisib, inavolisib, serabelisib, or a combination thereof.

Pictilisib refers to a compound of the following structure. Some embodiments are directed to a salt or enantiomer of pictilisib.

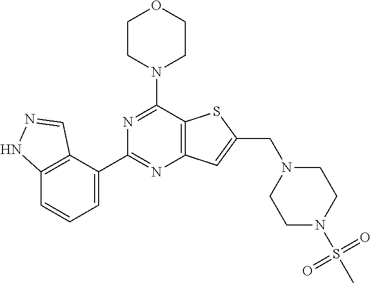 [SMILES: CS(=O)(=O)N1CCN(Cc2cc3nc(-c4cccc5[nH]ncc45)nc(N4CCOCC4)c3s2)CC1]

Samotolisib refers to a compound of the following structure. Some embodiments are directed to a salt or enantiomer of samotolisib.

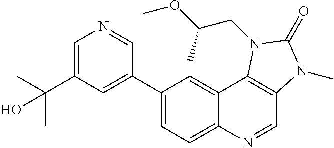 [SMILES: CO[C@@H](C)Cn1c(=O)n(C)c2cnc3ccc(-c4cncc(C(C)(C)O)c4)cc3c21]

Taselisib refers to a compound of the following structure. Some embodiments are directed to a salt or enantiomer of taselisib.

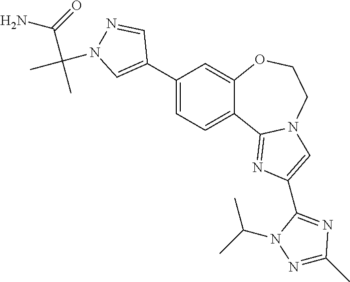 [SMILES: Cc1nc(-c2cn3c(n2)-c2ccc(-c4cnn(C(C)(C)C(N)=O)c4)cc2OCC3)n(C(C)C)n1]

Serabelisib refers to a compound of the following structure. Some embodiments are directed to a salt or enantiomer of serabelisib.

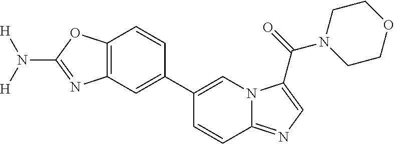 [SMILES: Nc1nc2cc(-c3ccc4ncc(C(=O)N5CCOCC5)n4c3)ccc2o1]

Idelalisib refers to a compound of the following structure. Some embodiments are directed to a salt or enantiomer of idelalisib.

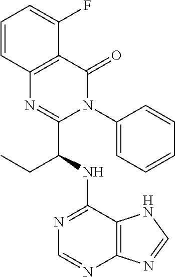 [SMILES: CC[C@H](Nc1ncnc2nc[nH]c12)c1nc2cccc(F)c2c(=O)n1-c1ccccc1]

Linperlisib refers to a compound of the following structure. Some embodiments are directed to a salt or enantiomer of linperlisib.

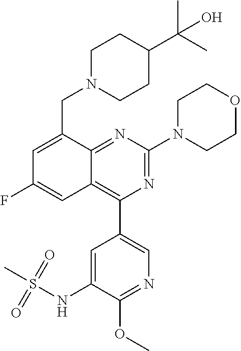 [SMILES: COc1ncc(-c2nc(N3CCOCC3)nc3c(CN4CCC(C(C)(C)O)CC4)cc(F)cc23)cc1NS(C)(=O)=O]

Inavolisib refers to a compound of the following structure. Some embodiments are directed to a salt or enantiomer of inavolisib.

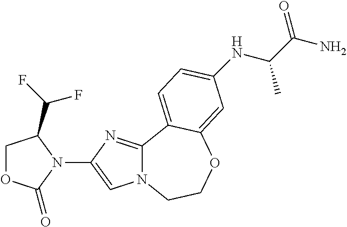 [SMILES: C[C@H](Nc1ccc2c(c1)OCCn1cc(N3C(=O)OC[C@H]3C(F)F)nc1-2)C(N)=O]

Leniolisib refers to a compound of the following structure. Some embodiments are directed to a salt or enantiomer of leniolisib.

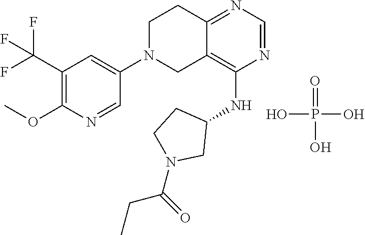 [SMILES: CCC(=O)N1CC[C@H](Nc2ncnc3c2CN(c2cnc(OC)c(C(F)(F)F)c2)CC3)C1 ; O=P(O)(O)O]

ZSTK474 refers to a compound of the following structure. Some embodiments are directed to a salt or enantiomer of ZSTK474.

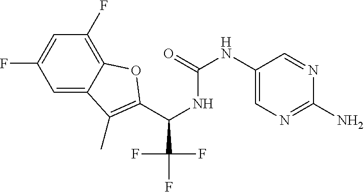 [SMILES: Cc1c([C@@H](NC(=O)Nc2cnc(N)nc2)C(F)(F)F)oc2c(F)cc(F)cc12]

In some embodiments, the PI3K-alpha inhibitor is alpelisib. Some embodiments comprise administering about 100 mg/day to about 400 mg/day, about 100 mg/day to about 350 mg/day, about 100 mg/day to about 300 mg/day, about 100 mg/day to about 250 mg/day, about 150 mg/day to about 400 mg/day, about 150 mg/day to about 350 mg/day, about 150 mg/day to about 300 mg/day, about 200 mg/day to about 400 mg/day, or about 200 mg/day to about 350 mg/day of the alpelisib. Some embodiments comprise administering about 150 mg/day to about 250 mg/day of the alpelisib. Some embodiments comprise administering about 100 mg/day, about 150 mg/day, about 200 mg/day, about 250 mg/day, about 300 mg/day, about 350 mg/day, or about 400 mg/day of the alpelisib. Some embodiments comprise administering about 200 mg/day of the alpelisib.

In some embodiments, the PI3K-alpha inhibitor is inavolisib. Some embodiments comprise administering about 5 mg/day to about 20 mg/day, about 5 mg/day to about 18 mg/day, about 5 mg/day to about 16 mg/day, about 5 mg/day to about 14 mg/day, about 7 mg/day to about 20 mg/day, about 7 mg/day to about 18 mg/day, about 7 mg/day to about 16 mg/day, about 9 mg/day to about 20 mg/day, or about 9 mg/day to about 18 mg/day of the inavolisib. Some embodiments comprise administering about 7 mg/day to about 12 mg/day of the inavolisib.

Some embodiments comprise administering about 5 mg/day, about 6 mg/day, about 7 mg/day, about 8 mg/day, about 9 mg/day, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, or about 20 mg/day of the inavolisib. Some embodiments comprise administering about 9 mg/day of the inavolisib.

In some embodiments, the PI3K-alpha inhibitor is serabelisib. Some embodiments comprise administering about 50 mg/day to about 600 mg/day, about 50 mg/day to about 500 mg/day, about 50 mg/day to about 400 mg/day, about 50 mg/day to about 300 mg/day, about 100 mg/day to about 600 mg/day, about 100 mg/day to about 500 mg/day, about 100 mg/day to about 400 mg/day, about 200 mg/day to about 600 mg/day, or about 200 mg/day to about 500 mg/day of the serabelisib. Some embodiments comprise administering about 100 mg/day to about 200 mg/day of the serabelisib. Some embodiments comprise administering about 150 mg/day to about 250 mg/day of the serabelisib. Some embodiments comprise administering about 50 mg/day, about 100 mg/day, about 150 mg/day, about 200 mg/day, about 250 mg/day, about 300 mg/day, about 350 mg/day, about 400 mg/day, about 450 mg/day, about 500 mg/day, about 550 mg/day, or about 600 mg/day of the serabelisib. In some embodiments, the serabelisib is administered at a dose of about 200 mg/day.

In some embodiments, the serabelisib is administered for two days per week. In some embodiments, the serabelisib is administered for three days per week. In some embodiments, the serabelisib is administered for four days per week. In some embodiments, the serabelisib is administered for two consecutive days per week. In some embodiments, the serabelisib is administered for three consecutive days per week. In some embodiments, the serabelisib is administered for four consecutive days per week.

In some embodiments, the serabelisib is administered at a dose of about 100 mg/day to about 300 mg/day for three consecutive days, followed the next day by four consecutive days without administering the serabelisib.

In some embodiments, the method comprises administering to the subject in need thereof:
(a) a therapeutically effective amount of a dual mTORC1 and mTORC2 inhibitor,
(b) a therapeutically effective amount of a PI3K-alpha inhibitor, and
(c) a therapeutically effective amount of one or more anti-cancer agents that do not directly inhibit enzymes in the PI3K/AKT/mTOR pathway.

In some embodiments, the one or more compounds that inhibit two or more enzymes of the PI3K/AKT/mTOR pathway comprise sapanisertib and serabelisib.

In some embodiments, the method comprises administering to the subject in need thereof:
(a) a therapeutically effective amount of sapanisertib,
(b) a therapeutically effective amount of serabelisib, and
(c) a therapeutically effective amount of one or more anti-cancer agents that do not directly inhibit enzymes in the PI3K/AKT/mTOR pathway.

In some embodiments, the PI3K-alpha inhibitor selectively inhibits a PI3K-alpha mutant. The term “PI3K-alpha mutant,” as used herein, refers to a variant of phosphoinositide 3-kinase alpha (PI3Kα) resulting from one or more genetic mutations in the PIK3CA gene, which encodes the catalytic subunit p110α of PI3Kα. Such mutations may include, but are not limited to, single nucleotide substitutions, insertions, deletions, amplifications, or structural alterations that affect PI3Kα activity, stability, or signaling. PI3Kα mutants may exhibit gain-of-function properties, leading to constitutive activation of the PI3K/AKT/mTOR pathway, increased oncogenic signaling, or resistance to regulatory feedback. The term encompasses common activating mutations such as H1047R, E545K, and E542K, as well as other known or novel mutations that alter PI3Kα function in physiological or pathological contexts, including cancer and other proliferative disorders.

In some embodiments, the PI3K-alpha mutant specific inhibitor is RLY-2608, STX-478, LY4045004, LOXO-783, OKI-219, or a combination thereof.

RLY-2608 refers to a compound of the following structure. Some embodiments are directed to a salt or enantiomer of RLY-2608.

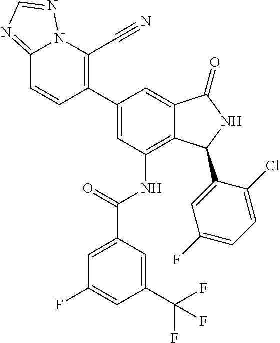 [SMILES: N#Cc1c(-c2cc(NC(=O)c3cc(F)cc(C(F)(F)F)c3)c3c(c2)C(=O)N[C@H]3c2cc(F)ccc2Cl)ccc2ncnn12]

STX-478 refers to a compound of the following structure. Some embodiments are directed to a salt or enantiomer of STX-478.

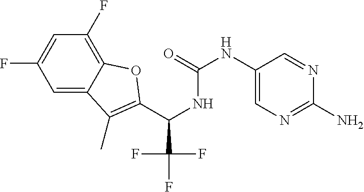 [SMILES: Cc1c([C@@H](NC(=O)Nc2cnc(N)nc2)C(F)(F)F)oc2c(F)cc(F)cc12]

### Anti-Cancer Agents

The term “anti-cancer agent,” as used herein, refers to a compound, molecule, biologic, or therapeutic intervention that inhibits, suppresses, or reduces the growth, proliferation, survival, or metastasis of cancer cells. Anti-cancer agents may act through various mechanisms, including but not limited to inducing apoptosis, inhibiting cell cycle progression, disrupting DNA replication or repair, targeting oncogenic signaling pathways, modulating the immune response, inhibiting angiogenesis, or altering the tumor microenvironment. The term encompasses chemotherapeutic agents, targeted therapies, immune checkpoint inhibitors, monoclonal antibodies, hormone therapies, anti-angiogenic agents, radiopharmaceuticals, epigenetic modulators, and other agents with anti-neoplastic activity. Unless otherwise specified, an anti-cancer agent may be used alone or in combination with other therapeutic modalities for cancer treatment purposes.

The phrase “one or more anti-cancer agents that do not directly inhibit enzymes in the PI3K/AKT/mTOR pathway,” as used herein, refers to one or more therapeutic compounds, biologics, or other agents that exhibit anti-cancer activity through mechanisms independent of direct inhibition of phosphoinositide 3-kinase (PI3K), protein kinase B (AKT), or mechanistic target of rapamycin (mTOR). Such agents may act through various alternative pathways, including but not limited to DNA damage induction, apoptosis activation, cell cycle arrest, immune modulation, angiogenesis inhibition, epigenetic regulation, disruption of microtubule dynamics, or targeting oncogenic proteins unrelated to the PI3K/AKT/mTOR signaling cascade. The term encompasses, but is not limited to, chemotherapeutic agents such as platinum-based compounds, taxanes, and topoisomerase inhibitors; targeted therapies such as tyrosine kinase inhibitors, RAS pathway inhibitors, and BCL-2 inhibitors; immune checkpoint inhibitors; monoclonal antibodies; and radiation-sensitizing agents. Unless otherwise specified, these agents may be used alone or in combination with PI3K/AKT/mTOR inhibitors for therapeutic purposes in cancer treatment. The action of these agents may indirectly increase or decrease signaling in the PI3K/AKT/mTOR pathway by virtue of their primary mechanism of action but are not per se direct pathway inhibitors that directly interact with pathway enzymes.

As used herein, the term “direct inhibitor of an enzyme or signaling protein” refers to a compound, molecule, or agent that physically interacts with and directly reduces the activity, function, or signaling of a specific enzyme or signaling protein, as opposed to indirect mechanisms that act through upstream or downstream regulatory effects. Direct inhibition may occur through competitive, non-competitive, uncompetitive, allosteric, irreversible, or reversible binding at an active site, allosteric site, or other functional domain of the target molecule. Such inhibitors may include, but are not limited to, small molecules, peptides, monoclonal antibodies, nucleic acid-based agents, or other biologics designed to bind specifically to the target enzyme or signaling protein. The degree of inhibition may be quantified using biochemical or cellular assays, including determination of IC₅₀, Ki, or other potency metrics. Unless otherwise specified, direct inhibitors may be used alone or in combination with other therapeutic agents for research, diagnostic, or therapeutic applications.

In some embodiments, the one or more anti-cancer agents that do not directly inhibit enzymes in the PI3K/AKT/mTOR pathway comprise a chemotherapeutic agent. The term “chemotherapeutic agent,” as used herein, refers to a compound, molecule, or pharmaceutical composition that inhibits, suppresses, or reduces the growth, proliferation, survival, or metastasis of cancer cells through cytotoxic or cytostatic mechanisms. Chemotherapeutic agents may act by directly damaging DNA, interfering with cell division, disrupting metabolic processes, or modulating signaling pathways implicated in tumor cell survival. The term includes, but is not limited to, alkylating agents, antimetabolites, anthracyclines, taxanes, vinca alkaloids, topoisomerase inhibitors, and other small-molecule drugs used in oncology. Unless otherwise specified, chemotherapeutic agents may be used alone or in combination with targeted therapies, immunotherapies, or other anti-cancer agents for therapeutic applications. In some embodiments, the chemotherapeutic agent is a taxane. In some embodiments, the chemotherapeutic agent is a platinum-based agent. In some embodiments, the chemotherapeutic agent is an alkylating agent, an antimetabolite, an anthracycline, a vinca alkaloid, a topoisomerase inhibitor, a proteasome inhibitor, a histone deacetylase (HDAC) inhibitor, an immunomodulatory drug (IMiD), a microtubule inhibitor, or any other cytotoxic or cytostatic agent used for the treatment of cancer. The chemotherapeutic agent may include, but is not limited to, alkylating agents such as cyclophosphamide, ifosfamide, melphalan, chlorambucil, busulfan, carmustine, lomustine, streptozocin, temozolomide, thiotepa, dacarbazine, procarbazine, mechlorethamine, and estramustine. Antimetabolites may include methotrexate, pemetrexed, pralatrexate, 5-fluorouracil (5-FU), capecitabine, cytarabine, gemcitabine, fludarabine, cladribine, pentostatin, decitabine, azacitidine, nelarabine, mercaptopurine, and thioguanine. Anthracyclines such as doxorubicin, daunorubicin, epirubicin, idarubicin, mitoxantrone, and valrubicin may also be used. Taxanes include paclitaxel, docetaxel, and cabazitaxel, while vinca alkaloids may consist of vincristine, vinblastine, vinorelbine, and vinflunine. Topoisomerase inhibitors such as irinotecan, topotecan, etoposide, and teniposide may be employed, as well as platinum-based agents including cisplatin, carboplatin, and oxaliplatin. Proteasome inhibitors, like bortezomib, carfilzomib, and ixazomib, as well as histone deacetylase (HDAC) inhibitors such as vorinostat, romidepsin, panobinostat, and belinostat, may also be included. Immunomodulatory drugs (IMiDs) such as thalidomide, lenalidomide, and pomalidomide, along with microtubule inhibitors like eribulin and estramustine, are also encompassed within the definition of chemotherapeutic agents. Other chemotherapeutic agents may include hydroxyurea, mitomycin C, actinomycin D, trabectedin, lurbinectedin, pegylated L-asparaginase, and arsenic trioxide. Unless otherwise specified, the chemotherapeutic agent may be used alone or in combination with other anti-cancer therapies, including targeted therapies, immunotherapies, hormone therapies, or radiation therapy.

The term “targeted therapy,” as used herein, refers to a therapeutic approach that selectively modulates the activity, function, or signaling of specific molecules, pathways, or cellular processes involved in disease pathogenesis, particularly in cancer. Unlike conventional chemotherapeutic agents, which broadly affect rapidly dividing cells, targeted therapies act on defined molecular targets such as oncogenic proteins, growth factor receptors, intracellular kinases, or epigenetic regulators. Targeted therapies may include, but are not limited to, small molecule inhibitors, monoclonal antibodies, antibody-drug conjugates, peptide-based agents, RNA-based therapies, and other biologics designed to interfere with tumor growth, survival, metastasis, or resistance mechanisms. Such therapies may act by inhibiting kinases, blocking ligand-receptor interactions, modulating protein degradation, interfering with DNA repair, altering immune evasion, or inducing synthetic lethality in tumor cells. Examples of targeted therapies include inhibitors of receptor tyrosine kinases, serine/threonine kinases, PI3K/AKT/mTOR pathway components, CDK4/6, BCL-2 family proteins, and immune checkpoint regulators such as PD-1, PD-L1, and CTLA-4. Unless otherwise specified, targeted therapies may be used alone or in combination with other therapeutic modalities, including chemotherapy, immunotherapy, radiation therapy, or surgical intervention, for therapeutic applications in oncology.

In some embodiments, the one or more anti-cancer agents that do not directly inhibit enzymes in the PI3K/AKT/mTOR pathway comprise a targeted therapy. The term “targeted therapy,” in reference to one or more anti-cancer agents that do not directly inhibit enzymes in the PI3K/AKT/mTOR pathway, refers to a therapeutic agent or modality designed to selectively interfere with oncogenic drivers, tumor-associated molecular targets, or dysregulated cellular pathways in cancer while specifically excluding compounds, biologics, or other interventions that directly inhibit phosphoinositide 3-kinase (PI3K), protein kinase B (AKT), or mechanistic target of rapamycin (mTOR), including their isoforms, regulatory subunits, and associated complexes. Such targeted therapies may act through mechanisms including, but not limited to, inhibition of receptor tyrosine kinases (e.g., EGFR, HER2, VEGFR, MET, and ALK), serine/threonine kinases outside the PI3K/AKT/mTOR pathway (e.g., CDK4/6, RAF, and MEK), oncogenic fusion proteins, epigenetic regulators, DNA damage response pathways, or immune checkpoint modulators. Examples of such therapies include EGFR inhibitors (e.g., erlotinib and osimertinib), BRAF/MEK inhibitors (e.g., dabrafenib and trametinib), CDK4/6 inhibitors (e.g., palbociclib and ribociclib), PARP inhibitors (e.g., olaparib and rucaparib), and immune checkpoint inhibitors (e.g., pembrolizumab and nivolumab). Unless otherwise specified, such targeted therapies may be used alone or in combination with other therapeutic agents, including chemotherapy, radiation therapy, hormone therapy, or immunotherapy, for the treatment of cancer. The preceding abbreviations refer to the following:
EGFR—Epidermal growth factor receptor (ErbB-1)
HER2—Receptor tyrosine-protein kinase erbB-2 (ErbB-2)
VEGFR—Vascular endothelial growth factor receptor (e.g., VEGFR1: FLT1, VEGFR2: KDR, VEGFR3: FLT4)
MET—Hepatocyte growth factor receptor (HGF receptor)
ALK—Anaplastic lymphoma kinase
CDK4/6—Cyclin-dependent kinase 4/Cyclin-dependent kinase 6
RAF—RAF proto-oncogene serine/threonine-protein kinase (includes ARAF, BRAF, and CRAF/RAFI)
MEK—Mitogen-activated protein kinase kinase (MAP2K1 for MEK1, MAP2K2 for MEK2)

In some embodiments, the one or more anti-cancer agents that do not directly inhibit enzymes in the PI3K/AKT/mTOR pathway comprise a taxane. The term “taxanes,” as used herein, refers to a class of chemotherapeutic agents that inhibit microtubule function, thereby disrupting mitotic spindle formation and preventing cell division. Taxanes act by stabilizing microtubules, preventing their depolymerization, and ultimately leading to mitotic arrest and apoptotic cell death. This class of agents includes, but is not limited to, paclitaxel, docetaxel, and cabazitaxel, which are widely used in the treatment of various cancers, including breast, ovarian, lung, and prostate cancers. Taxanes may be administered alone or in combination with other chemotherapeutic agents, targeted therapies, immunotherapies, or radiation therapy to enhance anti-cancer efficacy. Unless otherwise specified, taxanes may be used in conventional formulations or as nanoparticle-based or albumin-bound formulations to improve solubility, bioavailability, and therapeutic outcomes. In some embodiments, the taxane is paclitaxel, a naturally derived compound from the bark of the Pacific yew tree (Taxus brevifolia), as well as its semi-synthetic derivatives docetaxel and cabazitaxel, which are widely used in the treatment of breast, ovarian, lung, prostate, and other cancers. Albumin-bound paclitaxel, also known as nab-paclitaxel or Abraxane, is a nanoparticle formulation designed to improve solubility and bioavailability. Other taxane derivatives and analogs, including larotaxel and ortataxel, have been developed or investigated for their therapeutic potential. Unless otherwise specified, taxanes may be administered alone or in combination with other anti-cancer agents, including chemotherapy, targeted therapies, immunotherapies, or radiation therapy, in various formulations to enhance efficacy and reduce toxicity.

In some embodiments, the one or more anti-cancer agents that do not directly inhibit enzymes in the PI3K/AKT/mTOR pathway comprise paclitaxel, docetaxel, or a combination thereof.

In some embodiments, the one or more anti-cancer agents that do not directly inhibit enzymes in the PI3K/AKT/mTOR pathway comprise paclitaxel. Some embodiments comprise administering about 40 mg/m² to about 100 mg/m², about 40 mg/m² to about 90 mg/m², about 40 mg/m² to about 80 mg/m², about 40 mg/m² to about 70 mg/m², about 50 mg/m² to about 100 mg/m², about 50 mg/m² to about 90 mg/m², about 50 mg/m² to about 80 mg/m², about 60 mg/m² to about 100 mg/m², or about 60 mg/m² to about 90 mg/m² of the paclitaxel per week. Some embodiments comprise administering about 40 mg/m² to about 100 mg/m² of the paclitaxel per week. Some embodiments comprise administering about 60 mg/m² to about 90 mg/m² of the paclitaxel per week. Some embodiments comprise administering about 40 mg/m², about 50 mg/m², about 60 mg/m², about 70 mg/m², about 80 mg/m², about 90 mg/m², or about 100 mg/m² of the paclitaxel per week. Some embodiments comprise administering about 80 mg/m² of the paclitaxel per week. In some embodiments, the weekly dose of paclitaxel is administered on a single day of the week.

In some embodiments, the paclitaxel is administered at a dose of about 80 mg/m² to about 90 mg/m² once per week for three weeks. Some embodiments comprise administering about 100 mg/m² to about 250 mg/m² of the paclitaxel every 21 days. In some embodiments, the paclitaxel is administered at a dose of about 80 mg/m² to about 90 mg/m² once per week for three weeks followed by one week without administering paclitaxel. Some embodiments comprise administering about 100 mg/m² to about 250 mg/m² of the paclitaxel every 28 days.

In some embodiments, the one or more anti-cancer agents that do not directly inhibit enzymes in the PI3K/AKT/mTOR pathway comprise carboplatin. Carboplatin AUC (area under the concentration-time curve) 5-6 repeated every 21 days.

In some embodiments, the carboplatin is administered in combination with about 40 mg/m² to about 200 mg/m² of paclitaxel. In some embodiments, the carboplatin is administered in combination with about 100 mg/m² to about 150 mg/m² of paclitaxel. In some embodiments, the carboplatin is administered in combination with about 135 mg/m² of paclitaxel.

In some embodiments, the one or more anti-cancer agents that do not directly inhibit enzymes in the PI3K/AKT/mTOR pathway comprise a CDK4 inhibitor, a CDK6 inhibitor, a CDK4/6 inhibitor, or a combination thereof. The term “CDK4/6 inhibitor,” as used herein, refers to a compound, molecule, or agent that inhibits the activity, function, or signaling of cyclin-dependent kinase 4 (CDK4) and cyclin-dependent kinase 6 (CDK6), serine/threonine kinases that regulate cell cycle progression through the G1 phase by phosphorylating the retinoblastoma protein (RB1). CDK4/6 inhibitors act by competitively binding to the ATP-binding site of CDK4 and/or CDK6, thereby preventing RB1 phosphorylation and leading to cell cycle arrest. The term encompasses, but is not limited to, selective CDK4/6 inhibitors such as palbociclib, ribociclib, and abemaciclib, as well as other pharmacological agents, biologics, or nucleic acid-based therapies that modulate CDK4/6 activity. Unless otherwise specified, CDK4/6 inhibitors may be used alone or in combination with other anti-cancer therapies for therapeutic applications in cancers characterized by dysregulated CDK4/6 signaling.

In some embodiments, the one or more anti-cancer agents that do not directly inhibit enzymes in the PI3K/AKT/mTOR pathway comprise abemaciclib, palbociclib, ribociclib, or a combination thereof.

In some embodiments, the one or more anti-cancer agents that do not directly inhibit enzymes comprise abemaciclib. Some embodiments comprise administering about 200 mg/day to about 600 mg/day, about 200 mg/day to about 500 mg/day, about 200 mg/day to about 400 mg/day, about 200 mg/day to about 300 mg/day, about 250 mg/day to about 600 mg/day, about 250 mg/day to about 500 mg/day, about 250 mg/day to about 400 mg/day, about 300 mg/day to about 600 mg/day, or about 300 mg/day to about 500 mg/day of the abemaciclib. Some embodiments comprise administering about 200 mg/day to about 600 mg/day of the abemaciclib.

Some embodiments comprise administering about 250 mg/day to about 350 mg/day of the abemaciclib. Some embodiments comprise administering about 200 mg/day, about 250 mg/day, about 300 mg/day, about 350 mg/day, about 400 mg/day, about 450 mg/day, about 500 mg/day, about 550 mg/day, or about 600 mg/day of the abemaciclib. Some embodiments comprise administering about 300 mg/day of the abemaciclib.

In some embodiments, the one or more anti-cancer agents that do not directly inhibit enzymes comprise palbociclib. Some embodiments comprise administering about 75 mg/day to about 150 mg/day, about 75 mg/day to about 125 mg/day, about 75 mg/day to about 100 mg/day, about 75 mg/day to about 90 mg/day, about 100 mg/day to about 150 mg/day, about 100 mg/day to about 125 mg/day, about 100 mg/day to about 110 mg/day, about 110 mg/day to about 150 mg/day, or about 110 mg/day to about 125 mg/day of palbociclib. Some embodiments comprise administering about 75 mg/day to about 150 mg/day of palbociclib. Some embodiments comprise administering about 100 mg/day to about 125 mg/day of palbociclib. Some embodiments comprise administering about 75 mg/day, about 80 mg/day, about 90 mg/day, about 100 mg/day, about 110 mg/day, about 120 mg/day, or about 125 mg/day of palbociclib. Some embodiments comprise administering 125 mg/day of palbociclib.

In some embodiments, the palbociclib is administered in a repeating 28-day cycle comprising a palbociclib dose of about 75 mg/day to about 150 mg/day for three weeks followed by one week without administering the palbociclib.

In some embodiments, the one or more anti-cancer agents that do not directly inhibit enzymes comprise ribociclib. Some embodiments comprise administering about 200 mg/day to about 800 mg/day, about 200 mg/day to about 700 mg/day, about 200 mg/day to about 600 mg/day, about 200 mg/day to about 500 mg/day, about 300 mg/day to about 800 mg/day, about 300 mg/day to about 700 mg/day, about 300 mg/day to about 600 mg/day, about 400 mg/day to about 800 mg/day, or about 400 mg/day to about 700 mg/day of ribociclib. Some embodiments comprise administering about 200 mg/day to about 800 mg/day of ribociclib. Some embodiments comprise administering about 400 mg/day to about 600 mg/day of ribociclib. Some embodiments comprise administering about 200 mg/day, about 250 mg/day, about 300 mg/day, about 350 mg/day, about 400 mg/day, about 450 mg/day, about 500 mg/day, about 550 mg/day, about 600 mg/day, about 650 mg/day, about 700 mg/day, about 750 mg/day, or about 800 mg/day of ribociclib. Some embodiments comprise administering about 600 mg/day of ribociclib.

In some embodiments, the ribociclib is administered in a repeating 28-day cycle comprising a ribociclib dose of about 200 mg/day to about 800 mg/day for three weeks followed by one week without administering the ribociclib.

In some embodiments, the one or more anti-cancer agents that do not directly inhibit enzymes in the PI3K/AKT/mTOR pathway comprise a hormone receptor targeting therapeutic agent. The term “hormone receptor targeting therapeutic agent,” as used herein, refers to a compound, molecule, or biologic that modulates the activity, function, expression, or signaling of hormone receptors involved in disease pathogenesis, particularly in hormone-driven cancers. Such agents may act as antagonists, agonists, selective modulators, or degraders of receptors such as the estrogen receptor (ER), progesterone receptor (PR), androgen receptor (AR), or other hormone-related targets. Hormone receptor targeting therapeutic agents include, but are not limited to, selective estrogen receptor modulators (SERMs), such as tamoxifen, raloxifene, and lasofoxifene; selective estrogen receptor degraders (SERDs), such as fulvestrant, amcenestrant, AZD8421, aorestrant, brilanestrant, camizestrant, elacestrant, giredestrant, imlunestrant, LSZ102, palazestrant, rintodestrant, or taragarestrant; PROteolysis TArgeting Chimera (PROTAC) ER degraders, such as vepdegestrant; aromatase inhibitors, such as anastrozole, letrozole, and exemestane; androgen receptor antagonists, such as enzalutamide, apalutamide, and darolutamide; and luteinizing hormone-releasing hormone (LHRH) agonists or antagonists, such as leuprolide, goserelin, degarelix, and relugolix. These agents may be used alone or in combination with other anti-cancer therapies, including chemotherapy, targeted therapies, immunotherapies, or CDK inhibitors including (CDK6, CDK4, CDK2, CDK2/9, CDK7, CDK8/19, CDK9, CDK12/13, Pan-CDK, and CDK4/6 inhibitors), for the treatment of hormone receptor-positive cancers such as breast cancer, prostate cancer, and endometrial cancer. In some embodiments, the hormone receptor targeting therapeutic agent is tamoxifen, raloxifene, toremifene, fulvestrant, anastrozole, letrozole, exemestane, enzalutamide, apalutamide, darolutamide, bicalutamide, flutamide, nilutamide, abiraterone, ketoconazole, megestrol acetate, cyproterone acetate, medroxyprogesterone acetate, leuprolide, goserelin, triptorelin, histrelin, degarelix, relugolix, elagolix, danazol, mifepristone, onapristone, and tibolone.

In some embodiments, the one or more anti-cancer agents that do not directly inhibit enzymes in the PI3K/AKT/mTOR pathway comprise fulvestrant, elacestrant, or a combination thereof.

In some embodiments, the one or more anti-cancer agents that do not directly inhibit enzymes in the PI3K/AKT/mTOR pathway comprise fulvestrant. In some embodiments, the method comprises administering about 500 mg of fulvestrant twice in a four-week interval on day 1, and administering about 500 mg of fulvestrant every 28 days thereafter.

In some embodiments, the one or more anti-cancer agents that do not directly inhibit enzymes in the PI3K/AKT/mTOR pathway comprise elacestrant. Some embodiments comprise administering about 100 mg/day to about 600 mg/day, about 100 mg/day to about 500 mg/day, about 100 mg/day to about 400 mg/day, about 100 mg/day to about 300 mg/day, about 150 mg/day to about 600 mg/day, about 150 mg/day to about 500 mg/day, about 150 mg/day to about 400 mg/day, about 200 mg/day to about 600 mg/day, or about 200 mg/day to about 500 mg/day of the elacestrant. Some embodiments comprise administering about 100 mg/day to about 600 mg/day of the elacestrant. Some embodiments comprise administering about 250 mg/day to about 400 mg/day of the elacestrant. Some embodiments comprise administering about 100 mg/day, about 150 mg/day, about 200 mg/day, about 250 mg/day, about 300 mg/day, about 350 mg/day, about 400 mg/day, about 450 mg/day, about 500 mg/day, about 550 mg/day, or about 600 mg/day of the elacestrant. Some embodiments comprise administering about 350 mg/day of the elacestrant. Some embodiments comprise administering about 345 mg/day of the elacestrant.

In some embodiments, the one or more anti-cancer agents that do not directly inhibit enzymes in the PI3K/AKT/mTOR pathway comprise a modulator of p53 activity. The term “modulator of p53 activity,” as used herein, refers to a compound, molecule, or agent that directly or indirectly influences the activity, function, stability, expression, or signaling of p53, a tumor suppressor protein involved in regulating cell cycle arrest, apoptosis, DNA repair, and senescence in response to cellular stress or genomic damage. Modulation of p53 activity may include activation, stabilization, restoration, inhibition, or degradation of p53, depending on the therapeutic context. p53 modulators include, but are not limited to, small-molecule activators such as PRIMA-1, APR-246 (eprenetapopt), and CP-31398 that restore or enhance the function of mutant p53; MDM2 inhibitors such as nutlins, idasanutlin, siremadlin, and RG7112 that prevent p53 degradation by blocking the p53-MDM2 interaction; p53 stabilizers such as tenovin and MI-773; and inhibitors of p53 activity such as pifithrin-α and pifithrin-μ. Additionally, the term encompasses selective inhibitors of nuclear export (SINE) compounds such as selinexor, which inhibit exportin-1 (XPO1), thereby preventing nuclear export and degradation of p53, leading to increased nuclear retention and enhanced tumor suppressor activity. Unless otherwise specified, modulators of p53 activity may be used alone or in combination with other anti-cancer agents, including chemotherapy, targeted therapies, immunotherapies, or radiation therapy, for therapeutic applications in cancers with wild-type, mutant, or dysfunctional p53. In some embodiments, the modulator of p53 activity is PRIMA-1, APR-246 (eprenetapopt), CP-31398, nutlins, idasanutlin, siremadlin, RG7112, tenovin, MI-773, pifithrin-α, pifithrin-μ, selinexor, verdinexor, KPT-8602, RITA, MIRA-1, NSC-319726, SCH529074, HDM201, AMG-232, DS-3032, ALRN-6924, PK11007, arsenic trioxide, 2aG4, and ATSP-7041.

In some embodiments, the one or more anti-cancer agents that do not directly inhibit enzymes in the PI3K/AKT/mTOR pathway comprise selinexor. Some embodiments comprise administering about 60 mg/week to about 80 mg/week, about 60 mg/week to about 70 mg/week, about 60 mg/week to about 65 mg/week, about 65 mg/week to about 80 mg/week, about 65 mg/week to about 75 mg/week, or about 70 mg/week to about 80 mg/week of the selinexor. Some embodiments comprise administering about 60 mg/week to about 80 mg/week of the selinexor. Some embodiments comprise administering about 65 mg/week to about 75 mg/week of the selinexor. Some embodiments comprise administering about 60 mg/week, about 65 mg/week, about 70 mg/week, about 75 mg/week, or about 80 mg/week of the Selinexor.

Some embodiments comprise administering about 60 mg/week of the selinexor if the subject has a BMI<20 kg/m² or administering about 80 mg/week of the selinexor if the subject has a BMI>20 kg/m².

PRIMA-1, also known as 2,2-Bis(hydroxymethyl)-1-azabicyclo[2.2.2]octan-3-one, refers to a compound of the following structure. Some embodiments are directed to a salt or enantiomer of PRIMA-1.

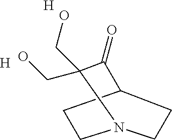

Eprenetapopt, also known as APR-246, refers to a compound of the following structure. Some embodiments are directed to a salt or enantiomer of eprenetapopt.

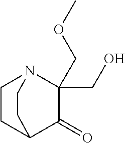

CP-31398 refers to a compound of the following structure. Some embodiments are directed to a salt or enantiomer of CP-31398.

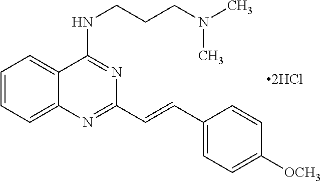 [SMILES: COc1ccc(/C=C/c2nc(NCCCN(C)C)c3ccccc3n2)cc1]

Nutlins refers to a compound of the following structure. Some embodiments are directed to a salt or enantiomer of nutlins.

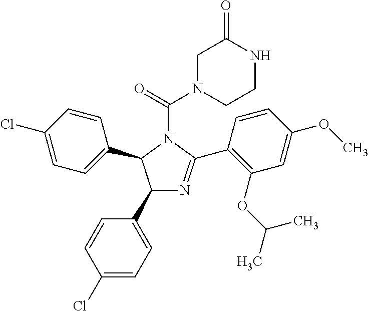 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

Siremadlin, also known as NVP-HDM201, refers to a compound of the following structure. Some embodiments are directed to a salt or enantiomer of siremadlin.

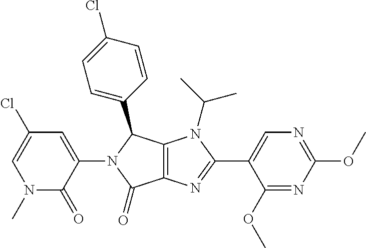 [SMILES: COc1ncc(-c2nc3c(n2C(C)C)[C@H](c2ccc(Cl)cc2)N(c2cc(Cl)cn(C)c2=O)C3=O)c(OC)n1]

RG7112 refers to a compound of the following structure. Some embodiments are directed to a salt or enantiomer of RG7112.

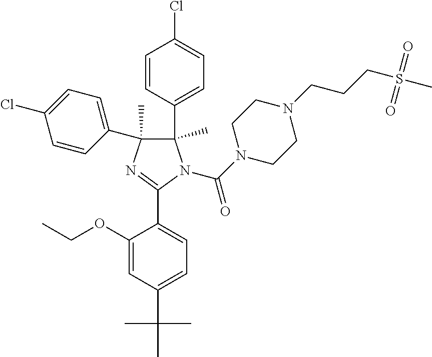 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

Tenovin refers to a compound of the following structure. Some embodiments are directed to a salt or enantiomer of tenovin.

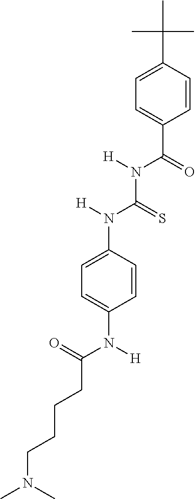 [SMILES: CN(C)CCCCC(=O)Nc1ccc(NC(=S)NC(=O)c2ccc(C(C)(C)C)cc2)cc1]

MI-773, also known as SAR405838, refers to a compound of the following structure. Some embodiments are directed to a salt or enantiomer of MI-773.

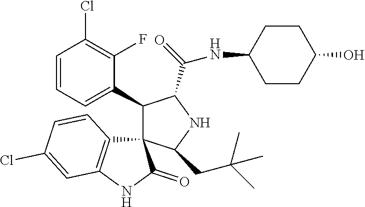 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

Pifithrin-α, also known as 2-phenylethynesulfonamide, refers to a compound of the following structure. Some embodiments are directed to a salt or enantiomer of pifithrin-α.

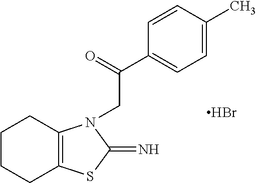 [SMILES: Cc1ccc(C(=O)Cn2c3c(sc2=N)CCCC3)cc1 ; Br]

Pifithrin-μ refers to a compound of the following structure. Some embodiments are directed to a salt or enantiomer of pifithrin-μ.

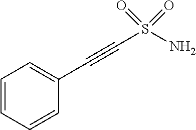 [SMILES: NS(=O)(=O)C#Cc1ccccc1]

Selinexor refers to a compound of the following structure. Some embodiments are directed to a salt or enantiomer of selinexor.

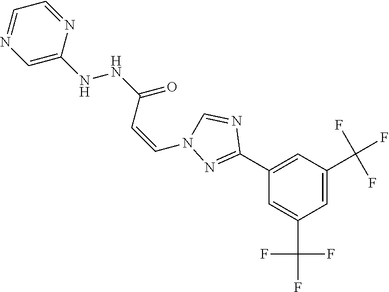 [SMILES: O=C(/C=C\n1cnc(-c2cc(C(F)(F)F)cc(C(F)(F)F)c2)n1)NNc1cnccn1]

Verdinexor, also known as KPT-335, refers to a compound of the following structure. Some embodiments are directed to a salt or enantiomer of verdinexor.

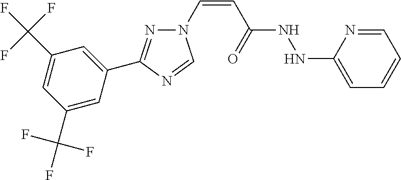 [SMILES: O=C(/C=C\n1cnc(-c2cc(C(F)(F)F)cc(C(F)(F)F)c2)n1)NNc1ccccn1]

Eltanexor, also known as KPT-8602, refers to a compound of the following structure. Some embodiments are directed to a salt or enantiomer of eltanexor.

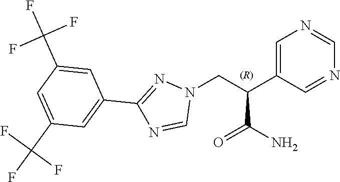

RITA, also known as NSC 652287, refers to a compound of the following structure. Some embodiments are directed to a salt or enantiomer of RITA.

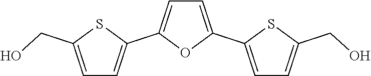 [SMILES: OCc1ccc(-c2ccc(-c3ccc(CO)s3)o2)s1]

MIRA-1, also known as NSC 19630, refers to a compound of the following structure. Some embodiments are directed to a salt or enantiomer of MIRA-1.

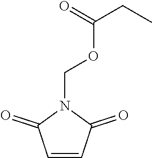

NSC-319726, also known as ZMC1, refers to a compound of the following structure. Some embodiments are directed to a salt or enantiomer of NSC-319726.

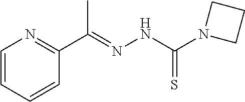 [SMILES: C/C(=N\NC(=S)N1CCC1)c1ccccn1]

SCH529074 refers to a compound of the following structure. Some embodiments are directed to a salt or enantiomer of SCH529074.

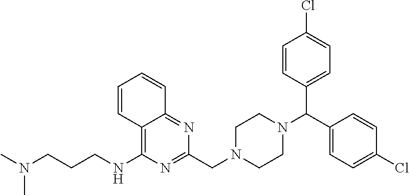 [SMILES: CN(C)CCCNc1nc(CN2CCN(C(c3ccc(Cl)cc3)c3ccc(Cl)cc3)CC2)nc2ccccc12]

HDM201, also known as siremadlin, refers to a compound of the following structure. Some embodiments are directed to a salt or enantiomer of HDM201.

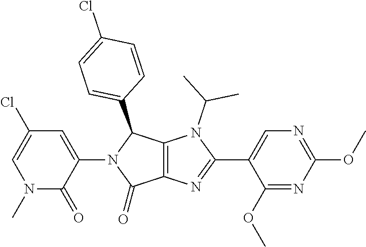 [SMILES: COc1ncc(-c2nc3c(n2C(C)C)[C@H](c2ccc(Cl)cc2)N(c2cc(Cl)cn(C)c2=O)C3=O)c(OC)n1]

AMG-232, also known as navtemadlin, refers to a compound of the following structure. Some embodiments are directed to a salt or enantiomer of AMG-232.

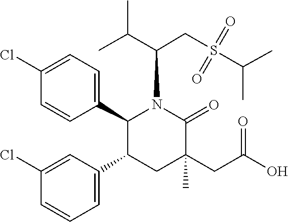 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

DS-3032, also known as milademetan, refers to a compound of the following structure. Some embodiments are directed to a salt or enantiomer of milademetan.

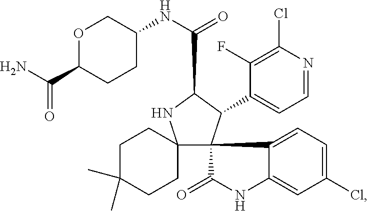 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

Amcenestrant, also known as SAR439859, refers to a compound having the IUPAC name 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid. Some embodiments are directed to a salt or enantiomer of amcenestrant.

AZD8421 refers to a compound having the following structure. Some embodiments are directed to a salt or enantiomer of AZD8421.

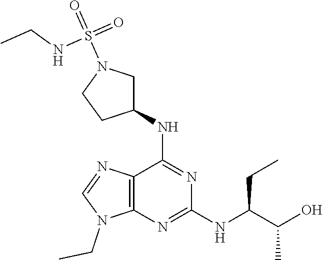 [SMILES: CCNS(=O)(=O)N1CC[C@H](Nc2nc(N[C@@H](CC)[C@@H](C)O)nc3c2ncn3CC)C1]

Borestrant, also known as ZB716, refers to a compound having the following chemical structure. Some embodiments are directed to a salt or enantiomer of borestrant.

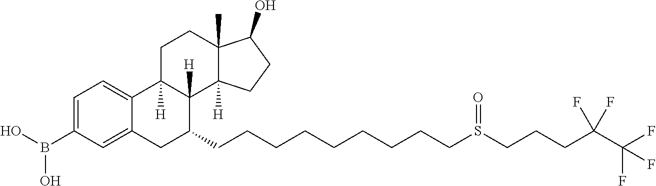

Brilanestrant, also known as GDC-0810, refers to a compound having the IUPAC name (2E)-3-{4-[(1E)-2-(2-chloro-4-fluorophenyl)-1-(1H-indazol-5-yl)but-1-en-1-yl]phenyl}prop-2-enoic acid. Some embodiments are directed to a salt or enantiomer of brilanestrant.

Camizestrant, also known as AZD9833, refers to a compound having the IUPAC name N-[1-(3-fluoropropyl)azetidin-3-yl]-6-[(6S,8R)-8-methyl-7-(2,2,2-trifluoroethyl)-3,6,8,9-tetrahydropyrazolo[4,3-f]isoquinolin-6-yl]pyridin-3-amine. Some embodiments are directed to a salt or enantiomer of camizestrant.

Giredestrant, also known as GDC-9545, refers to a compound having the IUPAC name 3-[(1R,3R)-1-[2,6-difluoro-4-[[1-(3-fluoropropyl)azetidin-3-yl]amino]phenyl]-3-methyl-1,3,4,9-tetrahydropyrido[3,4-b]indol-2-yl]-2,2-difluoro-propan-1-ol. Some embodiments are directed to a salt or enantiomer of giredestrant.

Imlunestrant, also known as LY3484356, refers to a compound having the IUPAC name (5R)-5-[4-[2-[3-(Fluoromethyl)azetidin-1-yl]ethoxy]phenyl]-8-(trifluoromethyl)-5H-chromeno[4,3-c]quinolin-2-ol. Some embodiments are directed to a salt or enantiomer of imlunestrant.

LSZ102 refers to a compound having the following structure. Some embodiments are directed to a salt or enantiomer of LSZ102.

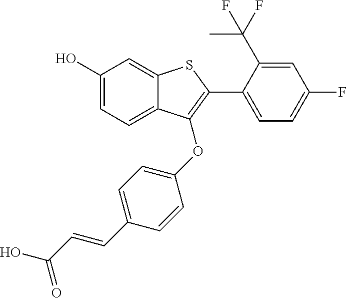 [SMILES: CC(F)(F)c1cc(F)ccc1-c1sc2cc(O)ccc2c1Oc1ccc(/C=C/C(=O)O)cc1]

Palazestrant, also known as OP-1250, refers to a compound having the IUPAC name (1R,3R)-2-(2-fluoro-2-methylpropyl)-3-methyl-1-[4-(1-propylazetidin-3-yl)oxyphenyl]-1,3,4,9-tetrahydropyrido[3,4-b]indole. Some embodiments are directed to a salt or enantiomer of palazestrant.

Rintodestrant, also known as G1T48, refers to a compound having the IUPAC name (E)-3-[4-[2-(4-fluoro-2,6-dimethyl-benzoyl)-6-hydroxy-benzothiophen-3-yl]oxyphenyl]prop-2-enoic acid. Some embodiments are directed to a salt or enantiomer of rintodestrant.

Taragarestrant, also known as D-0502, refers to a compound having the IUPAC name (E)-3-[3,5-dichloro-4-[(1R,3R)-2-(2-fluoro-2-methylpropyl)-3-methyl-1,3,4,9-tetrahydropyrido[3,4-b]indol-1-yl]phenyl]prop-2-enoic acid. Some embodiments are directed to a salt or enantiomer of taragarestrant.

Tegtociclib, also known as PF-07104091, refers to a compound having the IUPAC name [(1R,3S)-3-[3-[[5-(methoxymethyl)-2-methylpyrazole-3-carbonyl]amino]-1H-pyrazol-5-yl]cyclopentyl]N-propan-2-ylcarbamate. Some embodiments are directed to a salt or enantiomer of tegtociclib.

INX-315 refers to a compound having the following chemical structure. Some embodiments are directed to a salt or enantiomer of INX-315.

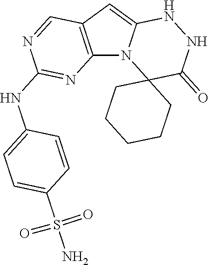 [SMILES: NS(=O)(=O)c1ccc(Nc2ncc3cc4n(c3n2)C2(CCCCC2)C(=O)NN4)cc1]

Cirtociclib, also known as BLU-222, refers to a compound having the following chemical structure. Some embodiments are directed to a salt or enantiomer of BLU-222.

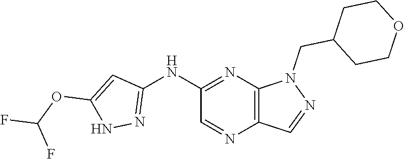 [SMILES: FC(F)Oc1cc(Nc2cnc3cnn(CC4CCOCC4)c3n2)n[nH]1]

Ebvaciclib, also known as PF-06873600, refers to a compound having the following chemical structure. Some embodiments are directed to a salt or enantiomer of ebvaciclib.

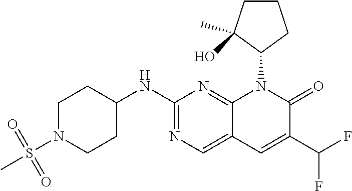 [SMILES: C[C@]1(O)CCC[C@@H]1n1c(=O)c(C(F)F)cc2cnc(NC3CCN(S(C)(=O)=O)CC3)nc21]

Fadraciclib, also known as CYC065, refers to a compound having the following chemical structure. Some embodiments are directed to a salt or enantiomer of fadraciclib.

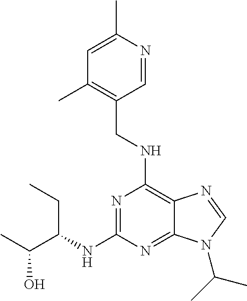 [SMILES: CC[C@H](Nc1nc(NCc2cnc(C)cc2C)c2ncn(C(C)C)c2n1)[C@@H](C)O]

Samuraciclib, also known as CT7001 and ICEC0942, refers to a compound having the following chemical structure. Some embodiments are directed to a salt or enantiomer of samuraciclib.

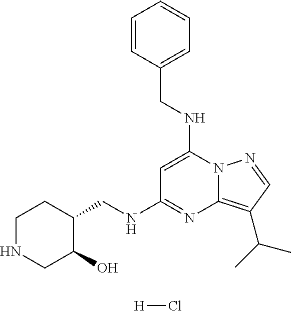 [SMILES: CC(C)c1cnn2c(NCc3ccccc3)cc(NC[C@H]3CCNC[C@@H]3O)nc12 ; Cl]

SY-5609 refers to a compound having the following chemical structure. Some embodiments are directed to a salt or enantiomer of SY-5609.

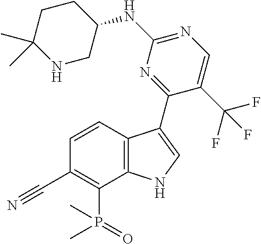 [SMILES: CC1(C)CC[C@H](Nc2ncc(C(F)(F)F)c(-c3c[nH]c4c(P(C)(C)=O)c(C#N)ccc34)n2)CN1]

Tacaciclib, also known as XL102, refers to a compound having the following chemical structure. Some embodiments are directed to a salt or enantiomer of XL102.

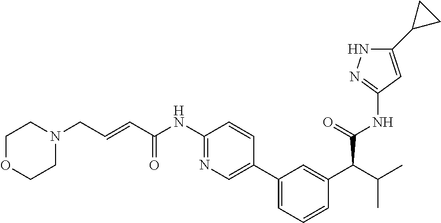 [SMILES: CC(C)[C@H](C(=O)Nc1cc(C2CC2)[nH]n1)c1cccc(-c2ccc(NC(=O)/C=C/CN3CCOCC3)nc2)c1]

LY3405105 refers to a compound having the following structure. Some embodiments are directed to a salt or enantiomer of LY3405105.

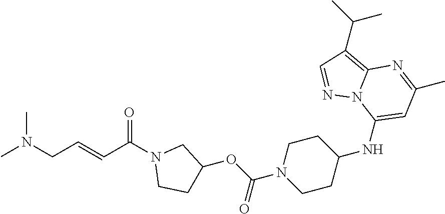

BTX-A51 refers to a compound having the following chemical structure. Some embodiments are directed to a salt or enantiomer of BTX-A51.

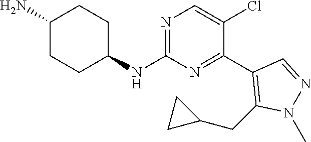 [SMILES: Cn1ncc(-c2nc(N[C@H]3CC[C@H](N)CC3)ncc2Cl)c1CC1CC1]

Romaciclib, also known as RVU120 and SEL120-34A, refers to a compound having the following chemical structure. Some embodiments are directed to a salt or enantiomer of romaciclib.

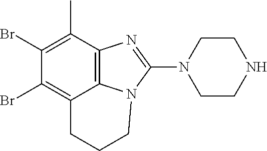 [SMILES: Cc1c(Br)c(Br)c2c3c1nc(N1CCNCC1)n3CCC2]

Enitociclib, also known as VIP152, refers to a compound having the IUPAC name 5-fluoro-4-(4-fluoro-2-methoxyphenyl)-N-[4-[(methylsulfonimidoyl)methyl]pyridin-2-yl]pyridin-2-amine. Some embodiments are directed to a salt or enantiomer of enitociclib.

KB-0742 refers to a compound having the following chemical structure. Some embodiments are directed to a salt or enantiomer of KB-0742.

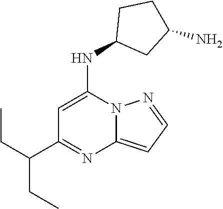 [SMILES: CCC(CC)c1cc(N[C@H]2CC[C@H](N)C2)n2nccc2n1]

Zotiraciclib, also known as TG02 and SB1317, refers to a compound having the IUPAC name (16E)-14-methyl-20-oxa-5,7,14,27-tetraazatetracyclo[19.3.1.1²,⁶. 1⁸,¹²] heptacosa-1(24),2,4,6(27),8,10,12(26),16,21(25),22-decaene. Some embodiments are directed to a salt or enantiomer of zotiraciclib.

AZD4573 refers to a compound having the following chemical structure. Some embodiments are directed to a salt or enantiomer of AZD4573.

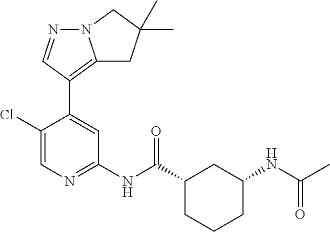 [SMILES: CC(=O)N[C@@H]1CCC[C@H](C(=O)Nc2cc(-c3cnn4c3CC(C)(C)C4)c(Cl)cn2)C1]

Voruciclib, also known as P1446A, refers to a compound having the following chemical structure. Some embodiments are directed to a salt or enantiomer of voruciclib.

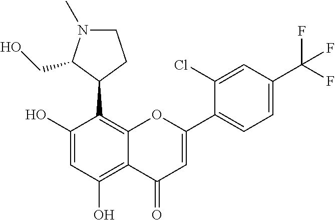 [SMILES: CN1CC[C@@H](c2c(O)cc(O)c3c(=O)cc(-c4ccc(C(F)(F)F)cc4Cl)oc23)[C@@H]1CO]

Alvocidib, also known as Flavopiridol, refers to a compound having the IUPAC name 2-(2-chlorophenyl)-5,7-dihydroxy-8-[(3S,4R)-3-hydroxy-1-methylpiperidin-4-yl]-4H-1-benzopyran-4-one. Some embodiments are directed to a salt or enantiomer of alvocidib.

Atuveciclib, also known as BAY 1143572, refers to a compound having the following chemical structure. Some embodiments are directed to a salt or enantiomer of atuveciclib.

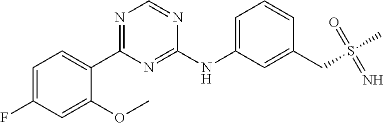 [SMILES: COc1cc(F)ccc1-c1ncnc(Nc2cccc(C[S@](C)(=N)=O)c2)n1]

SNS-032, also known as BMS-387032, refers to a compound having the IUPAC name N-(5-(((5-(1,1-dimethylethyl)-2-oxazolyl)methyl)thio)-2-thiazolyl)-4-piperidinecarboxamide. Some embodiments are directed to a salt or enantiomer of SNS-032.

SR-4835 refers to a compound having the following chemical structure. Some embodiments are directed to a salt or enantiomer of SR-4835.

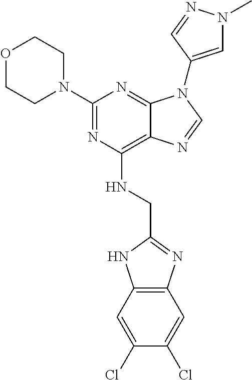 [SMILES: Cn1cc(-n2cnc3c(NCc4nc5cc(Cl)c(Cl)cc5[nH]4)nc(N4CCOCC4)nc32)cn1]

Dinaciclib, also known as SCH-727965, refers to a compound having the IUPAC name (S)-3-(((3-ethyl-5-(2-(2-hydroxyethyl)piperidin-1-yl)pyrazolo[1,5-a]yrimidin-7-yl)amino)methyl)pyridine-1-oxide. Some embodiments are directed to a salt or enantiomer of dinaciclib.

AT7519 refers to a compound having the IUPAC name 4-(2,6-dichlorobenzamido)-N-(piperidin-4-yl)-1H-pyrazole-3-carboxamide. Some embodiments are directed to a salt or enantiomer of AT7519.

Riviciclib, also known as P276-00, refers to a compound having the following chemical structure. Some embodiments are directed to a salt or enantiomer of P276-00.

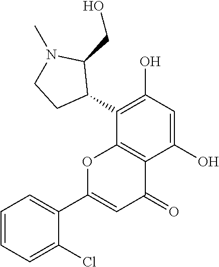 [SMILES: CN1CC[C@@H](c2c(O)cc(O)c3c(=O)cc(-c4ccccc4Cl)oc23)[C@@H]1CO]

Roniciclib, also known as BAY 1000394, refers to a compound having the IUPAC name S-cyclopropylidene-4-((4-(((2R,3R)-3-hydroxybutan-2-yl)oxy)-5-(trifluoromethyl)pyrimidin-2-yl)amino)benzenesulfinamide. Some embodiments are directed to a salt or enantiomer of roniciclib.

RGB-286638 refers to a compound having the following chemical structure. Some embodiments are directed to a salt or enantiomer of RGB-286638.

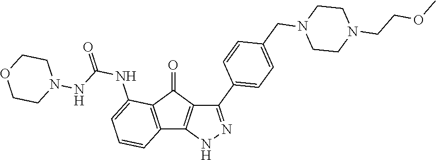 [SMILES: COCCN1CCN(Cc2ccc(-c3n[nH]c4c3C(=O)c3c(NC(=O)NN5CCOCC5)cccc3-4)cc2)CC1]

PHA-793887 refers to a compound having the following chemical structure. Some embodiments are directed to a salt or enantiomer of PHA-793887.

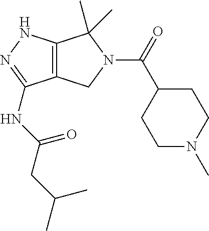 [SMILES: CC(C)CC(=O)Nc1n[nH]c2c1CN(C(=O)C1CCN(C)CC1)C2(C)C]

Seliciclib, also known as Roscovitine or CYC202, refers to a compound having the IUPAC name (2R)-2-{[6-(benzylamino)-9-(propan-2-yl)-9H-purin-2-yl]amino}butan-1-ol. Some embodiments are directed to a salt or enantiomer of seliciclib.

### Administration Schedules

The term “subject,” as used herein, refers to a human individual, including but not limited to a patient undergoing medical evaluation, diagnosis, or treatment. The term encompasses individuals with or without a diagnosed medical condition and may specifically refer to a patient affected by a disease, disorder, or condition relevant to the invention. In the context of oncology-related inventions, “subject” may denote a cancer patient, irrespective of disease stage, prior treatment history, or comorbidities. Unless otherwise specified, the term “subject” is interchangeable with “patient” and includes individuals receiving therapeutic interventions.

The term “administer,” as used herein, refers to the act of delivering a substance, composition, therapy, or intervention to a subject by any suitable route, including but not limited to oral, intravenous, subcutaneous, intramuscular, transdermal, topical, inhalational, or intrathecal administration. The term encompasses both direct administration by a healthcare provider and self-administration by the subject, as well as delivery through medical devices, sustained-release formulations, or other controlled-release mechanisms. Unless otherwise specified, “administer” includes single, repeated, or continuous dosing regimens as appropriate for the intended therapeutic purpose.

The term “with food” or “with a meal” as used herein refers to administration or consumption of a compound or drug to a subject within ten (10) minutes of the subject consuming a food item or meal. In a non-limiting example, an amount of serabelisib and an amount of sapanisertib may be administered to a subject with a meal by administering the amount of serabelisib and the amount of sapanisertib ten minutes before or after the subject consumes the meal.

The term “treat” or “treatment,” as used herein, refers to any act of managing, alleviating, reducing, delaying progression, or eliminating a disease, disorder, condition, or symptom in a subject. Treatment may involve the administration of a therapeutic agent, medical intervention, surgical procedure, lifestyle modification, or any combination thereof, with the goal of improving clinical outcomes, enhancing quality of life, or restoring physiological function. The term encompasses both curative and palliative approaches, as well as measures intended to avoid worsening of a symptom.

The term “therapeutically effective amount,” as used herein, refers to a quantity of an agent, compound, or composition sufficient to produce a desired therapeutic effect in a subject. The effect may include, but is not limited to, reducing the severity or progression of a disease, alleviating symptoms, enhancing biological function, or achieving a measurable clinical benefit. The specific amount may vary depending on factors such as the subject's condition, age, weight, disease severity, route of administration, and individual response. Unless otherwise specified, a therapeutically effective amount encompasses doses that achieve the intended effect without causing unacceptable toxicity or adverse effects.

The term “interval,” as used herein, refers to a unit of time in which a schedule of treatments occurs. For example, a four-week interval refers to four consecutive weeks in which a subject undergoes a series of treatments as described herein. In some embodiments, intervals are repeated. For example, a four-week interval comprising a treatment schedule may be followed by a second four-week interval comprising the treatment schedule.

In some embodiments, the method comprises administering one or more anti-cancer agents that do not directly inhibit enzymes in the PI3K/AKT/mTOR pathway (e.g., paclitaxel) for one day per week.

In some embodiments, after administering the one or more anti-cancer agents that do not directly inhibit enzymes in the PI3K/AKT/mTOR pathway (e.g., paclitaxel) for three consecutive weeks, the one or more anti-cancer agents that do not directly inhibit enzymes in the PI3K/AKT/mTOR pathway (e.g., paclitaxel) are not administered on a 4^{th} week immediately following the three consecutive weeks.

In some embodiments, the method comprises administering one or more compounds that inhibit two or more enzymes of the PI3K/AKT/mTOR pathway for three days per week. In some embodiments, the method comprises administering one or more compounds that inhibit two or more enzymes of the PI3K/AKT/mTOR pathway for three consecutive days per week.

In some embodiments, the method comprises administering one or more compounds that inhibit two or more enzymes of the PI3K/AKT/mTOR pathway with food. In some embodiments, the method comprises administering one or more compounds that inhibit two or more enzymes of the PI3K/AKT/mTOR pathway at the same time and with food. In some embodiments, the one or more compounds that inhibit two or more enzymes of the PI3K/AKT/mTOR pathway are administered with food. In some embodiments, the one or more compounds that inhibit two or more enzymes of the PI3K/AKT/mTOR pathway are administered with a meal. In some embodiments, the one or more compounds that inhibit two or more enzymes of the PI3K/AKT/mTOR pathway are serabelisib and/or sapanisertib.

In some embodiments, the method comprises administering one or more anti-cancer agents that do not directly inhibit enzymes in the PI3K/AKT/mTOR pathway for one day followed the next day by administering the one or more compounds that inhibit two or more enzymes of the PI3K/AKT/mTOR pathway for three consecutive days.

In some embodiments, the method comprises a four-week interval:
(a) wherein each week of the four-week interval comprises administering the one or more compounds that inhibit two or more enzymes of the PI3K/AKT/mTOR pathway for three consecutive days,
(b) the first three weeks of the four-week interval each comprises administering the one or more anti-cancer agents that do not directly inhibit enzymes in the PI3K/AKT/mTOR pathway for one day,
(c) and the 4^{th} week of the four-week interval does not comprise one or more anti-cancer agents that do not directly inhibit enzymes in the PI3K/AKT/mTOR pathway.

In some embodiments, the method comprises administering a dual mTORC1 and mTORC2 inhibitor (e.g., sapanisertib) for three days per week. In some embodiments, the method comprises administering a dual mTORC1 and mTORC2 inhibitor (e.g., sapanisertib) for three consecutive days per week.

In some embodiments, the method comprises administering one or more anti-cancer agents that do not directly inhibit enzymes in the PI3K/AKT/mTOR pathway (e.g., paclitaxel) for one day followed the next day by administering a dual mTORC1 and mTORC2 inhibitor (e.g., sapanisertib) for three consecutive days.

In some embodiments, the method comprises a four-week interval:
(a) wherein each week of the four-week interval comprises administering a dual mTORC1 and mTORC2 inhibitor (e.g., sapanisertib) for three consecutive days,
(b) the first three weeks of the four-week interval each comprises administering the one or more anti-cancer agents that do not directly inhibit enzymes in the PI3K/AKT/mTOR pathway (e.g., paclitaxel) for one day, and
(c) the 4^{th} week of the four-week interval does not comprise one or more anti-cancer agents that do not directly inhibit enzymes in the PI3K/AKT/mTOR pathway (e.g., paclitaxel).

In some embodiments, the method comprises administering a PI3K-alpha inhibitor (e.g., serabelisib) for three days per week. In some embodiments, the method comprises administering a PI3K-alpha inhibitor (e.g., serabelisib) for three consecutive days per week.

In some embodiments, the method comprises administering one or more anti-cancer agents that do not directly inhibit enzymes in the PI3K/AKT/mTOR pathway (e.g., paclitaxel) for one day followed the next day by administering a PI3K-alpha inhibitor (e.g., serabelisib) for three consecutive days.

In some embodiments, the method comprises a four-week interval:
(a) wherein each week of the four-week interval comprises administering a PI3K-alpha inhibitor (e.g., serabelisib) for three consecutive days,
(b) the first three weeks of the four-week interval each comprises administering the one or more anti-cancer agents that do not directly inhibit enzymes in the PI3K/AKT/mTOR pathway (e.g., paclitaxel) for one day, and
(c) the 4^{th} week of the four-week interval does not comprise one or more anti-cancer agents that do not directly inhibit enzymes in the PI3K/AKT/mTOR pathway (e.g., paclitaxel).

In some embodiments, the method comprises administering a dual mTORC1 and mTORC2 inhibitor (e.g., sapanisertib) and a PI3K-alpha inhibitor (e.g., serabelisib) for three days per week. In some embodiments, the method comprises administering a dual mTORC1 and mTORC2 inhibitor (e.g., sapanisertib) and a PI3K-alpha inhibitor (e.g., serabelisib) for three consecutive days per week.

In some embodiments, the method comprises administering one or more anti-cancer agents that do not directly inhibit enzymes in the PI3K/AKT/mTOR pathway (e.g., paclitaxel) for one day followed the next day by administering a dual mTORC1 and mTORC2 inhibitor (e.g., sapanisertib) and a PI3K-alpha inhibitor (e.g., serabelisib) for three consecutive days.

In some embodiments, the method comprises a four-week interval:
(a) wherein each week of the four-week interval comprises administering a dual mTORC1 and mTORC2 inhibitor (e.g., sapanisertib) and a PI3K-alpha inhibitor (e.g., serabelisib) for three consecutive days,
(b) the first three weeks of the four-week interval each comprises administering the one or more anti-cancer agents that do not directly inhibit enzymes in the PI3K/AKT/mTOR pathway (e.g., paclitaxel) for one day, and
(c) the 4^{th} week of the four-week interval does not comprise one or more anti-cancer agents that do not directly inhibit enzymes in the PI3K/AKT/mTOR pathway (e.g., paclitaxel).

In some embodiments, paclitaxel 40-100 mg/m² is administered on day 1, day 8 and day 15, serabelisib 100-300 mg and sapanisertib 1-4 mg is administered on day 2, day 3, day 4, day 9, day 10, day 11, day 16, day 17, day 18, day 23, day 24 and day 25, repeated in a monthly cycle.

In some embodiments, paclitaxel 40-100 mg/m² is administered on day 1, day 8 and day 15, serabelisib 100-300 mg and sapanisertib 1-4 mg is administered on day 2, day 3, day 4, day 9, day 10, day 11, day 16, day 17, day 18, day 23, day 24 and day 25, repeated in a 28-day cycle.

In some embodiments, paclitaxel 80 mg/m² is administered on day 1, day 8 and day 15, serabelisib 200 mg and sapanisertib 3 mg is administered on day 2, day 3, day 4, day 9, day 10, day 11, day 16, day 17, day 18, day 23, day 24 and day 25, repeated in a monthly cycle.

In some embodiments, paclitaxel 80 mg/m² is administered on day 1, day 8 and day 15, serabelisib 200 mg and sapanisertib 3 mg is administered on day 2, day 3, day 4, day 9, day 10, day 11, day 16, day 17, day 18, day 23, day 24 and day 25, repeated in a 28-day cycle.

In some embodiments, serabelisib 50 mg-600 mg is administered once per day. In some embodiments, serabelisib 50 mg-600 mg is administered once daily for three days per week followed by four days without serabelisib repeated in a weekly cycle. In some embodiments, Serabelisib 100 mg-300 mg is administered once daily for three days per week followed by four days without serabelisib repeated in a weekly cycle.

In some embodiments, sapanisertib 1-6 mg is administered once per day. In some embodiments, sapanisertib 1-6 mg is administered daily for three days per week followed by four days without sapanisertib repeated in a weekly cycle. In some embodiments, sapanisertib 1-3 mg is administered daily for three days per week followed by four days without sapanisertib repeated in a weekly cycle.

In some embodiments, paclitaxel 40-100 mg/m² is administered once per week. In some embodiments, paclitaxel 40-100 mg/m² is administered once per week for one week followed by one week without paclitaxel repeated in a two-week cycle. In some embodiments, paclitaxel 40-100 mg/m² is administered once per week for two weeks followed by two weeks without paclitaxel repeated in a four-week cycle.

In some embodiments, paclitaxel 40-100 mg/m² is administered once per week for three weeks followed by one week without paclitaxel repeated in a four-week cycle. In some embodiments, paclitaxel 80-90 mg/m² is administered once per week. In some embodiments, paclitaxel 80-90 mg/m² is administered once per week for one week followed by one week without paclitaxel repeated in a two-week cycle.

In some embodiments, paclitaxel 80-90 mg/m² is administered once per week for two weeks followed by two weeks without paclitaxel repeated in a four-week cycle. In some embodiments, paclitaxel 80-90 mg/m² is administered once per week for three weeks followed by one week without paclitaxel repeated in a four-week cycle. In some embodiments, paclitaxel 100-250 mg/m² administration is repeated every 21 days. In some embodiments, paclitaxel 100-250 mg/m² administration is repeated every 28 days.

In some embodiments, carboplatin is administered as 5-6 mg min/mL (milligrams per minute per millilitre). In some embodiments, carboplatin is administered as 5-6 mg min/mL, repeated every 21 days. In some embodiments, paclitaxel 100-250 mg/m² is administered to the subject with carboplatin mg min/mL every 21 days.

In some embodiments, a Fulvestrant 500 mg loading dose (divided into two 250 mg doses) is administered on day 1, day 15, day 29, and then a 500 mg maintenance dose is administered every 28 days thereafter.

In some embodiments, palbociclib 75-150 mg is administered once daily for 21 days and then palbociclib is not administered for seven days. In some embodiments, this is repeated on as a 28-day cycle.

In some embodiments, palbociclib 125 mg is administered once daily for 21 days and then palbociclib is not administered for seven days. In some embodiments, this is repeated on a 28-day cycle.

In some embodiments, elacestrant 100-600 mg is administered once daily. In some embodiments, elacestrant 345 mg is administered once daily.

In some embodiments, ribociclib 200-800 mg is administered once daily for 21 days and then ribociclib is not administered for seven days. In some embodiments, this is repeated on a 28-day cycle.

In some embodiments, ribociclib 600 mg is administered once daily for 21 days and then ribociclib is not administered for seven days. In some embodiments, this is repeated on a 28-day cycle repeated in a 28-day cycle.

In some embodiments, Abemaciclib is administered twice daily. For example, in some embodiments, about 200 mg/day to about 600 mg/day of Abemaciclib is administered twice per day. (E.g., about 100 mg twice per day to about 300 mg twice per day.) In some embodiments, abemaciclib 100-300 mg is administered twice daily. In some embodiments, abemaciclib 150 mg is administered twice daily.

In some embodiments, selinexor 60-80 mg is administered once weekly. In some embodiments, selinexor 80 mg is administered once weekly for subjects having a BMI≥20 kg/m². In some embodiments, Selinexor 60 mg is administered once weekly for subject having a BMI<20 kg/m².

In some embodiments, inavolisib 5-20 mg is administered daily. In some embodiments, inavolisib 9 mg is administered daily.

In some embodiments, alpelisib 100-400 mg is administered daily. In some embodiments, alpelisib 200-300 mg is administered daily.

In some embodiments, everolimus 2.5-10 mg is administered daily. In some embodiments, temsirolimus 25 mg is administered once weekly.

### Insulin-Suppressing Diet

In some embodiments, the methods of treating cancer herein further include administering an insulin-suppressing diet.

Some embodiments are directed to methods of treating cancer in a subject in need thereof, comprising administering to the subject in need thereof:
(a) a therapeutically effective amount of one or more compounds that inhibit two or more enzymes of the PI3K/AKT/mTOR pathway,
(b) a therapeutically effective amount of one or more anti-cancer agents that do not directly inhibit enzymes in the PI3K/AKT/mTOR pathway, and
(c) an insulin-suppressing diet.

Some embodiments are directed to methods of treating cancer in a subject in need thereof, comprising administering to the subject in need thereof:
(a) a therapeutically effective amount of a dual mTORC1 and mTORC2 inhibitor,
(b) a therapeutically effective amount of a PI3K-alpha inhibitor,
(c) a therapeutically effective amount of one or more anti-cancer agents that do not directly inhibit enzymes in the PI3K/AKT/mTOR pathway, and
(d) an insulin-suppressing diet.

The term “diet,” as used herein, refers to the nutritional intake consumed by the subject from meals, snacks, and drinks throughout the day. For example, the diet may be a food-based diet that comprises the subject's food and beverages over the course of a day. The diet may also comprise meal replacement products, medical food, or parenteral nutritional intake.

The term “insulin-suppressing diet,” as used herein, refers to a dietary regimen designed to reduce insulin secretion, lower circulating insulin levels, or improve insulin sensitivity in a subject. Such a diet typically consists of foods and macronutrient compositions that minimize postprandial insulin spikes, including but not limited to low-carbohydrate, ketogenic, low-glycemic index, high-fiber, or protein-modulated dietary approaches. An insulin-suppressing diet may restrict refined sugars, processed carbohydrates, and high-glycemic foods while emphasizing healthy fats, lean proteins, and complex carbohydrates with slow glucose release. The diet may have a specific ratio of fat to combined carbohydrates plus protein content on a weight/weight basis. The diet may be implemented for therapeutic or metabolic management purposes to modulate insulin signaling and glucose metabolism.

The term “macronutrient content,” as used herein, refers to the relative composition and proportion of the primary nutrients required for energy production and metabolic function in a diet, food product, or nutritional formulation. Macronutrient content includes carbohydrates, proteins, and fats, which collectively contribute to the total caloric intake of a subject. The specific macronutrient ratios may vary depending on dietary strategy, therapeutic goals, or metabolic requirements. Carbohydrates may include simple sugars, complex polysaccharides, and dietary fiber; proteins may be derived from animal or plant sources and provide essential and non-essential amino acids; and fats may include saturated, unsaturated, and polyunsaturated fatty acids. Unless otherwise specified, macronutrient content may be expressed as absolute amounts (grams) or relative percentages of total caloric intake and may be adjusted based on individualized dietary needs, metabolic conditions, or therapeutic interventions.

The term “by w/w % of the macronutrient content,” as used herein, refers to the proportion of each macronutrient—carbohydrates, proteins, and fats—expressed as a weight/weight percentage of the macronutrient composition of a food product, dietary formulation, or nutritional regimen. The w/w % of a macronutrient is calculated as the weight of the specific macronutrient divided by the total weight of the macronutrients, multiplied by 100. Unless otherwise specified, macronutrient content by w/w % may be used to define specific dietary compositions, such as low-carbohydrate, high-protein, or high-fat diets, and may be tailored for therapeutic, metabolic, or nutritional applications.

The term “total carbohydrates,” as used herein, refers to the sum of all carbohydrate-containing components in a food product, dietary formulation, or nutritional regimen, including but not limited to sugars, starches, and dietary fiber. Total carbohydrates may be expressed in absolute amounts (e.g., grams per serving) or as a percentage of the total composition by weight (w/w %) or caloric contribution. The measurement of total carbohydrates may include monosaccharides such as glucose and fructose, disaccharides such as sucrose and lactose, oligosaccharides, and polysaccharides such as starch and cellulose. Unless otherwise specified, total carbohydrates may be adjusted for net carbohydrate content by subtracting fiber and certain sugar alcohols, particularly in low-carbohydrate or insulin-suppressing dietary approaches, and may be tailored for metabolic, therapeutic, or nutritional applications.

In some embodiments, the methods described herein further comprise administering an insulin-suppressing diet to the subject in need thereof. In some embodiments, the insulin-suppressing diet prevents glucose and insulin increases in response to administration of PI3K/AKT/mTOR inhibitor(s) and improves antitumor efficacy of the PI3K/AKT/mTOR inhibitor(s).

In some embodiments, the insulin-suppressing diet comprises about 65% to about 80% of fat by w/w % of the macronutrient content. In some embodiments, the insulin-suppressing diet comprises about 8% to about 25% of protein by w/w % of the macronutrient content. In some embodiments, the insulin-suppressing diet comprises about 8% to about 15% of total carbohydrates by w/w % of the macronutrient content. In some embodiments, the insulin-suppressing diet comprises about 65% to about 80% of fat, about 8% to about 25% of protein, and about 8% to about 15% of total carbohydrates by w/w % of the macronutrient content.

In some embodiments, the insulin-suppressing diet comprises about 15% to about 35% of protein and total carbohydrates by w/w % of the macronutrient content.

### Cancer

In some embodiments, the cancer is uterine cancer, breast cancer, anal cancer, head and neck cancer, bladder cancer, colorectal cancer, skin cancer, ovarian cancer, ampullary cancer, glioma, small bowel cancer, cancer of unknown primary, lung cancer, esophagogastric cancer, renal cell carcinoma, or prostate cancer.

In some embodiments, the cancer is uterine cancer. In some embodiments, the uterine cancer is endometrial cancer. In some embodiments, the uterine cancer is uterine sarcoma. In some embodiments, the uterine cancer is cervical cancer.

In some embodiments, the cancer is breast cancer.

In some embodiments, the cancer is anal cancer.

In some embodiments, the cancer is head and neck cancer.

In some embodiments, the cancer is bladder cancer.

In some embodiments, the cancer is colorectal cancer.

In some embodiments, the cancer is skin cancer. In some embodiments, the skin cancer is melanoma.

In some embodiments, the cancer is ovarian cancer.

In some embodiments, the cancer is ampullary cancer.

In some embodiments, the cancer is glioma.

In some embodiments, the cancer is small bowel cancer.

In some embodiments, the cancer is cancer of unknown primary.

In some embodiments, the cancer is lung cancer. In some embodiments, the lung cancer is small cell lung cancer. In some embodiments, the lung cancer is non-small cell lung cancer.

In some embodiments, the cancer is esophagogastric cancer. In some embodiments, the esophagogastric cancer is esophageal cancer.

In some embodiments, the cancer is renal cell carcinoma.

In some embodiments, the cancer is prostate cancer.

The term mutation, as used herein refers to any alteration in the nucleotide sequence of a gene, genome, or genetic material, including substitutions, insertions, deletions, frameshift mutations, copy number variations, and structural variations such as translocations, inversions, or fusions. Mutations may occur spontaneously, arise from DNA replication errors, result from environmental exposure, or be induced through mutagens. The effects of mutations may include silent, missense, nonsense, gain-of-function, loss-of-function, or dominant-negative changes, impacting gene expression, protein function, or cellular behavior. The term further encompasses alterations in both coding and non-coding regions, including regulatory elements that modulate gene expression. Unless otherwise specified, mutations described herein may be naturally occurring, disease-associated or therapeutically targeted.

In some embodiments, the cancer comprises a PI3K/AKT/mTOR pathway mutation. The term “PI3K/AKT/mTOR pathway mutation,” as used herein, refers to any genetic alteration that affects the function, expression, regulation, or signaling of one or more components within the phosphoinositide 3-kinase (PI3K)/protein kinase B (AKT)/mechanistic target of rapamycin (mTOR) pathway. Such mutations may include, but are not limited to, single nucleotide substitutions, insertions, deletions, amplifications, gene fusions, structural variations, or post-transcriptional modifications that alter protein activity, stability, or interaction with regulatory factors. PI3K/AKT/mTOR pathway mutations may involve genes encoding PI3K isoforms (PIK3CA, PIK3CB, PIK3CG, PIK3CD, PIK3C2B), regulatory subunits (PIK3R1), AKT isoforms (AKT1, AKT2, AKT3), mTOR and its associated complex proteins (MTOR, RPTOR, RAPTOR, RICTOR, MLST8, DEPTOR), downstream effectors (S6K, 4E-BP1, AKT1S1), tumor suppressors and regulators (PTEN, STK11, TSC1, TSC2, PPP2R1A, INPP4B), signaling intermediates (PDK1, RHEB, AMPK, GSK3), and additional complex components (DEPDC5, NPRL2, NPRL3). Such mutations may result in constitutive activation or loss of function, leading to dysregulated cell growth, survival, metabolism, or therapeutic resistance in cancer and other diseases. Unless otherwise specified, PI3K/AKT/mTOR pathway mutations may be naturally occurring, somatic, germline, or targeted for therapeutic intervention. In some embodiments, the PI3K/AKT/mTOR pathway mutation is a mutation of PIK3CA, PIK3CB, PIK3CG, PIK3CD, PIK3C2B, PIK3R1, AKT1, AKT2, AKT3, MTOR, RPTOR, RAPTOR, RICTOR, MLST8, DEPTOR, S6K, 4E-BP1, AKT1S1, PTEN, STK11, TSC1, TSC2, PPP2R1A, INPP4B, PDK1, RHEB, AMPK, GSK3, DEPDC5, NPRL2, or NPRL3. The preceding abbreviations refer to the following:
PIK3CA—Phosphatidylinositol-4,5-bisphosphate 3-kinase catalytic subunit alpha
PIK3CB—Phosphatidylinositol-4,5-bisphosphate 3-kinase catalytic subunit beta
PIK3CG—Phosphatidylinositol-4,5-bisphosphate 3-kinase catalytic subunit gamma
PIK3CD—Phosphatidylinositol-4,5-bisphosphate 3-kinase catalytic subunit delta
PIK3C2B—Phosphatidylinositol-4-phosphate 3-kinase C2 domain-containing subunit beta
PIK3R1—Phosphatidylinositol 3-kinase regulatory subunit alpha
AKT1—RAC-alpha serine/threonine-protein kinase
AKT2—RAC-beta serine/threonine-protein kinase
AKT3—RAC-gamma serine/threonine-protein kinase
MTOR—Mechanistic target of rapamycin (serine/threonine kinase)
RPTOR—Regulatory-associated protein of mTOR
RAPTOR—Regulatory-associated protein of mTOR (alternative name for RPTOR)
RICTOR—Rapamycin-insensitive companion of mTOR
MLST8—MTOR-associated protein, LST8 homolog
DEPTOR—DEP domain-containing mTOR-interacting protein
S6K—Ribosomal protein S6 kinase beta-1 (RPS6KB1)
4E-BP1—Eukaryotic translation initiation factor 4E-binding protein 1 (EIF4EBP1)
AKT1S1—Proline-rich AKT substrate 1
PTEN—Phosphatase and tensin homolog
STK11—Serine/threonine-protein kinase 11
TSC1—Tuberous sclerosis complex 1 protein (Hamartin)
TSC2—Tuberous sclerosis complex 2 protein (Tuberin)
PPP2R1A—Serine/threonine-protein phosphatase 2A 65 kDa regulatory subunit A alpha isoform
INPP4B—Inositol polyphosphate-4-phosphatase type II B
PDK1—3-Phosphoinositide-dependent protein kinase 1
RHEB—Ras homolog enriched in brain
AMPK—5′-AMP-activated protein kinase
GSK3—Glycogen synthase kinase 3 (includes GSK3A and GSK3B isoforms)
DEPDC5—DEP domain-containing protein 5
NPRL2—Nitrogen permease regulator-like 2
NPRL3—Nitrogen permease regulator-like 3

In some embodiments, the PI3K/AKT/mTOR pathway mutation comprises one or more mutations in the following genes PIK3CA, MTOR, PIK3R1, PTEN, TSC1, TSC2, AKT1, AKT2, AKT3, PPP2R1A, PIK3C2B, INPP4B, PDK1, RHEB, STK11, RPTOR, DEPTOR, RAPTOR, RICTOR, MLST8, AKT1S1, DEPDC5, NPRL2, NPRL3, AMPK, or combinations thereof.

In some embodiments, the PI3K/AKT/mTOR pathway mutation comprises a PIK3CA amplification. In some embodiments, the cancer is not PIK3CA mutated.

In some embodiments, the PI3K/AKT/mTOR pathway mutation comprises a PTEN deletion.

In some embodiments, the PI3K/AKT/mTOR pathway mutation comprises a PIK3R1 deletion.

In some embodiments, the PI3K/AKT/mTOR pathway mutation comprises a PIK3CA a helical domain mutation. In some embodiments, the PI3K/AKT/mTOR pathway mutation comprises a PIK3CA kinase domain mutation.

In some embodiments, the cancer is ER positive. In some embodiments, the cancer is ER negative. In some embodiments, the cancer is HER2 positive. In some embodiments, the cancer is HER2 negative.

In some embodiments, the cancer was previously treated with a taxane. In some embodiments, the cancer is taxane resistant. The term “taxane resistant,” as used herein, refers to the condition in which cancer cells, tumors, or a subject's disease state exhibits reduced sensitivity or an impaired therapeutic response to taxane-based chemotherapy, including but not limited to paclitaxel, docetaxel, cabazitaxel, and their formulations such as albumin-bound paclitaxel. Taxane resistance may arise through various mechanisms, including overexpression of efflux transporters such as P-glycoprotein (ABCB1), alterations in microtubule structure or function, mutations in tubulin proteins, activation of alternative survival pathways such as the PI3K/AKT/mTOR pathway, increased anti-apoptotic signaling, or enhanced DNA damage repair mechanisms. Resistance may be intrinsic, occurring before treatment, or acquired following prolonged taxane exposure. Unless otherwise specified, taxane resistance may be partial or complete and may necessitate alternative therapeutic strategies, such as combination therapy with non-taxane chemotherapeutic agents, targeted inhibitors, immunotherapies, or agents designed to overcome resistance mechanisms. In some embodiments, the taxane was paclitaxel, docetaxel, or a combination thereof.

Phosphorylated 4E-BP1 is used as a biomarker in healthy cells or tissue to assess inhibition of the PI3K/AKT/mTOR pathway. The term “reduces phosphorylated 4E-BP1,” as used herein, refers to the ability of a compound, molecule, or agent to decrease the levels, activity, or cellular abundance of phosphorylated eukaryotic translation initiation factor 4E-binding protein 1 (4E-BP1), a downstream effector of the mechanistic target of rapamycin (mTOR) signaling pathway, compared to an untreated proliferating tumor. Reduction in phosphorylated 4E-BP1 may occur through direct or indirect mechanisms, including inhibition of upstream kinases such as mTORC1, modulation of regulatory proteins, enhancement of phosphatase activity, or interference with translation initiation processes. The reduction of phosphorylated 4E-BP1 may range from about 50% to about 100% relative to an untreated proliferating tumor, as determined by biochemical, molecular, or functional assays such as Western blotting, immunohistochemistry, mass spectrometry, or other quantification methods. Such a reduction may result in decreased cap-dependent protein translation, altered cellular metabolism, inhibition of tumor growth, or increased sensitivity to anti-cancer therapies. Unless otherwise specified, reducing phosphorylated 4E-BP1 may be achieved by pharmacological agents, biologics, RNA-based therapies, or other molecular interventions that modulate the PI3K/AKT/mTOR signaling pathway.

In some embodiments, the methods herein reduce phosphorylated 4E-BP1 relative to untreated tumor tissue. The term “untreated tumor tissue,” as used herein, refers to tumor tissue that has not been exposed to the therapeutic intervention, compound, or treatments described herein. Untreated tumor tissue serves as a control for evaluating the effects of the disclosed methods and may originate from in vivo models, ex vivo tumor samples, or in vitro cultured tumor cells. The term encompasses tumor tissue that has not been subjected to pharmacological agents, radiation therapy, genetic modifications, immunotherapies, or other experimental treatments, though it may include tissue maintained under standard growth conditions, including exposure to physiological or pathological factors such as nutrients, insulin, other growth factors, hypoxia, or immune interactions. Unless otherwise specified, untreated tumor tissue provides a baseline reference for assessing changes in biomarkers, including phosphorylated 4E-BP1 levels, and serves as a comparator to determine the relative efficacy of the disclosed methods in reducing phosphorylated 4E-BP1.

As described herein, an outcome measuring demonstrating the effect of the treatment includes Objective Response Rate (ORR), which refers to the proportion of subjects with measurable disease at baseline who upon treatment have confirmed complete response (CR) or partial response (PR). In some embodiments, the time frame for this outcome measure is two years.

Additionally, a second treatment outcome measure is progression-free survival (PFS), which refers to the time from first dose until the date of disease progression or death.

Furthermore, another treatment outcome measure is progression-free survival (PFS) at six months, which refers to the PFS rate at six months.

Overall survival (OS) is another outcome measure demonstrating the effect of the treatment. OS refers to the time from first dose to death.

Another treatment outcome measure is clinical benefit rate (CBR), which refers to the percentage of subjects who achieve complete response (CR), partial response (PR), or have stable disease (SD) of at least 16 or 24 weeks, where these outcomes are assessed by best overall response (BOR).

Another treatment outcome measure is disease control rate (DCR), which refers to the percentage of subjects who achieve complete response (CR), partial response (PR), or have stable disease (SD), where these outcomes are assessed by best overall response (BOR).

Yet another treatment outcome measure is duration of response (DoR), which, for participants with confirmed CR or PR, refers to the time from response until date of documented disease progression or death.

In some embodiments, cancer response and progression will be based on RECIST v1.1 criteria, which are described in the Examples. In some embodiments, ORR and PFS are assessed by RECIST v1.1.

Safety and tolerability of drugs by assessment of adverse events (AEs)/serious adverse events (SAEs) are graded according to the National Cancer Institute Common Terminology Criteria for Adverse Events (NCI CTCAE V5.0).

Any of the features of the methods described herein are also applicable to uses of compounds for treating cancer, uses in the manufacture of a medicament for treating cancer, compounds for use in treating cancer, and kits comprising compounds for treating cancer.

### Methods of Treating Endometrial Cancer

The present disclosure also provides a method of treating endometrial cancer in a human subject in need thereof, the method comprising administering to the subject a therapeutically effective amount of a combination comprising: an amount of orally administered sapanisertib, an amount of orally administered serabelisib, and an amount of intravenously administered paclitaxel.

In some embodiments, the treating comprises providing a progression-free survival (PFS) of at least six months, preferably at least nine months, more preferably at least 12 months to the subject.

In some embodiments, the treating comprises providing an overall survival (OS) of at least nine months, at least 12 months, or at least 15 months to the subject.

In some embodiments, the treating comprises providing a duration of response (DoR) of at least six months, at least nine months, at least 12 months, or at least 15 months to the subject.

In some embodiments, the treating comprises providing a complete response (CR) or a partial response (PR) to the subject.

In some embodiments, the treating comprises providing to the subject a progression-free survival of at least six months from the start of the treatment.

In some embodiments, the treating comprises providing a complete response (CR), a partial response (PR), or a stable disease (SD) of at least 16 to 24 weeks to the subject.

In some embodiments, the treating comprises providing the subject with an at least 35% likelihood, preferably an at least 40% likelihood, more preferably an at least 60% likelihood of complete response (CR) or partial response (PR) to the treatment.

In some embodiments, the treating comprises providing the subject with an at least 50% likelihood, preferably an at least 60% likelihood, more preferably an at least 80% likelihood of achieving progression-free survival (PFS) at six months from the start of the treatment.

In some embodiments, the treating comprises providing the subject with an at least 50% likelihood, preferably an at least 60% likelihood, more preferably an at least 80% likelihood of achieving a complete response (CR), a partial response (PR), or having stable disease (SD) of at least 16 to 24 weeks in response to the treatment.

In some embodiments, the subject is orally administered about 1-4 mg of sapanisertib, orally administered about 100-300 mg of serabelisib, and intravenously administered about 40-100 mg/m² of paclitaxel.

In some embodiments, the subject is orally administered about 3-4 mg of sapanisertib, orally administered about 200 mg of serabelisib, and intravenously administered about 80 mg/m² of paclitaxel.

In some embodiments, the amount of paclitaxel is administered, then the amount of serabelisib and the amount of sapanisertib are administered on each of the three days following the paclitaxel administration.

In some embodiments, the amount of serabelisib and the amount of sapanisertib are administered at about the same time.

In some embodiments, the method comprises repeating the administration of sapanisertib, serabelisib, and paclitaxel over the course of a treatment interval.

In some embodiments, the method comprises a four-week interval, wherein each week of the four-week interval comprises administering the amount of sapanisertib and the amount of serabelisib for each of three consecutive days, wherein the first three weeks of the four-week interval each comprises administering the amount of paclitaxel for one day, and wherein paclitaxel is not administered during the 4^{th} week of the four-week interval.

In some embodiments, 40-100 mg/m² paclitaxel is administered on day 1, day 8, and day 15 of the interval; and wherein 100-300 mg serabelisib and 1-4 mg sapanisertib are administered on day 2, day 3, day 4, day 9, day 10, day 11, day 16, day 17, day 18, day 23, day 24 and day 25 of the interval.

In some embodiments, the method further comprises administering an insulin suppressing diet to the subject in need thereof. In some embodiments, the insulin suppressing diet comprises about 65% to about 80% of fat by w/w % of the macronutrient content. In some embodiments, the insulin suppressing diet comprises about 8% to about 25% of protein by w/w % of the macronutrient content. In some embodiments, the insulin suppressing diet comprises about 8% to about 15% of total carbohydrates by w/w % of the macronutrient content. In some embodiments, the insulin suppressing diet comprises about 65% to about 80% of fat, about 8% to about 25% of protein, and about 8% to about 15% of total carbohydrates by w/w % of the macronutrient content. In some embodiments, the insulin suppressing diet comprises about 15% to about 35% of protein and total carbohydrates by w/w % of the macronutrient content.

In some embodiments, prior to the method of treating the subject previously received one to four prior lines of chemotherapy, preferably including a systemic therapy, more preferably including a platinum-based chemotherapy and/or a checkpoint inhibitor.

In some embodiments, the subject has a mutation identified in a gene of the PI3K/AKT/mTOR pathway, and/or has a mutated PI3K pathway.

In some embodiments, the subject comprises one or more mutations in at least one gene selected from the group consisting of PIK3CB, PIK3CG, PIK3CD, PIK3C2B, RPTOR, RAPTOR, RICTOR, MLST8, DEPTOR, S6K, 4E-BP1, AKT1S1, STK11, TSC2, INPP4B, PDK1, RHEB, AMPK, GSK3, DEPDC5, NPRL2, and NPRL3.

In some embodiments, the subject comprises one or more mutations in at least one gene selected from the group consisting of PIK3CA, PIK3R1, AKT1, AKT2, AKT3, MTOR, PTEN, TSC1, PPP2R1A, TP53, RB1, CCNE1, ERBB2, APC, NF1, MYCN, CDKN2A, FGFR1, CDKN2B, CCND1, MYC, EGFR, ERRF11, KRAS, CTNNB1, FBXW7, ABL1, CHEK2, and ARAF.

In some embodiments, the mutation comprises a PIK3CA amplification.

In some embodiments, the endometrial cancer is not PIK3CA mutated.

In some embodiments, the mutation comprises a PTEN deletion.

In some embodiments, the mutation comprises a PIK3R1 deletion.

In some embodiments, the mutation comprises a PIK3CA a helical domain mutation.

In some embodiments, the mutation comprises a PIK3CA kinase domain mutation.

In some embodiments, the endometrial cancer is ER positive or ER negative.

In some embodiments, the endometrial cancer is HER2 positive or HER2 negative.

In some embodiments, the endometrial cancer is advanced or recurrent endometrial cancer.

In some embodiments, the treating comprises the subject presenting no serious adverse events, more preferably no adverse events.

In some embodiments, the treating comprises the subject presenting no grade 2 hyperglycemia. In some embodiments, the treating comprises the subject presenting no grade 1 hyperglycemia. In some embodiments, the treating comprises the subject presenting no hyperglycemia.

In some embodiments, the treating comprises providing the subject with a less than 15% likelihood of presenting grade 1 or grade 2 hyperglycemia, more preferably a less than 10% likelihood of presenting grade 1 or grade 2 hyperglycemia.

In some embodiments, the treating comprises the subject presenting no grade 2 stomatitis.

In some embodiments, the treating comprises the subject presenting no grade 1 stomatitis. In some embodiments, the treating comprises the subject presenting no stomatitis.

In some embodiments, the treating comprises providing the subject with a less than 15% likelihood of presenting grade 1 or grade 2 stomatitis, more preferably a less than 10% likelihood of presenting grade 1 or grade 2 stomatitis.

In some embodiments, the treating comprises the subject presenting no grade 3 or grade 4 adverse events.

In some embodiments, the treating comprises the subject presenting no grade 3 or grade 4 hyperglycemia. In some embodiments, the treating comprises the subject presenting no grade 4 hyperglycemia. In some embodiments, the treating comprises the subject presenting no grade 3 hyperglycemia. In some embodiments, the treating comprises the subject presenting no hyperglycemia.

In some embodiments, the treating comprises providing the subject with a less than 30% likelihood of presenting hyperglycemia, preferably with a less than 20% likelihood of presenting hyperglycemia, more preferably with a less than 15% likelihood of presenting hyperglycemia.

In some embodiments, the treating comprises providing the subject with a less than 15% likelihood of presenting grade 3 or grade 4 hyperglycemia, preferably with a less than 10% likelihood of presenting grade 3 or grade 4 hyperglycemia, preferably with a less than 7% likelihood of presenting grade 3 or grade 4 hyperglycemia, more preferably with a less than 5% likelihood of presenting grade 3 or grade 4 hyperglycemia.

In some embodiments, the treating provides the subject with a lower likelihood of experiencing any grade of hyperglycemia than administration of a single-node PI3K pathway inhibitor (e.g., alpelisib). In some embodiments, the treating provides the subject with a lower likelihood of experiencing any grade of hyperglycemia than administration of serabelisib or sapanisertib given alone.

In some embodiments, the treating comprises the subject presenting no grade 3 or grade 4 stomatitis. In some embodiments, the treating comprises the subject presenting no grade 4 stomatitis. In some embodiments, the treating comprises the subject presenting no grade 3 stomatitis. In some embodiments, the treating comprises the subject presenting no stomatitis.

In some embodiments, the treating comprises providing the subject with a less than 80% likelihood of presenting stomatitis, preferably with a less than 65% likelihood of presenting stomatitis, preferably with a less than 50% likelihood of presenting stomatitis, more preferably with a less than 30% likelihood of presenting stomatitis, more preferably with a less than 20% likelihood of presenting stomatitis, more preferably with a less than 15% likelihood of presenting stomatitis.

In some embodiments, the treating comprises providing the subject with a less than 30% likelihood of presenting grade 3 or grade 4 stomatitis, preferably with a less than 20% likelihood of presenting grade 3 or grade 4 stomatitis, preferably with a less than 15% likelihood of presenting grade 3 or grade 4 stomatitis, more preferably with a less than 10% likelihood of presenting grade 3 or grade 4 stomatitis, more preferably with a less than 5% likelihood of presenting grade 3 or grade 4 stomatitis, more preferably with a less than 1% likelihood of presenting grade 3 or grade 4 stomatitis.

In some embodiments, the treating excludes administration of a steroid or corticosteroid. In some embodiments, administration of a steroid or corticosteroid is not prospectively prescribed as a prophylactic measure, e.g., to reduce or prevent stomatitis, mouth ulcers, and/or oral mucositis.

In some embodiments, the treating excludes administration of a prophylactic mouth rinse comprising a steroid or corticosteroid.

In some embodiments, the treating excludes any measures to prevent, ameliorate, treat, or reduce stomatitis.

The present disclosure also provides a method of treating endometrial cancer in a human subject in need thereof without inducing grade 4 stomatitis or without inducing grade 4 hyperglycemia, the method comprising
intravenously administering to the subject 40-100 mg/m² paclitaxel on day 1, day 8, and day 15 of a four-week treatment interval; and
orally administering to the subject 100-300 mg serabelisib and 1-4 mg sapanisertib on day 2, day 3, day 4, day 9, day 10, day 11, day 16, day 17, day 18, day 23, day 24 and day 25 of the four-week treatment interval.

In some embodiments, the method of treatment proceeds without inducing grade 4 stomatitis and without inducing grade 4 hyperglycemia.

In some embodiments, the method of treatment proceeds without inducing grade 4 stomatitis.

In some embodiments, the method of treatment proceeds without inducing grade 3 stomatitis.

In some embodiments, the method of treatment proceeds without inducing grade 4 hyperglycemia.

In some embodiments, the method of treatment proceeds without inducing grade 3 hyperglycemia.

In some embodiments, the treating comprises providing a progression-free survival (PFS) of at least six months, preferably at least nine months, more preferably at least 12 months to the subject.

In some embodiments, the treating comprises providing an overall survival (OS) of at least nine months, at least 12 months, or at least 15 months to the subject.

In some embodiments, the treating comprises providing a duration of response (DoR) of at least six months, at least nine months, at least 12 months, or at least 15 months to the subject.

In some embodiments, the treating comprises providing a complete response (CR), a partial response (PR), or a stable disease (SD) of at least 16 to 24 weeks to the subject.

In some embodiments, the method further comprises administering an insulin suppressing diet to the subject in need thereof.

In some embodiments, the serabelisib and sapanisertib are administered with food and/or with a meal.

Several treatment regimens may be utilized for the method of treating endometrial cancer comprising administration of sapanisertib, serabelisib, and paclitaxel.

In some embodiments, after administering paclitaxel for three consecutive weeks, paclitaxel is not administered on a 4^{th} week immediately following the three consecutive weeks.

In some embodiments, the method comprises administering sapanisertib and serabelisib for three days per week. In some embodiments, the method comprises administering sapanisertib and serabelisib for three consecutive days per week.

In some embodiments, the method comprises administering paclitaxel for one day followed the next day by administering sapanisertib and serabelisib for three consecutive days.

In some embodiments, the method comprises a four-week interval:
wherein each week of the four-week interval comprises administering sapanisertib and serabelisib for three consecutive days, the first three weeks of the four-week interval each comprises administering paclitaxel for one day, and the 4^{th} week of the four-week interval does not comprise administering paclitaxel.

In some embodiments, 40-100 mg/m² paclitaxel is administered on day 1, day 8 and day 15, 100-300 mg serabelisib and 1-4 mg sapanisertib are administered on day 2, day 3, day 4, day 9, day 10, day 11, day 16, day 17, day 18, day 23, day 24 and day 25, repeated in a monthly cycle.

In some embodiments, 40-100 mg/m² paclitaxel is administered on day 1, day 8 and day 15, 100-300 mg serabelisib and 1-4 mg sapanisertib are administered on day 2, day 3, day 4, day 9, day 10, day 11, day 16, day 17, day 18, day 23, day 24 and day 25, repeated in a 28-day cycle.

In some embodiments, 80 mg/m² paclitaxel is administered on day 1, day 8 and day 15, 200 mg serabelisib and sapanisertib 3 mg is administered on day 2, day 3, day 4, day 9, day 10, day 11, day 16, day 17, day 18, day 23, day 24 and day 25, repeated in a monthly cycle.

In some embodiments, paclitaxel 80 mg/m² is administered on day 1, day 8 and day 15, serabelisib 200 mg and 3 mg sapanisertib are administered on day 2, day 3, day 4, day 9, day 10, day 11, day 16, day 17, day 18, day 23, day 24 and day 25, repeated in a 28-day cycle.

In some embodiments, 50 mg-600 mg serabelisib is administered once per day.

In some embodiments, 50 mg-600 mg serabelisib is administered once daily for three days per week followed by four days without serabelisib repeated in a weekly cycle.

In some embodiments, 100 mg-300 mg serabelisib is administered once daily for three days per week followed by four days without serabelisib repeated in a weekly cycle.

In some embodiments, 1-6 mg sapanisertib is administered once per day.

In some embodiments, 1-6 mg sapanisertib is administered daily for three days per week followed by four days without sapanisertib repeated in a weekly cycle.

In some embodiments, 1-3 mg sapanisertib is administered daily for three days per week followed by four days without sapanisertib repeated in a weekly cycle.

In some embodiments, 40-100 mg/m² paclitaxel is administered once per week. In some embodiments, paclitaxel 40-100 mg/m² is administered once per week for one week followed by one week without paclitaxel repeated in a two-week cycle.

In some embodiments, 40-100 mg/m² paclitaxel is administered once per week for two weeks followed by two weeks without paclitaxel repeated in a four-week cycle.

In some embodiments, 40-100 mg/m² paclitaxel is administered once per week for three weeks followed by one week without paclitaxel repeated in a four-week cycle.

In some embodiments, 80-90 mg/m² paclitaxel is administered once per week.

In some embodiments, 80-90 mg/m² paclitaxel is administered once per week for one week followed by one week without paclitaxel repeated in a two-week cycle.

In some embodiments, 80-90 mg/m² paclitaxel is administered once per week for two weeks followed by two weeks without paclitaxel repeated in a four-week cycle.

In some embodiments, 80-90 mg/m² paclitaxel is administered once per week for three weeks followed by one week without paclitaxel repeated in a four-week cycle.

In some embodiments, 100-250 mg/m² paclitaxel administration is repeated every 21 days.

In some embodiments, 100-250 mg/m² paclitaxel administration is repeated every 28 days.

In some embodiments, the treatment of subjects results in an ORR of 35% or higher, 40% or higher, or 60% or higher. In some embodiments, the time frame for this outcome measure is two years.

In some embodiments, the treatment results in a PFS of at least six months, at least nine months, or at least 12 months. In some embodiments, the time frame for this outcome measure is five years.

In some embodiments, the treatment results in a progression-free survival (PFS) at six months in at least 50% of subjects, in at least 60% of subjects, in at least 80% of subjects. In some embodiments, the time frame for this outcome measure is five years.

In some embodiments, the treatment results in an OS of at least nine months, at least 12 months, or at least 15 months. In some embodiments, the time frame for this outcome measure is five years.

In some embodiments, the treatment of subjects results in a CBR of 50% or higher, 60% or higher, or 80% or higher. In some embodiments, the time frame for this outcome measure is five years.

In some embodiments, the treatment results in a DoR of at least six months, at least nine months, or at least 12 months. In some embodiments, the time frame for this outcome measure is five years.

Lastly, safety and tolerability of drugs by assessment of adverse events (AEs)/serious adverse events (SAEs) are graded according to the National Cancer Institute Common Terminology Criteria for Adverse Events (NCI CTCAE V5.0). In some embodiments, AEs and SAEs are measured within two years from the start of the treatment. In some embodiments, hyperglycemia is not detected in the treated subject. In some embodiments, no grade three or higher hyperglycemia events are detected in the treated subject. In some embodiments, stomatitis is not detected in the treated subject. In some embodiments, no grade three or higher stomatitis events are detected in the treated subject.

The method of treating endometrial cancer in a subject in need thereof, comprising administering to the subject in need thereof a therapeutically effective amount of a combination comprising an amount of sapanisertib, an amount of sapanisertib, and an amount of paclitaxel, and optionally placing the subject on an insulin-suppressing diet, may also be useful for a variety of cancers and cancer-related conditions, including, but not limited to genital diseases; endometrial neoplasms; advanced endometrial carcinoma; recurrent endometrial carcinoma; metastatic endometrial carcinoma; endometrial cancer; endometrioid carcinoma; cancer of endometrium; cancer of the endometrium; carcinoma of endometrium; endometrial carcinoma; and endometrium cancer, as well as uterine neoplasms; genital neoplasms, female; urogenital neoplasms; neoplasms by site; neoplasms; uterine diseases; genital diseases, female; female urogenital diseases; female urogenital diseases and pregnancy complications; urogenital diseases; adenocarcinoma; carcinoma; neoplasms, glandular and epithelial; neoplasms by histologic type; ovarian neoplasms; ovarian diseases; adnexal diseases; gonadal disorders; endocrine system diseases; nervous system malformations; nervous system diseases; heredodegenerative disorders, nervous system; neurodegenerative diseases; polyneuropathies; peripheral nervous system diseases; neuromuscular diseases; congenital abnormalities; congenital, hereditary, and neonatal diseases and abnormalities; genetic diseases, inborn; endometrial neoplasms; genital diseases; carcinoma, endometrioid; and hereditary sensory and autonomic neuropathies.

Any of the features of the methods described herein for treating endometrial cancer are also applicable to uses of compounds for treating endometrial cancer, uses in the manufacture of a medicament for treating endometrial cancer, compounds for use in treating endometrial cancer, and kits comprising compounds for treating endometrial cancer.

### Methods of Treating Breast Cancer (e.g., HR+/HER2− Advanced or Metastatic Breast Cancer)

The present disclosure also provides methods of treating breast cancer (e.g., HR+/HER2− advanced or metastatic breast cancer) in a human subject in need thereof, comprising administering to the human subject in need thereof an effective amount of: orally administered sapanisertib and orally administered serabelisib in combination with hormone receptor targeting therapeutic agents, cyclin-dependent kinase (CDK) inhibitors, and lysine acetyltransferase 6 (KAT6) inhibitors, or combinations thereof.

Resistance to CDK inhibitor and/or endocrine therapy has become an important unmet medical need in the treatment of HR+/HER2− metastatic breast cancer. Methods of the present disclosure are also useful for overcoming endocrine therapy resistance and/or CDK inhibitor resistance.

In some embodiments, after disease progression while on hormone receptor targeting therapeutic agents, adding PI3K/AKT/mTOR pathway inhibitors re-sensitizes tumors. In some embodiments, after disease progression while on CDK inhibitors, adding PI3K/AKT/mTOR pathway inhibitors re-sensitizes tumors. An intermittent dosing schedule facilitates chronic administration with less toxicity while preserving pathway suppression. Accordingly, the present disclosure also provides a method of increasing efficacy of a hormone receptor targeting therapeutic agent in a human subject in need thereof having breast cancer, the method comprising administering to the human subject in need thereof an effective amount of: orally administered sapanisertib and orally administered serabelisib in combination with the hormone receptor targeting therapeutic agent. Moreover, the present disclosure also provides a method of increasing efficacy of a CDK inhibitor in a human subject in need thereof having breast cancer, the method comprising administering to the human subject in need thereof an effective amount of: orally administered sapanisertib and orally administered serabelisib in combination with the CDK inhibitor.

In some embodiments, efficacy is determined by ORR, PFS, PFS at 6 months, OS, CBR, DCR, DoR, CR, ORR avoidance of AEs and/or SAEs such as hyperglycemia and/or stomatitis (e.g., oral mucositis).

In some embodiments, the sapanisertib and the serabelisib are administered in combination with one or more hormone receptors targeting therapeutic agents. In some embodiments, the sapanisertib and the serabelisib are administered in combination with one or more CDK inhibitors. In some embodiments, the sapanisertib and the serabelisib are administered in combination with one or more KAT6 inhibitors. In some embodiments, the sapanisertib and the serabelisib are administered in combination with one or more hormone receptors targeting therapeutic agents combined with one or more CDK inhibitors. In some embodiments, the sapanisertib and the serabelisib are administered in combination with one or more hormone receptors targeting therapeutic agents combined with one or more KAT6 inhibitors.

In some embodiments, the serabelisib and the sapanisertib are administered at about the same time. In some embodiments, the serabelisib and the sapanisertib are administered with food.

In some embodiments, the hormone receptor targeting therapeutic agent is an aromatase inhibitor. In some embodiments, the hormone receptor targeting therapeutic agent is anastrozole, letrozole, or exemestane.

In some embodiments, the hormone receptor targeting therapeutic agent is a Selective Estrogen Receptor Degrader (SERD). In some embodiments, the hormone receptor targeting therapeutic agent is amcenestrant, AZD8421, borestrant, brilanestrant, camizestrant, elacestrant, giredestrant, imlunestrant, LSZ102, palazestrant, rintodestrant, or taragarestrant. In some embodiments, the hormone receptor targeting therapeutic agent is orally administered elacestrant, imlunestrant, camizestrant, giredestrant, or palazestrant. In some embodiments, the hormone receptor targeting therapeutic agent is orally administered elacestrant or giredestrant. In some embodiments, the hormone receptor targeting therapeutic agent is orally administered elacestrant.

In some embodiments, the hormone receptor targeting therapeutic agent is intramuscularly administered fulvestrant.

In some embodiments, the method comprises repeating the administration of sapanisertib, serabelisib, and the hormone receptor targeting therapeutic agent over the course of a treatment interval. In some embodiments, the method comprises repeating the administration of sapanisertib, serabelisib, and fulvestrant over the course of a treatment interval. In some embodiments, the method comprises repeating the administration of sapanisertib, serabelisib, and elacestrant over the course of a treatment interval.

In some embodiments, the CDK inhibitor inhibits one of more of CDK2, CDK4, CDK6, CDK7, CDK8, CDK9, CDK19, CDK12, and CDK13. In some embodiments, the CDK inhibitor inhibits CDK4/6, CDK2/9, CDK8/19, or CDK12/13. In some embodiments, the CDK inhibitor is a Pan-CDK inhibitor.

In some embodiments, the CDK inhibitor is abemaciclib, alvocidib, AT7519, atuveciclib, AZD4573, BTX-A51, cirtociclib, dalpiciclib, dinaciclib, ebvaciclib, enitociclib, fadraciclib, INX-315, KB-0742, LY3405105, palbociclib, PHA-793887, RGB-286638, ribociclib, riviciclib, romaciclib, roniciclib, samuraciclib, seliciclib, SNS-032, SR-4835, SY-5609, tacaciclib, tegtociclib, trilaciclib, voruciclib, or zotiraciclib. In some embodiments, the CDK inhibitor is palbociclib, ribociclib, abemaciclib, trilaciclib, or dalpiciclib. In some embodiments, the CDK inhibitor is palbociclib, ribociclib, abemaciclib, or dalpiciclib. In some embodiments, the CDK inhibitor is orally administered palbociclib.

In some embodiments, the method the method comprises repeating the administration of sapanisertib, serabelisib, fulvestrant, and palbociclib over the course of a treatment interval.

In some embodiments, the method comprises a four-week interval: wherein each week of the four-week interval comprises administering the sapanisertib and the serabelisib for each of three consecutive days.

In some embodiments, 100-400 mg serabelisib and 1-5 mg sapanisertib are administered on each of the three consecutive days of each of the four-week interval.

In some embodiments, 100-400 mg serabelisib and 1-5 mg sapanisertib are administered on day 1, day 2, day 3, day 8, day 9, day 10, day 15, day 16, day 17, day 22, day 23 and day 24 of the four-week interval. In some embodiments, 50 mg, 100 mg, 200 mg, 300 mg, or 400 mg serabelisib and 1 mg, 2 mg, 3 mg, 4 mg, or 5 mg sapanisertib are administered on day 1, day 2, day 3, day 8, day 9, day 10, day 15, day 16, day 17, day 22, day 23 and day 24 of the four-week interval. Enumerated Embodiments V-20 to V-30 provide additional serabelisib and sapanisertib dosage regimens.

In some embodiments, 200-800 mg fulvestrant are administered. In some embodiments, 200 mg-800 mg, 200 mg-700 mg, 200 mg-600 mg, 200 mg-500 mg, 200 mg-400 mg, 200 mg-300 mg, 300 mg-800 mg, 300 mg-700 mg, 300 mg-600 mg, 300 mg-500 mg, 300 mg-400 mg, 400 mg-800 mg, 400 mg-700 mg, 400 mg-600 mg, 400 mg-500 mg, 500 mg-800 mg, 500 mg-700 mg, 500 mg-600 mg, 600 mg-800 mg, 600 mg-700 mg, 700 mg-800 mg fulvestrant are administered. In some embodiments, 200 mg, 300 mg, 400 mg, 500 mg, 600 mg, 700 mg, or 800 mg fulvestrant are administered. In some embodiments, 500 mg fulvestrant are administered.

In some embodiments, the fulvestrant is administered on day 1 and day 15 of the four-week interval. In some embodiments, the fulvestrant is administered once monthly after the four-week interval.

In some embodiments, 50 mg-500 mg serabelisib and 1 mg-5 mg sapanisertib are administered on day 1, day 2, day 3, day 8, day 9, day 10, day 15, day 16, day 17, day 22, day 23 and day 24 of the four-week interval and 200 mg-800 mg fulvestrant are administered on day 1 and day 15 of the four-week interval. In some embodiments, 50 mg, 100 mg, 200 mg, 300 mg, or 400 mg serabelisib and 1 mg, 2 mg, 3 mg, 4 mg, or 5 mg sapanisertib are administered on day 1, day 2, day 3, day 8, day 9, day 10, day 15, day 16, day 17, day 22, day 23 and day 24 of the four-week interval, and 200 mg, 300 mg, 400 mg, 500 mg, 600 mg, 700 mg, or 800 mg fulvestrant are administered on day 1 and day 15 of the four-week interval. Enumerated Embodiments V-37 to V-44 provide additional serabelisib and sapanisertib dosage regimens.

In some embodiments, 60 mg-240 mg, 60 mg-200 mg, 60 mg-160 mg, 60 mg-120 mg, 60 mg-80 mg, 100 mg-240 mg, 100 mg-200 mg, 100 mg-160 mg, 100 mg-120 mg, 140 mg-240 mg, 140 mg-200 mg, 140 mg-160 mg, 180 mg-240 mg, 180 mg-200 mg, 220 mg-240 mg palbociclib are administered.

In some embodiments, 60 mg, 70 mg, 80 mg, 90 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, or 240 mg palbociclib are administered.

In some embodiments, the palbociclib is administered once daily for each of 21 consecutive days of the four-week interval.

In some embodiments, 50 mg-500 mg serabelisib and 1 mg-5 mg sapanisertib are administered on day 1, day 2, day 3, day 8, day 9, day 10, day 15, day 16, day 17, day 22, day 23 and day 24 of the four-week interval and 60 mg-240 mg palbociclib are administered once daily for each of 21 consecutive days of the four-week interval. In some embodiments, 50 mg, 100 mg, 200 mg, 300 mg, or 400 mg serabelisib and 1 mg, 2 mg, 3 mg, 4 mg, or 5 mg sapanisertib are administered on day 1, day 2, day 3, day 8, day 9, day 10, day 15, day 16, day 17, day 22, day 23 and day 24 of the four-week interval, and 60 mg, 70 mg, 80 mg, 90 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, or 240 mg palbociclib are administered once daily for each of 21 consecutive days of the four-week interval. Enumerated Embodiments V-48 to V-54 provide additional serabelisib and sapanisertib dosage regimens.

In some embodiments, 50 mg-500 mg serabelisib and 1 mg-5 mg sapanisertib are administered on day 1, day 2, day 3, day 8, day 9, day 10, day 15, day 16, day 17, day 22, day 23 and day 24 of the four-week interval; 200 mg-800 mg fulvestrant are administered on day 1 and day 15 of the four-week interval; and 60 mg-240 mg palbociclib are administered once daily for each of 21 consecutive days of the four-week interval. In some embodiments, 50 mg, 100 mg, 200 mg, 300 mg, or 400 mg serabelisib and 1 mg, 2 mg, 3 mg, 4 mg, or 5 mg sapanisertib are administered on day 1, day 2, day 3, day 8, day 9, day 10, day 15, day 16, day 17, day 22, day 23 and day 24 of the four-week interval; 200 mg, 300 mg, 400 mg, 500 mg, 600 mg, 700 mg, or 800 mg fulvestrant are administered on day 1 and day 15 of the four-week interval; and 60 mg, 70 mg, 80 mg, 90 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, or 240 mg palbociclib are administered once daily for each of 21 consecutive days of the four-week interval. Enumerated Embodiments V-55 to V-62 provide additional serabelisib and sapanisertib dosage regimens.

In some embodiments, the treating comprises providing a progression-free survival (PFS) of at least six months, preferably at least nine months, more preferably at least 12 months to the subject. In some embodiments, the time frame for this outcome measure is five years.

In some embodiments, the treatment of subjects results in an ORR of 20% or higher, 35% or higher, 40% or higher, or 60% or higher.

In some embodiments, the treatment of subjects results in a CBR of 30% or higher, 50% or higher, 60% or higher, or 80% or higher. In some embodiments, the time frame for this outcome measure is five years.

In some embodiments, the treatment of subjects results in a DCR of 40% or higher, 50% or higher, 60% or higher, or 80% or higher. In some embodiments, the time frame for this outcome measure is five years.

In some embodiments, the treating comprises providing an overall survival (OS) of at least nine months, at least 12 months, or at least 15 months to the subject.

In some embodiments, the treating comprises providing a duration of response (DoR) of at least six months, at least nine months, at least 12 months, or at least 15 months to the subject.

In some embodiments, the treating comprises providing a complete response (CR) or a partial response (PR) to the subject.

In some embodiments, the treating is safe and tolerable by assessment of adverse events (AEs)/serious adverse events (SAEs). In some embodiments, the treating comprises the subject exhibiting tolerable AEs. In some embodiments, the treating comprises the subject presenting no SAEs. In some embodiments, AEs and SAEs are graded according to the National Cancer Institute (NCI CTCAE v5.0.)

In some embodiments, the treatment of subjects results in all grade hyperglycemia in less than 30%, less than 20%, less than 10%, or less than 5% of subjects.

In some embodiments, the treatment of subjects results in Grade 3 and Grade 4 hyperglycemia in less than 20%, less than 10%, or less than 5% of subjects.

In some embodiments, the treating comprises the subject presenting no grade 3 or grade 4 hyperglycemia.

In some embodiments, the treating comprises providing the subject with a less than 30% likelihood of presenting hyperglycemia, preferably with a less than 20% likelihood of presenting hyperglycemia, more preferably with a less than 15% likelihood of presenting hyperglycemia.

In some embodiments, the treating comprises providing the subject with a less than 15% likelihood of presenting grade 3 or grade 4 hyperglycemia, preferably with a less than 10% likelihood of presenting grade 3 or grade 4 hyperglycemia, preferably with a less than 7% likelihood of presenting grade 3 or grade 4 hyperglycemia, more preferably with a less than 5% likelihood of presenting grade 3 or grade 4 hyperglycemia.

In some embodiments, the treating excludes administration of a hypoglycemic agent (e.g., metformin.) In some embodiments, the treating excludes administration of sodium-glucose cotransporter-2 (SGLT2) inhibitors (e.g., empagliflozin, dapagliflozin, canagliflozin, or ertugliflozin). In some embodiments, the treating excludes any measures to prevent, ameliorate, treat, or reduce hyperglycemia.

In some embodiments, the treatment excludes administration to subjects having Type 1 or Type 2 diabetes.

In some embodiments, the treating includes measures to prevent, ameliorate, treat, or reduce hyperglycemia. In some embodiments, the treating includes administration of a hypoglycemic agent (e.g., metformin.) In some embodiments, the treating includes administration of a sodium-glucose cotransporter-2 (SGLT2) inhibitor (e.g., empagliflozin, dapagliflozin, canagliflozin, or ertugliflozin). In some embodiments, subject with HbA1C values ≥5.7 (prediabetes or diabetes) are administered metformin. In some embodiments, the treating includes 500 mg-1000 mg orally QD administered metformin. In some embodiments, the treating includes 500 mg orally QD administered metformin titrated up to 1000 mg-1000 mg orally QD administered metformin.

In some embodiments, the treatment comprises administration to subjects having Type 1 or Type 2 diabetes.

In some embodiments, sapanisertib and serabelisib administration is halted if hyperglycemia is Grade 3 or higher. In some embodiments, sapanisertib and serabelisib administration is halted until hyperglycemia is ≤Grade 2. In some embodiments, sapanisertib and serabelisib are resumed at the same dose and schedule after hyperglycemia is ≤Grade 2. In some embodiments, sapanisertib and serabelisib are resumed at the same dose if the hyperglycemia was Grade 3 or higher for ≤1 week. In some embodiments, sapanisertib is reduced by 1 dose level (e.g., by 1 mg or 2 mg) if the hyperglycemia was Grade 3 for >1 but ≤2 weeks. In some embodiments, sapanisertib and serabelisib are both reduced by 1 dose level (e.g., by 1 mg or 2 mg sapanisertib and by 100 mg or 200 mg serabelisib) if the hyperglycemia was Grade 3 for >2 weeks.

In some embodiments, the treating comprises the subject presenting no grade 3 or grade 4 stomatitis (e.g., mouth ulcers, and/or oral mucositis).

In some embodiments, the treating comprises providing the subject with a less than 80% likelihood of presenting stomatitis, preferably with a less than 65% likelihood of presenting stomatitis, preferably with a less than 50% likelihood of presenting stomatitis, more preferably with a less than 30% of presenting stomatitis, more preferably with a less than 20% likelihood of presenting stomatitis, more preferably with a less than 15% likelihood of presenting stomatitis.

In some embodiments, the treating comprises providing the subject with a less than 30% likelihood of presenting grade 3 or grade 4 stomatitis, preferably with a less than 20% likelihood of presenting grade 3 or grade 4 stomatitis, preferably with a less than 15% likelihood of presenting grade 3 or grade 4 stomatitis, more preferably with a less than 10% likelihood of presenting grade 3 or grade 4 stomatitis, more preferably with a less than 5% likelihood of presenting grade 3 or grade 4 stomatitis.

In some embodiments, the treating excludes administration of a steroid or corticosteroid. In some embodiments, the treating excludes administration of a prophylactic mouth rinse comprising a steroid or corticosteroid. In some embodiments, the treating excludes any measures to prevent, ameliorate, treat, or reduce stomatitis.

In some embodiments, the method of treatment proceeds without inducing grade 4 stomatitis and without inducing grade 4 hyperglycemia. In some embodiment, the method of treatment proceeds without inducing grade 3 stomatitis.

In some embodiments, the treating excludes administration of a steroid or corticosteroid. In some embodiments, the treating excludes administration of a prophylactic mouth rinse comprising a steroid or corticosteroid. In some embodiments, the treating excludes any measures to prevent, ameliorate, treat, or reduce stomatitis.

In some embodiments, sapanisertib and serabelisib administration is halted if oral mucositis is Grade 2 or higher. In some embodiments, sapanisertib and serabelisib administration is halted if oral mucositis is Grade 3 or higher. In some embodiments, sapanisertib and serabelisib administration is halted if oral mucositis is Grade 4. In some embodiments, sapanisertib and serabelisib administration is halted until oral mucositis is ≤Grade 2. In some embodiments, sapanisertib and serabelisib administration is halted until oral mucositis is ≤Grade 3. In some embodiments, sapanisertib and serabelisib administration is halted until oral mucositis is less than Grade 4. In some embodiments, sapanisertib and serabelisib are resumed at the same dose and schedule after hyperglycemia is ≤Grade 1. In some embodiments, sapanisertib and serabelisib are both reduced by 1 dose level (e.g., by 1 mg or 2 mg sapanisertib and by 100 mg or 200 mg serabelisib) after hyperglycemia is ≤Grade 1.

In some embodiments, the treating excludes administration of a proton pump inhibitors (PPIs), antacids, histamine H2 receptor antagonists, or other treatments to raise gastric pH during the study.

In some embodiments, the treating excludes administration of CYP3A4 inhibitors and inducers. In some embodiments, the treatment excludes administration of CYP1A2 inhibitors and inducers. In some embodiments, CYP3A4 inhibitors or inducers or CYP1A2 inhibitors or inducers are not administered within seven days before administration of the treatment.

In some embodiments, the treating excludes administration of radiation therapy within seven, 14, or 21 days before administration of the treatment. In some embodiments, the treating excludes administration of radiation therapy within 21 days before administration of the treatment.

In some embodiments, the combination increases PFS; increases DCR; increases ORR; increases DoR; decreases likelihood of presenting hyperglycemia; or decreases likelihood of present stomatitis when compared to administration of the hormone receptor targeting therapeutic agent without the orally administered sapanisertib and orally administered serabelisib.

In some embodiments, the combination increases PFS; increases DCR; increases ORR; increases OS; increases DoR; decreases likelihood of presenting hyperglycemia; or decreases likelihood of present stomatitis when compared to administration of the orally administered sapanisertib orally administered serabelisib without the hormone receptor targeting therapeutic agent.

In some embodiments, the combination increases PFS; increases DCR; increases ORR; increases OS; increases DoR; decreases likelihood of presenting hyperglycemia; or decreases likelihood of present stomatitis when compared to administration of the targeted therapy without the orally administered sapanisertib and orally administered serabelisib.

In some embodiments, the combination increases PFS; increases DCR; increases ORR; increases OS; increases DoR; decreases likelihood of presenting hyperglycemia; or decreases likelihood of present stomatitis when compared to administration of the orally administered sapanisertib orally administered serabelisib without the targeted therapy.

In some embodiments, the subject in need thereof has advanced or metastatic breast cancer.

In some embodiments, the subject has advanced or recurrent disease that is not amenable to surgery or radiation, such as for curative intent.

In some embodiments, the breast cancer is resistant to hormone receptor targeting therapeutic agent. In some embodiments, the breast cancer is resistant to an aromatase inhibitor.

In some embodiments, the breast cancer is characterized by upregulated expression of ESR1 gene.

In some embodiments, the breast cancer is ESR1 negative.

In some embodiments, the breast cancer is hormone receptor positive (HR+). In some embodiments, the breast cancer is human epidermal growth factor receptor 2 negative (HER2−).

In some embodiments, the breast cancer is HR+ and HER2−. In some embodiments, the subject has a histologically confirmed diagnosis of HR+/HER2− breast cancer according to American Society of Clinical Oncology/College of American Pathologists (ASCO/CAP) guidelines. In some embodiments, the breast cancer is not triple-negative breast cancer.

In some embodiments, the subject has a measurable disease comprising at least one lesion measuring in at least one dimension ≥20 mm with conventional imaging techniques or ≥10 mm with CT or MRI. In some embodiments, the subject has at least one measurable or evaluable target lesion according to RECIST v1.1. Protocols for RECISTS v1.1 are included in the examples.

In some embodiments, the subject has a mutation identified in a gene of the PI3K/AKT/mTOR pathway, and/or has a mutated PI3K pathway. In some embodiments, the subject comprises one or more mutations in at least one gene selected from the group consisting of PIK3CA, DEPTOR, PIK3C2B, PRKAB2, PTEN, PREX2, NPRL3, MLST8, RPTOR, RICTOR, PIK3R1, AKT1, AKT2, AKT3, TSC1, TSC2, MTOR, STK11, GSK3A, GSK3B, DEPDC5, PRKAA1, PRKAA2, AKT1S1, GOLPH3, PDPK1, PRKAG2, PRKAG1, PIK3CB, PIK3CD, PIK3CG, RHEB, INPP4B, PPP2R1A, PRKAB1, PRKAG3, MAPKAP1, and NPRL2. In some embodiments, the subject comprises a mutation of PIK3CA. In some embodiments, the subject comprises a mutation of AKT1. In some embodiments, the subject comprises a mutation of PTEN.

In some embodiments, the subject has wild type PIK3CA. In some embodiments, the subject has wild type AKT1. In some embodiments, the subject has wild type PTEN. In some embodiments, the subject has wild types of PIK3CA, DEPTOR, PIK3C2B, PRKAB2, PTEN, PREX2, NPRL3, MLST8, RPTOR, RICTOR, PIK3R1, AKT1, AKT2, AKT3, TSC1, TSC2, MTOR, STK11, GSK3A, GSK3B, DEPDC5, PRKAA1, PRKAA2, AKT1S1, GOLPH3, PDPK1, PRKAG2, PRKAG1, PIK3CB, PIK3CD, PIK3CG, RHEB, INPP4B, PPP2R1A, PRKAB1, PRKAG3, MAPKAP1, and NPRL2.

In some embodiments, prior to the method of treating, the subject previously received one to four prior lines of therapy. In some embodiments, the one to four prior lines of therapy comprise a hormone receptor targeting therapeutic agent. In some embodiments, the one to four prior lines of therapy comprise administering a PI3K/AKT/mTOR pathway inhibitor.

In some embodiments, the subject previously received at least one prior systemic therapy. In some embodiments, the subject received prior treatment with CDK 4/6 inhibitors (e.g., palbociclib, ribociclib, abemaciclib). In some embodiments, the subject received at least one line of endocrine therapy (e.g., aromatase inhibitors, fulvestrant). In some embodiments, the subject's most recent prior endocrine therapy PFS had to be equal to or exceed 4 months. In some embodiments, the subject received up to 1 prior chemotherapy. In some embodiments, the subject received prior treatment with mTORC1 inhibitors (e.g., everolimus, temsirolimus).

In some embodiments, the one to four prior lines of therapy do not comprise administering a PI3K/AKT/mTOR pathway inhibitor. In some embodiments, the one to four prior lines of therapy do not comprise administering a PI3K inhibitor. In some embodiments, the one to four prior lines of therapy do not comprise administering an AKT inhibitor. In some embodiments, the one to four prior lines of therapy do not comprise administering an mTORC1/2 inhibitor. In some embodiments, the one to four prior lines of therapy do not comprise administering a dual PI3K/mTOR inhibitor. In some embodiments, the subject previously did not receive a PI3K inhibitor, a KT inhibitor, a dual mTORC1/2 inhibitor, and/or a dual PI3K/mTOR inhibitor.

Any of the features of the methods for treating breast cancer described herein are also applicable to uses of compounds for treating breast cancer, uses in the manufacture of a medicament for treating breast cancer, compounds for use in treating breast cancer, and kits comprising compounds for treating breast cancer.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein refer to their well-known meanings in the art.

For the foregoing embodiments, each embodiment disclosed herein is contemplated as being applicable to each of the other disclosed embodiments.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Equivalents: While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

### ENUMERATED EMBODIMENTS

### Set I:

Embodiment I-1. A method of treating cancer in a subject in need thereof, comprising administering to the subject in need thereof:
(a) a therapeutically effective amount of one or more compounds that inhibit two or more enzymes of the PI3K/AKT/mTOR pathway, and
(b) a therapeutically effective amount of one or more anti-cancer agents that do not directly inhibit enzymes in the PI3K/AKT/mTOR pathway.

Embodiment I-2. The method of embodiment I-1, wherein the method comprises administering one or more anti-cancer agents that do not directly inhibit enzymes in the PI3K/AKT/mTOR pathway for one day per week.

Embodiment I-3. The method of embodiments I-1 and I-2, wherein after administering the one or more anti-cancer agents that do not directly inhibit enzymes in the PI3K/AKT/mTOR pathway for three consecutive weeks, the one or more anti-cancer agents that do not directly inhibit enzymes in the PI3K/AKT/mTOR pathway are not administered on a 4^{th} week immediately following the three consecutive weeks.

Embodiment I-4. The method of embodiments I-1 to I-3, wherein the method comprises administering one or more compounds that inhibit two or more enzymes of the PI3K/AKT/mTOR pathway for three days per week.

Embodiment I-5. The method of embodiments I-1 to I-4, wherein the method comprises administering one or more compounds that inhibit two or more enzymes of the PI3K/AKT/mTOR pathway for three consecutive days per week.

Embodiment I-6. The method of embodiments I-1 to I-5, wherein the method comprises administering one or more anti-cancer agents that do not directly inhibit enzymes in the PI3K/AKT/mTOR pathway for one day followed the next day by administering the one or more compounds that inhibit two or more enzymes of the PI3K/AKT/mTOR pathway for three consecutive days.

Embodiment I-7. The method of embodiments I-1 to I-6, wherein the method comprises a four-week interval:
(a) wherein each week of the four-week interval comprises administering the one or more compounds that inhibit two or more enzymes of the PI3K/AKT/mTOR pathway for three consecutive days,
(b) the first three weeks of the four-week interval each comprises administering the one or more anti-cancer agents that do not directly inhibit enzymes in the PI3K/AKT/mTOR pathway for one day, and
(c) the 4^{th} week of the four-week interval does not comprise one or more anti-cancer agents that do not directly inhibit enzymes in the PI3K/AKT/mTOR pathway.

Embodiment I-8. The method of embodiments I-1 to I-7, wherein the one or more compounds that inhibit two or more enzymes of the PI3K/AKT/mTOR pathway comprise a Pan-PI3K-Pan-mTOR inhibitor.

Embodiment I-9. The method of embodiment I-8, wherein the Pan-PI3K-Pan-mTOR inhibitor is apitolisib, bimiralisib, dactolisib, gedatolisib, omipalisib, PQR309, SF1126, voxtalisib, or a combination thereof.

Embodiment I-10. The method of embodiments I-1 to I-9, wherein the one or more compounds that inhibit two or more enzymes of the PI3K/AKT/mTOR pathway comprise an mTOR inhibitor.

Embodiment I-11. The method of embodiment I-10, wherein the mTOR inhibitor is temsirolimus.

Embodiment I-12. The method of embodiment I-11, comprising administering about 10 mg/week to about 50 mg/week of the temsirolimus.

Embodiment I-13. The method of embodiments I-10 and I-11, comprising administering about 25 mg/week of the temsirolimus.

Embodiment I-14. The method of embodiment I-10, wherein the mTOR inhibitor is an mTORC1 inhibitor.

Embodiment I-15. The method of embodiments I-10 and I-14, wherein the mTORC1 inhibitor is everolimus.

Embodiment I-16. The method of embodiment I-15, comprising administering about 2 mg/day to about 10 mg/day of the everolimus.

Embodiment I-17. The method of embodiments I-15 and I-16, comprising administering about 5 mg/day of the everolimus.

Embodiment I-18. The method of embodiment I-10, wherein the mTOR inhibitor is an mTORC2 inhibitor.

Embodiment I-19. The method of embodiment I-10, wherein the mTOR inhibitor is a dual mTORC1 and mTORC2 inhibitor.

Embodiment I-20. The method of embodiments I-1 to I-19, wherein the one or more compounds that inhibit two or more enzymes of the PI3K/AKT/mTOR pathway comprise an AKT inhibitor.

Embodiment I-21. The method of embodiment I-20, wherein the AKT inhibitor is capivasertib, ipatasertib, MK-2206, triciribine, ABTL0812, or a combination thereof.

Embodiment I-22. The method of embodiments I-1 to I-20, wherein the one or more compounds that inhibit two or more enzymes of the PI3K/AKT/mTOR pathway comprise a PI3K inhibitor.

Embodiment I-23. The method of 22, wherein the PI3K inhibitor is a PI3K-alpha inhibitor.

Embodiment I-24. The method of embodiments I-1 to I-23, wherein the method comprises administering to the subject in need thereof:
(a) a therapeutically effective amount of a dual mTORC1 and mTORC2 inhibitor,
(b) a therapeutically effective amount of a PI3K-alpha inhibitor, and
(c) a therapeutically effective amount of one or more anti-cancer agents that do not directly inhibit enzymes in the PI3K/AKT/mTOR pathway.

Embodiment I-25. The method of embodiments I-1 to I-24, wherein the dual mTORC1 and mTORC2 inhibitor is sapanisertib, vistusertib, onatasertib, XP-105, AVTX-006, or a combination thereof.

Embodiment I-26. The method of embodiment I-25, wherein the dual mTORC1 and mTORC2 inhibitor is sapanisertib, which is administered at a dose of about 1 mg/day to about 6 mg/day.

Embodiment I-27. The method of embodiments I-25 and I-26, wherein sapanisertib is administered at a dose of about 1 mg/day to about 3 mg/day for three consecutive days followed the next day by four consecutive days without administering the sapanisertib.

Embodiment I-28. The method of embodiments I-1 to I-27, wherein the PI3K-alpha inhibitor selectively inhibits a PI3K-alpha mutant.

Embodiment I-29. The method of embodiment I-28, wherein the PI3K-alpha inhibitor that selectively inhibits a PI3K-alpha mutant is RLY-2608, STX-478, LY4045004, LOXO-783, OKI-219, or a combination thereof.

Embodiment I-30. The method of embodiments I-1 to I-28, wherein the PI3K-alpha inhibitor is, alpelisib, buparlisib, copanlisib, duvelisib, idelalisib, inavolisib, linperlisib, leniolisib, pictilisib, samotolisib, serabelisib, taselisib, ZSTK474, or a combination thereof.

Embodiment I-31. The method of embodiment I-30, wherein the PI3K-alpha inhibitor is alpelisib.

Embodiment I-32. The method of embodiment I-31, comprising administering about 100 mg/day to about 400 mg/day of the alpelisib.

Embodiment I-33. The method of embodiments I-31 and I-32, comprising administering about 200 mg/day of the alpelisib.

Embodiment I-34. The method of embodiment I-30, wherein the PI3K-alpha inhibitor is inavolisib.

Embodiment I-35. The method of embodiment I-34, comprising administering about 5 mg/day to about 20 mg/day of the inavolisib.

Embodiment I-36. The method of embodiments I-34 and I-35, comprising administering about 9 mg/day of the inavolisib.

Embodiment I-37. The method of embodiment I-36, wherein the PI3K-alpha inhibitor is serabelisib.

Embodiment I-38. The method of embodiment I-37, wherein the serabelisib is administered at a dose of about 50 mg/day to about 600 mg/day.

Embodiment I-39. The method of embodiments I-37 and I-38, wherein the serabelisib is administered at a dose of about 100 mg/day to about 300 mg/day for three consecutive days followed the next day by four consecutive days without administering the serabelisib.

Embodiment I-40. The method of embodiments I-1 to I-39, wherein the one or more compounds that inhibit two or more enzymes of the PI3K/AKT/mTOR pathway comprise sapanisertib and serabelisib.

Embodiment I-41. The method of embodiments I-1 to I-40, wherein the one or more anti-cancer agents that do not directly inhibit enzymes in the PI3K/AKT/mTOR pathway comprise a chemotherapeutic agent.

Embodiment I-42. The method of embodiments I-1 to I-41, wherein the one or more anti-cancer agents that do not directly inhibit enzymes in the PI3K/AKT/mTOR pathway comprise a targeted therapy.

Embodiment I-43. The method of embodiments I-1 to I-42, wherein the one or more anti-cancer agents that do not directly inhibit enzymes in the PI3K/AKT/mTOR pathway comprise a taxane.

Embodiment I-44. The method of embodiment I-43, wherein the one or more anti-cancer agents that do not directly inhibit enzymes in the PI3K/AKT/mTOR pathway comprise paclitaxel, docetaxel, or a combination thereof.

Embodiment I-45. The method embodiment I-44, wherein the paclitaxel is administered at a dose of about 40 mg/m² to about 100 mg/m².

Embodiment I-46. The method of embodiments I-44 and I-45, wherein the method comprises administering about 100 mg/m² to about 250 mg/m² of the paclitaxel every 21 days.

Embodiment I-47. The method of embodiments I-44 to I-46, wherein the method comprises administering about 100 mg/m² to about 250 mg/m² of the paclitaxel every 28 days.

Embodiment I-48. The method of embodiments I-44 to I-47, wherein the paclitaxel is administered at a dose of about 80 mg/m² to about 90 mg/m² once per week for three weeks followed by one week without administering paclitaxel.

Embodiment I-49. The method of embodiments I-1 to I-50, wherein the one or more anti-cancer agents that do not directly inhibit enzymes in the PI3K/AKT/mTOR pathway comprise carboplatin.

Embodiment I-50. The method of embodiments I-44 to I-49, wherein the carboplatin is administered in combination with about 135 mg/m² of paclitaxel.

Embodiment I-51. The method of embodiments I-1 to I-20, wherein the one or more anti-cancer agents that do not directly inhibit enzymes in the PI3K/AKT/mTOR pathway comprise a CDK4 inhibitor, a CDK6 inhibitor, a CDK4/6 inhibitor, or a combination thereof.

Embodiment I-52. The method of embodiments I-1 to I-51, wherein the one or more anti-cancer agents that do not directly inhibit enzymes in the PI3K/AKT/mTOR pathway.

Embodiment I-53. The method of embodiment I-52, wherein the one or more anti-cancer agents that do not directly inhibit enzymes in the PI3K/AKT/mTOR pathway comprise abemaciclib, palbociclib, ribociclib, or a combination thereof.

Embodiment I-54. The method of embodiment I-53, comprising administering about 200 mg/day to about 600 mg/day of the abemaciclib.

Embodiment I-55. The method of embodiments I-53 and I-54, comprising administering about 300 mg/day of the abemaciclib.

Embodiment I-56. The method of embodiments I-53 to I-55, wherein the palbociclib is administered in a repeating 28-day cycle comprising a palbociclib dose of about 75 mg/day to about 150 mg/day for three weeks followed by one week without administering the palbociclib.

Embodiment I-57. The method of embodiments I-53 to I-56, wherein the palbociclib dose is about 125 mg/day.

Embodiment I-58. The method of embodiments I-53 to I-57, wherein the ribociclib is administered in a repeating 28-day cycle comprising a ribociclib dose of about 200 mg/day to about 800 mg/day for three weeks followed by one week without administering the ribociclib.

Embodiment I-59. The method of embodiments I-53 to I-58, wherein the ribociclib dose is about 600 mg/day.

Embodiment I-60. The method of embodiments I-1 to I-59, wherein the one or more anti-cancer agents that do not directly inhibit enzymes in the PI3K/AKT/mTOR pathway comprise a hormone receptor targeting therapeutic agent.

Embodiment I-61. The method of embodiment I-60, wherein the one or more anti-cancer agents that do not directly inhibit enzymes in the PI3K/AKT/mTOR pathway comprise fulvestrant or elacestrant.

Embodiment I-62. The method of embodiment I-61, wherein the method comprises:
(a) administering 500 mg of fulvestrant twice in a four-week interval on day, and
(b) administering 500 mg of fulvestrant every 28 days thereafter.

Embodiment I-63. The method of embodiments I-61 and I-62, comprising administering about 100 mg/day to about 600 mg/day of the elacestrant.

Embodiment I-64. The method of embodiments I-61 to I-63, comprising administering about 345 mg/day of the elacestrant.

Embodiment I-65. The method of embodiments I-1 to I-64, wherein the one or more anti-cancer agents that do not directly inhibit enzymes in the PI3K/AKT/mTOR pathway comprise a modulator of p53 activity.

Embodiment I-66. The method of embodiment I-65, wherein the one or more anti-cancer agents that do not directly inhibit enzymes in the PI3K/AKT/mTOR pathway comprise selinexor.

Embodiment I-67. The method of embodiment I-66, comprising administering about 60 mg/week to about 80 mg/week of the selinexor.

Embodiment I-68. The method of embodiments I-66 and I-67, comprising administering about 60 mg/week of the selinexor if the subject in need thereof has a BMI<20 kg/m² or administering about 80 mg/week of the selinexor if the subject in need thereof has a BMI>20 kg/m².

Embodiment I-69. The method of embodiments I-1 to I-68, further comprising administering an insulin suppressing diet to the subject in need thereof.

Embodiment I-70. The method of embodiment I-69, wherein the insulin suppressing diet comprises about 65% to about 80% of fat by w/w % of the macronutrient content.

Embodiment I-71. The method of embodiments I-69 and I-70, wherein the insulin suppressing diet comprises about 8% to about 25% of protein by w/w % of the macronutrient content.

Embodiment I-72. The method of embodiments I-69 to I-71, wherein the insulin suppressing diet comprises about 8% to about 15% of total carbohydrates by w/w % of the macronutrient content.

Embodiment I-73. The method of embodiments I-69 to I-72, wherein the insulin suppressing diet comprises about 65% to about 80% of fat, about 8% to about 25% of protein, and about 8% to about 15% of total carbohydrates by w/w % of the macronutrient content.

Embodiment I-74. The method of embodiments I-69 to I-73, wherein the insulin suppressing diet comprises about 15% to about 35% of protein and total carbohydrates by w/w % of the macronutrient content.

Embodiment I-75. The method of embodiments I-1 to I-74, wherein the cancer is uterine cancer, breast cancer, anal cancer, head and neck cancer, bladder cancer, colorectal cancer, skin cancer, ovarian cancer, ampullary cancer, glioma, small bowel cancer, cancer of unknown primary, lung cancer, esophagogastric cancer, renal cell carcinoma, or prostate cancer.

Embodiment I-76. The method of embodiment I-75, wherein the cancer is breast cancer.

Embodiment I-77. The method of embodiment I-75, wherein the cancer is endometrial cancer.

Embodiment I-78. The method of embodiments I-1 to I-77, wherein the cancer comprises a PI3K/AKT/mTOR pathway mutation.

Embodiment I-79. A method of embodiment I-78, wherein the PI3K/AKT/mTOR pathway mutation comprises one or more mutations in the following genes PIK3CA, PIK3CB, PIK3CG, PIK3CD, PIK3C2B, PIK3R1, AKT1, AKT2, AKT3, MTOR, RPTOR, RAPTOR, RICTOR, MLST8, DEPTOR, S6K, 4E-BP1, AKT1S1, PTEN, STK11, TSC1, TSC2, PPP2R1A, INPP4B, PDK1, RHEB, AMPK, GSK3, DEPDC5, NPRL2, NPRL3, or combinations thereof.

Embodiment I-80. A method of embodiments I-78 and I-79, wherein the PI3K/AKT/mTOR pathway mutation comprises a PIK3CA amplification.

Embodiment I-81. A method of embodiments I-78 and I-79, wherein the cancer is not PIK3CA mutated.

Embodiment I-82. A method of embodiments I-78 to I-81, wherein the PI3K/AKT/mTOR pathway mutation comprises a PTEN deletion.

Embodiment I-83. A method of embodiments I-78 to I-82, wherein the PI3K/AKT/mTOR pathway mutation comprises a PIK3R1 deletion.

Embodiment I-84. A method of embodiments I-78 to I-83, wherein the PI3K/AKT/mTOR pathway mutation comprises a PIK3CA a helical domain mutation.

Embodiment I-85. A method of embodiments I-78 to I-84, wherein the PI3K/AKT/mTOR pathway mutation comprises a PIK3CA kinase domain mutation.

Embodiment I-86. A method of embodiments I-1 to I-85, wherein the cancer is ER positive.

Embodiment I-87. A method of embodiments I-1 to I-85, wherein the cancer is ER negative.

Embodiment I-88. A method of embodiments I-1 to I-87, wherein the cancer is HER2 positive.

Embodiment I-89. A method of embodiments I-1 to I-87, wherein the cancer is HER2 negative.

Embodiment I-90. The method of embodiments I-1 to I-89, wherein the cancer was previously treated with a taxane.

Embodiment I-91. The method of embodiment I-90, wherein the taxane was paclitaxel, docetaxel, or a combination thereof.

Embodiment I-92. The method of embodiments I-1 to I-91, wherein the method reduces phosphorylated 4E-BP1 relative to untreated tumor tissue.

### Set II:

Embodiment II-1. A method of treating endometrial cancer in a human subject in need thereof, the method comprising administering to the subject a therapeutically effective amount of a combination comprising:
(a) an amount of orally administered sapanisertib,
(b) an amount of orally administered serabelisib, and
(c) an amount of intravenously administered paclitaxel.

Embodiment II-2. The method of embodiment II-1, wherein the treating comprises providing a progression-free survival (PFS) of at least six months, preferably at least nine months, more preferably at least 12 months to the subject.

Embodiment II-3. The method of embodiments II-1 and II-2, wherein the treating comprises providing an overall survival (OS) of at least nine months, at least 12 months, or at least 15 months to the subject.

Embodiment II-4. The method of embodiments II-1 to II-3, wherein the treating comprises providing a duration of response (DoR) of at least six months, at least nine months, at least 12 months, or at least 15 months to the subject.

Embodiment II-5. The method of embodiments II-1 to II-4, wherein the treating comprises providing a complete response (CR) or a partial response (PR) to the subject.

Embodiment II-6. The method of embodiments II-1 to II-5, wherein the treating comprises providing to the subject a progression-free survival of at least six months from the start of the treatment.

Embodiment II-7. The method of embodiments II-1 to II-6, wherein the treating comprises providing a complete response (CR), a partial response (PR), or a stable disease (SD) of at least 16 to 24 weeks to the subject.

Embodiment II-8. The method of embodiments II-1 to II-7, wherein the treating comprises providing the subject with an at least 35% likelihood, preferably an at least 40% likelihood, more preferably an at least 60% likelihood of complete response (CR) or partial response (PR) to the treatment.

Embodiment II-9. The method of embodiments II-1 to II-8, wherein the treating comprises providing the subject with an at least 50% likelihood, preferably an at least 60% likelihood, more preferably an at least 80% likelihood of achieving progression-free survival (PFS) at six months from the start of the treatment.

Embodiment II-10. The method of embodiments II-1 to II-9, wherein the treating comprises providing the subject with an at least 50% likelihood, preferably an at least 60% likelihood, more preferably an at least 80% likelihood of achieving a complete response (CR), a partial response (PR), or having stable disease (SD) of at least 16 to 24 weeks in response to the treatment.

Embodiment II-11. The method of embodiments II-1 to II-10, wherein the subject is orally administered about 1-4 mg of sapanisertib, orally administered about 100-300 mg of serabelisib, and intravenously administered about 40-100 mg/m² of paclitaxel.

Embodiment II-12. The method of embodiments II-1 to II-11, wherein the subject is orally administered about 3 mg-4 mg of sapanisertib, orally administered about 200 mg of serabelisib, and intravenously administered about 80 mg/m² of paclitaxel.

Embodiment II-13. The method of embodiments II-1 to II-12, wherein the amount of paclitaxel is administered, then the amount of serabelisib and the amount of sapanisertib are administered on each of the three days following the paclitaxel administration.

Embodiment II-14. The method of embodiments II-1 to II-13, wherein the amount of serabelisib and the amount of sapanisertib are administered at about the same time.

Embodiment II-15. The method of embodiments II-1 to II-14, wherein the amount of serabelisib and the amount of sapanisertib are administered with food and/or with a meal.

Embodiment II-16. The method of embodiments II-1 to II-15, wherein the method comprises repeating the administration of sapanisertib, serabelisib, and paclitaxel over the course of a treatment interval.

Embodiment II-17. The method of embodiments II-1 to II-16, wherein the method comprises a four-week interval:
(a) wherein each week of the four-week interval comprises administering the amount of sapanisertib and the amount of serabelisib for each of three consecutive days,
(b) wherein the first three weeks of the four-week interval each comprises administering the amount of paclitaxel for one day, and
(c) wherein paclitaxel is not administered during the 4^{th} week of the four-week interval.

Embodiment II-18. The method of embodiment II-17, wherein 40-100 mg/m² paclitaxel is administered on day 1, day 8, and day 15 of the interval; and wherein 100-300 mg serabelisib and 1-4 mg sapanisertib are administered on day 2, day 3, day 4, day 9, day 10, day 11, day 16, day 17, day 18, day 23, day 24 and day 25 of the interval.

Embodiment II-19. The method of embodiments II-1 to II-18, further comprising administering an insulin suppressing diet to the subject in need thereof.

Embodiment II-20. The method of embodiments II-1 to II-19, wherein prior to the method of treating the subject previously received one to four prior lines of chemotherapy, preferably including a systemic therapy, more preferably including a platinum-based chemotherapy and/or a checkpoint inhibitor.

Embodiment II-21. The method of embodiments II-1 to II-20, wherein the subject has a mutation identified in a gene of the PI3K/AKT/Mtor pathway, and/or has a mutated PI3K pathway.

Embodiment II-22. The method of embodiments II-1 to II-21, wherein the subject comprises one or more mutations in at least one gene selected from the group consisting of PIK3CB, PIK3CG, PIK3CD, PIK3C2B, RPTOR, RAPTOR, RICTOR, MLST8, DEPTOR, S6K, 4E-BP1, AKT1S1, STK11, TSC2, INPP4B, PDK1, RHEB, AMPK, GSK3, DEPDCS, NPRL2, and NPRL3.

Embodiment II-23. The method of embodiments II-1 to II-22, wherein the subject comprises one or more mutations in at least one gene selected from the group consisting of PIK3CA, PIK3R1, AKT1, AKT2, AKT3, MTOR, PTEN, TSC1, PPP2R1A, TP53, RB1, CCNE1, ERBB2, APC, NF1, MYCN, CDKN2A, FGFR1, CDKN2B, CCND1, MYC, EGFR, ERRF11, KRAS, CTNNB1, FBXW7, ABL1, CHEK2, and ARAF.

Embodiment II-24. The method of embodiments II-1 to II-23, wherein the endometrial cancer is advanced or recurrent endometrial cancer.

Embodiment II-25. The method of embodiments II-1 to II-24, wherein the treating comprises the subject presenting no serious adverse events, more preferably no adverse events.

Embodiment II-26. The method of embodiments II-1 to II-25, wherein the treating comprises the subject presenting no grade 3 or grade 4 adverse events.

Embodiment II-27. The method of embodiments II-1 to II-26, wherein the treating comprises the subject presenting no grade 3 or grade 4 hyperglycemia.

Embodiment II-28. The method of embodiments II-1 to II-27, wherein the treating comprises providing the subject with a less than 30% likelihood of presenting hyperglycemia, preferably with a less than 20% likelihood of presenting hyperglycemia, more preferably with a less than 15% likelihood of presenting hyperglycemia.

Embodiment II-29. The method of embodiments II-1 to II-28, wherein the treating comprises providing the subject with a less than 15% likelihood of presenting grade 3 or grade 4 hyperglycemia, preferably with a less than 10% likelihood of presenting grade 3 or grade 4 hyperglycemia, preferably with a less than 7% likelihood of presenting grade 3 or grade 4 hyperglycemia, more preferably with a less than 5% likelihood of presenting grade 3 or grade 4 hyperglycemia.

Embodiment II-30. The method of embodiments II-1 to II-29, wherein the treating comprises the subject presenting no grade 3 or grade 4 stomatitis.

Embodiment II-31. The method of embodiments II-1 to II-30, wherein the treating comprises providing the subject with a less than 80% likelihood of presenting stomatitis, preferably with a less than 65% likelihood of presenting stomatitis, preferably with a less than 50% likelihood of presenting stomatitis, more preferably with a less than 30% of presenting stomatitis, more preferably with a less than 20% likelihood of presenting stomatitis, more preferably with a less than 15% likelihood of presenting stomatitis.

Embodiment II-32. The method of embodiments II-1 to II-31, wherein the treating comprises providing the subject with a less than 30% likelihood of presenting grade 3 or grade 4 stomatitis, preferably with a less than 20% likelihood of presenting grade 3 or grade 4 stomatitis, preferably with a less than 15% likelihood of presenting grade 3 or grade 4 stomatitis, more preferably with a less than 10% likelihood of presenting grade 3 or grade 4 stomatitis, more preferably with a less than 5% likelihood of presenting grade 3 or grade 4 stomatitis.

Embodiment II-33. The method of embodiments II-1 to II-32, wherein the treating excludes administration of a steroid or corticosteroid.

Embodiment II-34. The method of embodiments II-1 to II-33, wherein the treating excludes administration of a prophylactic mouth rinse comprising a steroid or corticosteroid.

Embodiment II-35. The method of embodiments II-1 to II-34, wherein the treating excludes any measures to prevent, ameliorate, treat, or reduce stomatitis.

Embodiment II-36. A method of treating endometrial cancer in a human subject in need thereof without inducing grade 4 stomatitis or without inducing grade 4 hyperglycemia, the method comprising
(a) intravenously administering to the subject 40-100 mg/m² paclitaxel on day 1, day 8, and day 15 of a four-week treatment interval; and
(b) orally administering to the subject 100-300 mg serabelisib and 1-4 mg sapanisertib on day 2, day 3, day 4, day 9, day 10, day 11, day 16, day 17, day 18, day 23, day 24 and day 25 of the four-week treatment interval.

Embodiment II-37. The method of embodiment II-36, wherein the method of treatment proceeds without inducing grade 4 stomatitis and without inducing grade 4 hyperglycemia.

Embodiment II-38. The method of embodiments II-36 and II-37, wherein the method of treatment proceeds without inducing grade 3 stomatitis.

Embodiment II-39. The method of embodiments II-36 to II-38, wherein the method of treatment proceeds without inducing grade 3 hyperglycemia.

Embodiment II-40. The method of embodiments II-36 to II-39, wherein the treating comprises providing a progression-free survival (PFS) of at least six months, preferably at least nine months, more preferably at least 12 months to the subject.

Embodiment II-41. The method of embodiments II-36 to II-40, wherein the treating comprises providing an overall survival (OS) of at least nine months, at least 12 months, or at least 15 months to the subject.

Embodiment II-42. The method of embodiments II-36 to II-41, wherein the treating comprises providing a duration of response (DoR) of at least six months, at least nine months, at least 12 months, or at least 15 months to the subject.

Embodiment II-43. The method of embodiments II-36 to II-42, wherein the treating comprises providing a complete response (CR), a partial response (PR), or a stable disease (SD) of at least 16 to 24 weeks to the subject.

Embodiment II-44. The method of embodiments II-36 to II-43, further comprising administering an insulin suppressing diet to the subject in need thereof.

Embodiment II-45. The method of embodiments II-36 to II-44, wherein the serabelisib and sapanisertib are administered with food and/or with a meal.

### Set III:

Embodiment III-1. A method of treating endometrial cancer in a human subject in need thereof, the method comprising:
(a) intravenously administering to the subject 40-100 mg/m² paclitaxel on day 1, day 8, and day 15 of a four-week treatment interval; and
(b) orally administering to the subject 100-300 mg serabelisib and 1-4 mg sapanisertib on day 2, day 3, day 4, day 9, day 10, day 11, day 16, day 17, day 18, day 23, day 24 and day 25 of the four-week treatment interval,
(c) so as to treat endometrial cancer in the human subject.

Embodiment III-2. The method of embodiment III-1, which provides a partial response (PR) or stable disease (SD) of at least 16 weeks to the subject.

Embodiment III-3. The method of embodiment III-1, which provides a complete response (CR) to the subject.

Embodiment III-4. The method of embodiment III-1, which provides
(a) a progression-free survival (PFS) of at least 12 months to the subject;
(b) an overall survival (OS) of at least 15 months to the subject; and/or
(c) a duration of response (DoR) of at least 15 months to the subject.

Embodiment III-5. The method of embodiment III-1, wherein the serabelisib and the sapanisertib are administered at about the same time.

Embodiment III-6. The method of embodiment III-1, wherein the serabelisib and the sapanisertib are administered with food and/or with a meal.

Embodiment III-7. The method of embodiment III-1, wherein the subject is on an insulin suppressing diet.

Embodiment III-8. The method of embodiment III-1, wherein the subject previously received from one to four prior systemic therapies which include at least one platinum-based chemotherapy and at least one checkpoint inhibitor.

Embodiment III-9. The method of embodiment III-1, wherein the subject has a mutation identified in a gene of the PI3K/AKT/mTOR pathway, and/or has a mutated PI3K pathway.

Embodiment III-10. The method of embodiment III-1, wherein the subject comprises one or more mutations in at least one gene selected from the group consisting of PIK3CB, PIK3CG, PIK3CD, PIK3C2B, RPTOR, RAPTOR, RICTOR, MLST8, DEPTOR, S6K, 4E-BP1, AKT1S1, STK11, TSC2, INPP4B, PDK1, RHEB, AMPK, GSK3, DEPDC5, NPRL2, and NPRL3.

Embodiment III-11. The method of embodiment III-1, which does not induce a serious adverse event (SAE) in the human subject.

Embodiment III-12. The method of embodiment III-1, which does not induce a grade 3 adverse event or a grade 4 adverse event in the human subject.

Embodiment III-13. The method of embodiment III-1, which excludes administration of a steroid or corticosteroid, excludes administration of a prophylactic mouth rinse comprising a steroid or corticosteroid, and/or excludes a measure to prevent, ameliorate, treat, or reduce stomatitis.

Embodiment III-14. A method of treating endometrial cancer in a human subject in need thereof, the method comprising:
(a) intravenously administering to the subject 40-100 mg/m² paclitaxel on day 1, day 8, and day 15 of a four-week treatment interval;
(b) orally administering to the subject 100-300 mg serabelisib and 1-4 mg sapanisertib on day 2, day 3, day 4, day 9, day 10, day 11, day 16, day 17, day 18, day 23, day 24 and day 25 of the four-week treatment interval; and
(c) excluding a measure to prevent, ameliorate, treat, or reduce stomatitis, (d) so as to treat endometrial cancer in the human subject.

Embodiment III-15. The method of embodiment III-14, which excludes administration of a steroid or corticosteroid.

Embodiment III-16. The method of embodiment III-14, which excludes administration of a prophylactic mouth rinse comprising a steroid or corticosteroid.

Embodiment III-17. A method of treating endometrial cancer in a human subject in need thereof without inducing grade 4 stomatitis or without inducing grade 4 hyperglycemia, the method comprising:
(a) intravenously administering to the subject 40-100 mg/m² paclitaxel on day 1, day 8, and day 15 of a four-week treatment interval; and
(b) orally administering to the subject 100-300 mg serabelisib and 1-4 mg sapanisertib on day 2, day 3, day 4, day 9, day 10, day 11, day 16, day 17, day 18, day 23, day 24 and day 25 of the four-week treatment interval,
(c) so as to treat endometrial cancer in the human subject without inducing grade 4 stomatitis or without inducing grade 4 hyperglycemia.

Embodiment III-18. The method of embodiment III-17, which does not induce grade 4 stomatitis and does not induce grade 4 hyperglycemia.

Embodiment III-19. The method of embodiment III-17, which does not induce grade 3 stomatitis.

Embodiment III-20. The method of embodiment III-17, which does not induce grade 3 hyperglycemia.

### Set IV:

Embodiment IV-1. A method of treating cancer in a subject in need thereof, comprising administering to the subject in need thereof:
(a) a therapeutically effective amount of one or more compounds that inhibit two or more enzymes of the PI3K/AKT/mTOR pathway, and
(b) a therapeutically effective amount of one or more anti-cancer agents that do not directly inhibit enzymes in the PI3K/AKT/mTOR pathway.

Embodiment IV-2. The method of embodiment IV-1, wherein the cancer is breast cancer.

Embodiment IV-3. The method of embodiment IV-2, wherein the one or more compounds that inhibit two or more enzymes of the PI3K/AKT/mTOR pathway comprise sapanisertib and serabelisib,
(a) wherein about 1 mg/day to about 4 mg/day sapanisertib and about 50 mg/day to about 400 mg/day of the serabelisib is administered to the subject for three days per week.

Embodiment IV-4. The method of embodiments IV-2 and IV-3, wherein the one or more anti-cancer agents that do not directly inhibit enzymes in the PI3K/AKT/mTOR pathway comprise any one of fulvestrant, a cyclin-dependent kinase 4 (CDK4) inhibitor, a cyclin-dependent kinase 4 (CDK6) inhibitor, a CDK4/6 inhibitor, or a combination thereof.

Embodiment IV-5. The method of embodiment IV-4, wherein the one or more anti-cancer agents that do not directly inhibit enzymes in the PI3K/AKT/mTOR pathway comprises fulvestrant.

Embodiment IV-6. The method of embodiment IV-5, comprising administering to the subject about 500 mg of fulvestrant twice in a four-week interval on day 1, and administering about 500 mg of fulvestrant every 28 days thereafter, or administering a fulvestrant 500 mg loading dose on day 1, day 15, day 29, and then a 500 mg maintenance dose every 28 days thereafter.

Embodiment IV-7. The method of embodiments IV-2 to IV-6, wherein the one or more anti-cancer agents that do not directly inhibit enzymes in the PI3K/AKT/mTOR pathway comprises a CDK4/6 inhibitor.

Embodiment IV-8. The method of embodiment IV-7, wherein the CDK4/6 inhibitor is palbociclib, ribociclib, and/or abemaciclib.

Embodiment IV-9. The method of embodiment IV-8, wherein about 75 mg/day to about 150 mg/day palbociclib is administered to the subject.

Embodiment IV-10. The method of embodiments IV-8 and IV-9, wherein the palbociclib is administered in a repeating 28-day cycle comprising a palbociclib dose of about 75 mg/day to about 150 mg/day for three weeks followed by one week without administering the palbociclib.

Embodiment IV-11. The method of embodiments IV-8 to IV-10, wherein 125 mg palbociclib is administered once daily for 21 days and then palbociclib is not administered for seven days.

Embodiment IV-12. The method of embodiments IV-2 to IV-11, wherein the breast cancer is HER2 negative.

Embodiment IV-13. The method of embodiments IV-2 to IV-12, wherein the breast cancer is a hormone receptor-positive cancer.

Embodiment IV-14. The method of embodiments IV-1 to IV-13, wherein the one or more compounds that inhibit two or more enzymes of the PI3K/AKT/mTOR pathway comprise a Pan-PI3K-Pan-mTOR inhibitor, preferably wherein the Pan-PI3K-Pan-mTOR inhibitor is apitolisib, bimiralisib, dactolisib, gedatolisib, omipalisib, PQR309, SF1126, voxtalisib, or a combination thereof.

Embodiment IV-15. The method of embodiments IV-1 to IV-14, wherein the one or more compounds that inhibit two or more enzymes of the PI3K/AKT/mTOR pathway comprises temsirolimus and/or everolimus, preferably wherein about 10 mg/week to about 50 mg/week, more preferably about 25 mg/week, of the temsirolimus is administered to the subject; and/or about 2 mg/day to about 10 mg/day, more preferably about 5 mg/day, of the everolimus is administered to the subject.

Embodiment IV-16. The method of embodiments IV-1 to IV-15, wherein the one or more compounds that inhibit two or more enzymes of the PI3K/AKT/mTOR pathway comprises an AKT inhibitor, preferably wherein the AKT inhibitor is capivasertib, ipatasertib, MK-2206, triciribine, ABTL0812, or a combination thereof.

Embodiment IV-17. The method of embodiments IV-1 to IV-16, wherein the one or more compounds that inhibit two or more enzymes of the PI3K/AKT/mTOR pathway comprises
(a) sapanisertib, vistusertib, onatasertib, XP-105, AVTX-006, or a combination thereof;
(b) RLY-2608, STX-478, LY4045004, LOXO-783, OKI-219, or a combination thereof; and/or
(c) alpelisib, buparlisib, copanlisib, duvelisib, idelalisib, inavolisib, linperlisib, leniolisib, pictilisib, samotolisib, serabelisib, taselisib, ZSTK474, or a combination thereof.

Embodiment IV-18. The method of embodiment IV-17, comprising administering about 100 mg/day to about 400 mg/day of the alpelisib, more preferably about 200 mg/day of the alpelisib; and/or administering about 5 mg/day to about 20 mg/day of the inavolisib, more preferably about 9 mg/day of the inavolisib.

Embodiment IV-19. The method of embodiments IV-1 to IV-18, wherein the one or more anti-cancer agents that do not directly inhibit enzymes in the PI3K/AKT/mTOR pathway comprise paclitaxel, docetaxel, or a combination thereof, preferably wherein the method comprises administering about 100 mg/m² to about 250 mg/m² of the paclitaxel every 21 days or 28 days.

Embodiment IV-20. The method of embodiment IV-19, wherein carboplatin is administered in combination with about 135 mg/m² of paclitaxel.

Embodiment IV-21. The method of embodiments IV-1 to IV-20, wherein the one or more anti-cancer agents that do not directly inhibit enzymes in the PI3K/AKT/mTOR pathway comprise a CDK4 inhibitor, a CDK6 inhibitor, a CDK4/6 inhibitor, or a combination thereof.

Embodiment IV-22. The method of embodiment IV-1 to IV-21, wherein the one or more anti-cancer agents that do not directly inhibit enzymes in the PI3K/AKT/mTOR pathway comprise abemaciclib, palbociclib, ribociclib, or a combination thereof.

Embodiment IV-23. The method of embodiment IV-22, comprising administering about 200 mg/day to about 600 mg/day of the abemaciclib, preferably administering about 300 mg/day of the abemaciclib.

Embodiment IV-24. The method of embodiments IV-22 and IV-23, wherein the palbociclib is administered in a repeating 28-day cycle comprising a palbociclib dose of about 75 mg/day to about 150 mg/day for three weeks followed by one week without administering the palbociclib.

Embodiment IV-25. The method of embodiments IV-22 to IV-24, wherein the palbociclib dose is about 125 mg/day.

Embodiment IV-26. The method of embodiments IV-22 to IV-25, wherein the ribociclib is administered in a repeating 28-day cycle comprising a ribociclib dose of about 200 mg/day to about 800 mg/day for three weeks followed by one week without administering the ribociclib.

Embodiment IV-27. The method of embodiments IV-22 to IV-26, wherein the ribociclib dose is about 600 mg/day.

Embodiment IV-28. The method of embodiments IV-1 to IV-27, wherein the one or more anti-cancer agents that do not directly inhibit enzymes in the PI3K/AKT/mTOR pathway comprise a hormone receptor targeting therapeutic agent.

Embodiment IV-29. The method of embodiment IV-28, wherein the one or more anti-cancer agents that do not directly inhibit enzymes in the PI3K/AKT/mTOR pathway comprise fulvestrant or elacestrant.

Embodiment IV-30. The method of embodiment IV-29, wherein the method comprises:
(a) administering 500 mg of fulvestrant twice in a four-week interval on day, and
(b) administering 500 mg of fulvestrant every 28 days thereafter.

Embodiment IV-31. The method of embodiments IV-29 and IV-30, comprising administering about 100 mg/day to about 600 mg/day of the elacestrant, preferably comprising administering about 345 mg/day of the elacestrant.

Embodiment IV-32. The method of embodiments IV-1 to IV-31, wherein the one or more anti-cancer agents that do not directly inhibit enzymes in the PI3K/AKT/mTOR pathway comprise a modulator of p53 activity.

Embodiment IV-33. The method of embodiment IV-32, wherein the one or more anti-cancer agents that do not directly inhibit enzymes in the PI3K/AKT/mTOR pathway comprise selinexor.

Embodiment IV-34. The method of embodiment IV-33, comprising administering about 60 mg/week to about 80 mg/week of the selinexor.

Embodiment IV-35. The method of embodiments IV-33 and IV-34, comprising administering about 60 mg/week of the selinexor if the subject in need thereof has a BMI<20 kg/m² or administering about 80 mg/week of the selinexor if the subject in need thereof has a BMI>20 kg/m².

Embodiment IV-36. The method of embodiments IV-1 to IV-35, further comprising administering an insulin suppressing diet to the subject in need thereof.

Embodiment IV-37. The method of embodiment IV-36, wherein the insulin suppressing diet comprises about 65% to about 80% of fat by w/w % of the macronutrient content.

Embodiment IV-38. The method of embodiments IV-36 and IV-37, wherein the insulin suppressing diet comprises about 8% to about 25% of protein by w/w % of the macronutrient content.

Embodiment IV-39. The method of embodiments IV-36 to IV-38, wherein the insulin suppressing diet comprises about 8% to about 15% of total carbohydrates by w/w % of the macronutrient content.

Embodiment IV-40. The method of embodiments IV-36 to IV-39, wherein the insulin suppressing diet comprises about 65% to about 80% of fat, about 8% to about 25% of protein, and about 8% to about 15% of total carbohydrates by w/w % of the macronutrient content.

Embodiment IV-41. The method of embodiments IV-36 to IV-40, wherein the insulin suppressing diet comprises about 15% to about 35% of protein and total carbohydrates by w/w % of the macronutrient content.

Embodiment IV-42. The method of embodiment IV-1, wherein the cancer is uterine cancer, breast cancer, anal cancer, head and neck cancer, bladder cancer, colorectal cancer, skin cancer, ovarian cancer, ampullary cancer, glioma, small bowel cancer, cancer of unknown primary, lung cancer, esophagogastric cancer, renal cell carcinoma, endometrial, or prostate cancer.

Embodiment IV-43. The method of embodiments IV-1 to IV-42, wherein the cancer comprises one or more one or more mutations in the following genes PIK3CB, PIK3CG, PIK3CD, PIK3C2B, RPTOR, RAPTOR, RICTOR, MLST8, DEPTOR, S6K, 4E-BP1, AKT1S1, STK11, TSC2, INPP4B, PDK1, RHEB, AMPK, GSK3, DEPDC5, NPRL2, NPRL3, or combinations thereof.

Embodiment IV-44. A method of embodiment IV-1, wherein the cancer comprises a PI3K/AKT/mTOR pathway mutation selected from the group consisting of a PTEN deletion, a PIK3R1 deletion, a PIK3CA a helical domain mutation, and a PIK3CA kinase domain mutation.

Embodiment IV-45. The method of embodiments IV-1 to IV-44, wherein the cancer was previously treated with paclitaxel, docetaxel, or a combination thereof.

Embodiment IV-46. The method of embodiments IV-1 to IV-45, wherein the method reduces phosphorylated 4E-BP1 relative to untreated tumor tissue.

### Set V:

Embodiment V-1. A method of treating breast cancer in a human subject in need thereof, comprising administering to the human subject in need thereof an effective amount of:
(a) orally administered sapanisertib and orally administered serabelisib in combination with
(b) one or more of a hormone receptor targeting therapeutic agent, a cyclin-dependent kinase (CDK) inhibitor, and a lysine acetyltransferase 6 (KAT6) inhibitor.

Embodiment V-2. A method of increasing efficacy of a hormone receptor targeting therapeutic agent in a human subject in need thereof having breast cancer, the method comprising administering to the human subject an effective amount of:
(a) orally administered sapanisertib and orally administered serabelisib in combination with the hormone receptor targeting therapeutic agent.

Embodiment V-3. The method of embodiment V-1, wherein the method comprises administering the hormone receptor targeting therapeutic agent and one or more of the CDK inhibitor and the KAT6 inhibitor.

Embodiment V-4. The method of embodiment V-2, wherein the method further comprises administering one or more of a CDK inhibitor and a KAT6 inhibitor.

Embodiment V-5. The method of embodiments V-1 to V-4, wherein the serabelisib and the sapanisertib are administered at about the same time.

Embodiment V-6. The method of embodiments V-1 to V-5, wherein the serabelisib and the sapanisertib are administered with food.

Embodiment V-7. The method of embodiments V-1 to V-6, wherein the hormone receptor targeting therapeutic agent is an aromatase inhibitor.

Embodiment V-8. The method of embodiments V-1 to V-7, wherein the hormone receptor targeting therapeutic agent is anastrozole, letrozole, or exemestane.

Embodiment V-9. The method of embodiments V-1 to V-8, wherein the hormone receptor targeting therapeutic agent is a Selective Estrogen Receptor Degrader (SERD).

Embodiment V-10. The method of embodiments V-1 to V-9, wherein the hormone receptor targeting therapeutic agent is orally administered elacestrant, imlunestrant, camizestrant, giredestrant, or palazestrant.

Embodiment V-11. The method of embodiments V-1 to V-10, wherein the hormone receptor targeting therapeutic agent is intramuscularly administered fulvestrant.

Embodiment V-12. The method of embodiments V-1 to V-11, wherein the method comprises repeating the administration of sapanisertib, serabelisib, and fulvestrant over the course of a treatment interval.

Embodiment V-13. The method of embodiments V-1 to V-12, wherein the CDK inhibitor inhibits one of more of CDK2, CDK4, CDK6, CDK7, CDK8, CDK9, CDK19, CDK12, and CDK13.

Embodiment V-14. The method of embodiments V-1 to V-13, wherein the CDK inhibitor is palbociclib, ribociclib, abemaciclib, Trilaciclib, dalpiciclib.

Embodiment V-15. The method of embodiments V-1 to V-14, wherein the CDK inhibitor is orally administered palbociclib.

Embodiment V-16. The method of embodiments V-1 to V-15, wherein the method comprises repeating the administration of sapanisertib, serabelisib, fulvestrant, and palbociclib over the course of a treatment interval.

Embodiment V-17. The method of embodiments V-1 to V-16, wherein 100-400 mg serabelisib are administered.

Embodiment V-18. The method of embodiments V-1 to V-17, wherein 1-5 mg sapanisertib are administered.

Embodiment V-19. The method of embodiments V-1 to V-18, wherein the method comprises a four-week interval:
(a) wherein each week of the four-week interval comprises administering the sapanisertib and the serabelisib for each of three consecutive days.

Embodiment V-20. The method of embodiment V-19, wherein 100-400 mg serabelisib and 1-5 mg sapanisertib are administered on each of the three consecutive days of the four-week interval.

Embodiment V-21. The method of embodiment V-19, wherein 100-400 mg serabelisib and 1-5 mg sapanisertib are administered on day 1, day 2, day 3, day 8, day 9, day 10, day 15, day 16, day 17, day 22, day 23 and day 24 of the four-week interval.

Embodiment V-22. The method of embodiment V-19, wherein 100 mg serabelisib and 2 mg sapanisertib are administered on day 1, day 2, day 3, day 8, day 9, day 10, day 15, day 16, day 17, day 22, day 23 and day 24 of the four-week interval.

Embodiment V-23. The method of embodiment V-19, wherein 200 mg serabelisib and 2 mg sapanisertib are administered on day 1, day 2, day 3, day 8, day 9, day 10, day 15, day 16, day 17, day 22, day 23 and day 24 of the four-week interval.

Embodiment V-24. The method of embodiment V-19, wherein 300 mg serabelisib and 2 mg sapanisertib are administered on day 1, day 2, day 3, day 8, day 9, day 10, day 15, day 16, day 17, day 22, day 23 and day 24 of the four-week interval.

Embodiment V-25. The method of embodiment V-19, wherein 100 mg serabelisib and 3 mg sapanisertib are administered on day 1, day 2, day 3, day 8, day 9, day 10, day 15, day 16, day 17, day 22, day 23 and day 24 of the four-week interval.

Embodiment V-26. The method of embodiment V-19, wherein 200 mg serabelisib and 3 mg sapanisertib are administered on day 1, day 2, day 3, day 8, day 9, day 10, day 15, day 16, day 17, day 22, day 23 and day 24 of the four-week interval.

Embodiment V-27. The method of embodiment V-19, wherein 300 mg serabelisib and 3 mg sapanisertib are administered on day 1, day 2, day 3, day 8, day 9, day 10, day 15, day 16, day 17, day 22, day 23 and day 24 of the four-week interval.

Embodiment V-28. The method of embodiment V-19, wherein 100 mg serabelisib and 4 mg sapanisertib are administered on day 1, day 2, day 3, day 8, day 9, day 10, day 15, day 16, day 17, day 22, day 23 and day 24 of the four-week interval.

Embodiment V-29. The method of embodiment V-19, wherein 200 mg serabelisib and 4 mg sapanisertib are administered on day 1, day 2, day 3, day 8, day 9, day 10, day 15, day 16, day 17, day 22, day 23 and day 24 of the four-week interval.

Embodiment V-30. The method of embodiment V-19, wherein 300 mg serabelisib and 4 mg sapanisertib are administered on day 1, day 2, day 3, day 8, day 9, day 10, day 15, day 16, day 17, day 22, day 23 and day 24 of the four-week interval.

Embodiment V-31. The method of embodiments V-1 to V-30, wherein 200-800 mg fulvestrant are administered.

Embodiment V-32. The method of embodiments V-1 to V-31, wherein 500 mg fulvestrant are administered.

Embodiment V-33. The method of embodiments V-1 to V-32, wherein 200-800 mg fulvestrant are administered on day 1 and day 15 of the four-week interval.

Embodiment V-34. The method of embodiments V-1 to V-33, wherein 500 mg fulvestrant are administered on day 1 and day 15 of the four-week interval.

Embodiment V-35. The method of embodiments V-1 to V-34, wherein 200-800 mg fulvestrant are administered once monthly after the four-week interval.

Embodiment V-36. The method of embodiments V-1 to V-35, wherein 500 mg fulvestrant are administered once monthly after the four-week interval.

Embodiment V-37. The method of embodiment V-19, wherein 200 mg serabelisib and 2 mg sapanisertib are administered on day 1, day 2, day 3, day 8, day 9, day 10, day 15, day 16, day 17, day 22, day 23 and day 24 of the four-week interval and 500 mg fulvestrant are administered on day 1 and day 15 of the four-week interval.

Embodiment V-38. The method of embodiment V-19, wherein 300 mg serabelisib and 2 mg sapanisertib are administered on day 1, day 2, day 3, day 8, day 9, day 10, day 15, day 16, day 17, day 22, day 23 and day 24 of the four-week interval and 500 mg fulvestrant are administered on day 1 and day 15 of the four-week interval.

Embodiment V-39. The method of embodiment V-19, wherein 100 mg serabelisib and 3 mg sapanisertib are administered on day 1, day 2, day 3, day 8, day 9, day 10, day 15, day 16, day 17, day 22, day 23 and day 24 of the four-week interval and 500 mg fulvestrant are administered on day 1 and day 15 of the four-week interval.

Embodiment V-40. The method of embodiment V-19, wherein 200 mg serabelisib and 3 mg sapanisertib are administered on day 1, day 2, day 3, day 8, day 9, day 10, day 15, day 16, day 17, day 22, day 23 and day 24 of the four-week interval and 500 mg fulvestrant are administered on day 1 and day 15 of the four-week interval.

Embodiment V-41. The method of embodiment V-19, wherein 300 mg serabelisib and 3 mg sapanisertib are administered on day 1, day 2, day 3, day 8, day 9, day 10, day 15, day 16, day 17, day 22, day 23 and day 24 of the four-week interval and 500 mg fulvestrant are administered on day 1 and day 15 of the four-week interval.

Embodiment V-42. The method of embodiment V-19, wherein 100 mg serabelisib and 4 mg sapanisertib are administered on day 1, day 2, day 3, day 8, day 9, day 10, day 15, day 16, day 17, day 22, day 23 and day 24 of the four-week interval and 500 mg fulvestrant are administered on day 1 and day 15 of the four-week interval.

Embodiment V-43. The method of embodiment V-19, wherein 200 mg serabelisib and 4 mg sapanisertib are administered on day 1, day 2, day 3, day 8, day 9, day 10, day 15, day 16, day 17, day 22, day 23 and day 24 of the four-week interval and 500 mg fulvestrant are administered on day 1 and day 15 of the four-week interval.

Embodiment V-44. The method of embodiment V-19, wherein 300 mg serabelisib and 4 mg sapanisertib are administered on day 1, day 2, day 3, day 8, day 9, day 10, day 15, day 16, day 17, day 22, day 23 and day 24 of the four-week interval and 500 mg fulvestrant are administered on day 1 and day 15 of the four-week interval.

Embodiment V-45. The method of embodiments V-1 to V-44, wherein 60 mg to 240 mg palbociclib are administered.

Embodiment V-46. The method of embodiments V-1 to V-45, wherein 60 mg to 240 mg palbociclib are administered once daily for each of 21 consecutive days of the four-week interval.

Embodiment V-47. The method of embodiments V-1 to V-46, wherein 125 mg palbociclib is administered once daily for each of 21 consecutive days of the four-week interval.

Embodiment V-48. The method of embodiment V-19, wherein 300 mg serabelisib and 2 mg sapanisertib are administered on day 1, day 2, day 3, day 8, day 9, day 10, day 15, day 16, day 17, day 22, day 23 and day 24 of the four-week interval and 125 mg palbociclib is administered once daily for each of 21 consecutive days of the four-week interval.

Embodiment V-49. The method of embodiment V-19, wherein 100 mg serabelisib and 3 mg sapanisertib are administered on day 1, day 2, day 3, day 8, day 9, day 10, day 15, day 16, day 17, day 22, day 23 and day 24 of the four-week interval and 125 mg palbociclib is administered once daily for each of 21 consecutive days of the four-week interval.

Embodiment V-50. The method of embodiment V-19, wherein 200 mg serabelisib and 3 mg sapanisertib are administered on day 1, day 2, day 3, day 8, day 9, day 10, day 15, day 16, day 17, day 22, day 23 and day 24 of the four-week interval and 125 mg palbociclib is administered once daily for each of 21 consecutive days of the four-week interval.

Embodiment V-51. The method of embodiment V-19, wherein 300 mg serabelisib and 3 mg sapanisertib are administered on day 1, day 2, day 3, day 8, day 9, day 10, day 15, day 16, day 17, day 22, day 23 and day 24 of the four-week interval and 125 mg palbociclib is administered once daily for each of 21 consecutive days of the four-week interval.

Embodiment V-52. The method of embodiment V-19, wherein 100 mg serabelisib and 4 mg sapanisertib are administered on day 1, day 2, day 3, day 8, day 9, day 10, day 15, day 16, day 17, day 22, day 23 and day 24 of the four-week interval and 125 mg palbociclib is administered once daily for each of 21 consecutive days of the four-week interval.

Embodiment V-53. The method of embodiment V-19, wherein 200 mg serabelisib and 4 mg sapanisertib are administered on day 1, day 2, day 3, day 8, day 9, day 10, day 15, day 16, day 17, day 22, day 23 and day 24 of the four-week interval and 125 mg palbociclib is administered once daily for each of 21 consecutive days of the four-week interval.

Embodiment V-54. The method of embodiment V-19, wherein 300 mg serabelisib and 4 mg sapanisertib are administered on day 1, day 2, day 3, day 8, day 9, day 10, day 15, day 16, day 17, day 22, day 23 and day 24 of the four-week interval and 125 mg palbociclib is administered once daily for each of 21 consecutive days of the four-week interval.

Embodiment V-55. The method of embodiment V-19, wherein 200 mg serabelisib and 2 mg sapanisertib are administered on day 1, day 2, day 3, day 8, day 9, day 10, day 15, day 16, day 17, day 22, day 23 and day 24 of the four-week interval; 500 mg fulvestrant are administered on day 1 and day 15 of the four-week interval; and 125 mg palbociclib is administered once daily for each of 21 consecutive days of the four-week interval.

Embodiment V-56. The method of embodiment V-19, wherein 300 mg serabelisib and 2 mg sapanisertib are administered on day 1, day 2, day 3, day 8, day 9, day 10, day 15, day 16, day 17, day 22, day 23 and day 24 of the four-week interval; 500 mg fulvestrant are administered on day 1 and day 15 of the four-week interval; and 125 mg palbociclib is administered once daily for each of 21 consecutive days of the four-week interval.

Embodiment V-57. The method of embodiment V-19, wherein 100 mg serabelisib and 3 mg sapanisertib are administered on day 1, day 2, day 3, day 8, day 9, day 10, day 15, day 16, day 17, day 22, day 23 and day 24 of the four-week interval; 500 mg fulvestrant are administered on day 1 and day 15 of the four-week interval; and 125 mg palbociclib is administered once daily for each of 21 consecutive days of the four-week interval.

Embodiment V-58. The method of embodiment V-19, wherein 200 mg serabelisib and 3 mg sapanisertib are administered on day 1, day 2, day 3, day 8, day 9, day 10, day 15, day 16, day 17, day 22, day 23 and day 24 of the four-week interval; 500 mg fulvestrant are administered on day 1 and day 15 of the four-week interval; and 125 mg palbociclib is administered once daily for each of 21 consecutive days of the four-week interval.

Embodiment V-59. The method of embodiment V-19, wherein 300 mg serabelisib and 3 mg sapanisertib are administered on day 1, day 2, day 3, day 8, day 9, day 10, day 15, day 16, day 17, day 22, day 23 and day 24 of the four-week interval; 500 mg fulvestrant are administered on day 1 and day 15 of the four-week interval; and 125 mg palbociclib is administered once daily for each of 21 consecutive days of the four-week interval.

Embodiment V-60. The method of embodiment V-19, wherein 100 mg serabelisib and 4 mg sapanisertib are administered on day 1, day 2, day 3, day 8, day 9, day 10, day 15, day 16, day 17, day 22, day 23 and day 24 of the four-week interval; 500 mg fulvestrant are administered on day 1 and day 15 of the four-week interval; and 125 mg palbociclib is administered once daily for each of 21 consecutive days of the four-week interval.

Embodiment V-61. The method of embodiment V-19, wherein 200 mg serabelisib and 4 mg sapanisertib are administered on day 1, day 2, day 3, day 8, day 9, day 10, day 15, day 16, day 17, day 22, day 23 and day 24 of the four-week interval; 500 mg fulvestrant are administered on day 1 and day 15 of the four-week interval; and 125 mg palbociclib is administered once daily for each of 21 consecutive days of the four-week interval.

Embodiment V-62. The method of embodiment V-19, wherein 300 mg serabelisib and 4 mg sapanisertib are administered on day 1, day 2, day 3, day 8, day 9, day 10, day 15, day 16, day 17, day 22, day 23 and day 24 of the four-week interval; 500 mg fulvestrant are administered on day 1 and day 15 of the four-week interval; and 125 mg palbociclib is administered once daily for each of 21 consecutive days of the four-week interval.

Embodiment V-63. The method of embodiments V-1 to V-62, wherein the treating comprises providing a progression-free survival (PFS) of at least six months, preferably at least nine months, more preferably at least 3 months, at least 6 months, or at least 12 months to the subject.

Embodiment V-64. The method of embodiments V-1 to V-63, wherein the treating comprises providing an overall survival (OS) of at least nine months, at least 12 months, or at least 15 months to the subject.

Embodiment V-65. The method of embodiments V-1 to V-64, wherein the treating comprises providing a duration of response (DoR) of at least six months, at least nine months, at least 12 months, or at least 15 months to the subject.

Embodiment V-66. The method of embodiments V-1 to V-65, wherein the treating comprises providing a complete response (CR) or a partial response (PR) to the subject.

Embodiment V-67. The method of embodiments V-1 to V-66, wherein the treating comprises the subject presenting no grade 3 or grade 4 hyperglycemia.

Embodiment V-68. The method of embodiments V-1 to V-67, wherein the treating comprises providing the subject with a less than 30% likelihood of presenting hyperglycemia, preferably with a less than 20% likelihood of presenting hyperglycemia, more preferably with a less than 15% likelihood of presenting hyperglycemia.

Embodiment V-69. The method of embodiments V-1 to V-68, wherein the treating comprises providing the subject with a less than 15% likelihood of presenting grade 3 or grade 4 hyperglycemia, preferably with a less than 10% likelihood of presenting grade 3 or grade 4 hyperglycemia, preferably with a less than 7% likelihood of presenting grade 3 or grade 4 hyperglycemia, more preferably with a less than 5% likelihood of presenting grade 3 or grade 4 hyperglycemia.

Embodiment V-70. The method of embodiments V-1 to V-69, wherein the treating comprises the subject presenting no grade 3 or grade 4 stomatitis.

Embodiment V-71. The method of embodiments V-1 to V-70, wherein the treating comprises providing the subject with a less than 80% likelihood of presenting stomatitis, preferably with a less than 65% likelihood of presenting stomatitis, preferably with a less than 50% likelihood of presenting stomatitis, more preferably with a less than 30% of presenting stomatitis, more preferably with a less than 20% likelihood of presenting stomatitis, more preferably with a less than 15% likelihood of presenting stomatitis.

Embodiment V-72. The method of embodiments V-1 to V-71, wherein the treating comprises providing the subject with a less than 30% likelihood of presenting grade 3 or grade 4 stomatitis, preferably with a less than 20% likelihood of presenting grade 3 or grade 4 stomatitis, preferably with a less than 15% likelihood of presenting grade 3 or grade 4 stomatitis, more preferably with a less than 10% likelihood of presenting grade 3 or grade 4 stomatitis, more preferably with a less than 5% likelihood of presenting grade 3 or grade 4 stomatitis.

Embodiment V-73. The method of embodiments V-1 to V-72, wherein the treating excludes administration of a steroid or corticosteroid.

Embodiment V-74. The method of embodiments V-1 to V-73, wherein the treating excludes administration of a prophylactic mouth rinse comprising a steroid or corticosteroid.

Embodiment V-75. The method of embodiments V-1 to V-74, wherein the treating excludes any measures to prevent, ameliorate, treat, or reduce stomatitis.

Embodiment V-76. The method of embodiments V-1 to V-75, wherein the method of treatment proceeds without inducing grade 4 stomatitis and without inducing grade 4 hyperglycemia.

Embodiment V-77. The method of embodiments V-1 to V-76, wherein the method of treatment proceeds without inducing grade 3 stomatitis.

Embodiment V-78. The method of embodiments V-1 to V-77, wherein the treating excludes administration of a steroid or corticosteroid.

Embodiment V-79. The method of embodiments V-1 to V-78, wherein the treating excludes administration of a prophylactic mouth rinse comprising a steroid or corticosteroid.

Embodiment V-80. The method of embodiments V-1 to V-79, wherein the treating excludes any measures to prevent, ameliorate, treat, or reduce stomatitis.

Embodiment V-81. The method of embodiments V-1 to V-80, wherein the combination increases PFS; increases DCR; increases ORR; increases CBR; increases OS; increases DoR; decreases likelihood of presenting hyperglycemia; or decreases likelihood of presenting stomatitis when compared to administration of the hormone receptor targeting therapeutic agent without the orally administered sapanisertib and orally administered serabelisib.

Embodiment V-82. The method of embodiments V-1 to V-81, wherein the combination increases PFS; increases DCR; increases ORR; increases CBR; increases OS; increases DoR; decreases likelihood of presenting hyperglycemia; or decreases likelihood of presenting stomatitis when compared to administration of the orally administered sapanisertib and orally administered serabelisib without the hormone receptor targeting therapeutic agent.

Embodiment V-83. The method of embodiments V-1 to V-82, wherein the combination increases PFS; increases DCR; increases ORR; increases CBR; increases OS; increases DoR; decreases likelihood of presenting hyperglycemia; or decreases likelihood of presenting stomatitis when compared to administration of the CDK inhibitor or the KAT6 inhibitor without the orally administered sapanisertib and orally administered serabelisib.

Embodiment V-84. The method of embodiments V-1 to V-83, wherein the combination increases PFS; increases DCR; increases ORR; increases CBR; increases OS; increases DoR; decreases likelihood of presenting hyperglycemia; or decreases likelihood of presenting stomatitis when compared to administration of the orally administered sapanisertib and orally administered serabelisib without the CDK inhibitor or the KAT6 inhibitor.

Embodiment V-85. The method of embodiments V-1 to V-84, wherein the subject in need thereof has advanced or metastatic breast cancer.

Embodiment V-86. The method of embodiments V-1 to V-85, wherein the breast cancer is resistant to the hormone receptor targeting therapeutic agent.

Embodiment V-87. The method of embodiments V-1 to V-86, wherein the breast cancer is resistant to an aromatase inhibitor.

Embodiment V-88. The method of embodiments V-1 to V-87, wherein the breast cancer is characterized by upregulated expression of ESR1 gene.

Embodiment V-89. The method of embodiments V-1 to V-88, wherein the breast cancer is ESR1 negative.

Embodiment V-90. The method of embodiments V-1 to V-89, wherein the cancer is hormone receptor positive (HR+).

Embodiment V-91. The method of embodiments V-1 to V-90, wherein the cancer is human epidermal growth factor receptor 2 negative (HER2−).

Embodiment V-92. The method of embodiments V-1 to V-91, wherein the cancer is HR+ and HER2−.

Embodiment V-93. The method of embodiments V-1 to V-92, wherein the subject has a mutation identified in a gene of the PI3K/AKT/MTOR pathway, and/or has a mutated PI3K pathway.

Embodiment V-94. The method of embodiments V-1 to V-93, wherein the subject comprises one or more mutations in at least one gene selected from the group consisting of PIK3CA, DEPTOR, PIK3C2B, PRKAB2, PTEN, PREX2, NPRL3, MLST8, RPTOR, RICTOR, PIK3R1, AKT1, AKT2, AKT3, TSC1, TSC2, MTOR, STK11, GSK3A, GSK3B, DEPDC5, PRKAA1, PRKAA2, AKT1S1, GOLPH3, PDPK1, PRKAG2, PRKAG1, PIK3CB, PIK3CD, PIK3CG, RHEB, INPP4B, PPP2R1A, PRKAB1, PRKAG3, MAPKAP1, and NPRL2.

Embodiment V-95. The method of embodiments V-1 to V-94, wherein the subject comprises a mutation of PIK3CA.

Embodiment V-96. The method of embodiments V-1 to V-94, wherein the subject comprises wild type PIK3CA.

Embodiment V-97. The method of embodiments V-1 to V-95, wherein the subject comprises a mutation of AKT1.

Embodiment V-98. The method of embodiments V-1 to V-95, wherein the subject comprises wild type AKT1.

Embodiment V-99. The method of embodiments V-1 to V-96, wherein the subject comprises a mutation of PTEN.

Embodiment V-100. The method of embodiments V-1 to V-96, wherein the subject comprises wild type PTEN.

Embodiment V-101. The method of embodiments V-1 to V-92, wherein the subject comprises wild types of PIK3CA, DEPTOR, PIK3C2B, PRKAB2, PTEN, PREX2, NPRL3, MLST8, RPTOR, RICTOR, PIK3R1, AKT1, AKT2, AKT3, TSC1, TSC2, MTOR, STK11, GSK3A, GSK3B, DEPDC5, PRKAA1, PRKAA2, AKT1S1, GOLPH3, PDPK1, PRKAG2, PRKAG1, PIK3CB, PIK3CD, PIK3CG, RHEB, INPP4B, PPP2R1A, PRKAB1, PRKAG3, MAPKAP1, and NPRL2.

Embodiment V-102. The method of embodiments V-1 to V-101, wherein prior to the method of treating, the subject previously received one to four prior lines of therapy.

Embodiment V-103. The method embodiment V-102, wherein the one to four prior lines of therapy comprise a hormone receptor targeting therapeutic agent.

Embodiment V-104. The method of embodiments V-102 and V-103, wherein the one to four prior lines of therapy comprise administering a PI3K/AKT/MTOR pathway inhibitor.

Embodiment V-105. The method of embodiments V-102 and V-103, wherein the one to four prior lines of therapy do not comprise administering a PI3K/AKT/MTOR pathway inhibitor.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLES

### Example 1: A Multi-Node Inhibitor Strategy Utilizing Sapanisertib and Serabelisib Achieves Improved PI3K/AKT/mTOR Pathway Inhibition Versus Single Node Inhibitors Alpelisib, Capivasertib and Everolimus

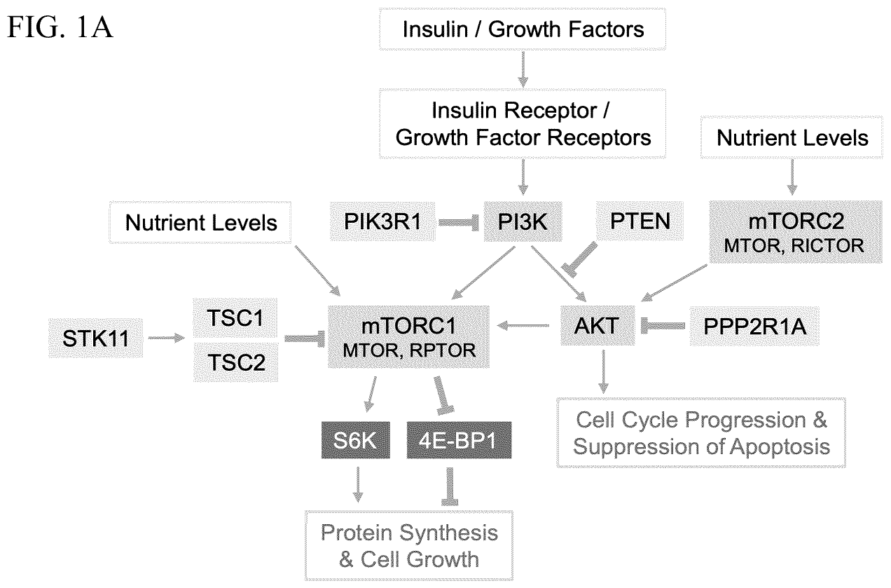

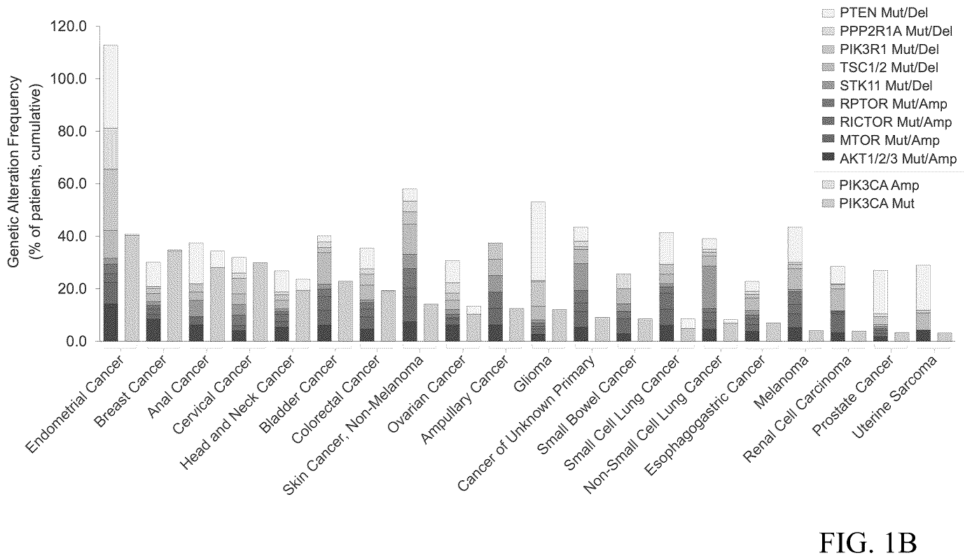

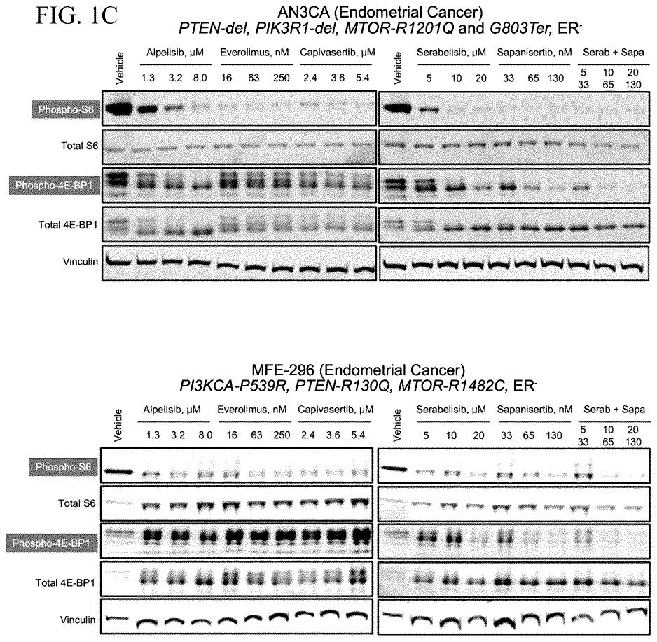

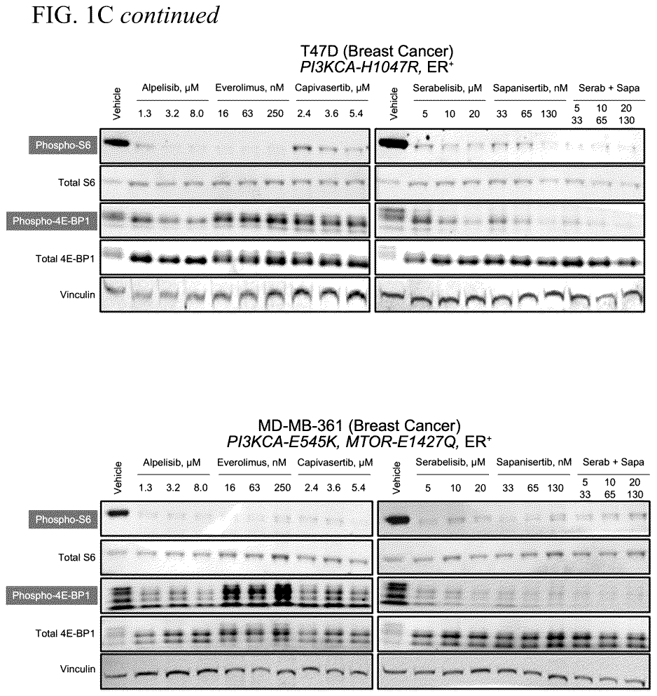

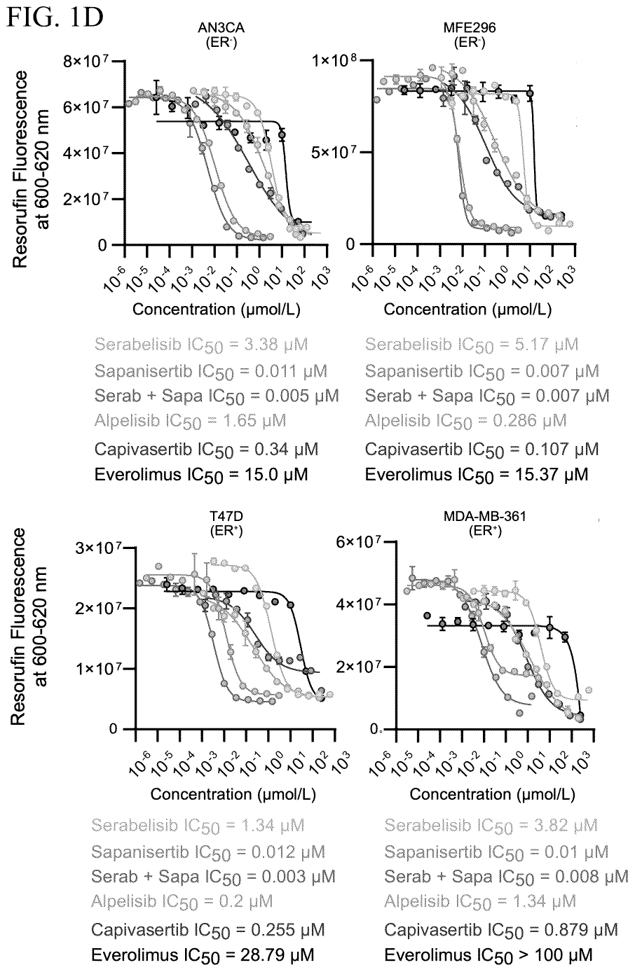

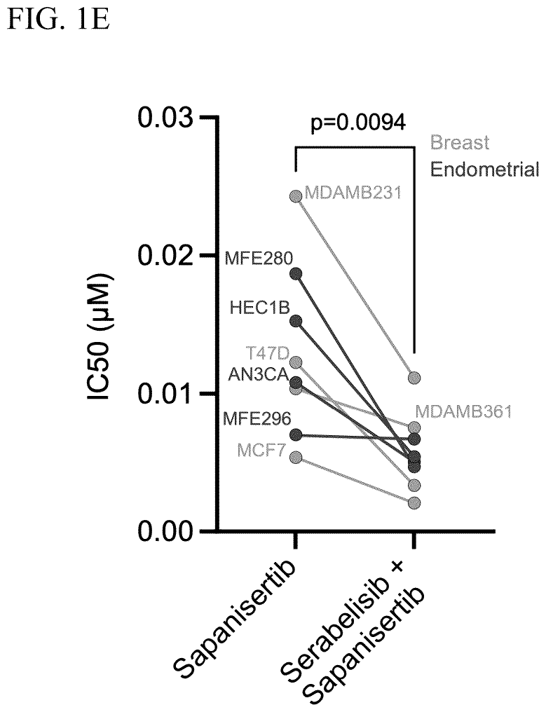

The PI3K/AKT/mTOR signaling pathway represents the major control hub for metabolic signaling in mammalian biology. This multi-node pathway senses nutrient and growth factor levels, and under favorable conditions signals to activate protein translation to support growth and proliferation, while simultaneously promoting cell cycle progression and inhibiting apoptosis (FIG. 1A). Consistent with its role in controlling cellular growth and proliferation, mutations that activate PI3K/AKT/mTOR signaling are frequent genetic alterations in cancer. While PIK3CA (the gene encoding PI3Kα) mutation is the most frequent PI3K pathway-related individual genetic alteration, broader pathway mutations cumulatively outweigh PIK3CA mutation in most solid tumors (FIG. 1).

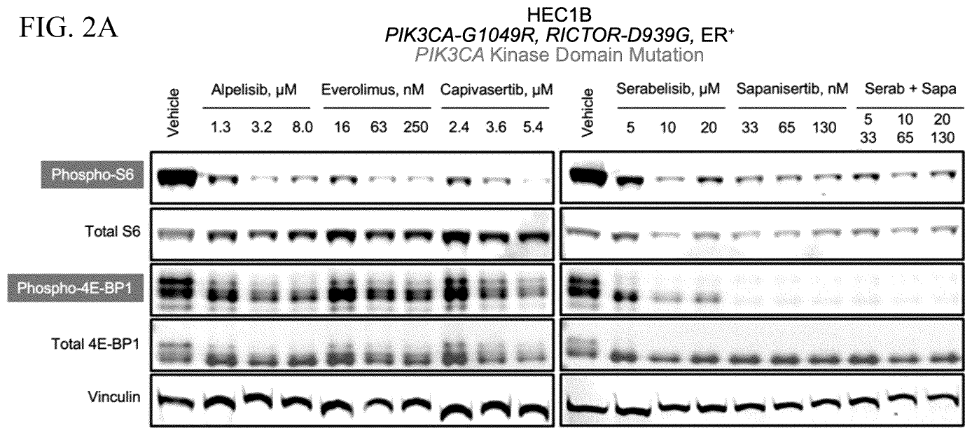

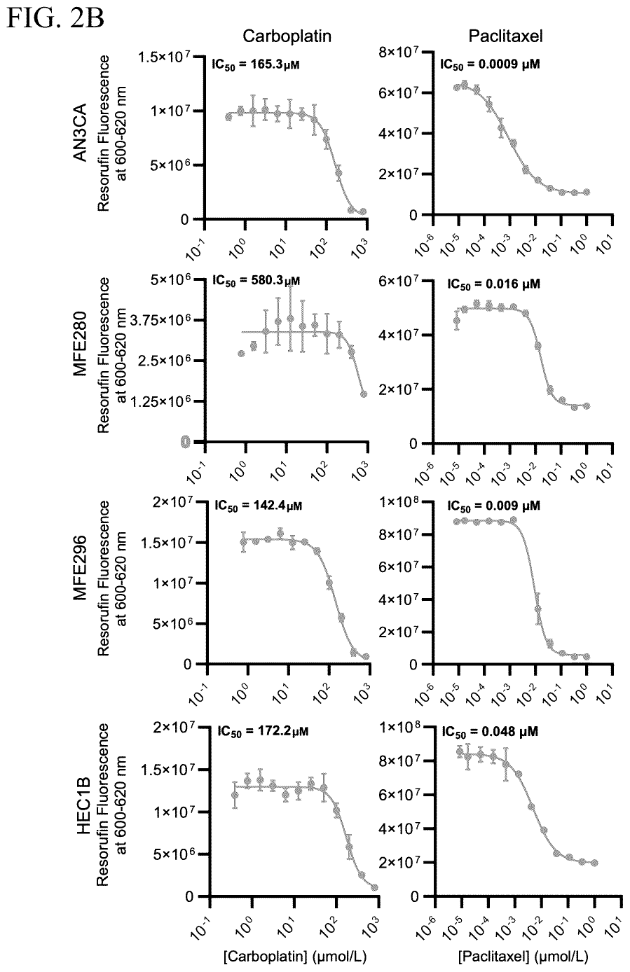

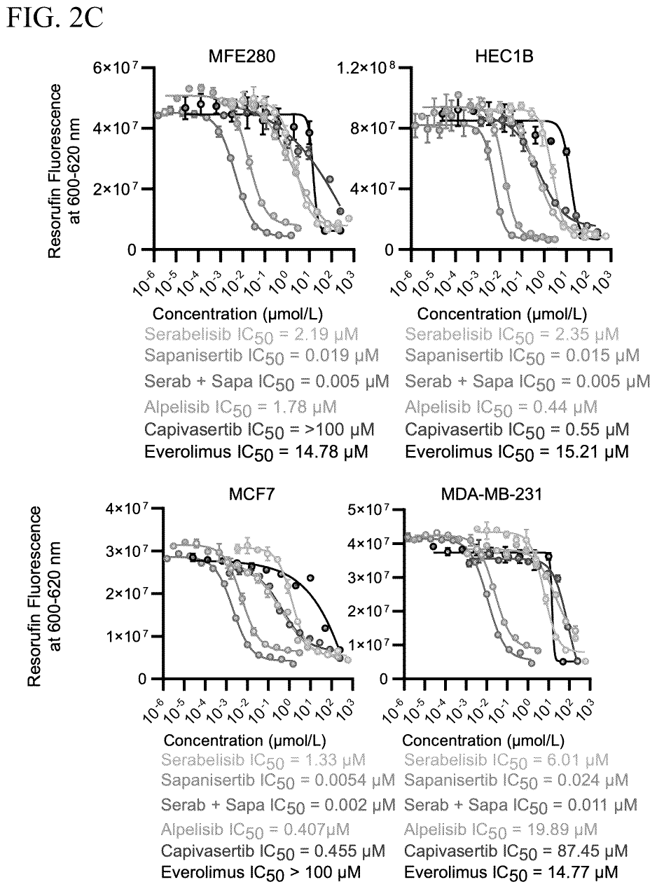

Endometrial and breast cancer cells harboring a range of PI3K/AKT/mTOR pathway mutations were treated in vitro with concentrations of drug that reflect free-drug exposures in patients treated with these agents (i.e., clinically relevant concentrations of each agent, representing C_{average}, Cₘₐₓᵢₘᵤₘ and mid-point of these). Western blots show that while approved SNIs alpelisib, everolimus and capivasertib decreased S6 phosphorylation (signifying lower pro-growth signaling), they showed very limited ability to prevent 4E-BP1 phosphorylation (i.e., pro-growth phosphorylated-4E-BP1 signal was maintained) (FIG. 1C and FIG. 2A). As an SNI, the α-specific PI3K inhibitor serabelisib was also able to inhibit S6 activation to a greater extent than 4E-BP1 activity—albeit serabelisib tended to inhibit 4E-BP1 phosphorylation to a greater extent than the α-specific PI3K inhibitor alpelisib. In contrast, sapanisertib, a second-generation Mtor inhibitor which targets both Mtorc1 and Mtorc2, achieved excellent inhibition of S6 phosphorylation and much improved inhibition of 4E-BP1 phosphorylation versus single-node inhibitors. By far the most complete pathway inhibition (i.e., simultaneously reduced signal for phosphorylated-S6 and phosphorylated 4E-BP1) was achieved by the combination of sapanisertib and serabelisib. This multi-node approach was able to dramatically inhibit PI3K/AKT/mTOR pathway activity even in cell lines with 2-3 PI3K/AKT/mTOR pathway mutations (FIG. 1C and FIG. 2A).

### Example 2: Sapanisertib and Serabelisib Potently Inhibit Cancer Cell Growth Individually and in Combination

Carboplatin and paclitaxel are mainstays in the early-line treatment of advanced/recurrent endometrial cancer. In endometrial cancer cell lines, carboplatin and paclitaxel mean IC₅₀s were 265 Mm and 18.5 Nm respectively (FIG. 2B). Among the PI3K/AKT/mTOR pathway inhibitors tested, sapanisertib showed lowest single agent IC₅₀ (average=13 Nm, similar to paclitaxel) (FIG. 1D and FIG. 2C). Furthermore, the combination of sapanisertib plus serabelisib, which had the greatest impact on 4E-BP1 phosphorylation, had an even lower mean IC₅₀ of 5.5 Nm, a significant improvement on sapanisertib alone (FIG. 1D and FIG. 2C), and on average more potent than the other targeted and chemotherapies tested, and within the clinical exposure levels for these agents.

### Example 3: Sapanisertib Plus Serabelisib Achieves Improved PI3K/AKT/Mtor Pathway Inhibition Versus Single Node Inhibitors Inavolisib, RLY-2608 and STX-478

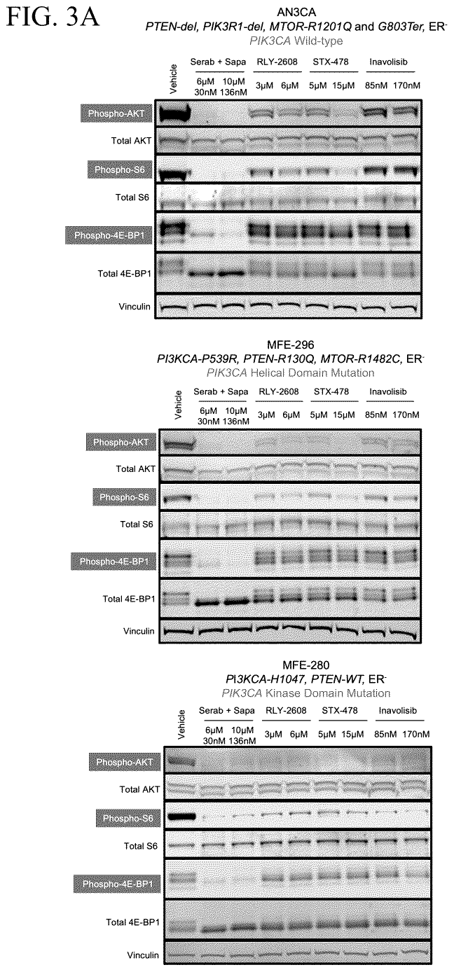

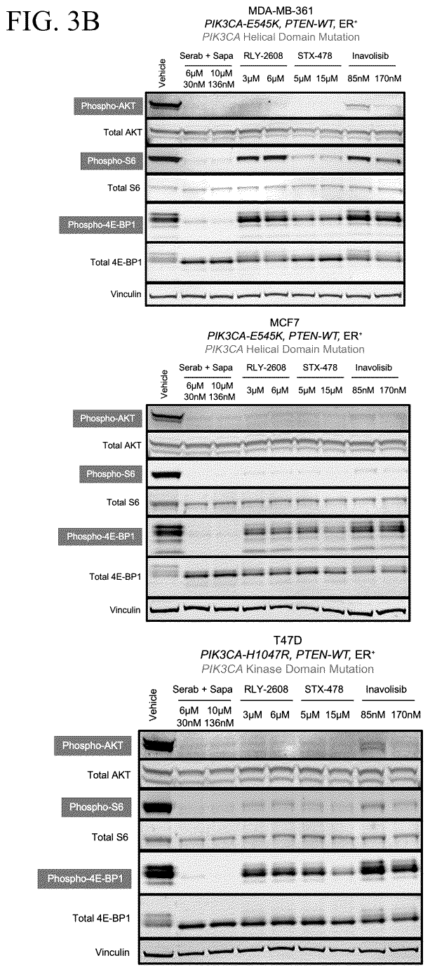

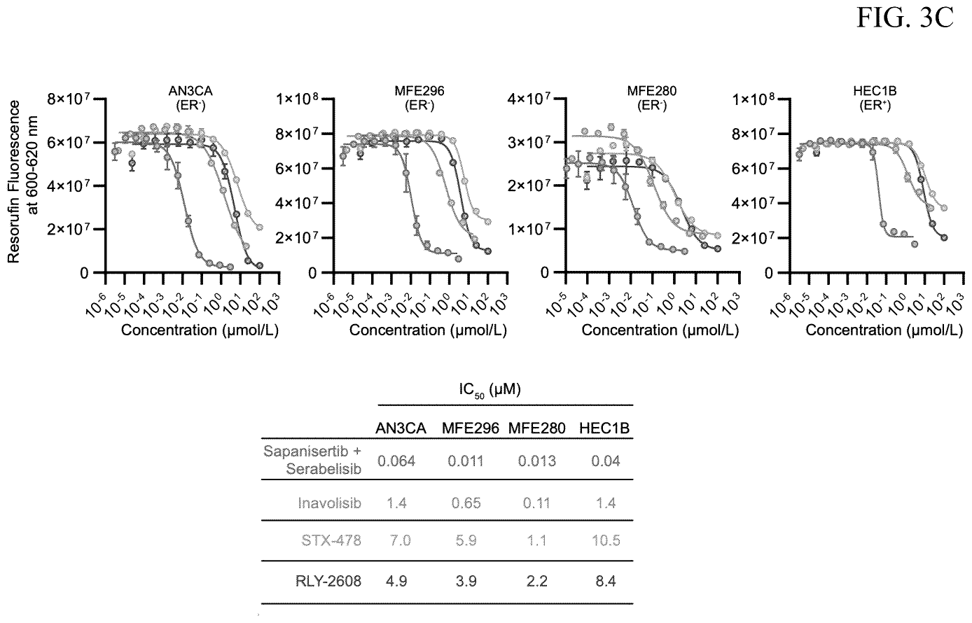

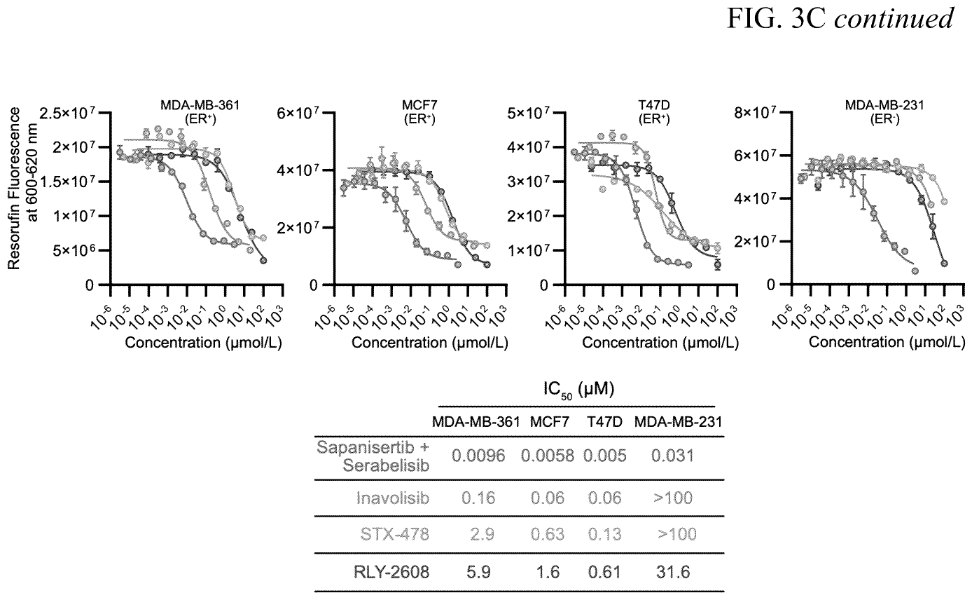

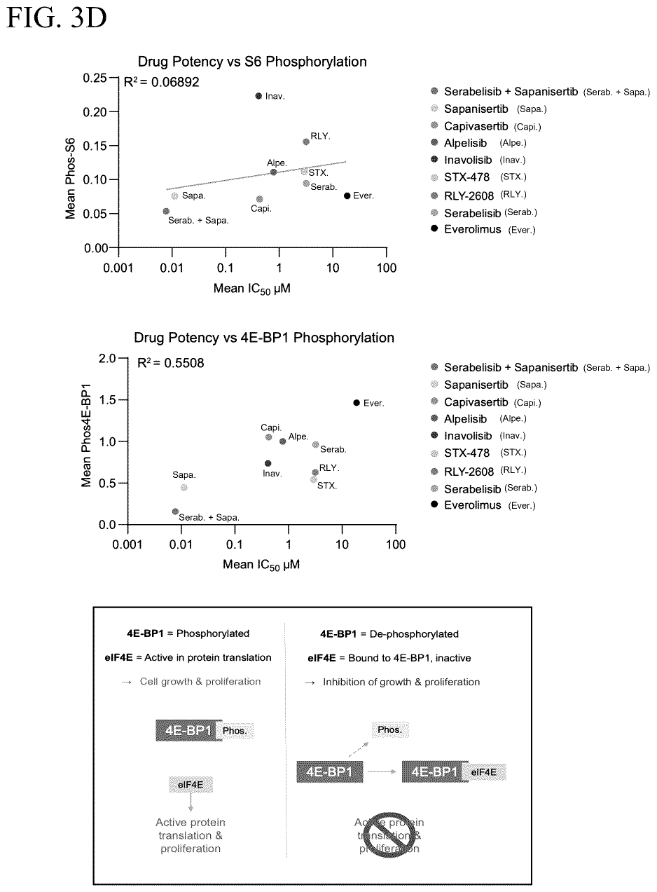

Inavolisib (α-specific PI3K inhibitor/degrader), RLY-2608 and STX-478 (mutant specific PI3K inhibitors) are SNIs of the PI3K/AKT/Mtor pathway currently in clinical development. In similarity to clinically approved SNIs (i.e., alpelisib, everolimus and capivasertib) inavolisib, RLY-2608 and STX-478 showed only limited ability to inhibit PI3K/AKT/Mtor pathway signaling in breast and endometrial cancer cells (FIG. 3A and FIG. 3B). At clinically relevant concentrations these SNIs generally achieved variable degrees of inhibition of S6 and AKT phosphorylation but had little impact on 4E-BP1-phosphorylation. In contrast, the MNI combination of sapanisertib plus serabelisib generally showed a greater inhibition of S6 and AKT phosphorylation and substantially greater inhibition of 4E-BP1 phosphorylation in endometrial and breast cancer cells. Notably the MNI approach outperformed the SNIs independently of whether the cells harbored wild-type PIK3CA, helical domain mutations of PIK3CA, kinase domain mutations of PIK3CA, or PTEN loss-of-function mutations. Also of note, despite the substantial cross-talk between Estrogen Receptor (ER) signaling and the PI3K/AKT/Mtor pathway, ER status did not impact the potency of serabelisib and sapanisertib in breast and endometrial cancer cell lines.

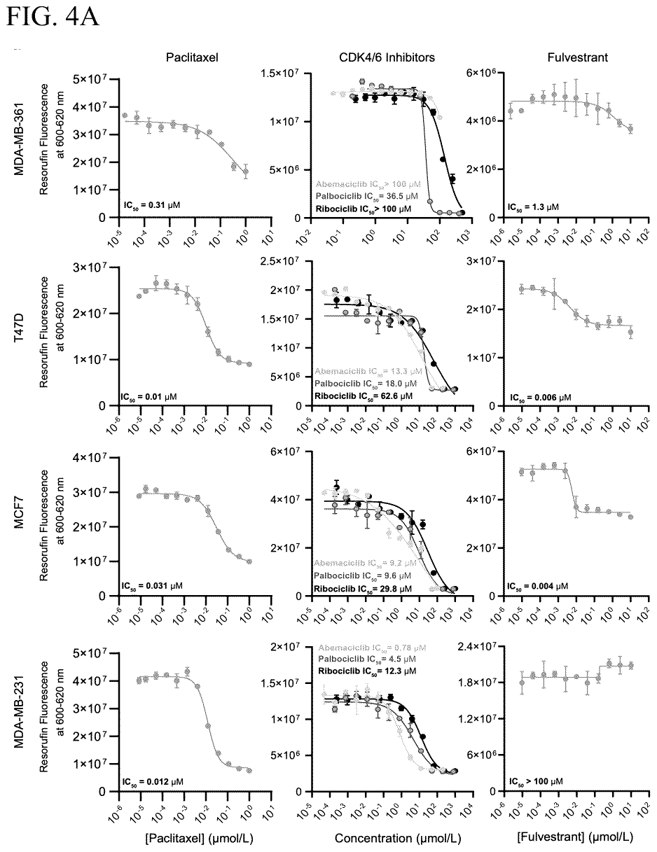

Compared to mutant-specific PI3Kα inhibitors (RLY-2608 & STX-478) and PI3Kα inhibitor/degrader inavolisib, the sapanisertib plus serabelisib combination had a substantially lower mean IC₅₀ i.e., 32.0 Nm versus 0.89 Mm (inavolisib), 6.13 Mm (STX-478), 4.85 Mm (RLY-2608) (FIG. 3C). A very similar pattern of results was observed in a range of breast cancer cell lines. Mean IC₅₀ of sapanisertib alone (12.9 Nm) or in combination with serabelisib (6 Nm) was superior to other clinically relevant agents such as paclitaxel (90.8 Nm), fulvestrant (437 Nm for ER+ cell lines), alpelisib (5.5 Mm), capivasertib (22.3 Mm), everolimus (60.9 Mm) and CDK4/6 inhibitors abemaciclib (30.8 Mm), palbociclib (17.2 Mm) & ribociclib (51.2 Mm) (FIG. 3C and FIG. 4A. Compared to mutant-specific PI3Kα inhibitors and inavolisib, which had micromolar IC₅₀, sapanisertib/serabelisib also had a substantially lower (i.e., nanomolar) IC₅₀ (FIG. 3C).

### Example 4: Inhibition of 4E-BP1 (but not S6) Phosphorylation Correlates with Drug Potency

Given the marked difference between MNI and SNI approaches with respect to suppression of 4E-BP1 phosphorylation, we hypothesized that inhibition of 4E-BP1 phosphorylation, rather than inhibition of S6 phosphorylation, would be correlated with dose-response. Notably, there was a strong correlation between drug potency (IC₅₀) and inhibition of 4E-BP1 phosphorylation, whereas inhibition of S6 phosphorylation did not correlate with drug potency (FIG. 3D).

### Example 5: Sapanisertib Plus Serabelisib Inhibits Cancer Cell Growth in Combination with a Range of Targeted and Chemotherapeutic Anticancer Agents

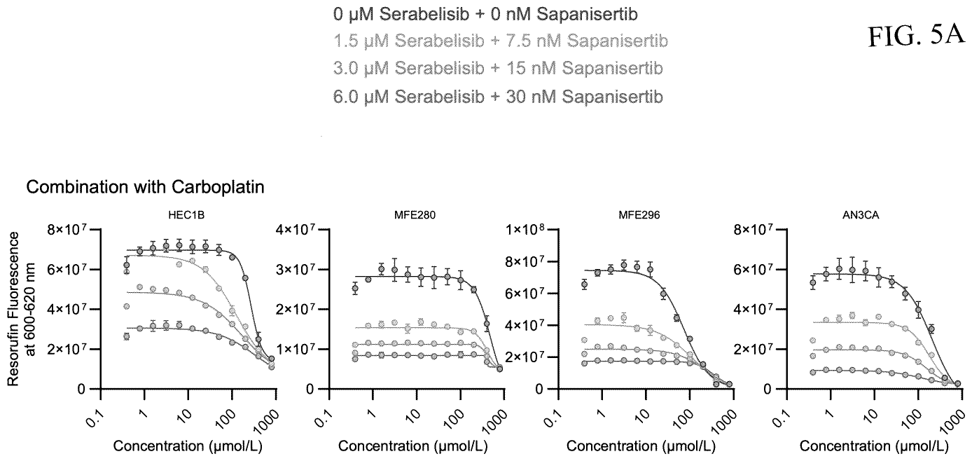

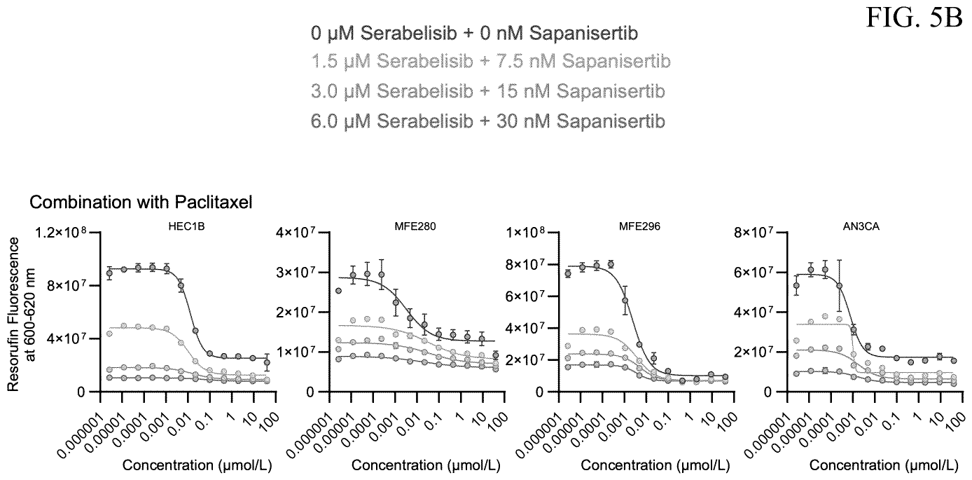

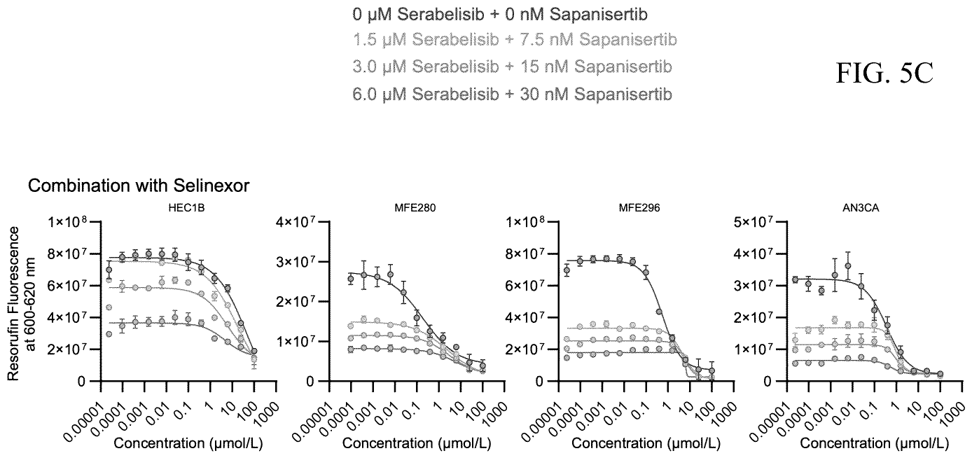

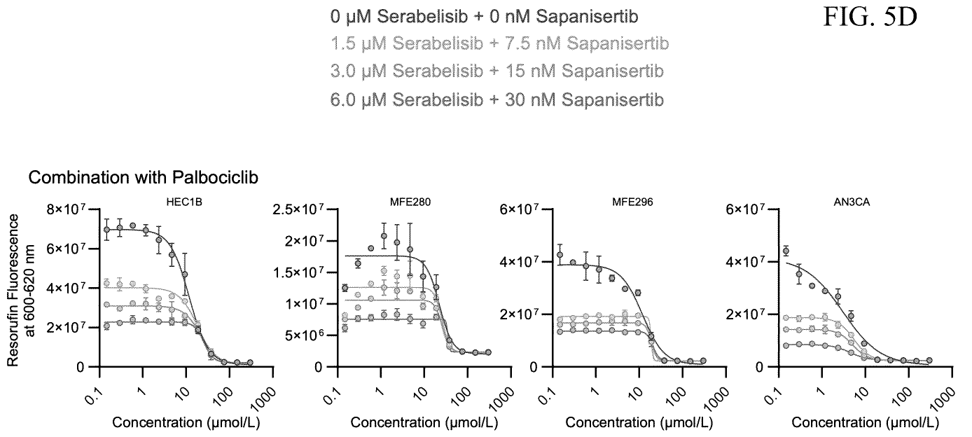

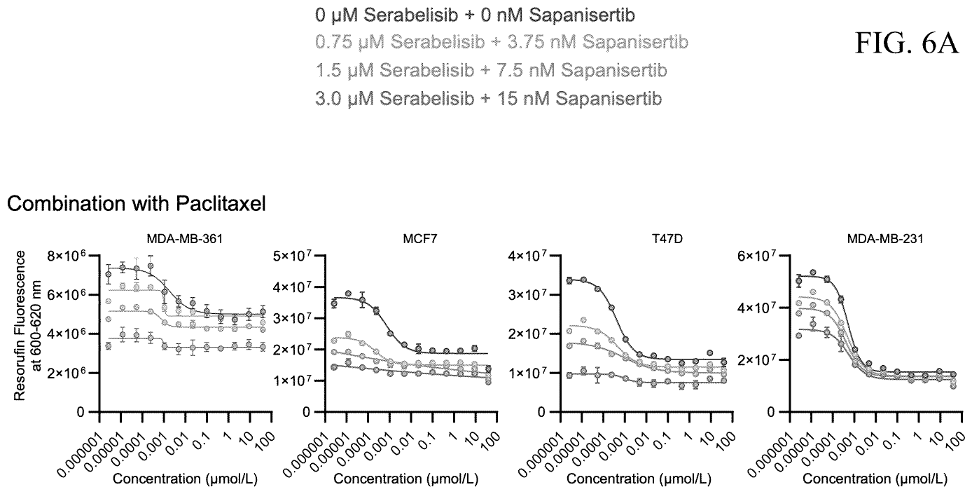

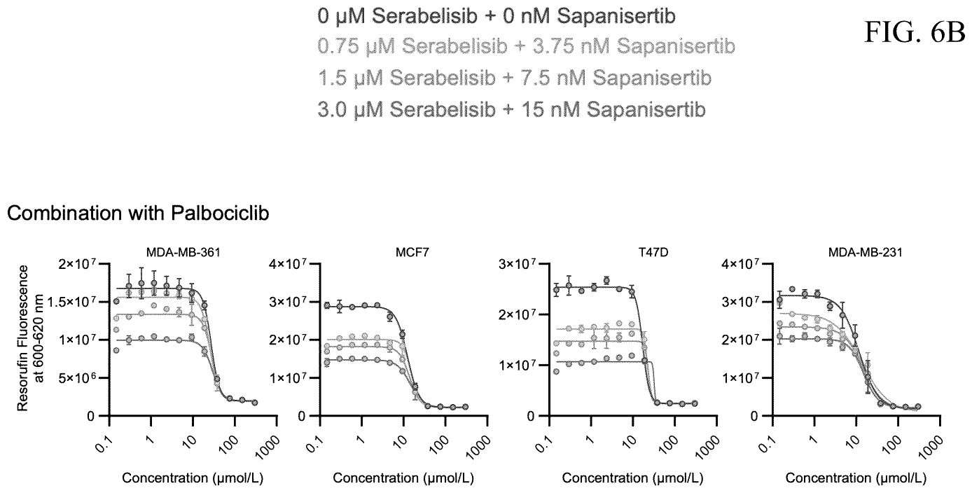

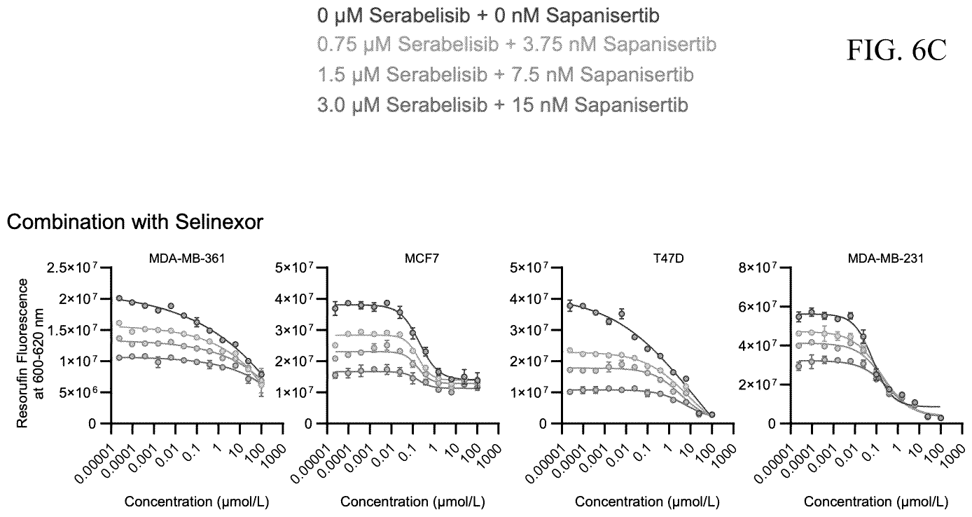

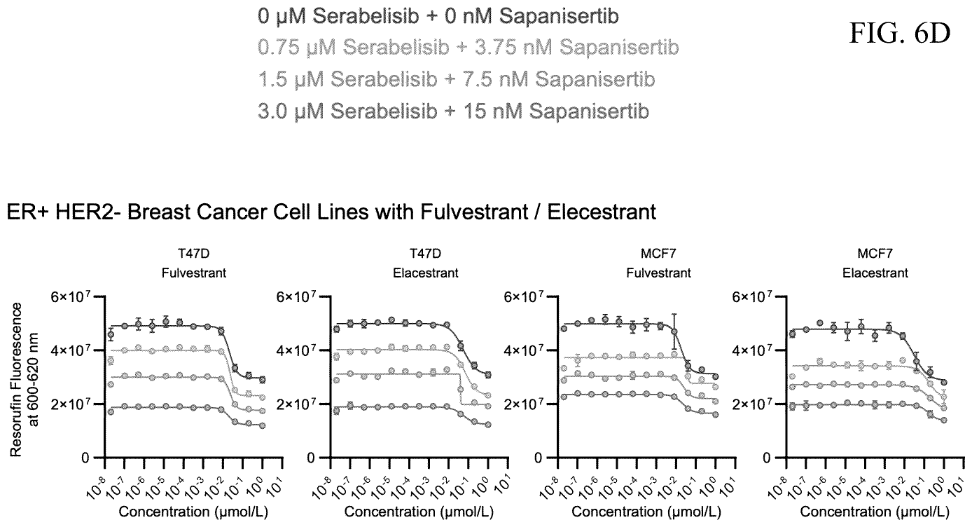

There is an increasing reliance on therapeutic combinations in cancer treatment. We sought to better understand the ability of sapanisertib plus serabelisib to combine with agents with different mechanisms of action in a range of endometrial (AN3CA, MFE296, MFE280 & HEC1B) and breast (MDA-MB-361, MCF7, T47D & MDA-MB-231) cancer cell lines. Increasing clinically relevant doses of sapanisertib and serabelisib produced both down- and left-shifted growth inhibition curves for carboplatin, paclitaxel, selinexor (selective inhibitor of nuclear export being developed for TP53 wild-type endometrial cancer) and palbociclib (CDK4/6 inhibitor) in endometrial cancer lines (FIG. 5A, FIG. 5B, FIG. 5C and FIG. 5D). This trend was consistent across all four endometrial cancer cell lines tested, and there was no evidence of agent-to-agent antagonism (i.e., to right/upward-shift of curves). Overall, these data support the ability to combine sapanisertib plus serabelisib with a range of established/emerging therapeutics for endometrial cancer. A very similar set of results was obtained with breast cancer cell lines, where inhibition curves were down-/left-shifted in combination with paclitaxel, palbociclib, selinexor, fulvestrant and elacestrant (FIG. 6A, FIG. 6B, FIG. 6C and FIG. 6D).

### Example 6: Taxane Resistance Activates the PI3K/AKT/Mtor Pathway, and Taxane Resistant Cells Retain Sensitivity to Sapanisertib Plus Serabelisib

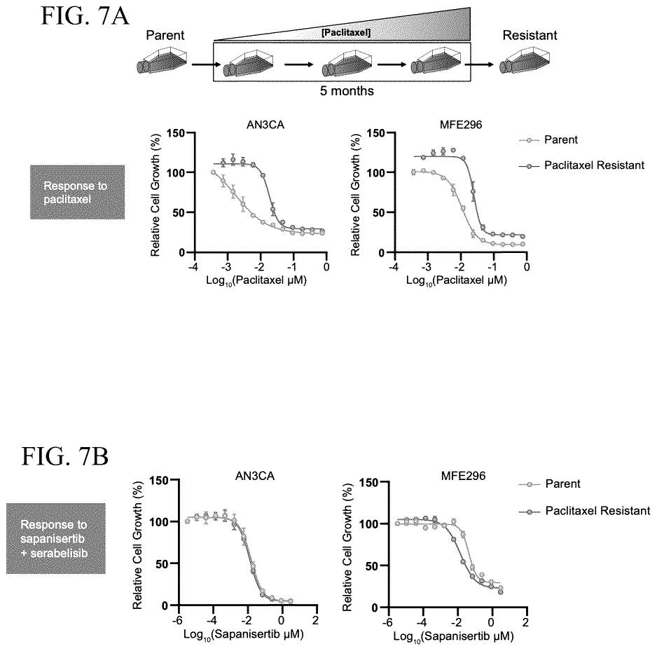

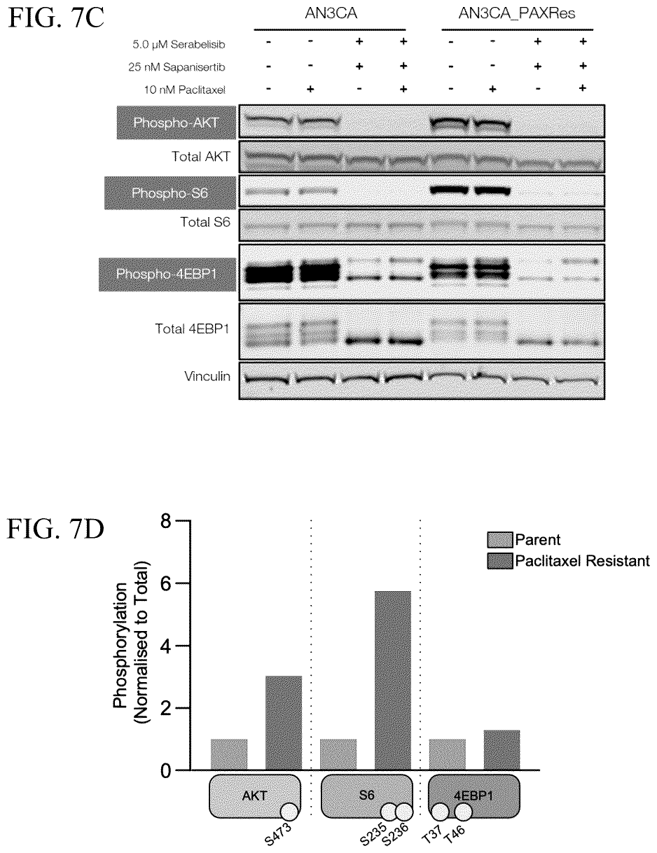

Given the positive in vitro combination data in the present study, and efficacy signal seen in a Phase 1b clinical trial combining sapanisertib, serabelisib and paclitaxel, in which several patients who had received paclitaxel in prior lines of therapy had partial or complete clinical responses, we sought to further explore the relationship between paclitaxel treatment and the PI3K/AKT/Mtor pathway. After five months' exposure to increasing concentrations of paclitaxel the endometrial cancer cell lines AN3CA and MFE296 acquired paclitaxel resistance, as demonstrated by substantial right-shift in the paclitaxel growth inhibition curves (FIG. 7A). Despite acquiring taxane resistance, the cells either remained sensitive (AN3CA), or displayed increased sensitivity (left-shifted curve, MFE296 cells) to sapanisertib plus serabelisib (FIG. 7B).

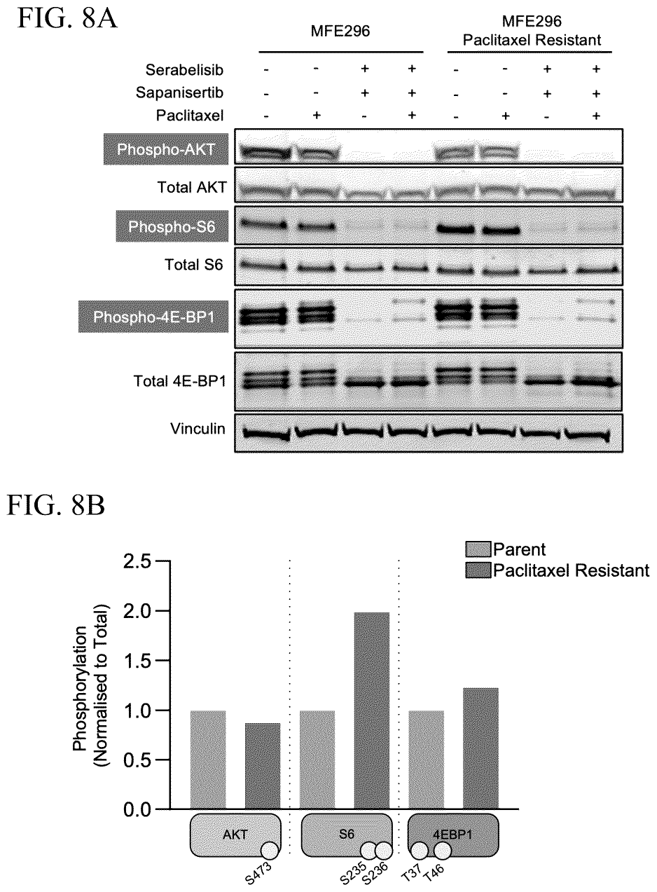

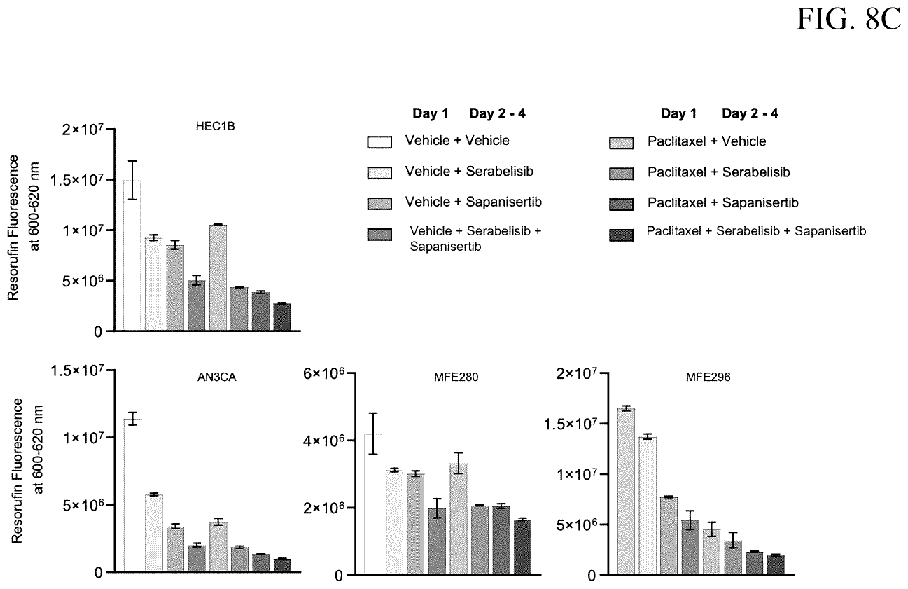

Analysis of PI3K/AKT/Mtor pathway signaling revealed that taxane-resistant endometrial cancer cells had increased pathway activity: taxane-resistant AN3CA cells displayed six-fold higher phosphorylated-S6 and three-fold elevation in phosphorylated-AKT (FIG. 7C and FIG. 7D). The effect was more modest in MFE296 cells, which showed a two-fold increase in phosphorylated-S6 (FIG. 8A and FIG. 8B). Notably, despite the increases in phosphorylation of pathway effectors, the sapanisertib plus serabelisib combination was able to inhibit signaling equally as well in parental cells or taxane resistant cells (FIG. 7C, FIG. 7D, FIG. 8A and FIG. 8B). Taken together these data suggest that taxane resistance in endometrial cancer cells involves upregulated PI3K/AKT/Mtor pathway activity; however, this increase does not reduce—but rather potentially increases—sensitivity to sapanisertib plus serabelisib.

To replicate a clinically relevant dosing regimen for sapanisertib, serabelisib and paclitaxel in vitro, we conducted an experiment where drugs were given sequentially and for a discrete time period. I.e., paclitaxel was given on day 1 (and removed after 24h), and sapanisertib plus serabelisib were given on days 2, 3 and 4. As with other in vitro experiments, clinically relevant concentrations of sapanisertib and serabelisib were used. Paclitaxel was used at low doses to allow for the scale of sensitization to be revealed. In the four endometrial cancer cell lines tested, the sapanisertib, serabelisib and paclitaxel combination caused substantial growth inhibition and outperformed single drug and double-drug combinations (FIG. 8C).

### Example 7: Sapanisertib and Serabelisib in Combination with Paclitaxel Strongly Suppress Tumor Growth of In Vivo Models of Endometrial and Breast Cancer, which is Further Enhanced by an Insulin-Suppressing Diet

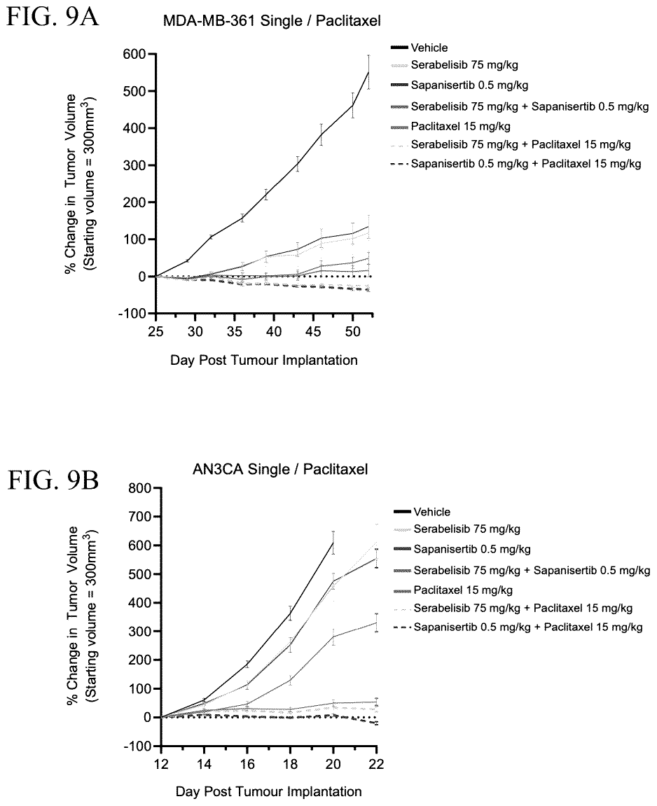

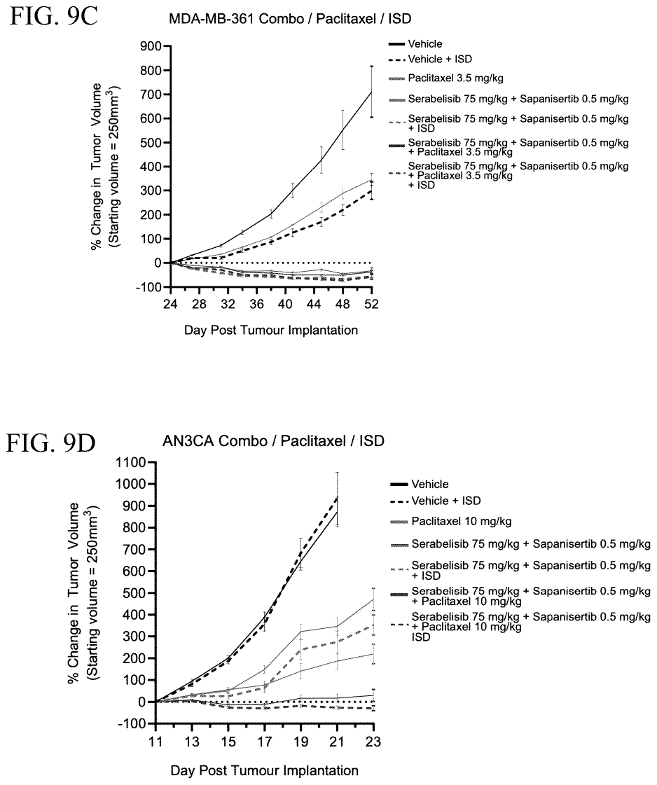

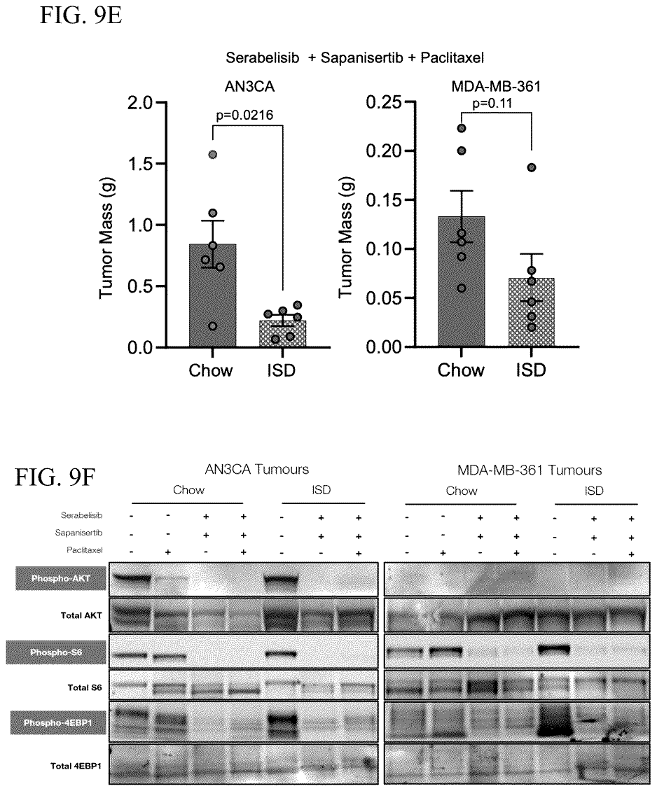

Given the in vitro mechanistic data demonstrating cooperativity between sapanisertib, serabelisib and paclitaxel, we conducted in vivo efficacy experiments using endometrial and breast cancer xenografts. In the first set of experiments, we used single- and double-drug combinations (FIG. 9A and FIG. 9B), in the second set of experiments we used double- and triple-drug combinations with or without an insulin-suppressing diet (FIG. 9C and FIG. 9D). Doses used represent the mouse equivalent area under the curve (AUC) exposure for 200 mg serabelisib and 3 mg sapanisertib in humans (RP2D for sapanisertib plus serabelisib combination), administered in the clinically relevant dosing schedule (paclitaxel once per week on day 1, sapanisertib and serabelisib (PO) three days per week on days 2, 3 and 4). Drug treatments were initiated once tumors were well-established and growing rapidly (250-300 mm³).

As single agents, sapanisertib and serabelisib showed substantial inhibition of tumor growth in MDA-MB-361 tumors, and activity in AN3CA tumors. This result correlates with Western blot data (FIG. 1C) showing 4E-BP1 inhibition by sapanisertib and serabelisib was achieved in both cell lines but was more complete in MDA-MB-361 cells. In combination, sapanisertib plus serabelisib showed increased efficacy in both models versus single agents, with near complete tumor growth inhibition in MDA-MB-361 cells (FIG. 9A and FIG. 9B). Both models were relatively sensitive to paclitaxel (at 15 mg/kg dose), which caused substantial tumor growth inhibition as monotherapy. Notably, paclitaxel monotherapy was less effective than sapanisertib and serabelisib combination for MDA-MB-361 cells. Doublets of sapanisertib+paclitaxel (15 mg/kg) or serabelisib+paclitaxel (15 mg/kg) showed very robust tumor growth inhibition in both models. In AN3CA cells the sapanisertib+paclitaxel combination was especially effective, causing tumor regression. In MDA-MB-361 cells both doublet drug combinations produced tumor regression.

Given the potential for sapanisertib and serabelisib to sensitize tumors to paclitaxel we sought to assess the triplet combination (sapanisertib, serabelisib, paclitaxel). As the doublet combinations (i.e., sapanisertib plus paclitaxel or serabelisib plus paclitaxel) were highly efficacious (FIG. 9A and FIG. 9B), we selected reduced doses of the taxane (10 mg/kg in AN3CA and 3.5 mg/kg in MDA-MB-361) for the triplet experiments to allow for the scale of sensitization to be revealed (FIG. 9C and FIG. 9D). In both xenograft models the drug triplet caused profound tumor growth inhibition, with tumor regression seen in both models, especially MDA-MB-361. This degree of tumor growth inhibition was particularly notable given the lowered doses of paclitaxel and the starting tumor volume (250 mm³).

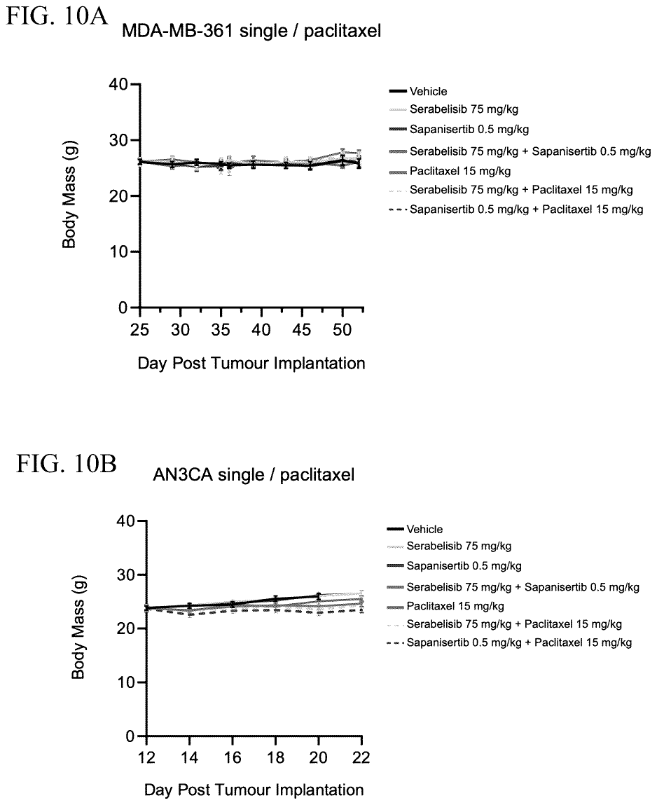

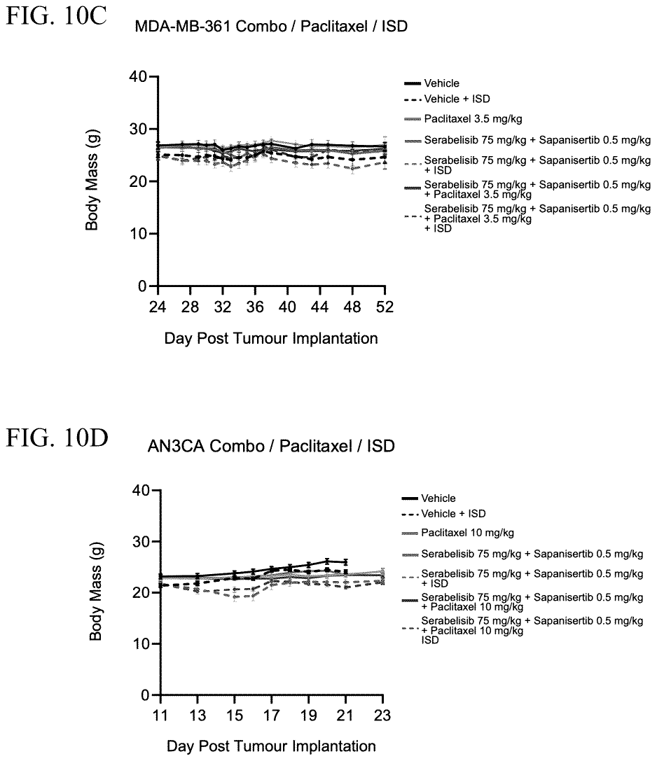

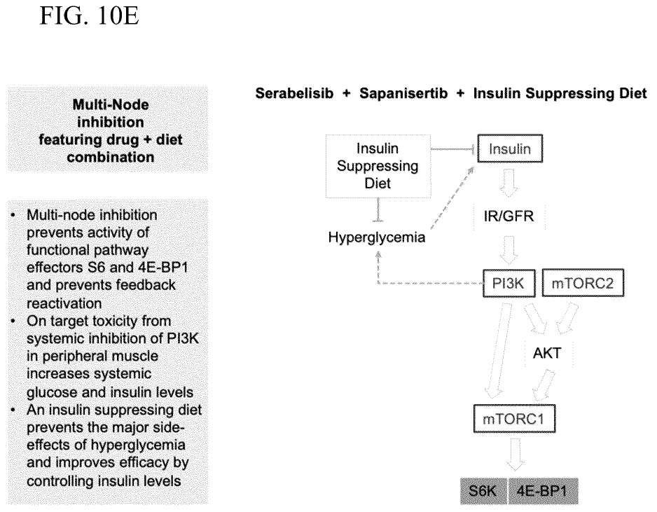

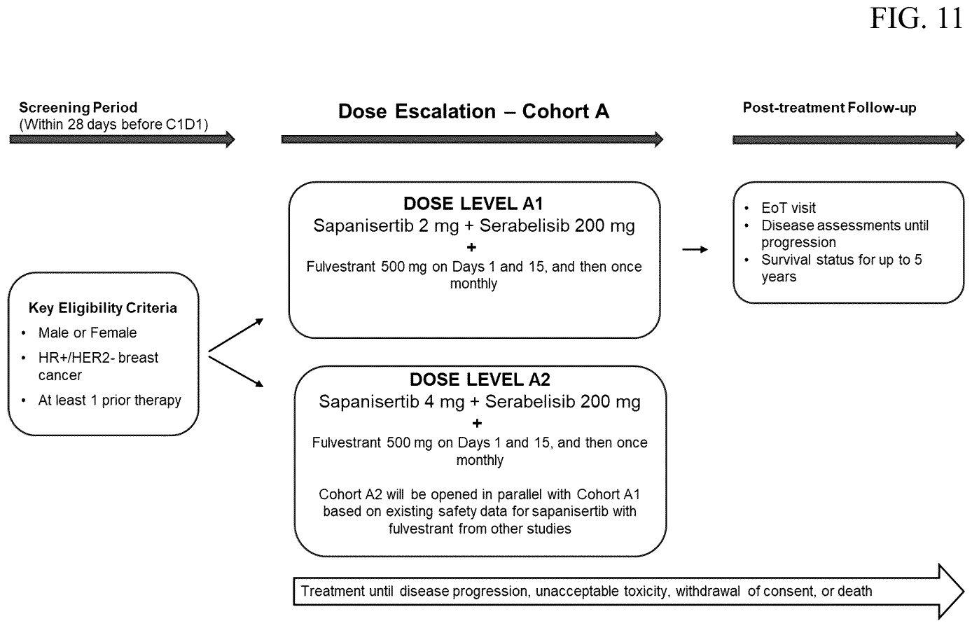

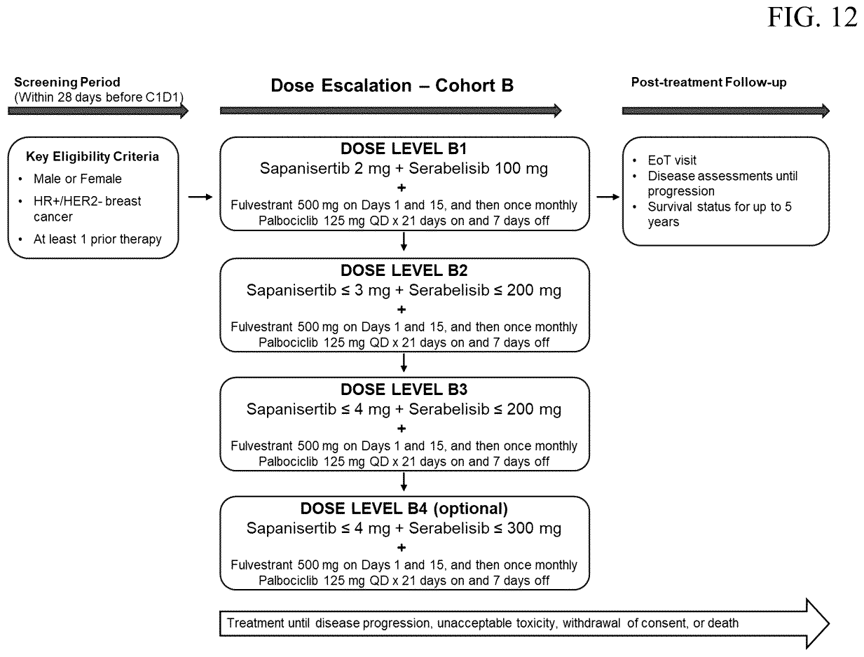

We next sought to establish the impact on efficacy of adding ISD to the sapanisertib, serabelisib and paclitaxel regimen. Notably ISD alone had a major impact on MDA-MB-361 xenografts, inhibiting tumor growth to a greater extent than 3.5 mg/kg paclitaxel (FIG. 9C). When added to the sapanisertib plus serabelisib doublet, or sapanisertib, serabelisib and paclitaxel triplet, ISD enhanced anti-tumor efficacy (FIG. 9C and FIG. 9D). When added to the sapanisertib plus serabelisib doublet, or sapanisertib, serabelisib and paclitaxel triplet in AN3CA xenografts, ISD enhanced anti-tumor efficacy, resulting in lower tumor mass at endpoint (FIG. 9E). As seen with in vitro experiments, tumors treated with sapanisertib plus serabelisib showed a substantial decrease in AKT, S6 and 4E-BP1 phosphorylation (FIG. 9F). The drug-drug/drug-diet combinations were well-tolerated FIG. 10A, FIG. 10B, FIG. 10C and FIG. 10D) while some weight loss was seen in ISD groups initially this tended to stabilize over the course of the study.

Taken together these data show that sapanisertib plus serabelisib, with the optional addition of ISD, represents a comprehensive multi-nodal approach for the effective inhibition of PI3K/AKT/Mtor signaling in tumors in vivo (FIG. 10E). Furthermore, combination of multi-node PI3K/AKT/Mtor inhibition with paclitaxel—even at paclitaxel doses with relatively modest monotherapy efficacy—produced profound tumor growth inhibition and tumor regression in preclinical models of endometrial and breast cancer.

### Example 8: Methods Used to Perform In Vitro Analysis of PI3K/AKT/Mtor Signaling Inhibition by Western Blot (FIG. 1C, FIG. 2A, FIG. 3A, FIG. 3B, FIG. 3D, FIG. 7C, FIG. 7D, FIG. 8A, FIG. 8B and FIG. 9F)

Drugs and Compounds: The following compounds, >95% purity, were used in this study (supplier information in parentheses): serabelisib (Carbogen), sapanisertib (Cayman Chemical, 11811), paclitaxel (Apex Bio, A4393), carboplatin (Sigma, BP711), alpelisib (Cayman Chemical, 16986), capivasertib (Biorbyt, ORB1307109), everolimus (Cayman Chemical, 11597), inavolisib (Cayman Chemical, 37576), RLY-2608 (Medchem Express, HY-153306), STX-478 (Medchem Express, HY-156681) fulvestrant (Cayman Chemical, 10011269), and elacestrant hydrochloride (Cayman Chemical 37298). Stock solutions of each compound were made in the appropriate vehicle and stored at −20° C.

Cell culture: The following cell lines were used, the supplier from which each cell line was purchased is indicated in parentheses: AN3CA (ATCC), HEC1B (ATCC), MFE296 (ECACC), MFE280 (ECACC), MCF7 (ECACC), T47D (ECACC), MDA-MB-361 (ATCC), MDA-MB-361-Luc (JCRB), and MDA-MB-231Cas9 (BPS Biosciences). AN3CA, HEC1B, MFE296, MFE280, MCF7, T47D, MDA-MB-231 cells were cultured in high glucose DMEM with 10% FBS+2 Mm glutamine. MDA-MD-361 cells were cultured in RPMI with 20% FBS+2 Mm glutamine. All cell lines were authenticated by STR genotyping and were free from Mycoplasma, as assessed by Mycoprobe (R&D Systems). Cells were grown in 21% O₂, 5% CO₂ at 37° C.

Immunoblotting: Cells were seeded in 10 cm dishes. The following day, media was replaced with serum-free media overnight (10-12 hours). Depending on the experiment, insulin was added before or after drug treatment, as indicated in the figure legends. Cells were then washed rapidly with ice-cold PBS and harvested on ice by scraping. Cell pellets were flash frozen. Protein was extracted with RIPA buffer+HALT protease and phosphatase inhibitor (Thermo Fisher). Protein was quantified with the Bradford assay (Thermo Fisher) and 20 μg of each protein sample was subjected to SDS-PAGE. For immunoblot on tumor tissue, tumors were homogenized by grinding with a pestle and mortar on dry ice to a fine powder. Protein was then extracted and processed as above. Immunoblot analysis was performed using primary antibodies against the following targets: panAKT (Cell Signaling Technologies, 2920), phosAKT-Ser473 (Cell Signaling Technologies, 4058), S6 (Cell Signaling Technologies, 2317), phosS6-Ser235/Ser236 (Cell Signaling Technologies, 2211), 4E-BP1 (ProteinTech, 60246-1-Ig), phos4E-BP1-T37/46 (Cell Signaling Technologies, 2855), and vinculin (Cell Signaling Technologies, 13901). Secondary antibodies goat anti-rabbit-800 (926-32211) and donkey anti-mouse-680RD (926-68072) from Li-core were used. Membranes were visualized on a Licor Odyssey CLx imaging system. Quantification of immunoblots was performed using Licor Image Studio software.

### Example 9: Methods Used to Perform In Vitro Analysis of Dose-Response of Cancer Cells to Single-Node and Multi-Node Inhibition of the PI3K/AKT/Mtor Pathway (FIG. 1D, FIG. 1E, FIG. 2B, FIG. 2C, FIG. 3C, FIG. 3D, FIG. 4A, FIGS. 5A-D, FIGS. 6A-D, FIG. 7A and FIG. 7B)

Drugs and Compounds: The following compounds, >95% purity, were used in this study (supplier information in parentheses): serabelisib (Carbogen), sapanisertib (Cayman Chemical, 11811), paclitaxel (Apex Bio, A4393), carboplatin (Sigma, BP711), alpelisib (Cayman Chemical, 16986), capivasertib (Biorbyt, ORB1307109), everolimus (Cayman Chemical, 11597), inavolisib (Cayman Chemical, 37576), RLY-2608 (Medchem Express, HY-153306), STX-478 (Medchem Express, HY-156681) fulvestrant (Cayman Chemical, 10011269), and elacestrant hydrochloride (Cayman Chemical 37298). Stock solutions of each compound were made in the appropriate vehicle and stored at −20° C.

Cell culture: The following cell lines were used, the supplier from which each cell line was purchased is indicated in parentheses: AN3CA (ATCC), HEC1B (ATCC), MFE296 (ECACC), MFE280 (ECACC), MCF7 (ECACC), T47D (ECACC), MDA-MB-361 (ATCC), MDA-MB-361-Luc (JCRB), and MDA-MB-231Cas9 (BPS Biosciences). AN3CA, HEC1B, MFE296, MFE280, MCF7, T47D, MDA-MB-231 cells were cultured in high glucose DMEM with 10% FBS+2 Mm glutamine. MDA-MD-361 cells were cultured in RPMI with 20% FBS+2 Mm glutamine. All cell lines were authenticated by STR genotyping and were free from Mycoplasma, as assessed by Mycoprobe (R&D Systems). Cells were grown in 21% O₂, 5% CO₂ at 37° C.

Cell-based dose-response curves: Cells were seeded in 96-well plates. 24 hours later, dilution series of compounds indicated in each figure were added to the appropriate wells. Cells were then cultured for a further 72 hours followed by the addition of resazurin for 3-4 hours. Alive cells were quantified by measuring the fluorescence of resorufin (reduced resazurin) at 600-620 nm.

Statistical Analyses: Statistical analyses were performed in GraphPad Prism 10 software. Comparisons were assessed using an unpaired two-tailed Student's t-test, with Welch's correction when appropriate. Error bars are shown as standard deviation (SD) unless otherwise indicated in figure descriptions and all experiments were performed at least twice. Sample sizes were chosen based on previous experience of in vitro and in vivo experiments.

### Example 10: Methods Used to Generate Paclitaxel-Resistant Endometrial Cancer Cell Lines (FIG. 7A-D and FIG. 8A and FIG. 8B)

Generation of paclitaxel-resistant endometrial cancer cell lines: AN3CA and MFE296 were cultured in gradually increasing doses of paclitaxel, starting from 2 Nm, until colonies formed over a period of 3-4 months. Paclitaxel-resistant cultures became established over the following month and were then maintained in 10 Nm (AN3CA) or 18 Nm (MFE296) paclitaxel continuously. Paclitaxel resistance was confirmed by performing dose-response curves against parental lines.

Drugs and Compounds: The following compounds, >95% purity, were used in this study (supplier information in parentheses): serabelisib (Carbogen), sapanisertib (Cayman Chemical, 11811), paclitaxel (Apex Bio, A4393), carboplatin (Sigma, BP711), alpelisib (Cayman Chemical, 16986), capivasertib (Biorbyt, ORB1307109), everolimus (Cayman Chemical, 11597), inavolisib (Cayman Chemical, 37576), RLY-2608 (Medchem Express, HY-153306), STX-478 (Medchem Express, HY-156681) fulvestrant (Cayman Chemical, 10011269), and elacestrant hydrochloride (Cayman Chemical 37298). Stock solutions of each compound were made in the appropriate vehicle and stored at −20° C.

Cell culture: The following cell lines were used, the supplier from which each cell line was purchased is indicated in parentheses: AN3CA (ATCC), HEC1B (ATCC), MFE296 (ECACC), MFE280 (ECACC), MCF7 (ECACC), T47D (ECACC), MDA-MB-361 (ATCC), MDA-MB-361-Luc (JCRB), and MDA-MB-231Cas9 (BPS Biosciences). AN3CA, HEC1B, MFE296, MFE280, MCF7, T47D, MDA-MB-231 cells were cultured in high glucose DMEM with 10% FBS+2 Mm glutamine. MDA-MD-361 cells were cultured in RPMI with 20% FBS+2 Mm glutamine. All cell lines were authenticated by STR genotyping and were free from Mycoplasma, as assessed by Mycoprobe (R&D Systems). Cells were grown in 21% O₂, 5% CO₂ at 37° C.

### Example 11: Methods Used to Perform In Vitro Analysis of Response of Cancer Cells to Time-Specific Drug Exposure and Clinical Drug Sequencing of Serabelisib, Sapanisertib, and Paclitaxel (FIG. 8C)

Cells were seeded in 96-well plates on day 0. 24 hours later (day 1), cells were treated with 10 Nm paclitaxel or vehicle (DMSO). After 24 hours (day 2), media was replaced, followed by the addition of either vehicle (DMSO), 5.9 μM serabelisib, 30 Nm sapanisertib or 5.9 μM serabelisib+30 Nm sapanisertib until day 4. On day 4, resazurin was added for 4 hours and live cells were quantified by measuring the fluorescence of resorufin (reduced resazurin) at 600-620 nm.

Cell culture: The following cell lines were used, the supplier from which each cell line was purchased is indicated in parentheses: AN3CA (ATCC), HEC1B (ATCC), MFE296 (ECACC), MFE280 (ECACC), MCF7 (ECACC), T47D (ECACC), MDA-MB-361 (ATCC), MDA-MB-361-Luc (JCRB), and MDA-MB-231Cas9 (BPS Biosciences). AN3CA, HEC1B, MFE296, MFE280, MCF7, T47D, MDA-MB-231 cells were cultured in high glucose DMEM with 10% FBS+2 Mm glutamine. MDA-MD-361 cells were cultured in RPMI with 20% FBS+2 Mm glutamine. All cell lines were authenticated by STR genotyping and were free from Mycoplasma, as assessed by Mycoprobe (R&D Systems). Cells were grown in 21% O₂, 5% CO₂ at 37° C.

Drugs and Compounds: The following compounds, >95% purity, were used in this study (supplier information in parentheses): serabelisib (Carbogen), sapanisertib (Cayman Chemical, 11811), paclitaxel (Apex Bio, A4393), carboplatin (Sigma, BP711), alpelisib (Cayman Chemical, 16986), capivasertib (Biorbyt, ORB1307109), everolimus (Cayman Chemical, 11597), inavolisib (Cayman Chemical, 37576), RLY-2608 (Medchem Express, HY-153306), STX-478 (Medchem Express, HY-156681) fulvestrant (Cayman Chemical, 10011269), and elacestrant hydrochloride (Cayman Chemical 37298). Stock solutions of each compound were made in the appropriate vehicle and stored at −20° C.

### Example 12: Methods Used to Perform In Vivo Analysis of Efficacy (Tumor Growth Inhibition) and Tolerability (Body Weight) of Sapanisertib/Serabelisib/Paclitaxel/Insulin-Suppressing Diet Alone or in Combination to Treat Endometrial or Breast Cancer Xenografts

Tumor xenografts: In vivo mouse xenograft experiments were performed at a contract research organization (CRO) according to the approved Institutional Animal Care and Use Committee (IACUC) guidelines following the Association for Assessment and Accreditation of Laboratory Animal Care (AAALAC) guidance. Female BALB/c nude mice 6-8 weeks of age were used for all in vivo experiments. Prior to implantation, AN3CA cells were cultured in vitro in EMEM medium supplemented with 10% heat inactivated FBS and MDA-MB-361 cells in L-15 medium supplemented with 20% heat inactivated FBS. Cells were in the exponential growth phase when harvested for implantation. Tumor cells were injected subcutaneously on the right flank of female BALB/c nude mice. For AN3CA, 5 million cells were implanted in 0.1 ml of growth medium; for MDA-MB-361, 10 million cells were implanted in 0.1 ml of 1:1 growth medium: Matrigel. The day before implantation of MDA-MB-361 cells, mice were given 40 g of subcutaneous oestradiol benzoate and then twice per week thereafter for the duration of the experiment.

Groups of 7-8 mice with tumors of the requisite size were recruited into treatment arms. Mice that lost condition due to bladder calculi resulting from oestradiol injections were excluded from the study, as were mice that lost condition due to rolling in the ISD, which unlike regular chow pellets is a paste. Serabelisib was formulated in 5% DMSO, 5% Tween 80, 40% PEG300, 50% water, Ph=3.0 and administered by oral gavage 3 times per week at the doses indicated in each figure. Sapanisertib was formulated in 0.05% Tween 80 and 0.5% CMC in water and administered by oral gavage 3 times per week at the doses indicated in each figure. Paclitaxel was formulated in normal saline and administered intravenously via the tail vein, once per week at the indicated doses in each figure. Body weight of all mice was measured two to three times per week. Tumor dimensions were measured regularly with a calliper and tumor volume was estimated using the formula: TV=a×b²/2, where “a” and “b” are long and short diameters of a tumor, respectively. Unless otherwise stated, mice were fed standard chow for the duration of the experiment. For pharmaco-dietary experiments, a high fat insulin-suppressing diet was used (H1F93.5, Dyets, w/w % of macronutrients; protein=8.9%, total carbohydrates (digestible and non-digestible)=12.8%, fat=78.4%), the control group received normal chow diet (Beijing Keao Xielie Feed Co. Ltd.). In the relevant groups, ISD was introduced two days before drug treatment was initiated.

### Example 13: Sapanisertib and Serabelisib (PIKTOR) with Paclitaxel and a Substudy with an Insulin-Suppressing Diet in Patients with Advanced/Recurrent Endometrial Cancer

Brief Summary: A Phase 2, multicenter, open-label, single-arm study is conducted to evaluate the efficacy and safety of sapanisertib and serabelisib (PIKTOR) with paclitaxel in participants with advanced or recurrent endometrial cancer. More specifically, the study is a Phase 2, multicenter, open-label, single-arm study to evaluate the efficacy and safety of sapanisertib and serabelisib (PIKTOR) with paclitaxel in participants with advanced or recurrent endometrial cancer who have failed prior systemic therapies, including a platinum-based therapy and an immune checkpoint inhibitor, either separately or together.
Conditions: Endometrial cancers
Intervention/Treatment:
(a) Drug: Sapanisertib;
(b) Drug: Serabelisib;
(c) Drug: Paclitaxel.
Ages Eligible for Study: 18 years and older (Adult, Older Adult).
Sexes Eligible for Study: Female.
Accepts Healthy Volunteers: No.
Inclusion Criteria:
(a) Histologically confirmed diagnosis of endometrioid endometrial carcinoma.
(b) Documented evidence of advanced or recurrent endometrial cancer that is not amenable to surgery/radiation for curative intent.
(c) Participant has received at least one but not more than four prior systemic therapies. Prior therapy must include platinum-based chemotherapy and a checkpoint inhibitor, either separately or in combination. If a subject has been unable to be treated with checkpoint inhibitor in the past due to medical contraindications, a consultation with a Medical Monitor is conducted.
(d) PI3K/AKT/Mtor pathway gene alteration identified.
(e) At least one measurable target lesion according to RECIST v1.1.
(f) Eastern Cooperative Oncology Group (ECOG) performance status of ≤1 at Screening.
(g) Non-pregnant, non-lactating females who are postmenopausal, surgically sterile or who agree to use effective contraceptive methods.
Exclusion Criteria:
(a) Participants with central nervous system metastases are not eligible, unless they have completed local therapy and have discontinued the use of corticosteroids for this indication for at least four weeks before starting treatment in this study.
(b) Active malignancy (except for endometrial cancer, definitively treated in-situ carcinomas [e.g., breast, cervix, bladder], or basal or squamous cell carcinoma of the skin) within the past 24 months prior to treatment. Fully resected localized malignancies are eligible.
(c) Gastric feeding tube (gastrostomy tube), gastrointestinal malabsorption, gastrointestinal anastomosis, bowel obstruction, or any other condition that might affect the absorption of study treatment.
(d) Clinically significant (per Investigator judgement) hemoptysis or tumor bleeding.
(e) Significant cardiovascular impairment.
(f) Active, uncontrolled (requiring systemic antimicrobial therapy) infection.
(g) Concurrent participation in another therapeutic clinical trial.
(h) Prior radiation therapy within 21 days prior to start of study treatment.
(i) Strong CYP3A4 inhibitors and inducers are prohibited during the study. Strong CYP1A2 inhibitors as well as CYP1A2 inducers should be administered with caution and at the discretion of the Investigator. Alternative treatments, if available, should be considered. Additionally, strong CYP3A4 inhibitors or inducers should not be taken within seven days before the first dose of study intervention.
(j) Participants who require PPIs or chronic use of antacids, histamine H2 receptor blockers, or other treatments to raise gastric Ph.
(k) Prolongation of QTc interval to >480 ms.
(l) HbA1c≥8.0% or fasting serum glucose >160 mg/D1 or fasting triglycerides >300 mg/D1 or receiving treatment with insulin.
Enrollment (estimated): 40
Study type: Interventional
Primary Purpose: Treatment
Interventional Model: Single Group Assignment
Masking: None (Open Label)

TABLE 1
Arms and Interventions

| Participant Group / Arm | Intervention / Treatment |
|---|---|
| Experimental: sapanisertib and serabelisib | Drug: Sapanisertib |
| (PIKTOR) with paclitaxel. | Oral |
| Subjects receive doses of sapanisertib and | Other Names: |
| serabelisib (PIKTOR) administered orally and | FTH-003 |
| paclitaxel administered intravenously. | MLN0128 |
| | Drug: Serabelisib |
| | Oral |
| | Other Names: |
| | FTH-001 |
| | MLN1117 |
| | Drug: Paclitaxel |
| | Infusion |

TABLE 2
Primary Outcome Measure

| Outcome Measure | Matter Description | Time Frame |
|---|---|---|
| Objective Response | Defined as the proportion of | Up to 2 years |
| Rate (ORR) | participants with measurable | |
| | disease at baseline who have | |
| | confirmed complete response | |
| | (CR) or partial response (PR). | |

TABLE 3
Secondary Outcome Measures

| Outcome Measure | Matter Description | Time Frame |
|---|---|---|
| Progression-free | Defined as the time from | Up to 5 years. |
| survival (PFS) | first dose until the date of | |
| | disease progression or death. | |
| Progression-free | Defined as PFS rate at | Up to 5 years. |
| survival (PFS) at | 6 months. | |
| 6 months | | |
| Overall survival (OS) | Defined as the time from | Up to 5 years. |
| | first dose to death. | |
| Clinical benefit | Defined as the percentage of | Up to 5 years. |
| rate (CBR) | participants who achieve | |
| | complete response (CR), | |
| | partial response (PR), | |
| | or have stable disease (SD) | |
| | of at least 16 or 24 weeks. | |
| Duration of response | Defined for participants | Up to 5 years. |
| (DoR) | with confirmed CR or PR as | |
| | the time from response until | |
| | date of documented disease | |
| | progression or death. | |
| Safety and tolerability | Graded according to the | Up to 2 years. |
| of drugs by assessment | National Cancer Institute | |
| of adverse events | Common Terminology Criteria | |
| (Aes) / serious adverse | for Adverse Events | |
| events (SAEs) | (NCI CTCAE V5.0). | |

Sample Size Determination: Sample size was calculated based on the primary endpoint of ORR in the all-treated population. The null hypothesis for this study is ORR less than or equal to 18%. The total sample size is about 40 subjects. With this sample size, using an overall type-1 error of 10% (1-sided), the study has greater than 80% power to reject the null hypothesis described above, assuming the ORR is greater than or equal to 33%. A final ORR of 28% (11/40) is sufficient to reject the null hypothesis.

In a two-year time frame, the ORR of the about 40 patients is greater than 35%.

In a five-year time frame, the PFS is greater than 12 months in a subset of patients.

In a five-year time frame, the progression-free survival rate at six months of the about 40 patients is greater than 60%.

In a five-year time frame, OS is greater than nine months in a subset of patients.

In a five-year time frame, the CBR of the about 40 patients is greater than 80%.

In a five-year time frame, the DoR is greater than six months in a subset of patients.

In a two-year time frame, hyperglycemia is experienced in less than 15% of the about 40 patients.

In a two-year time frame, grade 3 hyperglycemia is experienced in less than 5% of the about 40 patients.

In a two-year time frame, grade 4 hyperglycemia is not experienced in the about 40 patients.

In a two-year time frame, stomatitis is experienced in less than 15% of the about 40 patients.

In a two-year time frame, grade 3 or grade 4 stomatitis is not experienced in the about 40 patients.

No prophylactic measures or treatment, such as a mouth rinse with a steroid or corticosteroid, is needed because treatment emergent stomatitis is not detected at substantial levels. Specifically, the treatment does not cause stomatitis, including mouth ulcers or oral mucositis, at significant levels. Accordingly, because stomatitis, including mouth ulcers or oral mucositis, do not occur upon treatment, no warnings or prophylaxis regimens or recommendations for treating or preventing stomatitis is required in connection with the sapanisertib and serabelisib (PIKTOR)/paclitaxel combination treatment described herein. For example, no regimen of prophylactic use of a corticosteroid or steroid-containing mouthwash or rinse is needed when starting the treatment. As a non-limiting example, no structured regimen of stomatitis prophylaxis (e.g., dexamethasone 0.5 mg/5 Ml alcohol-free mouthwash (10 Ml swished for two minutes and spat, four times daily for eight weeks)) is required with the treatment. As an additional non-limiting example, no recommendation for concomitant administration of stomatitis prophylaxis (e.g., use of a steroid-containing mouthwash (dexamethasone oral solution 0.1 mg/Ml) administer four times daily and as needed) is required with the sapanisertib and serabelisib (PIKTOR)/paclitaxel combination treatment described herein.

TABLE 4
Stomatitis detection

| | Number of | Total AE | |
|---|---|---|---|
| | patients with AE | evaluable patients | Rate of AE (%) |
| All Grade | 3 | 22 | 13.6% |
| Grade 1-2 | 3 | 22 | 13.6% |
| Grade 3 | 0 | 22 | 0.0% |
| Grade 4 | 0 | 22 | 0.0% |

TABLE 5
Hyperglycemia detection

| | Number of | Total AE | |
|---|---|---|---|
| | patients with AE | evaluable patients | Rate of AE (%) |
| All Grade | 3 | 22 | 13.6% |
| Grade 1-2 | 2 | 22 | 9.1% |
| Grade 3 | 1 | 22 | 4.5% |
| Grade 4 | 0 | 22 | 0.0% |

### DISCUSSION

Notably, the effectiveness of cancer treatments has been shown to be entirely unreliable unless demonstrated in a properly powered analysis, such as the study described in this Example. Although Phase I studies may purport to demonstrate “promising” preliminary results relating to treatment effectiveness, such results are severely diminished in weight because of small sample sizes and the structure of the study itself. Indeed, Phase I studies are designed to determine safety and dose tolerability (e.g., the maximum tolerated dose of a drug), not efficacy. Non-limiting examples of studies discussing efficacy results which with diminished weight include Manuel Hidalgo & Eric K. Rowinsky, “The Rapamycin-sensitive Signal Transduction Pathway as a Target for Cancer Therapy,” 19 Oncogene 6680, 6680-86 (2000); and Ezzie Hutchinson, “CCI-779: A New Targeted Anti-cancer Agent,” 1 The Lancet 198, 198 (2000). Similarly, another Phase I study has reported on the safety of an endometrial cancer treatment and speculated on its efficacy (Starks et al., “Phase I dose escalation study of dual PI3K/Mtor inhibition by Sapanisertib and Serabelisib in combination with paclitaxel in patients with advanced solid tumors,” Gynecologic Oncology 166, 403-409 (2022). However, the short-time frame and extremely low sample size analyzed in Starks et al. cannot serve as a basis to reasonably predict the treatment's efficacy and indeed was not sufficient for regulatory bodies to approve the treatment.

Systematic reviews of clinical trial data underscore the unpredictability of a treatment's success based on preliminary Phase I results. As shown in Thomas et al. “Clinical Development Success Rates 2006-2015,” BIOIndustry Analysis, June 2016, pages 1-26, the likelihood of approval of oncology treatments from Phase I studies is only 5.1%. Other published studies of clinical trial success rates also demonstrate a high likelihood of failure (e.g., Wong et al. “Estimation of clinical trial success rates and related parameters” Biostatistics 20, 2, 273-286 (2019), indicating a 3.4% probability of oncology clinical trial success from phase I; and Dowden and Munro “Trends in clinical success rates and therapeutic focus” Nat. Rev. Drug Discovery, Vol. 18, 495-496 (2019), which points out that there was only a 9% probability of launch from Phase I for anticancer treatments). Indeed, although attempted treatments demonstrate high potency/specificity, low tissue exposure/selectivity, high plasma exposure, good safety profile and are typically advertised in phase I studies with high hope, they very often fail in later studies due to lack of efficacy. Sun et al., “Why 90% of clinical drug development fails and how to improve it?” Acta Pharm Sin B. 12(7):3049-3062 (2022). Thus, any alleged efficacy results relating to cancer treatment that lack proper statistical power and sufficient sample sizes should be viewed with caution and skepticism.

Regarding safety and tolerability, the lack of treatment emergent adverse effects such as stomatitis, in the present study, is particularly surprising considering the high incidence of stomatitis caused by other cancer treatment regimens involving inhibition of the PI3K/AKT/mTOR pathway. Notably, a clinical trial for patients having renal, endometrial, or bladder cancer demonstrated that administration of sapanisertib alone resulted in 45% of the patients experiencing stomatitis. See Voss et al. “Phase 1 study of mTORC1/2 inhibitor sapanisertib (TAK-228) in advanced solid tumors, with an expansion phase in renal, endometrial, or bladder cancer” Br. J. Cancer, (2020); 123(11), 1590-1598. Additionally, another study detected stomatitis in nearly 70% of patients receiving the PI3K/AKT/mTOR pathway inhibitor gedatolisib, with nearly 20% of those experiencing at least grade 3 stomatitis. See Hurvitz et al. “Gedatolisib Plus Fulvestrant, With & Without Palbociclib, vs Fulvestrant in Patients With HR+/HER2−/PIK3CA Wild-Type Advanced Breast Cancer: First Results from VIKTORIA-1” 2025 ESMO Congress, Berlin, Germany, LBA17, Oct. 18, 2025. The stomatitis that was noted in association with that treatment was so common and significant that prophylactic and/or treatment-emergent use of a steroid-containing mouth-rinse regimen was required in an effort to mitigate stomatitis severity. Similarly, other inhibitors of the PI3K/AKT/mTOR pathway, such as alpelisib (targeting PI3Kα) and everolimus (mTORC1 inhibitor) are known to cause significant stomatitis. Indeed, everolimus products contain an on-label warning indicating that stomatitis (including mouth ulcers and oral mucositis) occurs in subjects treated with everolimus and also list a corticosteroid mouthwash regimen (e.g., dexamethasone 0.5 mg/5 mL alcohol-free mouthwash (10 mL swished for two minutes and spat, four times daily for eight weeks)) as a stomatitis prophylaxis to be administered with everolimus treatment. In contrast, because there is insignificant treatment-emergent stomatitis detected after administration of the sapanisertib, serabelisib, and paclitaxel endometrial cancer treatment regimen as described herein, this treatment advantageously does not require any stomatitis-related preventive or treatment measures whatsoever.

### Example 14: Sapanisertib and Serabelisib (PIKTOR) in Various Combinations in Patients with HR+/HER2− Advanced/Metastatic Breast Cancer

### Protocol Summary

Protocol Title: Open-Label Umbrella Study to Evaluate Safety and Efficacy of Sapanisertib and Serabelisib (PIKTOR) in Various Combinations in Patients with HR+/HER2− Advanced or Metastatic Breast Cancer
Brief Title: Sapanisertib and Serabelisib (PIKTOR) in Various Combinations in Patients with HR+/HER2− Advanced/Metastatic Breast Cancer
Phase: 1b/2
Scope: The purpose of this study is to evaluate whether the combination of sapanisertib, an mTORC1 and mTORC2 inhibitor, and serabelisib, a PI3Kα inhibitor (PIKTOR), with fulvestrant or fulvestrant+palbociclib has an acceptable safety profile in participants with advanced or metastatic breast cancer who have failed prior systemic therapies. Participants will be eligible to enroll in the study regardless of whether they have a PI3K/AKT/mTOR pathway gene alteration.

This umbrella trial is designed to investigate the safety, tolerability, and preliminary efficacy of serabelisib and sapanisertib (PIKTOR) in combination with standard endocrine therapy backbones, beginning with fulvestrant and expanding to oral SERDS (e.g., elacestrant, giredestrant) and combinations with cyclin-dependent kinase (CDK) inhibitors, including palbociclib, and/or other anticancer therapies. The trial framework allows for future exploration of additional backbones and rational combinations in selected subpopulations.

### Number of Participants

Assuming five dose levels will be explored in the dose-escalation stage in Cohorts A and B, with an average of six participants per dose level, approximately 30 DLT-evaluable participants will be needed. Assuming 10% of participants are not DLT evaluable, approximately 33 participants will be enrolled. In addition, up to 15 participants at each dose level may be enrolled to further explore the safety and pharmacodynamics of PIKTOR with fulvestrant palbociclib. The total number of participants is not expected to exceed 108.

### Trial Population

Participants with a histologically confirmed diagnosis of HR+/HER2− advanced/metastatic breast cancer. Participants must be ≥18 years of age and have received prior systemic therapies for advanced/recurrent disease.

### Study Interventions, Dosages, Modes of Administration

Study interventions are administered in 28-day cycles as follows

Each cohort and dose level: Sapanisertib capsule dosed orally on Days 1-3, 8-10, 15-17, and 22-24 with food
Each cohort and dose level: Serabelisib tablets dosed orally on Days 1-3, 8-10, 15-17, and 22-24 with food
Cohort A: Fulvestrant 500 mg intramuscularly (IM) on Days 1 and 15 of Cycle 1, and then once monthly
Cohort B: Fulvestrant 500 mg IM on Days 1 and 15 of Cycle 1, and then once monthly, plus palbociclib 125 mg dosed once daily (QD) with food for 21 days on and seven days off
   Cohort A Dosing Scheme (28-day cycle for PIKTOR and Fulvestrant:

| Dose | | | | |
|---|---|---|---|---|
| level | Sapanisertib | Serabelisib | Fulvestrant | Notes |
| A1 | 2 mg orally | 200 mg PO | 500 mg IM | Cohorts A1 and A2 |
| | (PO) QD, | QD, 3 d/wk | on D 1 & | will be initiated in |
| | 3 d/wk | | D 15 then | parallel. Participants |
| A2 | 4 mg PO QD, | 200 mg PO | Q28 d | will be assigned by |
| | 3 d/wk | QD, 3 d/wk | | the Sponsor. |

A third dose level (A3) of sapanisertib ≤3 mg PO QD, 3 d/wk and serabelisib ≤200 mg PO QD, 3 d/wk may be initiated based on review of safety data from dose levels A1 and A2.

Cohort B Dosing Scheme (28-day cycle) for PIKTOR, Fulvestrant, and Palbociclib:

| Dose | Sapani- | Sera- | | | |
|---|---|---|---|---|---|
| level | sertib | belisib | Fulvestrant | Palbociclib | Notes |
| B1 | 2 mg | 100 mg | 500 mg IM | 125 mg QD | DLT |
| | PO QD, | PO QD, | on D 1 & | with food | observation |
| | 3 d/wk | 3 d/wk | D 15 then | for 21 days | period will |
| | | | Q28 d | on and | be for |
| | | | | seven days | 28 days |
| B2 | ≤3 mg | ≤200 mg | | off | If not |
| | PO QD, | PO QD, | | | tolerable, |
| | 3 d/wk | 3 d/wk | | | reduce |
| | | | | | sapanisertib |
| | | | | | dose |
| B3 | ≤4 mg | ≤200 mg | | | |
| | PO QD, | PO QD, | | | |
| | 3 d/wk | 3 d/wk | | | |

A fourth dose level (B4) of sapanisertib ≤4 mg PO QD, 3 d/wk and serabelisib ≤300 mg PO QD, 3 d/wk may be initiated based on review of safety data from dose levels B1 through B3.
Doses of sapanisertib and serabelisib may be modified based on emerging safety and PK data.

In Cohort B, following safety clearance at each dose level, additional participants (approximately 15) may be enrolled to further explore the safety and pharmacodynamics of PIKTOR with fulvestrant and palbociclib

### Objectives and Endpoints

| Objectives | Endpoints |
|---|---|
| Primary | |
| To evaluate the safety and tolerability of | Incidence of dose-limiting toxicities in the |
| PIKTOR with fulvestrant and/or other | first cycle of treatment (28 days) |
| anticancer therapies and to determine the | Adverse events (AEs) as characterized by |
| maximum tolerated doses | type, frequency, timing, relationship to |
| (MTDs)/recommended phase 2 doses | treatment, seriousness, and severity (graded |
| (RP2Ds) of combination treatment | according to National Cancer Institute |
| | Common Terminology Criteria for Adverse |
| | Events [NCI CTCAE] V5.0) |
| | Proportion of participants discontinuing study |
| | treatment due to treatment-emergent AEs |
| | (TEAEs) and treatment-related AEs (TRAEs) |
| | Laboratory abnormalities as characterized by |
| | type, timing, frequency, and severity (as |
| | graded by NCI CTCAE V5.0) |
| Secondary | |
| To evaluate the preliminary efficacy of | Objective response rate (ORR) according to |
| PIKTOR with fulvestrant and/or other | RECIST v1.1 as confirmed by the Investigator |
| anticancer therapies\| | Progression-free survival (PFS) according to |
| | RECIST v1.1 as confirmed by the Investigator |
| | PFS at six months |
| | Overall survival (OS) |
| | Clinical benefit rate (CBR) according to |
| | RECIST v1.1 as confirmed by the Investigator |
| | Duration of response (DoR) according to |
| | RECIST v1.1 as confirmed by the Investigator |
| Exploratory | |
| To assess the pharmacokinetics (PK) of | Plasma PK of sapanisertib and serabelisib will |
| sapanisertib and serabelisib with other | be assessed using a population PK approach. |
| anticancer therapies (e.g., with palbociclib | Concentrations of palbociclib will be |
| in Cohort B) and to assess palbociclib | evaluated to assess steady-state |
| steady-state exposure | |
| To assess metabolic changes related to | Changes from baseline in blood markers of |
| systemic PI3K-pathway inhibition | metabolism, e.g., β-hydroxybutyrate (BHB), |
| | glucose, and insulin |
| To evaluate whether baseline circulating | Secondary efficacy endpoints analyzed in |
| tumor DNA (ctDNA)/next generation | biomarker subgroups |
| sequencing (NGS)-detected | |
| PI3K/AKT/mTOR gene alterations predict | |
| response to PIKTOR-based therapy | |

Abbreviations:
AEs = adverse events;
AKT = protein kinase B;
BHB = β-hydroxybutyrate;
CBR = clinical benefit rate;
ctDNA = circulating tumor DNA;
DoR = duration of response;
MTD = maximum tolerated dose;
mTOR = mechanistic target of rapamycin;
NCI CTCAE = National Cancer Institute Common Terminology Criteria for Adverse Events;
NGS = next generation sequencing;
ORR = objective response rate;
OS = overall survival;
PFS = progression-free survival;
PI3K = phosphoinositide 3-kinase;
PK = pharmacokinetics;
RP2D = recommended Phase 2 dose;
TEAEs = treatment-emergent adverse events;
TRAEs = treatment-related adverse events

TABLE 6
Schedule of Activities (SoA)

| | Trial Period | | | | | | | | |
|---|---|---|---|---|---|---|---|---|---|
| | | | | | | | | Post-treatment | |
| | | | | | | | | Follow-up Period | |
| | | Treatment Period | | | | | End of | Long-term | |
| | Screening | Treatment Cycle | | | | | Treatment | Follow-up | Survival |
| | Period | | | | | | (EoT) | (LTFU) | Follow-up |
| | Screening | Cycle 1 | | Cycle 2 | | | 30 days after | Every 8 | Every 12 |
| | (Day −28 to | | D 15 ± | D 1 ± | D 15 ± | Cycle 3+ | last dose | weeks ± | weeks ± |
| Procedures | Day −1) | D 1 | 2 D | 1 D | 2 D | D 1 ± 2 D | (±7 D) | 2 weeks | 3 weeks |
| Informed consent | X | | | | | | | | |
| Inclusion/exclusion | X | | | | | | | | |
| criteria | | | | | | | | | |
| HR+/HER2− status | X | | | | | | | | |
| Demographics | X | | | | | | | | |
| Medical history | X | | | | | | | | |
| Tumor tissue | X | | | | | | | | |
| Physical exam | X | X | X | X | X | X | X | | |
| (including height | | | | | | | | | |
| and weight) | | | | | | | | | |
| Vital signs | X | X | X | X | X | X | X | | |
| Single-12-lead ECG | X | X | | | | | X | | |
| ECOG performance | X | X | X | X | X | X | X | | |
| status | | | | | | | | | |
| Hematology/ | X | X | X | X | X | X | X | | |
| coagulation/ blood | | | | | | | | | |
| chemistry | | | | | | | | | |
| Metabolic-related | X | | X | | X | | | | |
| labs | | | | | | | | | |
| HbA1c | X | X | | | | X (every other | X | | |
| | | | | | | cycle [C3, C5, | | | |
| | | | | | | etc.]) | | | |
| Urinalysis | X | | | | | | X | | |
| PK sampling | | X | | | | | | | |
| (Cohorts A and B) | | | | | | | | | |
| Blood collection for | X | | | | | X (C3 only) | X | | |
| research samples | | | | | | | | | |
| (ctDNA) | | | | | | | | | |
| Radiographic | X | Every 8 weeks (±1 week) starting at C1D1 through | | | | | X^{l} | X^{m} | |
| imaging and | | week 32, then every 12 weeks (±1 week) thereafter | | | | | | | |
| RECIST v1.1 | | | | | | | | | |
| response assessment | | | | | | | | | |
| Pregnancy test | X | X | | X | | X | X | | |
| (WOCBP only) | | | | | | | | | |
| Fulvestrant | | Dosing per approved package insert | | | | | | | |
| administration | | | | | | | | | |
| (Cohorts A and B) | | | | | | | | | |
| Palbociclib | | Dosing per approved package insert | | | | | | | |
| administration | | | | | | | | | |
| (Cohort B) | | | | | | | | | |
| Dispense serabelisib | | X | | | | | | | |
| Dispense | | X | | | | | | | |
| sapanisertib | | | | | | | | | |
| Prior and | Recorded from the time the ICF is signed through EoT | | | | | | | | |
| concomitant | | | | | | | | | |
| medications | | | | | | | | | |
| SAE collection | Recorded from the time the ICF is signed through EoT^{p} | | | | | | | | |
| AE collection | | Recorded from the first dose through EoT | | | | | | | |
| Subsequent | | | | | | | X | X | X |
| anticancer therapy/ | | | | | | | | | |
| procedures and | | | | | | | | | |
| survival follow-up | | | | | | | | | |

Abbreviations:
AE = adverse event;
ctDNA = circulating tumor DNA;
ECG = electrocardiogram;
ECOG = Eastern Cooperative Oncology Group;
EoT = end of treatment (visit);
HbA1c glycosylated hemoglobin;;
PK = pharmacokinetics;
RECIST = Response Evaluation Criteria in Solid Tumors v1.1;
SAE = serious adverse event;
WOCBP = woman of childbearing potential
Note:
A delay in study treatment or performance of study visits due to holidays, weekends, inclement weather, or other unforeseen circumstances will be permitted and not considered a protocol deviation. However, significant delays (i.e., greater than one week) due to these reasons should be discussed with the Medical Monitor to reach consensus on subsequent scheduling. See Sections below for management of dosing delays due to toxicity.

### Study Design

### Overall Design

The study is a Phase 1b/2, multi-center, open-label dose escalation trial evaluating the safety and preliminary efficacy of PIKTOR with fulvestrant and/or other anticancer therapies in participants with HR+/HER2− advanced or metastatic breast cancer. All participants must have received at least one but not more than three prior lines of therapy to treat advanced disease. The study will evaluate escalating doses of PIKTOR with fulvestrant (Cohort A) or fulvestrant plus palbociclib (Cohort B). After confirmation of eligibility, an average of 6 participants will be enrolled in each dose level. Additional participants may be enrolled in each dose level that is deemed safe for further exploration of safety and pharmacodynamics. Once the RP2D has been determined for each cohort in Phase 1b, approximately 60 participants (approximately 30 in each expansion cohort) will be enrolled under a future amendment for Phase 2.

The study comprises a screening period, a treatment period, and a post-treatment follow-up period. The treatment duration will be until disease progression, death, unacceptable toxicity, withdrawal of consent, or the participant is lost to follow-up. Collection of survival data may continue for up to 5 years following the EoT visit.

The study will evaluate the safety and tolerability of PIKTOR treatment with other anticancer therapies to determine the optimal doses (RP2D) to be evaluated in Phase 2. A BOIN design will be used to identify the MTD of each combination, which refers to the highest dose of each component of PIKTOR that yields a target of approximately 27.5% probability of DLT

The study will use an SMC to evaluate safety throughout the study.

### Screening Period

During the screening period, participants will provide written informed consent. No study-specific procedures that are not part of standard-of-care should be performed prior to informed consent.

### Treatment Period

After eligibility has been confirmed, study treatment will be administered in 28-day cycles according to the schedule provided in Table 7. Details for each cohort are provided in Table 8 and Table 9.

Participants may receive treatment until disease progression, death, unacceptable toxicity, death, withdrawal of consent, or the participant is lost to follow-up or study closure.

TABLE 7
Treatment Administration Schedule per 28-day Cycle

| | Cycle Day | | | | |
|---|---|---|---|---|---|
| Treatment | 1-3 | 8-10 | 15-17 | 22-24 | 25-28 |
| Sapanisertib | X | X | X | X | |
| Serabelisib | X | X | X | X | |
| Fulvestrant 500 mg IM on Days 1, 15 | X | | | | |
| of Cycle 1, and then once monthly | | | | | |

| | Days 1-21 | 22-28 |
|---|---|---|
| Palbociclib 125 mg QD | X | |

Note:
anti-emetics are recommended as needed per institutional standards

TABLE 8
Phase 1b Cohort A Dosing Scheme (28-
day cycle) for PIKTOR and Fulvestrant

| Dose | | | | |
|---|---|---|---|---|
| level | Sapanisertib | Serabelisib | Fulvestrant | Notes |
| A1 | 2 mg PO | 200 mg PO | 500 mg IM | Cohorts A1 and A2 |
| | QD, 3 d/wk | QD, 3 d/wk | D 1 & D 15 | will be initiated in |
| A2 | 4 mg PO | 200 mg PO | then Q28 d | parallel. Participants |
| | QD, 3 d/wk | QD, 3 d/wk | | will be assigned by |
| | | | | the Sponsor. |

A third dose level (A3) of sapanisertib ≤3 mg PO QD, 3 d/wk and serabelisib ≤200 mg PO QD, 3 d/wk may be initiated based on review of safety data from Cohorts A1 and A2.

TABLE 9
Phase 1b Cohort B Dosing Scheme (28-day cycle)
for PIKTOR, Fulvestrant, and Palbociclib

| Dose | Sapani- | Sera- | | | |
|---|---|---|---|---|---|
| level | sertib | belisib | Fulvestrant | Palbociclib | Notes |
| B1 | 2 mg PO | 100 mg | 500 mg IM | 125 mg QD | DLT |
| | QD, | PO QD, | on D 1 & | with food | observation |
| | 3 d/wk | 3 d/wk | D 15 then | for 21 days | period will |
| | | | Q28 d | on and | be for 28 |
| | | | | seven days | days |
| B2 | ≤3 mg | ≤200 mg | | off | If not |
| | PO QD, | PO QD, | | | tolerable, |
| | 3 d/wk | 3 d/wk | | | reduce |
| B3 | ≤4 mg | ≤200 mg | | | sapanisertib |
| | PO QD, | PO QD, | | | dose |
| | 3 d/wk | 3 d/wk | | | |

A fourth dose level (B34) of sapanisertib ≤4 mg PO QD, 3 d/wk and serabelisib ≤300 mg PO QD, 3 d/wk may be initiated based on review of safety data from dose levels B1 through B3.
Doses of sapanisertib and serabelisib may be modified based on emerging safety and PK data.

### Study Population

The characteristics of the study population are summarized below.

### Inclusion Criteria

To be eligible to participate in this study, an individual must meet all the following criteria:

### Type of Participant and Disease Characteristics

1. Participant must provide written informed consent, stating understanding of the procedures and investigational nature of the study treatment, and willingness to comply with study requirements.
2. Histologically confirmed diagnosis of HR+/HER2− breast cancer according to American Society of Clinical Oncology/College of American Pathologists (ASCO/CAP) guidelines
3. Documented evidence of advanced or recurrent disease that is not amenable to surgery/radiation for curative intent.
4. Participant has received at least one prior systemic therapy. Prior treatment with CDK 4/6 inhibitors (e.g., palbociclib, ribociclib, abemaciclib), at least one line of endocrine therapy (e.g., aromatase inhibitors, fulvestrant; most recent prior endocrine therapy PFS had to be equal to or exceed 4 months), and up to 1 prior chemotherapy for advanced disease is permitted. Prior treatment with mTORC1 inhibitors (everolimus, temsirolimus) is permitted.
5. At least one measurable or evaluable target lesion according to RECIST v1.1 as confirmed by the Investigator including the following criteria:
   Measurable disease (based on RECIST 1.1): refers to at least one lesion that can be accurately measured in at least one dimension ≥20 mm with conventional imaging techniques or ≥10 mm with CT or MRI,
   OR
   Evaluable disease: bone lesions—lytic or mixed (lytic+sclerotic) in the absence of measurable disease
6. Life expectancy of at least six months in the opinion of the Investigator.

### Demographics

7. Participant is ≥18 years of age.
8. Eastern Cooperative Oncology Group (ECOG) performance status of ≤1 at Screening.
9. Non-pregnant, non-lactating females who are postmenopausal, surgically sterile or who agree to use effective contraceptive methods as defined by the protocol and who agree not to donate eggs (ova or oocytes) for the purpose of reproduction are eligible:
   Age ≥60 years
   Age <60 years and amenorrhea for 12 or more months (in the absence of chemotherapy, tamoxifen, toremifene, or ovarian suppression) or a follicle-stimulating hormone (FSH) value >40 mIU/mL and an estradiol value <40 pg/mL (140 pmol/L) or in postmenopausal ranges per local reference ranges
   Documentation of prior surgical sterilization (i.e., bilateral tubal ligation, total hysterectomy, or bilateral oophorectomy, at least one month before first dose of trial therapy).
   Premenopausal and perimenopausal women (who do not fit postmenopausal criteria) and men must be concurrently receiving a luteinizing hormone-releasing hormone (LHRH) agonist initiated at least four weeks before the start of trial therapy and are planning to continue LHRH agonist treatment during the study treatment.
   For perimenopausal women to be considered of non-childbearing potential, FSH levels must be >40 mIU/ml.
   Women of childbearing potential must have a negative serum human chorionic gonadotropin pregnancy test at Screening and a negative urine pregnancy test predose at C1D1.

### Additional Criteria

10. At the time of study treatment, resolution of toxic effect(s) of prior therapies to ≤Grade 1 or baseline (except alopecia), and meet the following criteria:
   ≥2 weeks from last non-cytotoxic therapy (see criterion #4 above)
   ≥4 weeks or 5 half-lives, whichever is shorter, from last cytotoxic therapy
   ≥30 days from last investigational agent. Note: Participants who have entered the follow-up phase of an investigational study may participate as long as it has been 30 days after the last dose of the previous investigational agent.
   Note: Toxicity that has not recovered to ≤Grade 1 is allowed if it meets the inclusion requirements for laboratory parameters described below.
11. Agreement to adhere to lifestyle considerations throughout study duration.

### Adequate Systemic Functions

12. Adequately controlled blood pressure (BP) with or without antihypertensive medications, defined as BP≤160/95 mm Hg at Screening and no change in antihypertensive medications within one week before Cycle 1 Day 1.
13. Have adequate organ function

### Exclusion Criteria

An individual who meets any of the following criteria will be excluded from participation in this study:

### Medical Conditions

1. Participants with triple-negative breast cancer.
2. Participants with central nervous system metastases are not eligible, unless they have completed local therapy (e.g., whole brain radiation therapy [WBRT], surgery or radiosurgery) and have discontinued the use of corticosteroids for this indication for at least four weeks before starting treatment in this study. Any signs (e.g., radiologic) or symptoms of brain metastases must be stable for at least four weeks before starting study treatment.
3. Active malignancy (except for breast cancer, definitively treated in-situ carcinomas [e.g., breast, cervix, bladder], or basal or squamous cell carcinoma of the skin) within the past 24 months prior to treatment. Fully resected localized malignancies are eligible.
4. Gastric feeding tube (gastrostomy tube), gastrointestinal malabsorption, gastrointestinal anastomosis, bowel obstruction, or any other condition that might affect the absorption of study treatment.
5. Significant cardiovascular impairment within six months of the first dose of study intervention: such as history of congestive heart failure greater than New York Heart Association Class II, unstable angina, myocardial infarction, or cerebrovascular accident stroke, or cardiac arrhythmia associated with hemodynamic instability or symptomatic pulmonary embolism.
6. Active, uncontrolled infection (if requiring systemic antimicrobial therapy, discuss with the Medical Monitor).
7. Participants with progressive and rapid clinical deterioration that in the opinion of the Investigator is likely to worsen during the first cycle of treatment.
8. Known, symptomatic/uncontrolled infection with human immunodeficiency virus (HIV) or acquired immunodeficiency syndrome (AIDS). No testing is required unless mandated by the local health authority.
9. Known active infection with hepatitis B (e.g., HBsAg reactive) or hepatitis C (e.g., HCV RNA [qualitative] is detected). No testing for hepatitis B or C is required unless mandated by local health authority.
10. Has a history or current evidence of any condition, therapy, or laboratory abnormality that might confound the results of the trial, interfere with the participant's participation for the full duration of the trial, or is not in the best interest of the participant to participate, in the opinion of the treating Investigator.
11. Has a condition the Investigator believes would interfere with the ability to provide informed consent or comply with study instructions, or that might confound the interpretation of the study results or put the participant at undue risk.
12. Concurrent participation in another therapeutic clinical trial.

### Prior/Concomitant Therapy

13. Prior radiation therapy within 21 days prior to start of study treatment.
14. Strong CYP3A4 inhibitors and inducers are prohibited during the study. Strong CYP1A2 inhibitors as well as CYP1A2 inducers should be administered with caution and at the discretion of the Investigator. Alternative treatments, if available, should be considered. Additionally, strong CYP3A4 inhibitors or inducers should not be taken within seven days before the first dose of study intervention.
15. Participants who require PPIs or chronic use of antacids, histamine H2 receptor antagonists, or other treatments to raise gastric pH during the study.
16. Suspected or known hypersensitivity to study treatment (or any of the excipients).
   PI3K, AKT, and/or mTOR Prior Therapies
17. Participants who received a prior PI3K inhibitor (pan-isoform, isoform-specific, or mutant-specific).
18. Participants who received a prior AKT inhibitor.
19. Participants who received a prior dual mTORC1/2 inhibitor.
20. Participants who received a prior dual PI3K/mTOR inhibitor; however, prior treatment with an mTORC1 inhibitor (e.g., everolimus, temsirolimus) is allowed.

### Diagnostic Assessments

21. Prolongation of QTc interval to >480 ms.
22. Type 1 or Type 2 diabetes mellitus on insulin, or patients with/without type 2 diabetes mellitus who have HbA1c >7.0.%, fasting serum glucose >140 mg/dL, or fasting triglycerides >300 mg/dL or receiving treatment with insulin. For participants with HbA1c >7.0%, strongly consider an endocrine consultation prior to enrolling the participant in the study to develop a management plan. For participants on sulfonylureas, consult with an endocrinologist for alternative regimens for blood sugar control.

### Study Interventions and Concomitant Therapy

Study interventions are all prespecified investigational products intended to be administered to the study participants during the study conduct. For this protocol, study intervention refers to sapanisertib, serabelisib, fulvestrant, and palbociclib. Other medications used as support for preventive, diagnostic, or therapeutic reasons or medications given to ensure adequate medical care is provided are considered non-investigational products.

### Administration

Participants will be provided with sufficient quantities of oral study interventions (sapanisertib, serabelisib, and palbociclib) until the next study intervention dispensation visit. Participants will be instructed to bring their unused study interventions and all containers (empty, partially used, and/or unopened) to the next scheduled visit for reconciliation by the site.

Administration instructions for oral medication are detailed below. A treatment cycle is 28 days.

### Sapanisertib and Serabelisib:

Sapanisertib and serabelisib will be administered orally with food QD×3D QW on a 28-day cycle. Participants will take sapanisertib and serabelisib within ~30 minutes of starting a meal (grapefruit and Seville oranges are not allowed). Participants will be informed to take sapanisertib and serabelisib with food.
Participants will swallow the capsules and tablets whole and will not manipulate or chew them prior to swallowing. A missed dose can be taken later in the same day.
If a participant vomits any time after taking a dose, they should be instructed not to “make it up” but resume subsequent doses as instructed.
Anti-emetics are recommended as needed per institutional standards.

### Fulvestrant:

Fulvestrant will be administered in the clinic by study site staff according to local prescribing information.

### Palbociclib:

Palbociclib will be administered orally with food QD for 21 days on and seven days off in each 28-day treatment cycle according to local prescribing information
Participants will swallow the tablets whole and will not manipulate or chew them prior to swallowing.
If a participant vomits or misses a dose, they should be instructed not to “make it up” but resume subsequent doses as instructed.

### Dose Modification

### Dose Modifications—General Considerations

Every effort should be made to adhere to the treatment schedule. Missed doses may be taken later in the day but otherwise should not be made up. Treatment with sapanisertib and/or serabelisib may continue if the other therapy(s) are discontinued.

### Intraparticipant Dose Escalation

After completing the initial DLT observation period, participants in Cohort A may, with Medical monitor approval, receive sapanisertib at the highest dose deemed to be safe by the SMC.

### Dose Modifications for AEs

Any participant receiving treatment will be evaluable for toxicity. Toxicity will be assessed according to NCI CTCAE V5.0. Guidance is provided in the following sections for management and treatment protocols for the potential adverse reactions associated with the combination of PIKTOR. If a decision is made to stop treatment due to toxicity or AEs, the Medical Monitor must be informed.

Note: Participants who discontinue from study treatment for reasons other than disease progression should be followed for progression.

### Dose Reductions and Delays

### Fulvestrant

The dose of fulvestrant can be modified based on the local prescribing information.

### Management of Hyperglycemia

Hyperglycemia and hyperinsulinemia are toxicities associated with inhibition of mTOR and related pathways.

Participants with HbA1C values ≥5.7 (prediabetes or diabetes) are encouraged to start metformin 500 mg orally QD and titrate up to a maximum of 1000 mg orally BID, as tolerated.

Participants developing hyperglycemia during the study should have their glucose closely monitored by study staff The Investigator may choose to continue close monitoring of participants who develop hyperglycemia (fasting glucose >ULN or random glucose >180 mg/dL). For persistent hyperglycemia, treatment with an oral hypoglycemic agent, such as metformin is recommended. All participants with persistent hyperglycemia or more severe elevations (fasting glucose >160 mg/dL or random glucose >250 mg/dL) should be treated aggressively with oral hypoglycemic agents to prevent the need for insulin therapy. The Investigator should consult an endocrinologist, if needed, to aid in optimizing the participant's hyperglycemia treatment plan.

It is recommended that participants be initially treated with metformin at 500 mg orally QD and titrated up to a maximum of 1000 mg orally BID as tolerated. Concurrent addition to metformin of sodium-glucose cotransporter-2 (SGLT2) inhibitors (e.g., empagliflozin, dapagliflozin, canagliflozin, or ertugliflozin) should also be considered. Dipeptidyl peptidase-4 (DPP-4) inhibitors (e.g., sitagliptin or vildagliptin) may also be considered. Sulfonylureas (e.g., glipizide, glyburide, or other sulfonylureas) are discouraged but allowed if no other alternatives are available for the participant) for additional glycemic control. Of note, sulfonylureas can induce hypoglycemia in participants, especially if taken on days when PIKTOR is held. The dose of oral hypoglycemic agents should be adjusted in participants with renal insufficiency.

If any fasting serum glucose reading performed at the site indicates hyperglycemia (>ULN or ≥110 mg/dL), the study staff should first confirm that the participant was fasting at the time of blood specimen collection.

Guidance on study intervention dose modification for participants with hyperglycemia is provided in Table 1.

TABLE 1
Management of Hyperglycemia

| Grade | Treatment | Dose Modification |
|---|---|---|
| 1 (abnormal glucose | Continue close monitoring of blood glucose. | None |
| above baseline with | Encourage oral hydration.^{a} | |
| no medical | | |
| intervention) | | |
| 2 (change in daily | If clinically indicated and if not well controlled, initiate | None |
| management from | oral hypoglycemic agent or adjust if already taking oral | |
| baseline; oral | hypoglycemic agent; if not well controlled, consider | |
| antihyperglycemic | adding a second oral agent. | |
| agent initiated) | Consult Medical Monitor for guidance on anti- | |
| | hyperglycemic agents | |
| | Endocrinology consultation if not already done. | |
| | Encourage oral hydration. | |
| ≥3 (insulin therapy | Potential hospitalization and treatment per in-patient | Hold sapanisertib and |
| initiated; | team for acute management | serabelisib until ≤Grade |
| hospitalization | | 2 (and insulin has been |
| indicated) | | discontinued). |
| | | Resume sapanisertib and |
| | | serabelisib based on |
| | | timing of recovery after |
| | | maximal treatment: |
| | | ≤1 week: resume |
| | | sapanisertib and |
| | | serabelisib at same |
| | | dose and schedule. |
| | | >1 but ≤2 weeks: |
| | | reduce sapanisertib |
| | | by 1 dose level. |
| | | >2 weeks: reduce |
| | | both sapanisertib |
| | | and serabelisib by 1 |
| | | dose level |

Prevention/Prophylaxis:
Follow fasting glucose levels during clinic visits.
Check HbA1c levels every three months during therapy.
Recommend lifestyle modifications, as appropriate (e.g., avoidance of sugar-sweetened beverages and processed high carbohydrate snacks, limited alcohol consumption, increased physical activity).
Most episodes of Grade 1 or 2 hyperglycemia respond quickly to oral metformin. Early initiation and titration of metformin is recommended to prevent higher grade hyperglycemia.

TABLE 2
Management of Oral Mucositis

| Grade | Treatment | Dose Modification |
|---|---|---|
| 1 | Non-alcoholic mouth wash, | None |
| | or 0.9% saltwater rinse. | |
| | Consider topical | |
| | corticosteroids at earliest | |
| | signs of mucositis. | |
| 2 | Topical analgesic mouth | Maintain sapanisertib and serabelisib dose if tolerable |
| | treatments. | Hold only sapanisertib and serabelisib if intolerable until |
| | Topical corticosteroids. | recovery to ≤Grade 1, then restart at same dose. |
| | Initiate antiviral or antifungal | |
| | therapy, if indicated. | |
| 3 | Same as for Grade 2. | Hold sapanisertib and serabelisib until recovery to ≤Grade |
| | Consider intralesional | 1, then restart sapanisertib at the next lower dose level and |
| | corticosteroids. | serabelisib at the same dose level |
| 4 | Same as for Grade 2 | Stop sapanisertib and/or serabelisib and discuss with the |
| | Consider intra-lesional | Medical Monitor |
| | corticosteroids | |

Prevention/Prophylaxis:
Initiation of a non-alcoholic mouth wash, or 0.9% saltwater rinses 4 to 6 times daily is strongly recommended at the start of therapy before signs of mucositis develop.
Avoid using agents containing hydrogen peroxide, iodine, and thyme derivatives in management of stomatitis, as they may worsen mouth ulcers.

### Efficacy Assessments

### Imaging and Tumor Response Assessments

Disease response and progression will be based on RECIST v1.1 criteria. These data will constitute the primary assessment for ORR and PFS analyses. Following each scan, the Investigator will assess for progression and assign a response criteria category using RECIST v1.1. Participants being considered for study entry should have documented evidence of HR+/HER2− advanced or metastatic breast cancer, radiographic evidence of disease progression on or after a prior systemic regimen, and at least one measurable or evaluable target lesion according to RECIST v1.1 as confirmed by the Investigator.

Tumor imaging will consist of radiological assessments using CT (with contrast) or MRI scans of chest, abdomen, and pelvis. The method used for a participant [CT or MRI] must be the same throughout the study. Bone scans should be conducted in participants with bone disease only.

Tumor assessments will be performed during screening (baseline), during treatment, whenever disease progression is suspected, at the EoT visit (if not done in the previous four weeks), and during LTFU. If the reason for treatment discontinuation was other than radiographically confirmed disease progression, tumor assessments should continue until radiographic disease progression or withdrawal of consent is given, according to the SoA.

The review of radiographic and clinical data by the study Investigators will be performed on an ongoing basis. Confirmation should be obtained at a subsequent time point (at least four weeks after the first record of response). De-identified images should be provided to the Sponsor to be held for central, independent review.

### Survival Assessment

The survival status of each participant will be monitored during the study intervention and after treatment discontinuation for any reason. Survival status will be documented during the Survival Follow-Up, which will be monitored every 12 weeks from EoT if the participant did not enter the LTFU period, or from the last LTFU visit during which radiographic disease progression was confirmed according to the SoA. The date and cause of death will be recorded. Survival follow-up will continue until death, loss to follow-up, withdrawal of consent, or study closure, whichever occurs first.

### Safety Assessments

Planned timepoints for all safety assessments are provided in the SoA. Unscheduled safety assessments may be obtained at any time during the study to assess any perceived safety issues.

### Physical Examinations

A complete physical examination will include, at a minimum, assessments of the cardiovascular, respiratory, GI, and neurological systems and should consider sites of metastases.

Investigators should pay special attention to clinical signs related to previous serious illnesses.

Physical examinations may be conducted by a physician, trained physician's assistant, or nurse practitioner as acceptable according to local regulations.

Height will be assessed at Screening only and weight will be measured at the timepoints indicated in the SoA.

Physical examination findings collected during the study will be considered source data and will not be required to be reported, unless otherwise noted. Any untoward physical examination findings that meet the definition of an AE or SAE must be reported.

### ECOG Performance Status

Scoring for the ECOG performance status assessment is provided below

### Vital Signs

Vital signs will be collected at the times specified in the SoA.

Vital sign measurement consists of the following variables: blood pressure (diastolic and systolic), heart rate, respiratory rate, SpO₂, and temperature.

Resting vital sign measurements will be performed after the participant has been at rest for a minimum of five minutes. Vital signs will be measured prior to any blood draws that occur at the same study visit.

Blood pressure may be measured manually or with an automated device, preferably in the nondominant arm. The same measurement position and technique for all vital sign evaluations should be used throughout the study for each participant and should be documented on the CRF.

Abnormal readings that are clinically significant should be repeated and confirmed.

Any untoward vital sign findings that meet the definition of an AE or SAE must be reported.

### Adverse Events, Serious Adverse Events, and Other Safety Reporting

AEs should be documented and recorded at visits in alignment with the SoA. AEs will be reported by the participant (or, when appropriate, by a caregiver, surrogate, or the participant's legally authorized representative).

During the AE and SAE collection period, each participant or legally authorized representative will be questioned about the occurrence of AEs in a nonleading manner.

The method of recording, evaluating, and assessing causality of AEs and SAEs and the procedures for completing and transmitting SAE reports are provided below. The causality of AEs should be assessed for all interventions.

### Time Period and Frequency for Collecting AE and SAE Information

All SAEs will be collected from the time the participant provides informed consent, which is obtained before undergoing any study-related screening procedures and/or receiving study intervention.

If a participant is assessed as a screen failure, subsequent reporting of SAEs will no longer be done in the CRF or reported to the Sponsor.

All AEs will be collected from the start of intervention until the EoT visit at the timepoints specified in the SoA.

Medical occurrences that begin before the start of study intervention but after obtaining informed consent will be recorded as medical history/current medical conditions, not as AEs.

All SAEs that are reportable to the sponsor based on the reporting periods defined in this protocol must be reported on an SAE Report Form and submitted within 24 hours of knowledge of the event. The initial notification should be as complete as possible with the information available. Follow up will continue until SAE resolution.

Investigators are not obligated to actively seek information on AEs or SAEs after conclusion of the study participation. However, if the Investigator learns of any SAE, including a death, at any time after a participant has been discharged from the study, and he/she considers the event to be reasonably related to the study intervention or study participation, the Investigator must promptly notify the Sponsor.

### Method of Detecting AEs and SAEs

The method of recording, evaluating and assessing causality of AE and SAE and the procedures for completing and transmitting SAE reports are provided below.

Care will be taken not to introduce bias when detecting AEs and/or SAEs. Open-ended and nonleading verbal questioning of the participant is the preferred method to inquire about AE occurrences.

### Follow-up of AEs and SAEs

After the initial AE/SAE report, the Investigator is required to proactively follow each participant at subsequent visits/contacts. All AEs/SAEs will be followed until resolution, stabilization, the event is otherwise explained, or the participant is lost to follow-up. Further information on follow-up procedures is provided below.

If a participant begins a new anticancer therapy, the AE reporting period for non-SAEs ends at the time the new treatment is started.

### Pharmacokinetics

For sapanisertib and serabelisib PK in Cohorts A and B and palbociclib PK in Cohort B, venous blood samples will be collected at each PK sampling day specified in the SoA for measurement of plasma concentrations of sapanisertib, serabelisib, and palbociclib.

For Cohort A, a single pre-dose PK sample should be collected from all participants on D2 or D3 of the sapanisertib/serabelisib dosing schedule (equivalent to Days 9 or 10 or Days 16 or 17 of the 28-day cycle) in the second or third week of Cycle 1 and Cycle 2.

For Cohort B dose levels B2-B4, a single pre-dose PK sample should be collected from all participants on D2 or D3 of the sapanisertib/serabelisib dosing schedule (equivalent to Days 9 or 10 or Days 16 or 17 of the 28-day cycle) in the second or third week of Cycle 1 and Cycle 2. Sampling should occur pre-dose, 2, and 4 hours after dosing with food. For Cohort B1 (first 12 participants), sampling should occur pre-dose, 0.5, 1, 2, 4, 8, and 24 hours after dosing during Cycle 1 only.

During PK sampling days, the participant will take sapanisertib and serabelisib, and palbociclib as applicable, in the clinic with food. Date and time of drug administration as well as PK sampling should be recorded.

Additional samples may be collected at additional timepoints during the study if warranted and agreed upon between the Investigator and the Sponsor for further assessment of an observed toxicity or unexpected SAE, or AE that led to discontinuation.

Instructions for the collection and handling of biological samples will be provided by the Sponsor. The actual date and time (24-hour clock time) of each sample will be recorded. The actual dosing time on the day of PK sampling and the day preceding PK sampling will also be recorded on the CRF.

Samples will be used to evaluate the PK of sapanisertib, serabelisib, and palbociclib. Samples collected for analyses of sapanisertib and serabelisib and palbociclib concentration may also be used to evaluate safety or efficacy aspects related to concerns arising during or after the study.

Sapanisertib, serabelisib, and palbociclib PK concentrations will be reported descriptively. The population PK of sapanisertib and serabelisib (e.g., AUC, C_{trough}, Cₘₐₓ) and covariates that affect PK (e.g., body weight, race) will be assessed. Exposure-response (safety and efficacy) analysis will be performed as appropriate.

### Biomarkers

Exploratory: ctDNA

A whole blood sample will be collected for ctDNA isolation at the timepoints specified in the SoA. Samples will be analyzed. Investigations will be conducted of various forms of genetic alterations, including point mutations and gene amplifications, as well as markers of DNA repair capacity such as microsatellite instability. Data from participants who have (and consent to the use of) existing molecular testing data (e.g., from commercial mutation profiling panels) may also be used for analyses.

Various gene signatures (as individual genes and in combinations), representing varying degrees of pathway coverage, will be explored. Mutations in genes of the direct therapeutic targets of serabelisib and sapanisertib (i.e., PIK3CA and mTOR), other core pathway genes (e.g., AKT1, AKT2, AKT3, TSC1 and TSC2), pathway regulators (e.g., PTEN and PIK3R1) and ultimately all PI3K/AKT/mTOR pathway related genes will be assessed. Further exploratory analyses that test associations of genes beyond the PI3K/AKT/mTOR pathway with clinical response may be conducted.

### Exploratory: Effects of Treatment on Metabolism Markers

Inhibition of the PI3K/AKT/mTOR pathway has impacts on systemic metabolism, leading to changes in blood chemistry, especially hyperglycemia (elevated glucose levels). PI3K/AKT/mTOR pathway inhibition causes elevation of systemic insulin and that excess insulin degrades PI3K/AKT/mTOR pathway inhibitor efficacy. Effects of treatment on the relevant metabolism parameters will be evaluated.

### Statistical Considerations

Detailed plans for analysis will be summarized in a separate Statistical Analysis Plan. Summaries of the statistical aspects of the design and intended analyses are provided below.

### Analysis Sets

The study populations will be as follows:
All Treated population (Full Analysis Set [FAS]): all participants who receive a dose of any study intervention
DLT Evaluable Set (DLTS): All treated participants who are DLT evaluable:
   Has received at least 80% of the intended dose of sapanisertib and serabelisib during the DLT window or received less than 80% of the intended dose due to toxicity
   Has completed the follow-up during the DLT window without experiencing a DLT, or experienced a DLT
Response-evaluable population (secondary analysis): participants with measurable disease who receive at least one dose of any study intervention and have at least one post-baseline disease assessment, or participants who receive at least 1 dose of any study intervention and leave the study early due to study-related toxicity (including death).
Disease Control Response-evaluable population (secondary analysis): participants with evaluable (measurable and non-measurable) disease who receive at least one dose of any study intervention and have at least one post-baseline disease assessment, or participants who receive at least one dose of any study intervention and leave the study early due to study-related toxicity (including death).

### Statistical Analyses

### General Considerations

Unless otherwise specified, efficacy and safety analyses will be performed using the All Treated population by cohort and by dose level. In general, descriptive summaries will be presented for efficacy and safety variables collected. Continuous variables will be summarized using mean, standard deviation, minimum, median, and maximum. Categorical variables will be summarized using frequency counts and percentages.

### Safety Endpoint Analysis

Safety analyses will evaluate AEs as characterized by type, frequency, timing, relationship to treatment, seriousness, and severity. Event severity will be graded according to NCI CTCAE V5.0. All analyses will be summarized using descriptive statistics. The number and percentage of participants who experienced any DLT, TEAE, SAE, or TRAE will be summarized according to worst NCI CTCAE grades. The frequency and severity of TRAEs and the proportion of participants discontinuing study treatment due to TEAEs and TRAEs will be summarized. Laboratory abnormalities as characterized by type, timing, frequency, and severity (as graded by NCI CTCAE V5.0) will also be summarized. Safety data will be reported in tables and listings.

### Efficacy Endpoint Analyses

The secondary endpoints in the study are described below:
ORR is the proportion of participants from the Response-Evaluable population who have confirmed best overall response of either CR or PR according to RECIST v1.1 as confirmed by the Investigator. A confirmatory scan at ≥4 weeks in cases of partial or complete response at the prior scan, or in cases of suspected disease progression, is required. A minimum interval time of six weeks (42 days) between C1D1 and the imaging timepoint is required to declare stable disease (SD).
PFS, which is the time from first dose to the date of the first evidence of disease progression or death from any cause (whichever occurs first).
PFS at six months, is the PFS rate at six months.
OS is the time from first dose to the date of death due to any cause regardless of whether a participant is receiving a subsequent anticancer treatment.
Clinical Benefit Rate (CBR) is the percentage of participants from the All Treated population who achieve CR, PR, or SD as follows:
   CBR-16: percentage of participants who achieve CR or PR or have SD of at least 16 weeks
   CBR-24: percentage of participants who achieve CR or PR or have SD of at least 24 weeks
DoR is for participants with a confirmed CR or PR as the time from response to the date of first evidence of disease progression or death from any cause (whichever occurs first).

### ORR

ORR and CBR will be presented along with its 95% CI that will be calculated using the Clopper-Pearson method.

PFS, DoR and OS will be analyzed through Kaplan-Meier (KM) technique. Rates at specific timepoints and medians will be reported along with 95% CI. KM curves also will be displayed.

### Exploratory Analyses

### Pharmacokinetics of Sapanisertib, Serabelisib, and Palbociclib

Based on the individual plasma concentration-time data, using the actual medication intake and sampling time, individual PK parameters (AUC, C_{trough} and Cₘₐₓ) will be derived through a population PK modeling approach. The influence of covariates such as weight, race, etc. on the population PK of sapanisertib and serabelisib will be investigated. Descriptive statistics will be reported for the individual PK parameters. Exposure-response (safety and efficacy) analyses will be performed as appropriate.

Concentrations of palbociclib will be summarized and descriptive statistics will be reported.

Details of the population PK modeling and exposure-response analyses will be specified in a separate analysis plan.

Efficacy of PIKTOR with Fulvestrant or Fulvestrant+Palbociclib for Participants with PI3K/AKT/mTOR Pathway Genetic Alterations

The same analysis methods as described above will be used for sensitivity analyses to evaluate efficacy responses in participants with specific PI3K/AKT/mTOR pathway genetic alterations.

### Metabolite Levels and Tumor Genetic/Epigenetic Alterations

Analysis summaries of baseline ctDNA levels and changes from baseline (where appropriate) will be reported. Additional exploratory biomarker analyses involving metabolite levels and tumor genetic/epigenetic alterations (including association with efficacy outcomes) may be analyzed if sufficient data are available.

### PK Analyses

Plasma concentration data of sapanisertib, serabelisib, and palbociclib will be listed, summarized, and plotted.

Based on the individual plasma concentration-time data, using the actual medication intake and sampling time, individual PK parameters (AUC, C_{trough} and Cₘₐₓ) will be derived through a population PK modeling approach. The influence of covariates such as weight, race, etc. on the population PK of sapanisertib and serabelisib will be investigated. Descriptive statistics will be reported for the individual PK parameters. Exposure-response (safety and efficacy) analysis will be performed as appropriate.

Details of the population PK modeling and exposure-response analysis will be specified in a separate analysis plan.

### Sample Size Determination

The number of participants to be enrolled in this trial will depend upon the observed safety profile, the number of dose escalations, the number of additional participants, and the BOIN COMBO algorithm.

However, it is estimated that up to 108 patients may be dosed in this trial based upon the following:
Assuming five dose levels will be explored in the dose-escalation stage in Cohorts A and B, with an average of six participants per dose level, approximately 30 DLT-evaluable participants will be needed. Assuming 1000 of participants are not DLT evaluable, approximately 33 participants will be enrolled.
Up to 15 participants at each dose level may be enrolled to further explore the safety and pharmacodynamics of PIKTOR with fulvestrant ±palbociclib.

### Clinical Laboratory Tests

The following laboratory tests (Table) and metabolism-related tests (Table) will be performed by a local laboratory at times defined in the SoA. Additional tests may be performed at any time during the study as determined necessary by the Investigator or required by local regulations.

TABLE 12
Protocol-Required Laboratory Tests
Laboratory

| Tests | Parameters | | | |
|---|---|---|---|---|
| Hematology | Platelet count | RBC indices: | | White blood cell (WBC) |
| (local laboratory) | Red blood cell (RBC) count | Mean corpuscular volume | | count with percent and |
| | Hemoglobin | (MCV) | | absolute differential: |
| | Hematocrit | Mean corpuscular hemoglobin | | Neutrophils |
| | White blood cells | (MCH) | | Lymphocytes |
| | | Mean corpuscular hemoglobin | | Monocytes |
| | | concentration | | Eosinophils |
| | | Red cell distribution width | | Basophils |
| Coagulation | PT and/or INR (screening only) | | | |
| (local laboratory) | | | | |
| Clinical chemistry | Blood urea nitrogen | Potassium | AST/serum | Total bilirubin |
| (local laboratory)^{a} | (BUN) | | glutamic-oxaloacetic | and/or direct |
| | | | transaminase | bilirubin, if |
| | | | (SGOT) | measured. |
| | Creatinine | Sodium | ALT/serum | Total protein |
| | | | glutamic-pyruvic | |
| | | | transaminase (SGPT) | |
| | Glucose | Calcium | Alkaline | |
| | | | phosphatase^{b} | |
| | LDH | Chloride | Bicarbonate | |
| | Phosphate | Magnesium | Albumin | |
| Clinical chemistry | HbA1c | | | |
| (local laboratory) | | | | |

| | |
|---|---|
| Routine urinalysis | Specific gravity |
| (local laboratory) | pH, glucose, protein, blood, ketones, nitrite, leukocyte esterase by dipstick |
| | Microscopic examination (if blood or protein is abnormal) |
| Pregnancy testing | Highly sensitive serum human chorionic gonadotropin (hCG) pregnancy test for WOCB at |
| (local laboratory) | Screening; urine pregnancy test for WOCBP predose on Cycle 1 Day 1 unless the screening |
| | serum test was performed within two days of C1D1, A urine pregnancy test (predose) or |
| | serum pregnancy test (within one day before dosing, or per institutional guidelines) should |
| | be performed at subsequent visits |

^{a}All events of ALT [or AST] ≥ 3 × upper limit of normal (ULN) and total bilirubin ≥ 2 × ULN (>35% direct bilirubin) or ALT [or AST] ≥ 3 × ULN and international normalized ratio (INR) > 1.5 (if INR measured), which may indicate severe liver injury (Mpossible Hy's law), must be reported to Sponsor in an expedited manner.
^{b}If alkaline phosphatase is elevated, consider fractionating.

TABLE 13
Metabolism-Related Laboratory Tests

| Laboratory Tests | Metabolism-Related Parameters |
|---|---|
| Metabolism-related tests | beta hydroxybutyrate (BHB) |
| (local laboratory) | glucose |
| | serum insulin |

AEs and SAEs: Definitions and Procedures for Recording, Evaluating, Follow-up, and Reporting

### Adverse Events (AEs)

AE Definition

An AE is any untoward medical occurrence in a clinical study participant, temporally
associated with the use of study intervention, whether or not considered related to the
study intervention.
NOTE: An AE can therefore be any unfavorable and unintended sign (including an
abnormal laboratory finding), symptom, or disease (new or exacerbated) temporally
associated with the use of study intervention.

Events Meeting the AE Definition

Any abnormal laboratory test results (hematology, clinical chemistry, or urinalysis) or
other safety assessments (e.g., ECG, radiological scans, vital signs measurements),
including those that worsen from baseline, considered clinically significant in the
medical and scientific judgment of the Investigator.
Exacerbation of a chronic or intermittent pre-existing condition including either an
increase in frequency and/or intensity of the condition.
New condition detected or diagnosed after study intervention administration even
though it may have been present before the start of the study.
Signs, symptoms, or the clinical sequelae of a suspected intervention-intervention
interaction.
Signs, symptoms, or the clinical sequelae of a suspected overdose of either study
intervention or a concomitant medication. Overdose per se will not be reported as an
AE/SAE unless it is an intentional overdose taken with possible suicidal/self-harming
intent. Such overdoses should be reported regardless of sequelae.

Events NOT Meeting the AE Definition

Any clinically significant abnormal laboratory findings or other abnormal safety
assessments that are associated with the underlying disease, unless judged by the
Investigator to be more severe than expected for the participant's condition.
The disease/disorder being studied or expected progression, signs, or symptoms of the
disease/disorder being studied, unless more severe than expected for the participant's
condition. Clinical signs or symptoms of progression that cannot be determined as
reasonably due to progression of the underlying malignancy or do not fit the expected
pattern of progression for the disease under study should be reported as an AE.
Radiological disease progression should not be reported as an AE.
Medical or surgical procedure (e.g., endoscopy, appendectomy): the condition that
leads to the procedure is the AE.
Situations in which an untoward medical occurrence did not occur (social and/or
convenience admission to a hospital).
Anticipated day-to-day fluctuations of pre-existing disease(s) or condition(s) present
or detected at the start of the study that do not worsen.

### Serious Adverse Events (SAEs)

An SAE is defined as any untoward medical occurrence that,
at any dose, meets one or more of the criteria listed:

| | |
|---|---|
| a. | Results in death |
| b. | Is life threatening |

The term life threatening in the definition of serious refers to an event in which the participant
was at risk of death at the time of the event. It does not refer to an event, which hypothetically
might have caused death, if it were more severe.

| c. | Requires inpatient hospitalization or prolongation of existing hospitalization | |
|---|---|---|
| | | In general, hospitalization signifies that the participant has been admitted (usually |
| | | involving at least an overnight stay) at the hospital or emergency ward for observation |
| | | and/or treatment that would not have been appropriate in the physician's office or |
| | | outpatient setting. Complications that occur during hospitalization are AEs. If a |
| | | complication prolongs hospitalization or fulfills any other serious criteria, the event is |
| | | serious. When in doubt as to whether hospitalization occurred or was necessary, the AE |
| | | should be considered serious. |
| | | Hospitalization for elective treatment of a pre-existing condition that did not worsen |
| | | from baseline is not considered an AE. |
| d. | | Results in persistent or significant disability/incapacity |
| | | The term disability means a substantial disruption of a person's ability to conduct |
| | | normal life functions. |
| | | This definition is not intended to include experiences of relatively minor medical |
| | | significance such as uncomplicated headache, nausea, vomiting, diarrhea, influenza, |
| | | and accidental trauma (e.g., sprained ankle) that may interfere with or prevent everyday |
| | | life functions but do not constitute a substantial disruption. |

| | |
|---|---|
| e. | Is a congenital anomaly/birth defect |
| f. | Other situations: |

Medical or scientific judgment should be exercised by the Investigator in deciding
whether SAE reporting is appropriate in other situations such as significant medical
events that may jeopardize the participant or may require medical or surgical
intervention to prevent one of the other outcomes listed in the above definition. These
events should usually be considered serious.
Examples of such events include invasive or malignant cancers, intensive treatment for
allergic bronchospasm, blood dyscrasias, convulsions or development of intervention
dependency or intervention abuse.

Recording and Follow-Up of AE and/or SAE

```
AE and SAE Recording
  When an AE/SAE occurs, it is the responsibility of the Investigator to review all
  documentation (e.g., hospital progress notes, laboratory reports, and diagnostics
  reports) related to the event.
  The Investigator will then record all relevant AE/SAE information.
  It is not acceptable for the Investigator to send photocopies of the participant's medical
  records to the Sponsor in lieu of completion of the CRF/required form.
  There may be instances when copies of medical records for certain cases are requested
  by the Sponsor. In this case, all participant identifiers, with the exception of the
  participant number, will be redacted on the copies of the medical records before
  submission to the Sponsor.
  The Investigator will attempt to establish a diagnosis of the event based on signs,
  symptoms, and/or other clinical information. Whenever possible, the diagnosis (not the
  individual signs/symptoms) will be documented as the AE/SAE.
Assessment of Intensity
The Investigator will make an assessment of intensity for each AE and SAE reported during
the study and assign it to one of the following categories as defined by NCI CTCAE V5.0:
  Grade 1, Mild: Asymptomatic or mild symptoms; clinical or diagnostic observations
  only; intervention not indicated.
  Grade 2, Moderate: Minimal, local or noninvasive intervention indicated; limiting age-
  appropriate instrumental Activities of Daily Living (ADL). Instrumental ADL refers to
  preparing meals, shopping for groceries or clothes, using the telephone, managing
  money, etc.
  Grade 3, Severe: Severe or medically significant but not immediately life-threatening;
  hospitalization or prolongation of hospitalization indicated; disabling, limiting self-care
  ADL. Self-care ADL refers to bathing, dressing and undressing, feeding self, using the
  toilet, taking medications, and not bedridden.
  Grade 4; Life threatening consequences; urgent intervention indicated.
  Grade 5: Death related to AE.
```

Assessment of Causality

The Investigator is obligated to assess the relationship between study intervention and
each occurrence of each AE/SAE. The Investigator will use clinical judgment to
determine the relationship.
A reasonable possibility of a relationship conveys that there are facts, evidence, and/or
arguments to suggest a causal relationship, rather than a relationship cannot be ruled
out.
Alternative causes, such as underlying disease(s), concomitant therapy, and other risk
factors, as well as the temporal relationship of the event to study intervention
administration, will be considered and investigated.
The Investigator will also consult the IB and/or product information, for marketed
products, in his/her assessment.
For each AE/SAE, the Investigator must document in the medical notes that he/she has
reviewed the AE/SAE and has provided an assessment of causality.
There may be situations in which an SAE has occurred and the Investigator has
minimal information to include in the initial report to the Sponsor. However, it is very
important that the Investigator always make an assessment of causality for every event
before the initial transmission of the SAE data to the Sponsor.
The Investigator may change his/her opinion of causality in light of follow-up
information and send an SAE follow-up report with the updated causality assessment.
The causality assessment is one of the criteria used when determining regulatory
reporting requirements.

Follow-up of AEs and SAEs

The Investigator is obligated to perform or arrange for the conduct of supplemental
measurements and/or evaluations as medically indicated or as requested by the Sponsor
to elucidate the nature and/or causality of the AE or SAE as fully as possible. This may
include additional laboratory tests or investigations, histopathological examinations, or
consultation with other health care professionals.
If a participant dies during participation in the study or during a recognized follow-up
period, the Investigator will provide the Sponsor with a copy of any postmortem
findings including histopathology.
Investigator is obligated to provide all relevant updates to the Sponsor in a timely
manner.

| Grade | Description |
|---|---|
| 0 | Normal activity. Fully active, able to carry on all predisease performance |
| | without restriction |
| 1 | Symptoms but ambulatory. Restricted in physically strenuous activity, but |
| | ambulatory and able to carry out work of a light or sedentary nature (e.g., |
| | light housework, office work) |
| 2 | In bed <50% of the time. Ambulatory and capable of all self-care, but unable |
| | to carry out any work activities. Up and about more than 50% of waking |
| | hours. |
| 3 | In bed >50% of the time. Capable of only limited self-care, confined to bed |
| | or chair more than 50% of waking hours. |
| 4 | 100% bedridden. Completely disabled. Cannot carry on any self-care. Totally |
| | confined to bed or chair |
| 5 | Dead |

Source: Oken M M, Creech R H, Tormey D C, et al. Toxicity and response criteria of the Eastern Cooperative Oncology Group. Am J Clin Oncol 1982; 5(6): 649-55, herein incorporated by reference.

### Dose-limiting Toxicity Criteria

### DLT Criteria

DLTs will be evaluated during the first 28-day treatment cycle.

The participant population evaluable for DLT assessment will include a) participants enrolled and treated prior to determination of the MTD and who have received ≥80% of the planned doses of treatment, b) the Sponsor and Investigator consider the safety data adequate to conclude a DLT did or did not occur, and c) the participant is observed for at least 28 days following the first dose of study treatment. Participants who do not receive at least 80% of their planned dose for reasons other than toxicity are not DLT evaluable and should be replaced.

The Medical Monitor should be consulted before a DLT is declared, and once declared, the Safety Monitoring Committee should be notified within 24 hours.

Dose-limiting toxicities are defined per AEs (graded according to NCI CTCAE v5.0) with onset during the DLT evaluation period that are possibly, probably, or definitely related to the study drug (e.g., excluding disease progression or extraneous causes) including:
Any death clearly not due to the underlying disease or extraneous causes.
Any clinically significant toxicity that results in a >14-day treatment delay.

### Tumor Imaging and RECIST v1.1 Disease Assessments

Baseline: Baseline is defined as the most recent assessment performed before the first dose of study treatment. Baseline assessments must be performed within the screening period defined in the protocol. At baseline, tumor lesions/lymph nodes will be categorized as measurable or non-measurable.

Measurable Lesions: Except for lymph nodes (described below), measurable lesions are defined as those that can be accurately measured in at least one dimension (longest diameter to be recorded) with a minimum size of: 10 mm by CT scan (CT scan slice thickness no greater than 5 mm, if CT scans have slice thickness greater than 5 mm, the minimum size of a measurable lesion is twice the slice thickness), or 10 mm caliper measurement by clinical exam (lesions which cannot be accurately measured with calipers should be recorded as non-measurable).

To be considered pathologically enlarged and measurable, a lymph node must be ≥15 mm in short axis when assessed by CT scan (CT scan slice thickness recommended to be no greater than 5 mm). At baseline and in follow-up, only the short axis will be measured and followed.

CT is the best currently available and reproducible method to measure lesions selected for response assessment. MRI may be used instead of CT in selected instances. Measurements of pulmonary parenchymal lesions as well as mediastinal disease are optimally performed with CT of the chest. MRI of the chest should only be performed in extenuating circumstances. Even if IV contrast cannot be administered (for example, in the situation of allergy to contrast), a non-contrast CT of the chest is still preferred over MRI.

Non-measurable Lesions: all other lesions, including small lesions (longest diameter <10 mm or pathological lymph nodes with ≥10 to <15 mm short axis), as well as truly non-measurable lesions (e.g. ascites, pleural effusion, lymphangitic involvement of skin or lung, etc.) are considered non-measurable. Lytic bone lesions or mixed lytic-blastic lesions, with identifiable soft tissue components, that can be evaluated by cross-sectional imaging techniques such as CT or MRI can be considered as measurable lesions if the soft tissue component meets the definition of measurability described above. Blastic bone lesions are non-measurable. Lesions situated in a previously irradiated area, or in an area subjected to other locoregional therapy, are usually not considered measurable unless there has been demonstrated progression in the lesion.

Target Lesions: All measurable lesions up to a maximum of two lesions per organ and five lesions in total, representative of all involved organs, are to be identified as target lesions and measured and recorded at baseline.

Target lesions are to be selected on the basis of their size (lesions with the longest diameter) to represent all involved organs, and to be those that lend themselves to reproducible repeated measurements. It may be the case that on occasion, the largest lesion does not lend itself to reproducible measurement in which circumstance the next largest lesion which can be measured reproducibly should be selected.

Target lesions will be measured at each assessment (longest axis for non-nodal lesions, shortest axis for measurable malignant nodal lesions).

Non-target Lesions: All other lesions (or sites of disease) including all non-measurable lesions (including pathological lymph nodes with ≥10 to <15 mm short axis) and all measurable lesions over and above the five target lesions are to be identified as non-target lesions and recorded at baseline. Measurements of these lesions are not required, but the presence, absence, and/or unequivocal progression of each is to be recorded throughout follow-up.

### Special Considerations:

Cystic Lesions: Cystic lesions thought to represent cystic metastases can be considered as measurable lesions if they meet the definition of measurability described above. However, if non-cystic lesions are present in the same participant, these are preferred for selection as target lesion.
Bone Lesions: Bone scan, PET scan or plain films are not considered adequate imaging techniques to measure bone lesions. However, these techniques can be used to confirm the presence or disappearance of bone lesions.

### Special Notes on Assessment of Progression of Non-target Disease

To achieve ‘unequivocal progression’ on the basis of the non-target disease, there must be an overall level of substantial worsening in non-target disease such that, even in presence of SD or PR in target disease, the overall tumor burden has increased sufficiently to merit discontinuation of therapy. A modest ‘increase’ in the size of one or more non-target lesions is usually not sufficient to qualify for unequivocal progression status. The designation of overall progression solely on the basis of change in non-target disease in the face of SD or PR of target disease will therefore be extremely rare.

### Imaging Methods

The same method of assessment and the same technique used to characterize each identified and reported lesion at baseline should be used during each follow-up assessment. Imaging-based evaluation is preferred to evaluation by clinical examination unless the lesion(s) being followed cannot be imaged but are assessable by clinical exam.

Other methods of assessment, PET-CT, ultrasound and FDG-PET should not be used for response assessment in this study.

### CA-125

Tumor marker CA-125 alone cannot be used to assess response or determine progression. However, a serum CA-125 assay may be helpful in monitoring clinical response.

### Time Point Assessments

All tumor measurements must be recorded in millimeters. At each assessment, a sum of the diameters (longest for non-nodal lesions, short axis for nodal lesions) for all target lesions will be calculated and reported. The baseline sum of the longest diameters (SLD) will be used as reference to further characterize any objective tumor regression in the measurable dimension of the disease. The lowest SLD (nadir) since, and including, the baseline value will be used as reference for evaluating progression.

After baseline, the actual size of the target lesion should be documented, if possible, even if the lesions become very small. If in the opinion of the radiologist, the lesion has likely disappeared, 0 mm should be recorded. If the lesion is present but too small to measure, an indicator of “too small to measure” will be provided on the CRY (a default value of 5 mm will be imputed during analysis). Non-target lesions are to be assessed qualitatively (present, resolved, or unequivocal progression) and new lesion, if any, are to be documented separately.

At each evaluation, a time point response is to be determined for target lesions, non-target lesions, new lesions and overall.

### Time Point Response Criteria

Target Lesion Time Point Response (TPR)

| | |
|---|---|
| Complete | Disappearance of all target lesions. All pathological lymph nodes (whether target or |
| Response (CR) | non-target) must have reduction in short axis to <10 mm. |
| Partial | At least 30% decrease in the sum of diameters (SoD) of target lesions, taking as |
| Response (PR) | reference the baseline SoD |
| Progressive | At least a 20% increase in the SoD of target lesion, taken as reference the smallest |
| Disease (PD) | (nadir) SoD since and including baseline. In addition to the relative increase of 20%, |
| | the SoD must also demonstrate an absolute increase of at least 5 mm. |
| Stable | Neither sufficient shrinkage to qualify for PR nor sufficient increase to qualify for PD. |
| Disease (SD) | |
| Not Applicable | No target lesions identified at baseline |
| (N/A) | |
| Unable to | One or more target lesions are not imaged and the remainder of the SoD compared |
| Evaluate (UE) | with the nadir SoD does not meet the criteria for progressive disease (PD) |

If the target lesion for a participant meets the criteria for both PR
and PD at a given time point, the target lesion response is PD.
If the nadir SoD is 0 (i.e., the participant had a prior target lesion CR),
the re-appearance of any prior target lesions to any degree constitutes PD.
Non-Target Lesion TPR

| | |
|---|---|
| Complete | Disappearance of all non-target lesions and normalization of tumor marker level if |
| Response (CR) | tumor marker at baseline is above the upper normal limit. All lymph nodes must be |
| | non-pathological in size (<10 mm short axis). |
| Non-CR/Non-PD | Persistence of one or more non-target lesion(s) and/or maintenance of CA- 125 above |
| | the normal limits if CA-125 at baseline is above the upper normal limit. |

Target Lesion Time Point Response (TPR)

| | |
|---|---|
| Progressive | Unequivocal progression of non-target lesions. Unequivocal progression |
| Disease (PD) | should not normally trump target lesion status. It must be representative |
| | of overall disease status change, not a single lesion increase. |
| Not Applicable | No non-target lesions identified at baseline |
| (N/A) | |
| Unable to | One or more non-target lesions are not imaged and the remaining |
| Evaluate (UE) | non-target lesions do not meet the criteria for PD |

If the target lesion for a participant meets the criteria for both
PR and PD at a given time point, the target lesion response is PD.
If the nadir SoD is 0 (i.e., the participant had a prior target lesion CR),
the re-appearance of any prior target lesions to any degree constitutes PD.

New Lesion TPR

| | |
|---|---|
| Yes | Lesion present at follow-up visit either for the very first |
| | time or re- appearing (i.e., lesion was present at baseline, |
| | disappeared at a follow-up visit and re-appeared later). |
| No | No new lesions present at follow-up |
| Unable to | Participant non assessed or incompletely assessed for new lesion |
| Evaluate (UE) | |

### Determining Overall TPR

| Target Lesion | Non-Target | New Lesions | Overall |
|---|---|---|---|
| TPR | TPR | TPR | TPR |
| CR | CR or NA | No | CR* |
| CR | Non-CR/non-PD | No | PR* |
| CR | UE | No | PR* |
| PR | Non-PD or | No | PR* |
| | NA or UE | | |
| SD | Non-PD or | No | SD |
| | NA or UE | | |
| UE | Non-PD | No | UE |
| PD | Any | No or Yes | PD |
| | | or UE | |
| Any | PD | No or Yes | PD |
| | | or UE | |
| Any | Any | Yes | PD |
| NA | CR | No | CR* |
| NA | Non-CR/non-PD | No | Non-CR/non-PD |
| Non-PD | Non-PD | UE | UE |

CR = complete response; PR = partial response; SD = stable disease; PD = progressive disease; UE = unable to evaluate; NA = not applicable (no such lesions at Screening); Any, CR, PR, SD, PD, NA or UE.
The overall response at a given time point does not depend upon the overall response assigned at any prior time point.
*Participants with an overall response of CR or PR must have a repeat tumor assessment performed no less than four weeks after the criteria for response are first met.

Best Overall Response: Best overall response, incorporating confirmation requirements, is derived during statistical analysis from the series of time point responses. For SD, a minimum time interval of 49 days (seven weeks) is required between the first dose date and tumor measurements.

Maximum tolerated doses (MTDs) of combinations will be identified. From this, treatment doses of combinations will be identified that are safe and tolerable by assessment of adverse events (AEs)/serious adverse events (SAEs) as graded according to the National Cancer Institute (NCI CTCAE v5.0.)

Additional outcomes on which the trial may be evaluated include:
ORR of the patients on the treatment, e.g., greater than 20%.
PFS greater than six months in a subset of patients on the treatment, in a five-year time frame
DCR for patients on the treatment greater than 40%
Median OS greater than 15 months in a subset of patients on the treatment, in a five-year time frame
CBR of the patients on the treatment greater than 30%, in a five-year time frame
DoR greater than eight months in a subset of patients on the treatment, in a five-year time frame
All Grade Hyperglycemia in less than 30% of the patients on the treatment, in a two-year time frame
Grade 3 and Grade 4 hyperglycemia in less than 10% of the patients on the treatment, in a two-year time frame
All Grade stomatitis in less than 30% of the patients on the treatment, in a two-year time frame
Grade 3 and Grade 4 Stomatitis in less than 15% of the patients on the treatment, in a two-year time frame

What is claimed is:

1. A method of treating endometrial cancer in a human subject in need thereof, the method comprising:
intravenously administering to the subject 40-100 mg/m² paclitaxel on day 1, day 8, and day 15 of a four-week treatment interval; and
orally administering to the subject 100-300 mg serabelisib and 1-4 mg sapanisertib on day 2, day 3, day 4, day 9, day 10, day 11, day 16, day 17, day 18, day 23, day 24 and day 25 of the four-week treatment interval,
so as to treat endometrial cancer in the human subject.

2. The method of claim 1, which provides a partial response (PR) or stable disease (SD) of at least 16 weeks to the subject.

3. The method of claim 1, which provides a complete response (CR) to the subject.

4. The method of claim 1, which provides
a) a progression free survival (PFS) of at least twelve months to the subject;
b) an overall survival (OS) of at least 15 months to the subject; and/or
c) a duration of response (DoR) of at least fifteen months to the subject.

5. The method of claim 1, wherein the serabelisib and the sapanisertib are administered at about the same time.

6. The method of claim 1, wherein the serabelisib and the sapanisertib are administered with food and/or with a meal.

7. The method of claim 1, wherein the subject is on an insulin suppressing diet.

8. The method of claim 1, wherein the subject previously received from one to four prior systemic therapies which include at least one platinum-based chemotherapy and at least one checkpoint inhibitor.

9. The method of claim 1, wherein the subject has a mutation identified in a gene of the PI3K/AKT/mTOR pathway, and/or has a mutated PI3K pathway.

10. The method of claim 1, wherein the subject comprises one or more mutations in at least one gene selected from the group consisting of PIK3CB, PIK3CG, PIK3CD, PIK3C2B, RPTOR, RAPTOR, RICTOR, MLST8, DEPTOR, S6K, 4E-BP1, AKT1S1, STK11, TSC2, INPP4B, PDK1, RHEB, AMPK, GSK3, DEPDC5, NPRL2, and NPRL3.

11. The method of claim 1, which does not induce a serious adverse event (SAE) in the human subject.

12. The method of claim 1, which does not induce a grade 3 adverse event or a grade 4 adverse event in the human subject.

13. The method of claim 1, which excludes administration of a steroid or corticosteroid, excludes administration of a prophylactic mouth rinse comprising a steroid or corticosteroid, and/or excludes a measure to prevent, ameliorate, treat, or reduce stomatitis.

14. A method of treating endometrial cancer in a human subject in need thereof, the method comprising:
intravenously administering to the subject 40-100 mg/m² paclitaxel on day 1, day 8, and day 15 of a four-week treatment interval;
orally administering to the subject 100-300 mg serabelisib and 1-4 mg sapanisertib on day 2, day 3, day 4, day 9, day 10, day 11, day 16, day 17, day 18, day 23, day 24 and day 25 of the four-week treatment interval; and
excluding a measure to prevent, ameliorate, treat, or reduce stomatitis,
so as to treat endometrial cancer in the human subject.

15. The method of claim 14, which excludes administration of a steroid or corticosteroid.

16. The method of claim 14, which excludes administration of a prophylactic mouth rinse comprising a steroid or corticosteroid.

17. A method of treating endometrial cancer in a human subject in need thereof without inducing grade 4 stomatitis or without inducing grade 4 hyperglycemia, the method comprising:
intravenously administering to the subject 40-100 mg/m² paclitaxel on day 1, day 8, and day 15 of a four-week treatment interval; and
orally administering to the subject 100-300 mg serabelisib and 1-4 mg sapanisertib on day 2, day 3, day 4, day 9, day 10, day 11, day 16, day 17, day 18, day 23, day 24 and day 25 of the four-week treatment interval,
so as to treat endometrial cancer in the human subject without inducing grade 4 stomatitis or without inducing grade 4 hyperglycemia.

18. The method of claim 17, which does not induce grade 4 stomatitis and does not induce grade 4 hyperglycemia.

19. The method of claim 17, which does not induce grade 3 stomatitis.

20. The method of claim 17, which does not induce grade 3 hyperglycemia.

21. A method of treating breast cancer in a subject in need thereof, comprising administering to the subject in need thereof:
(i) a therapeutically effective amount of one or more compounds that inhibit two or more enzymes of the PI3K/AKT/mTOR pathway, and
(ii) a therapeutically effective amount of one or more anti-cancer agents that do not directly inhibit enzymes in the PI3K/AKT/mTOR pathway.

22. The method of claim 21, wherein the one or more compounds that inhibit two or more enzymes of the PI3K/AKT/mTOR pathway comprise sapanisertib and serabelisib, wherein about 1 mg/day to about 4 mg/day sapanisertib and about 50 mg/day to about 400 mg/day of the serabelisib is administered to the subject for three days per week.

23. The method of claim 21, wherein the one or more anti-cancer agents that do not directly inhibit enzymes in the PI3K/AKT/mTOR pathway comprise any one of fulvestrant, a cyclin-dependent kinase 4 (CDK4) inhibitor, a cyclin-dependent kinase 4 (CDK6) inhibitor, a CDK4/6 inhibitor, or a combination thereof.

24. The method of claim 21, wherein the one or more anti-cancer agents that do not directly inhibit enzymes in the PI3K/AKT/mTOR pathway comprises fulvestrant.

25. The method of claim 24, comprising administering to the subject about 500 mg of fulvestrant twice in a 4-week interval on day 1, and administering about 500 mg of fulvestrant every 28 days thereafter, or administering a fulvestrant 500 mg loading dose on day 1, day 15, day 29, and then a 500 mg maintenance dose every 28 days thereafter.

26. The method of claim 21, wherein the one or more anti-cancer agents that do not directly inhibit enzymes in the PI3K/AKT/mTOR pathway comprises a CDK4/6 inhibitor.

27. The method of claim 26, wherein the CDK4/6 inhibitor is palbociclib and about 75 mg/day to about 150 mg/day palbociclib is administered to the subject.

28. The method of claim 27, wherein the palbociclib is administered in a repeating 28-day cycle comprising a palbociclib dose of about 75 mg/day to about 150 mg/day for 3 weeks followed by 1 week without administering the palbociclib.

29. The method of claim 27, wherein 125 mg palbociclib is administered once daily for 21 days and then palbociclib is not administered for 7 days.

30. The method of claim 21, wherein the breast cancer is HER2 negative and hormone receptor-positive.
